SEQUENCE LISTING (164 sequences)

SEQ ID NO: 1 - DNA, 1846 bp, Thielavia terrestris
aattgaaggagggagtggcggagtggccaccaagtcaggcggctgtcaactaaccaaggatgggaacagttcggctcgccttgcccgagggcagcgttccctgatggggacgaaccatgggactggggtcagctgctgtataaaagttcaaatcgatgatctctcagatggcgctgctggggtgttctgcgcttttccatcctcgcaacctggtatcccactagtccagcgttcggcaccatgaagtcgttcaccattgccgccttggcagccctatgggcccaggaggccgccgcccacgcgaccttccaggacctctggattgatggagtcgactacggctcgcaatgtgtccgcctcccggcgtccaactcccccgtcaccaatgttgcgtccgacgatatccgatgcaatgtcggcacctcgaggcccaccgtcaagtgcccggtcaaggccggctccacggtcacgatcgagatgcaccaggttcgcacgcctctctgcgtaggccccccagctactatatggcactaacacgacctccagcaacctggcgaccggtcttgcgccaacgaggctatcggcggcgaccactacggccccgtaatggtgtacatgtccaaggtcgatgacgcggtgacagccgacggttcatcgggctggttcaaggtgttccaggacagctgggccaagaacccgtcgggttcgacgggcgacgacgactactggggcaccaaggacctcaactcgtgctgcggcaagatgaacgtcaagatccccgaagacatcgagccgggcgactacctgctccgcgccgaggttatcgcgctgcacgtggccgccagctcgggcggcgcgcagttctacatgtcctgctaccagctgaccgtgacgggctccggcagcgccaccccctcgaccgtgaatttcccgggcgcctactcggccagcgacccgggcatcctgatcaacatccacgcgcccatgtcgacctacgtcgtcccgggcccgaccgtgtacgcgggcggctcgaccaagtcggctggcagctcctgctccggctgcgaggcgacctgcacggttggttccggccccagcgcgacactgacgcagcccacctccaccgcgaccgcgacctccgcccctggcggcggcggctccggctgcacggcggccaagtaccagcagtgcggcggcaccggctacactgggtgcaccacctgcgctgtaagttccctcgtgatatgcagcggaacaccgtctggactgttttgctaactcgcgtcgtagtccgggtctacctgcagcgccgtctcgcctccgtactactcgcagtgcctctaagccgggagcgcttgctcagcgggctgctgtgaaggagctccatgtccccatgccgccatggccggagtaccgggctgagcgcccaattcttgtatatagttgagttttcccaatcatgaatacatatgcatctgcatggactgttgcgtcgtcagtctacatcctttgctccactgaactgtgagaccccatgtcatccggaccattcgatcggtgctcgctctaccatctcggttgatgggtctgggcttgagagtcactggcacgtcctcggcggtaatgaaatgtggaggaaagtgtgagctgtctgacgcactcggcgctgatgagacgttgagcgcggcccacactggtgttctgtaagccagcacacaaaagaatactccaggatggcccatagcggcaaatatacagtatcagggatgcaaaaagtgcaaaagtaaggggctcaatcggggatcgaacccgagacctcgcacatgacttatttcaagtcaggggt

SEQ ID NO: 2 - protein, 326 aa, Thielavia terrestris
MKSFTIAALAALWAQEAAAHATFQDLWIDGVDYGSQCVRLPASNSPVTNVASDDIRCNVGTSRPTVKCPVKAGSTVTIEMHQQPGDRSCANEAIGGDHYGPVMVYMSKVDDAVTADGSSGWFKVFQDSWAKNPSGSTGDDDYWGTKDLNSCCGKMNVKIPEDIEPGDYLLRAEVIALHVAASSGGAQFYMSCYQLTVTGSGSATPSTVNFPGAYSASDPGILINIHAPMSTYVVPGPTVYAGGSTKSAGSSCSGCEATCTVGSGPSATLTQPTSTATATSAPGGGGSGCTAAKYQQCGGTGYTGCTTCASGSTCSAVSPPYYSQCL

SEQ ID NO: 3 - DNA, 880 bp, Thielavia terrestris
accccgggatcactgcccctaggaaccagcacacctcggtccaatcatgcggttcgacgccctctccgccctcgctcttgcgccgcttgtggctggccacggcgccgtgaccagctacatcatcggcggcaaaacctatcccggctacgagggcttctcgcctgcctcgagcccgccgacgatccagtaccagtggcccgactacaacccgaccctgagcgtgaccgacccgaagatgcgctgcaacggcggcacctcggcagagctcagcgcgcccgtccaggccggcgagaacgtgacggccgtctggaagcagtggacccaccagcaaggccccgtcatggtctggatgttcaagtgccccggcgacttctcgtcgtgccacggcgacggcaagggctggttcaagatcgaccagctgggcctgtggggcaacaacctcaactcgaacaactggggcaccgcgatcgtctacaagaccctccagtggagcaacccgatccccaagaacctcgcgccgggcaactacctcatccgccacgagctgctcgccctgcaccaggccaacacgccgcagttctacgccgagtgcgcccagctggtcgtctccggcagcggctccgccctgcccccgtccgactacctctacagcatccccgtctacgcgccccagaacgaccccggcatcaccgtgagtgggcttccgttccgcggcgagctctgtggaaatcttgctgacgatgggctaggttgacatctacaacggcgggcttacctcctacaccccgcccggcggccccgtctggtctggcttcgagttttaggcgcattgagtcgggggctacgaggggaaggcatctgttcgcatgagcgtgggtac

SEQ ID NO: 4 - protein, 239 aa, Thielavia terrestris
MRFDALSALALAPLVAGHGAVTSYIIGGKTYPGYEGFSPASSPPTIQYQWPDYNPTLSVTDPKMRCNGGTSAELSAPVQAGENVTAVWKQWTHQQGPVMVWMFKCPGDFSSSHGDGKGWFKIDQLGLWGNNLNSNNWGTAIVYKTLQWSNPIPKNLAPGNYLIRHELLALHQANTPQFYAECAQLVVSGSGSALPPSDYLYSIPVYAPQNDPGITVDIYNGGLTSYTPPGGPVWSGFEF

SEQ ID NO: 5 - DNA, 1000 bp, Thielavia terrestris
ctcctgttcctgggccaccgcttgttgcctgcactattggtagagttggtctattgctagagttggccatgcttctcacatcagtcctcggctcggctgccctgcttgctagcggcgctgcggcacacggcgccgtgaccagctacatcatcgccggcaagaattacccggggtgggtagctgattattgagggcgcattcaaggttcataccggtgtgcatggctgacaaccggctggcagataccaaggcttttctcctgcgaactcgccgaacgtcatccaatggcaatggcatgactacaaccccgtcttgtcgtgcagcgactcgaagcttcgctgcaacggcggcacgtcggccaccctgaacgccacggccgcaccgggcgacaccatcaccgccatctgggcgcagtggacgcacagccagggccccatcctggtgtggatgtacaagtgcccgggctccttcagctcctgtgacggctccggcgctggctggttcaagatcgacgaggccggcttccacggcgacggcgtcaaggtcttcctcgacaccgagaacccgtccggctgggacatcgccaagctcgtcggcggcaacaagcagtggagcagcaaggtccccgagggcctcgcccccggcaactacctcgtccgccacgagttgatcgccctgcaccaggccaacaacccgcagttctacccggagtgcgcccaggtcgtcatcaccggctccggcaccgcgcagccggatgcctcatacaaggcggctatccccggctactgcaaccagaatgacccgaacatcaaggtgagatccaggcgtaatgcagtctactgctggaaagaaagtggtccaagctaaaccgcgctccaggtgcccatcaacgaccactccatccctcagacctacaagattcccggccctcccgtcttcaagggcaccgccagcaagaaggcccgggacttcaccgcctgaagttgttgaatcgatggag

SEQ ID NO: 6 - protein, 258 aa, Thielavia terrestris
MLLTSVLGSAALLASGAAAHGAVTSYIIAGKNYPGYQGFSPANSPNVIQWQWHDYNPVLSCSDSKLRCNGGTSATLNATAAPGDTITAIWAQWTHSQGPILVWMYKCPGSFSSCDGSGAGWFKIDEAGFHGDGVKVFLDTENPSGWDIAKLVGGNKQWSSKVPEGLAPGNYLVRHELIALHQANNPQFYPECAQVVITGSGTAQPDASYKAAIPGYCNQNDPNIKVPINDHSIPQTYKIPGPPVFKGTASKKARDFTA

SEQ ID NO: 7 - DNA, 681 bp, Thielavia terrestris
atgctcgcaaacggtgccatcgtcttcctggccgccgccctcggcgtcagtggccactacacctggccacgggttaacgacggcgccgactggcaacaggtccgtaaggcggacaactggcaggacaacggctacgtcggggatgtcacgtcgccacagatccgctgtttccaggcgaccccgtccccggccccatccgtcctcaacaccacggccggctcgaccgtgacctactgggccaaccccgacgtctaccaccccgggcctgtgcagttttacatggcccgcgtgcccgatggcgaggacatcaactcgtggaacggcgacggcgccgtgtggttcaaggtgtacgaggaccatcctacctttggcgctcagctcacatggcccagcacgggcaagagctcgttcgcggttcccatccccccgtgcatcaagtccggctactacctcctccgggcggagcaaatcggcctgcacgtcgcccagagcgtaggcggagcgcagttctacatctcatgcgcccagctcagcgtcaccggcggcggcagcaccgagccgccgaacaaggtggccttccccggcgcttacagtgcgacggacccgggcattctgatcaacatctactaccctgttcccacgtcctaccagaaccccggcccggccgtcttcagctgctga

SEQ ID NO: 8 - protein, 226 aa, Thielavia terrestris
MLANGAIVFLAAALGVSGHYTWPRVNDGADWQQVRKADNWQDNGYVGDVTSPQIRCFQATPSPAPSVLNTTAGSTVTYWANPDVYHPGPVQFYMARVPDGEDINSWNGDGAVWFKVYEDHPTFGAQLTWPSTGKSSFAVPIPPCIKSGYYLLRAEQIGLHVAQSVGGAQFYISCAQLSVTGGGSTEPPNKVAFPGAYSATDPGILINIYYPVPTSYQNPGPAVFSC

SEQ ID NO: 9 - DNA, 960 bp, Thielavia terrestris
atgaagggacttttcagtgccgccgccctctccctggccgtcggccaggcttcggcccattacatcttccagcaactctccatcaacgggaaccagtttccggtgtaccaatatattcgcaagaacaccaattataacagtcccgttaccgatctcacgtccgacgatcttcggtgcaatgtcggcgcccagggtgctgggacagacaccgtcacggtgaaggccggcgaccagttcaccttcacccttgacacccctgtttaccaccaggggcccatctccatctacatgtccaaggccccgggcgcggcgtcagactacgatggcagcggcggctggttcaagatcaaggactggggcccgactttcaacgccgacggcacggccacctgggacatggccggctcatacacctacaacatcccgacctgcattcccgacggcgactatctgctccgcatccagtcgctggccatccacaacccctggccggcgggcatcccgcagttctacatctcctgcgcccagatcaccgtgaccggcggcggcaacggcaaccctggcccgacggccctcatccccggcgccttcaaggacaccgacccgggctacacggtgaacatctacacgaacttccacaactacacggttcccggcccggaggtcttcagctgcaacggcggcggctcgaacccgcccccgccggtgagtagcagcacgcccgcgaccacgacgctggtcacgtcgacgcgcaccacgtcctccacgtcctccgcctcgacgccggcctcgaccggcggctgcaccgtcgccaagtggggccagtgcggcggcaacgggtacaccggctgcacgacctgcgcggccgggtccacctgcagcaagcagaacgactactactcgcagtgcttgtaagggaggccgcaaagcatgaggtgtttgaagaggaggagaggggtc

SEQ ID NO: 10 - protein, 304 aa, Thielavia terrestris
MKGLFSAAALSLAVGQASAHYIFQQLSINGNQFPVYQYIRKNTNYNSPVTDLTSDDLRCNVGAQGAGTDTVTVKAGDQFTFTLDTPVYHQGPISIYMSKAPGAASDYDGSGGWFKIKDWGPTFNADGTATWDMAGSYTYNIPTCIPDGDYLLRIQSLAIHNPWPAGIPQFYISCAQITVTGGGNGNPGPTALIPGAFKDTDPGYTVNIYTNFHNYTVPGPEVFSCNGGGSNPPPPVSSSTPATTTLVTSTRTTSSTSSASTPASTGGCTVAKWGQCGGNGYTGCTTCAAGSTCSKQNDYYSQCL

SEQ ID NO: 11 - DNA, 954 bp, Thielavia terrestris
atgaagggcctcagcctcctcgccgctgcgtcggcagcgactgctcataccatcttcgtgcagctcgagtcagggggaacgacctatccggtatcctacggcatccgggaccctagctacgacggtcccatcaccgacgtcacctccgactcactggcttgcaatggtcccccgaaccccacgacgccgtccccgtacatcatcaacgtcaccgccggcaccacggtcgcggcgatctggaggcacaccctcacatccggccccgacgatgtcatggacgccagccacaaggggccgaccctggcctacctcaagaaggtcgatgatgccttgaccgacacgggtatcggcggcggctggttcaagatccaggaggccggttacgacaatggcaattgggctaccagcacggtgatcaccaacggtggcttccaatatattgacatccccgcctgcattcccaacggccagtatctgctccgcgccgagatgatcgcgctccacgccgccagcacgcagggtggtgcccagctctacatggagtgcgcgcagatcaacgtggtgggcggctccggcagcgccagcccgcagacgtacagcatcccgggcatctaccaggcaaccgacccgggcctgctgatcaacatctactccatgacgccgtccagccagtacaccattccgggtccgcccctgttcacctgcagcggcagcggcaacaacggcggcggcagcaacccgtcgggcgggcagaccacgacggcgaagcccacgacgacgacggcggcgacgaccacctcctccgccgctcctaccagcagccaggggggcagcagcggttgcaccgttccccagtggcagcagtgcggtggcatctcgttcaccggctgcaccacctgcgcggcgggctacacctgcaagtatctgaacgactattactcgcaatgccagtaa

SEQ ID NO: 12 - protein, 317 aa, Thielavia terrestris
MKGLSLLAAASAATAHTIFVQLESGGTTYPVSYGIRDPSYDGPITDVTSDSLACNGPPNPTTPSPYIINVTAGTTVAAIWRHTLTSGPDDVMDASHKGPTLAYLKKVDDALTDTGIGGGWFKIQEAGYDNGNWATSTVITNGGFQYIDIPACIPNGQYLLRAEMIALHAASTQGGAQLYMECAQINVVGGSGSASPQTYSIPGIYQATDPGLLINIYSMTPSSQYTIPGPPLFTCSGSGNNGGGSNPSGGQTTTAKPTTTTAATTTSSAAPTSSQGGSSGCTVPQWQQCGGISFTGCTTCAAGYTCKYLNDYYSQCQ

SEQ ID NO: 13 - DNA, 799 bp, Thermoascus aurantiacus
atgtccttttccaagataattgctactgccggcgttcttgcctctgcttctctagtggctggccatggcttcgttcagaacatcgtgattgatggtaaaaagtatgtcattgcaagacgcacataagcggcaacagctgacaatcgacagttatggcgggtatctagtgaaccagtatccatacatgtccaatcctccagaggtcatcgcctggtctactacggcaactgatcttggatttgtggacggtactggataccaaaccccagatatcatctgccataggggcgccaagcctggagccctgactgctccagtctctccaggaggaactgttgagcttcaatggactccatggcctgattctcaccatggcccagttatcaactaccttgctccgtgcaatggtgattgttccactgtggataagacccaattagaattcttcaaaattgccgagagcggtctcatcaatgatgacaatcctcctgggatctgggcttcagacaatctgatagcagccaacaacagctggactgtcaccattccaaccacaattgcacctggaaactatgttctgaggcatgagattattgctcttcactcagctcagaaccaggatggtgcccagaactatccccagtgcatcaatctgcaggtcactggaggtggttctgataaccctgctggaactcttggaacggcactctaccacgataccgatcctggaattctgatcaacatctatcagaaactttccagctatatcatccctggtcctcctctgtatactggttaa

SEQ ID NO: 14 - protein, 249 aa, Thermoascus aurantiacus
MSFSKIIATAGVLASASLVAGHGFVQNIVIDGKYYGGYLVNQYPYMSNPPEVIAWSTTATDLGFVDGTGYQTPDIICHRGAKPGALTAPVSPGGTVELQWTPWPDSHHGPVINYLAPCNGDCSTVDKTQLEFFKIAESGLINDDNPPGIWASDNLIAANNSWTVTIPTTIAPGNYVLRHEIIALHSAQNQDGAQNYPQCINLQVTGGGSDNPAGTLGTALYHDTDPGILINIYQKLSSYIIPGPPLYTG

SEQ ID NO: 15 - DNA, 1172 bp, Trichoderma reesei
ggatctaagccccatcgatatgaagtcctgcgccattcttgcagcccttggctgtcttgccgggagcgttctcggccatggacaagtccaaaacttcacgatcaatggacaatacaatcagggtttcattctcgattactactatcagaagcagaatactggtcacttccccaacgttgctggctggtacgccgaggacctagacctgggcttcatctcccctgaccaatacaccacgcccgacattgtctgtcacaagaacgcggccccaggtgccatttctgccactgcagcggccggcagcaacatcgtcttccaatggggccctggcgtctggcctcacccctacggtcccatcgttacctacgtggctgagtgcagcggatcgtgcacgaccgtgaacaagaacaacctgcgctgggtcaagattcaggaggccggcatcaactataacacccaagtctgggcgcagcaggatctgatcaaccagggcaacaagtggactgtgaagatcccgtcgagcctcaggcccggaaactatgtcttccgccatgaacttcttgctgcccatggtgcctctagtgcgaacggcatgcagaactatcctcagtgcgtgaacatcgccgtcacaggctcgggcacgaaagcgctccctgccggaactcctgcaactcagctctacaagcccactgaccctggcatcttgttcaacccttacacaacaatcacgagctacaccatccctggcccagccctgtggcaaggctagatccaggggtacggtgttggcgttcgtgaagtcggagctgttgacaaggatatctgatgatgaacggagaggactgatgggcgtgactgagtgtatatatttttgatgaccaaattgtatacgaaatccgaacgcatggtgatcattgtttatccctgtagtatattgtctccaggctgctaagagcccaccgggtgtattacggcaacaaagtcaggaatttgggtggcaatgaacgcaggtctccatgaatgtatatgtgaagaggcatcggctggcatgggcattaccagatataggccctgtgaaacatatagtacttgaacgtgctactggaacggatcataagcaagtcatcaacatgtgaaaaaacactacatgtaaaaaaaaaaaaaaaaaaaaa

SEQ ID NO: 16 - protein, 249 aa, Trichoderma reesei
MKSCAILAALGCLAGSVLGHGQVQNFTINGQYNQGFILDYYYQKQNTGHFPNVAGWYAEDLDLGFISPDQYTTPDIVCHKNAAPGAISATAAAGSNIVFQWGPGVWPHPYGPIVTYVVECSGSCTTVNKNNLRWVKIQEAGINYNTQVWAQQDLINQGNKWTVKIPSSLRPGNYVFRHELLAAHGASSANGMQNYPQCVNIAVTGSGTKALPAGTPATQLYKPTDPGILFNPYTTITSYTIPGPALWQG

SEQ ID NO: 17 - DNA, 924 bp, Myceliophthora thermophila
atgaagttcacctcgtccctcgctgtcctggccgctgccggcgcccaggctcactgttagtcgaccctcgaacccaacacccccctccccccttttctcctccatctcctcggcctcacttagtagccgctgacaacgactagataccttccctagggccggcactggtggctcgctctctggcgagtgggaggtggtccgcatgaccgagaaccattactcgcacggcccggtcaccgatgtcaccagccccgagatgacctgctatcagtccggcgtgcagggtgcgccccagaccgtccaggtcaaggcgggctcccaattcaccttcagcgtggatccctcgatcggccaccccggccctctccagttctacatggctaaggtgccgtcgggccagacggccgccacctttgacggcacgggagccgtgtggttcaagatctaccaagacggcccgaacggcctcggcaccgacagcattacctggcccagcgccggttcgtgacttcctccccactcgcttttttttttttattttttatttttttttctttcggaactcaagaatctttctctctctctcccgtctttggccttgaacaacactaaaactcttccttactgtattaattaggcaaaaccgaggtctcggtcaccatccccagctgcatcgatgatggcgagtacctgctccgggtcgagcacatcgcgctccacagcgccagcagcgtgggcggcgctcagttctacattgcctgcgcccagctctccgtcaccggcggctccggcaccctcaacacgggctcgctcgtctccctgcccggcgcctacaaggccaccgacccgggcatcctcttccagctctactggcccatcccgaccgagtacatcaaccccggcccggcccccgtctcttgctaa

SEQ ID NO: 18 - protein, 232 aa, Myceliophthora thermophila
MKFTSSLAVLAAAGAQAHYTFPRAGTGGSLSGEWEVVRMTENHYSHGPVTDVTSPEMTCYQSGVQGAPQTVQVKAGSQFTFSVDPSIGHPGPLQFYMAKVPSGQTAATFDGTGAVWFKIYQDGPNGLGTDSITWPSAGKTEVSVTIPSCIDDGEYLLRVEHIALHSASSVGGAQFYIACAQLSVTGGSGTLNTGSLVSLPGAYKATDPGILFQLYWPIPTEYINPGPAPVSC

SEQ ID NO: 19 - DNA, 854 bp, Myceliophthora thermophila
atgaaggccctctctctccttgcggctgcctcggcagtctctgcgcataccatcttcgtccagctcgaagcagacggcacgaggtacccggtctcgtacgggatccgggacccaagctacgacggccccatcaccgacgtcacatccaacgacgttgcttgcaacggcgggccgaacccgacgaccccctccagcgacgtcatcaccgtcaccgcgggcaccacggtcaaggccatctggaggcacaccctccaatccggcccggacgatgtcatggacgccagccacaagggcccgaccctggcctacctcaagaaggtcggcgatgccaccaaggactcgggcgtcggcggtggctggttcaagattcaggaggacggctacaacaacggccagtggggcaccagcaccgttatctccaacggcggcgagcactacatgtgagccattcctccgagagaagaccaagactcttgacgatctcgctgacccgtgcaacaagtgacatcccggcctgcatccccgagggtcagtacctcctccgcgccgagatgatcgccctccacgcggccgggtcccccggcggtgcccagctctacgtaagcctctgcccttccccccttcctcttgatcgaatcggactgcccaccccccttttcgactccgactaacaccgttgccagatggaatgtgcccagatcaacatcgtcggcggctccggctcggtgcccagctcgaccgtcagcttccccggcgcgtacagccccaacgacccgggtctcctcatcaacatctattccatgtcgccctcgagctcgtacaccatcccgggcccgcccgtcttcaagtgctag

SEQ ID NO: 20 - protein, 235 aa, Myceliophthora thermophila
MKALSLLAAASAVSAHTIFVQLEADGTRYPVSYGIRDPSYDGPITDVTSNDVACNGGPNPTTPSSDVITVTAGTTVKAIWRHTLQSGPDDVMDASHKGPTLAYLKKVGDATKDSGVGGGWFKIQEDGYNNGQWGTSTVISNGGEHYIDIPACIPEGQYLLRAEMIALHAAGSPGGAQLYMECAQINIVGGSGSVPSSTVSFPGAYSPNDPGLLINIYSMSPSSSYTIPGPPVFKC

SEQ ID NO: 21 - DNA, 1242 bp, Myceliophthora thermophila
atgaagtccttcgccctcaccactctggccgccctggccggcaacgccgccgctcacgcgaccttccaggccctctgggtcgacggcgtcgactacggcgcgcagtgtgcccgtctgcccgcgtccaactccccggtcaccgacgtgacctccaacgcgatccgctgcaacgccaacccgtcgcccgctcggggcaagtgcccggtcaaggccggctcgaccgttacggtcgagatgcatcaggtacgttggatgaatgaaaggggaaaggaagcagaggcagaaggggaaggcgaagggaaagaaaaagaaaaagaaatggaaaagaaaaagaaatggaaaagaaaaagaaaaatgaaaaagaaagtggaaaccgtcagactaactggggctcctcccccccacccctcctttgatatcagcaacccggtgaccggtcgtgcagcagcgaggcgatcggcggggcgcactacggccccgtcatggtgtacatgtccaaggtgtcggacgcggcgtcggcggacgggtcgtcgggctggttcaaggtgttcgaggacggctgggccaagaacccgtccggcgggtcgggcgacgacgactactggggcaccaaggacctgaactcgtgctgcgggaagatgaacgtcaagatccccgccgacctgccctcgggcgactacctgctccgggccgaggccctcgcgctgcacacggcgggcagcgccggcggcgcccagttctacatgacgtgctaccagctcaccgtgacgggctccggcagcgccagcccgcccaccgtctccttcccgggcgcctacaaggccaccgacccgggcatcctcgtcaacatccacgccccgctgtccggctacaccgtgcccggcccggccgtctactccggcggctccaccaagaaggccggcagcgcctgcaccggctgcgagtccacctgcgccgtcggctccggccccaccgccaccgtctcccagtcgcccggttccaccgccacctccgcccccggcggcggcggcggctgcaccgtccagaagtaccagcagtgcggcggcgagggctacaccggctgcaccaactgcgcggtacgtttttcaaccccgtttttttttttccttccctaccttatttggttacctaattaattactttccggctgctgactttttgctttagtccggctctacctgcagcgccgtctcgccgccctactactcgcagtgcgtctaa

SEQ ID NO: 22 - protein, 323 aa, Myceliophthora thermophila
MKSFALTTLAALAGNAAAHATFQALWVDGVDYGAQCARLPASNSPVTDVTSNAIRCNANPSPARGKCPVKAGSTVTVEMHQQPGDRSCSSEAIGGAHYGPVMVYMSKVSDAASADGSSGWFKVFEDGWAKNPSGGSGDDDYWGTKDLNSCCGKMNVKIPADLPSGDYLLRAEALALHTAGSAGGAQFYMTCYQLTVTGSGSASPPTVSFPGAYKATDPGILVNIHAPLSGYTVPGPAVYSGGSTKKAGSACTGCESTCAVGSGPTATVSQSPGSTATSAPGGGGGCTVQKYQQCGGEGYTGCTNCASGSTCSAVSPPYYSQCV

SEQ ID NO: 23 - DNA, 1253 bp, Myceliophthora thermophila
atgaagccttttagcctcgtcgccctggcgaccgccgtgagcggccatgccatcttccagcgggtgtcggtcaacgggcaggaccagggccagctcaagggggtgcgggcgccgtcgagcaactccccgatccagaacgtcaacgatgccaacatggcctgcaacgccaacattgtgtaccacgacagcaccatcatcaaggtgcccgcgggagcccgcgtcggcgcgtggtggcagcacgtcatcggcgggccgcagggcgccaacgacccggacaacccgatcgcggcctcccacaagggtatgatgatcgatgatgcctctctcttcccccgttcttgatggacaggcgatggctcccaggaacacgcgtgactgaccaccgaatccaggccccatccaggtctacctggccaaggtggacaacgcggcgacggcgtcgccgtcgggcctcaggtggttcaaggtggccgagcgcggcctgaacaacggcgtgtgggccgtcgatgagctcatcgccaacaacggctggcactacttcgacctgccgtcgtgcgtggcccccggccagtacctgatgcgcgtcgagctgctcgccctgcacagcgcctcaagccccggcggcgcccagttctacatgggctgcgcacagatcgaaggtgcgtcgatctttgttctccttccgtgtcctctctgatcctttctctcttctttttctttcttttactccctttccttccatcttcggagaagcaacgaagggggaaagggatagaagagaggaatgagagacgacgaaagagaggattggggaaagacaagacagggaaaaaaagacaagaaaaaaaaaaaaaaaaaaaaacagagtgagctaacaagaacaatcagtcactggctccggcaccaactcgggctccgactttgtctcgttccccggcgcctactcggccaacgatccgggcatcttgctaagcatctacgacagctcgggcaagcccaccaacggcgggcgctcgtacccgatccccggcccgcgccccatctcctgctccggcagcggcgacggcggcaacaacggcggcggcggcgacgacaacaacaataacaacggtggtggcaacaacggcggcggcggcggcggcagcgtccccctgtacgggcagtgcggcggcatcggctacacgggcccgaccacctgtgcccagggaacttgcaaggtgtcgaacgaatactacagccagtgcctcccctag

SEQ ID NO: 24 - protein, 310 aa, Myceliophthora thermophila
MKPFSLVALATAVSGHAIFQRVSVNGQDQGQLKGVRAPSSNSPIQNVNDANMACNANIVYHDSTIIKVPAGARVGAWWQHVIGGPQGANDPDNPIAASHKGPIQVYLAKVDNAATASPSGLRWFKVAERGLNNGVWAVDELIANNGWHYFDLPSCVAPGQYLMRVELLALHSASSPGGAQFYMGCAQIEVTGSGTNSGSDFVSFPGAYSANDPGILLSIYDSSGKPTNGGRSYPIPGPRPISCSGSGDGGNNGGGGDDNNNNNGGGNNGGGGGGSVPLYGQCGGIGYTGPTTCAQGTCKVSNEYYSQCLP

SEQ ID NO: 25 - DNA, 814 bp, Myceliophthora thermophila
atgaagctctccctcttctccgtcctggccactgccctcaccgtcgaggggcatgccatcttccagaaggtctccgtcaacggagcggaccagggctccctcaccggcctccgcgctcccaacaacaacaaccccgtgcaggatgtcaacagccaggacatgatctgcggccagtcgggatcgacgtcgaacactatcatcgaggtcaaggccggcgataggatcggtgcctggtatcagcatgtcatcggcggtgcccagttccccaacgacccagacaacccgattgccaagtcgcacaagggccccgtcatggcctacctcgccaaggttgacaatgccgcaaccgccagcaagacgggcctgaagtggtatgtattcccgcggcccgagggacatcgggttgggcaagtcgagactgacggagctcgcttctccgtataggttcaagatttgggaggatacctttaatcccagcaccaagacctggggtgtcgacaacctcatcaataacaacggctgggtgtacttcaacctcccgcagtgcatcgccgacggcaactacctcctccgcgtcgaggtcctcgctctgcactcggcctactctcagggccaggctcagttctaccagtcctgcgcccagatcaacgtatccggcggcggctccttcacaccgccgtcgactgtcagcttcccgggtgcctacagcgccagcgaccccggtatcctgatcaacatctacggcgccaccggccagcccgacaacaacggccagccgtacactgcccctgggcccgcgcccatctcctgctga

SEQ ID NO: 26 - protein, 246 aa, Myceliophthora thermophila
MKLSLFSVLATALTVEGHAIFQKVSVNGADQGSLTGLRAPNNNNPVQDVNSQDMICGQSGSTSNTIIEVKAGDRIGAWYQHVIGGAQFPNDPDNPIAKSHKGPVMAYLAKVDNAATASKTGLKWFKIWEDTFNPSTKTWGVDNLINNNGWVYFNLPQCIADGNYLLRVEVLALHSAYSQGQAQFYQSCAQINVSGGGSFTPPSTVSFPGAYSASDPGILINIYGATGQPDNNGQPYTAPGPAPISC

SEQ ID NO: 27 - DNA, 1115 bp, Thermoascus aurantiacus
atgtcgttctcgaagattgctgcgatcaccggggccattacctatgcgtctctggccgccgctcacggttatgttacaggaatcgtagccgatggcacctagtatgtaacgctcatgccaagatccgcattgctgtactaacaattagcagctacgggggctatatcgtgacccaatacccctacatgtcgacaccgccggatgtcatcgcctggtctaccaaagcaactgatcttggtttcgtggatcccagtagctatgcttcgtctgatattatctgccacaagggtgctgagcctggtgccctgagcgccaaggtggctgctggagggaccgtcgagctgcagtggacggattggcctgagagtcacaagggcccggtcattgactacctcgccgcctgtaacggggactgctcgactgtcgacaagaccaaactagagttcttcaagattgatgagagtggcctaattgacggcagcagcgccccaggcacatgggcctctgacaacttgattgccaataacaacagctggaccgtcaccatcccgagcacgattgctcccggcaactatgtcctgagacatgaaatcattgccctccactccgccggaaatacaaatggtgctcagaactacccccagtgtatcaaccttgaggtcacaggcagtggcaccgacacccctgccggcaccctcggaacggagctttataaggcaacggaccctggcattctggtcaacatctaccagaccctgaccagctacgatattcccggccctgctctgtacaccggtggtagctctggtagctctggttcctccaacaccgccaaggccaccacttcgacggcttctagctctatcgtgaccccgacgcctgttaacaacccaaccgttactcagactgccgttgttgatgtcacccagactgtttcccagaatgctgccgtcgccaccacgactccggcctccactgcagttgctacagctgtcccaacgggaaccacctttagctttgattcgatgacctcggatgaattcgtcagcctgatgcgtgcgaccgtgaattggctgctttctaacaagaagcatgcccgggatctttcttactaa

SEQ ID NO: 28 - protein, 354 aa, Thermoascus aurantiacus
MSFSKIAAITGAITYASLAAAHGYVTGIVADGTYYGGYIVTQYPYMSTPPDVIAWSTKATDLGFVDPSSYASSDIICHKGAEPGALSAKVAAGGTVELQWTDWPESHKGPVIDYLAACNGDCSTVDKTKLEFFKIDESGLIDGSSAPGTWASDNLIANNNSWTVTIPSTIAPGNYVLRHEIIALHSAGNTNGAQNYPQCINLEVTGSGTDTPAGTLGTELYKATDPGILVNIYQTLTSYDIPGPALYTGGSSGSSGSSNTAKATTSTASSSIVTPTPVNNPTVTQTAVVDVTQTVSQNAAVATTTPASTAVATAVPTGTTFSFDSMTSDEFVSLMRATVNWLLSNKKHARDLSY

SEQ ID NO: 29 - DNA, 862 bp, Aspergillus fumigatus
atgactttgtccaagatcacttccattgctggccttctggcctcagcgtctctcgtggctggccacggctttgtttctggcattgttgctgatgggaaatagtatgtgcttgaaccacacaaatgacagctgcaacagctaacttctattccagttacggagggtaccttgttaaccaatacccctacatgagcaaccctcccgacaccattgcctggtccaccaccgccaccgacctcggctttgtggacggcaccggctaccagtctccggatattatctgccacagagacgcaaagaatggcaagttgaccgcaaccgttgcagccggttcacagatcgaattccagtggacgacgtggccagagtctcaccatggaccggtacgacgccgaagagaagagaacatattgtgaccagataggctaacatagcatagttgattacttacctcgctccatgcaacggcgactgtgccaccgtggacaagaccaccctgaagtttgtcaagatcgccgctcaaggcttgatcgacggctccaacccacctggtgtttgggctgatgatgaaatgatcgccaacaacaacacggccacagtgaccattcctgcctcctatgcccccggaaactacgtccttcgccacgagatcatcgcccttcactctgcgggtaacctgaacggcgcgcagaactacccccagtgtttcaacatccaaatcaccggtggcggcagtgctcagggatctggcaccgctggcacgtccctgtacaagaatactgatcctggcatcaagtttgacatctactcggatctgagcggtggataccctattcctggtcctgcactgttcaacgcttaa

SEQ ID NO: 30 - protein, 250 aa, Aspergillus fumigatus [sequence omitted]

SEQ ID NO: 31 - DNA, 1021 bp, Penicillium pinophilum
atgccttctactaaagtcgctgccctttctgctgttctagctttggcctccacggttgctggccatggttttgtgcaaaacatcgttatcgacggtaaatcgtaagcagtgatgcatccattattaaactagacatgcttacaaaaaaatcagttactctggataccttgtgaatcagttcccctacgagtccaacccaccagctgttattgggtgggcaacaactgcaaccgacctgggattcgtcgctcccagtgagtacaccaatgcagacattatctgccacaagaacgccacacctggcgcgctttctgctccagttgctgcagggggcactgtcgagctccagtggactacatggcccgatagtcatcacggtcctgtcatcagctacctcgccaactgcaatggcaattgttctaccgtggataagactaagctagactttgtcaagattgaccaaggtggtttgatcgacgatactacccccccgggtacatgggcttccgacaaacttatcgctgccaacaacagctggactgtaactatcccctccaccatcgcgcctggaaactacgttttgcgccacgaaatcattgctcttcactccgctggaaacgcagacggtgcccaaaactaccctcaatgcatcaacttggagatcaccggcagcggaaccgccgctccctctggtaccgctggcgaaaagctctacacctctactgaccccggtatcttggtcaatatctaccaatccttgtcgacctacgttattcccggaccaactctgtggagcggtgctgccaatggcgctgttgccactggttctgctactgcggttgctacgactgccactgcttctgcgaccgctactcctaccacacttgttacctctgtcgctccagcttcatctacctttgccactgctgttgtgaccactgtcgctcctgcagtaactgatgtcgtgactgtcaccgatgtagttaccgtgaccaccgtcatcaccactactgtcctttga

SEQ ID NO: 32 - protein, 322 aa, Penicillium pinophilum
MPSTKVAALSAVLALASTVAGHGFVQNIVIDGKSYSGYLVNQFPYESNPPAVIGWATTATDLGFVAPSEYTNADIICHKNATPGALSAPVAAGGTVELQWTTWPDSHHGPVISYLANCNGNCSTVDKTKLDFVKIDQGGLIDDTTPPGTWASDKLIAANNSWTVTIPSTIAPGNYVLRHEIIALHSAGNADGAQNYPQCINLEITGSGTAAPSGTAGEKLYTSTDPGILVNIYQSLSTYVIPGPTLWSGAANGAVATGSATAVATTATASATATPTTLVTSVAPASSTFATAVVTTVAPAVTDVVTVTDVVTVTTVITTTVL

SEQ ID NO: 33 - DNA, 1486 bp, Thermoascus sp.
atgttgtcgttcgcttctgccaagtcagctgtgctgacgacccttctacttcttggatccgctcaggctcacactttgatgaccaccctgtttgtggatggcgtcaatcagggagatggtgtctgtattcgcatgaacaacaacggtagtactgccaacacctatatccagcctgtcacgagcaaggatattgcctgcggtaagtacagtaccggtccagatatcatactctatttcaatccgacaacagtcagagctggagagcaatgctaaacatccccaggcattcaaggcgaaattggcgccgctcgagtctgtccagccaaggcttcatccaccctcacgttccaattccgagagcagccatccaacccgaattccgctcctctcgatccctcgcacaaaggccccgctgcggtgtacctgaaaaaggtagactccgccatcgcgagcaacaacgccgctggagacggctggttcaagatctgggagtccgtctacgacgagtccacgggcaaatggggtacgaccaagatgatcgagaacaacgggcacatctctgtcaaggtccccgacgatatcgagggtgggtattatctcgcgcgtacggagcttctggcgctgcacgcggcgaacgaaggggatccgcagttctacgttggctgcgcgcagctgttcatcgattcagcggggacagcgaaaccgcctactgtctctattggagaggggacctacgatctgagcatgcctgccatgacgtacaatatctaccagactccgttggctctaccatacccgatgtatgggcctcctgtctacacacctggctctggctcgggttctggctctggttccgggtcagcttctgcaacgagatcttctgctattcctactgccaccgctgttacggactgttcttccgaagaggacagggaagactcagtcatggcaaccggtgttcccgttgcaagaagcacactcagaacctgggttgacagactgtcatggcatggtaaggcccgtgagaacgtgaaaccagccgccaggagaagcgcccttgtccagaccgagggtctgaagccggaaggctgcatcttcgtcaacggcaactggtgcggtttcgaggtccccgattacaacgatgcggaaagctgctgggctgtacgttcccgtctaattacttaaaacgaaataaaagctaacagtacttttctttttctaatcccaggcctccgacaactgctggaaacagtccgactcgtgctggaaccagacccagcccaccggctacaacaactgccagatctggcaagaccagaaatgcaagcccatccaggactcgtgtagccaatccaacccgactggaccgccgaacaagggcaaggatataactccaacgtggccgcccctggagggctcgatgaagaccttcaccaagcgcactgtcagttaccgtgattggattatgaaaaggaaaggagcataa

SEQ ID NO: 34 - protein, 444 aa, Thermoascus sp.
MLSFASAKSAVLTTLLLLGSAQAHTLMTTLFVDGVNQGDGVCIRMNNNGSTANTYIQPVTSKDIACGIQGEIGAARVCPAKASSTLTFQFREQPSNPNSAPLDPSHKGPAAVYLKKVDSAIASNNAAGDGWFKIWESVYDESTGKWGTTKMIENNGHISVKVPDDIEGGYYLARTELLALHAANEGDPQFYVGCAQLFIDSAGTAKPPTVSIGEGTYDLSMPAMTYNIYQTPLALPYPMYGPPVYTPGSGSGSGSGSGSASATRSSAIPTATAVTDCSSEEDREDSVMATGVPVARSTLRTWVDRLSWHGKARENVKPAARRSALVQTEGLKPEGCIFVNGNWCGFEVPDYNDAESCWAASDNCWKQSDSCWNQTQPTGYNNCQIWQDQKCKPIQDSCSQSNPTGPPNKGKDITPTWPPLEGSMKTFTKRTVSYRDWIMKRKGA

SEQ ID NO: 35 - DNA, 835 bp, Penicillium sp.
atgctgtcttcgacgactcgcaccctcgcctttacaggccttgcgggccttctgtccgctcccctggtcaaggcccatggctttgtccagggcattgtcatcggtgaccaattgtaagtccctctcttgcagttctgtcgattaactgctggactgcttgcttgactccctgctgactcccaacagctacagcgggtacatcgtcaactcgttcccctacgaatccaacccaccccccgtcatcggctgggccacgaccgccaccgacctgggcttcgtcgacggcacaggataccaaggcccggacatcatctgccaccggaatgcgacgcccgcgccgctgacagcccccgtggccgccggcggcaccgtcgagctgcagtggacgccgtggccggacagccaccacggacccgtcatcacctacctggcgccgtgcaacggcaactgctcgaccgtcgacaagacgacgctggagttcttcaagatcgaccagcagggcctgatcgacgacacgagcccgccgggcacctgggcgtcggacaacctcatcgccaacaacaatagctggaccgtcaccattcccaacagcgtcgcccccggcaactacgtcctgcgccacgagatcatcgccctgcactcggccaacaacaaggacggcgcccagaactacccccagtgcatcaacatcgaggtcacgggcggcggctccgacgcgcctgagggtactctgggcgaggatctctaccatgacaccgacccgggcattctggtcgacatttacgagcccattgcgacgtataccattccggggccgcctgagccgacgttctag

SEQ ID NO: 36 - protein, 253 aa, Penicillium sp.
MLSSTTRTLAFTGLAGLLSAPLVKAHGFVQGIVIGDQFYSGYIVNSFPYESNPPPVIGWATTATDLGFVDGTGYQGPDIICHRNATPAPLTAPVAAGGTVELQWTPWPDSHHGPVITYLAPCNGNCSTVDKTTLEFFKIDQQGLIDDTSPPGTWASDNLIANNNSWTVTIPNSVAPGNYVLRHEIIALHSANNKDGAQNYPQCINIEVTGGGSDAPEGTLGEDLYHDTDPGILVDIYEPIATYTIPGPPEPTF

SEQ ID NO: 37 - DNA, 977 bp, Thielavia terrestris
atgaagctgtcatcccagctcgccgccctcacgctggccgcggcctccgtgtcaggccactacatcttcgagcagattgcccatggcggcaccaagttcccaccttacgagtacatccgaagaaacacgaactataacagccctgtcaccagtctctcgtcgaacgacctgcgatgcaacgtaggcggcgagacggctggcaacacgaccgtcctcgacgtgaaggcgggcgactccttcaccttctactcggacgtggccgtgtaccaccaggggcccatctcactgtgcgtgccccgggccaactttgatcagtcccaagcggactgtccgctcgcctggataaccacaattgactgacagcccgcacagctacatgtccaaggctcccggctccgtcgtggactacgacggctccggcgactggttcaagatccacgactggggcccgaccttcagcaacggccaggcctcgtggccgctgcggggtgcgtcccttccctttccctcccccttcctcccccttcctccccccctttccccccttttctgtctggtcgcacgccctgctgacgtccccgtagacaactaccagtacaacatcccgacgtgcatcccgaacggcgagtacctgctgcgcatccagtcgctggcgatccacaacccgggcgccacgccgcagttctacatcagctgcgcgcaggtccgggtctcgggcggcggcagcgcctccccctccccaacggccaagatccccggcgcgttcaaggcgaccgatcccgggtataccgcgaatgtgagtgccctatgttccttgcgctccttgttccttgctccttgctcggcgtgcttgaacgctacgggctgtggagggagggatggatggatgaataggatgctgactgatggtgggacaccagatttacaataacttccactcgtatacggtgccgggtccggcggtctttcagtgctag

SEQ ID NO: 38 - protein, 223 aa, Thielavia terrestris
MKLSSQLAALTLAAASVSGHYIFEQIAHGGTKFPPYEYIRRNTNYNSPVTSLSSNDLRCNVGGETAGNTTVLDVKAGDSFTFYSDVAVYHQGPISLYMSKAPGSVVDYDGSGDWFKIHDWGPTFSNGQASWPLRDNYQYNIPTCIPNGEYLLRIQSLAIHNPGATPQFYISCAQVRVSGGGSASPSPTAKIPGAFKATDPGYTANIYNNFHSYTVPGPAVFQC

SEQ ID NO: 39 - DNA, 878 bp, Thielavia terrestris
atgaagttctcactggtgtctctgctggcttacggcctctcggtcgaggcgcactccatcttccaggttcgtctcgcacatcacgctcaactcggctcgtggcgtaagggcaaggattaacacggccggcagagagtctcggtcaacggccaagaccaaggcctgctcaccggcctccgcgctccaagcaacaacaacccagtgcaagatgtcaacagccagaacatgatttgcggccagtcgggctccaagtcgcagaccgttatcaacgtcaaggccggcgacaggatcggctcgctctggcagcatgtcatcggcggcgcccagttttcgggtgacccggacaacccgatcgcccactcgcacaagggccccgtgatggcgtaccttgctaaggtcgacaatgccgcgtccgcgagccaaacgggtctgaagtggtaagtagcgggcgacgctcaggggacggggatcgggggcctgctccatccgagactaacaccgtggacaggttcaagatctggcaggacgggttcgataccagcagcaagacatggggcgtcgacaacctgatcaagaacaacggctgggtgtacttccacctgccgcagtgcctcgctccgggccagtatctcctgcgcgtcgaggttctggcgctgcactcggcgtaccagcagggccaggcccagttctaccagtcctgcgcccagatcaacgtctccggctccgggtccttcagcccgtcccagacggtcagcatcccgggcgtctacagcgccaccgacccgagcatcctcatcaacatctacggcagcacggggcagcccgacaacggcggcaaggcttacaacccccctggacccgccccgatctcctgctga

SEQ ID NO: 40 - protein, 246 aa, Thielavia terrestris
MKFSLVSLLAYGLSVEAHSIFQRVSVNGQDQGLLTGLRAPSNNNPVQDVNSQNMICGQSGSKSQTVINVKAGDRIGSLWQHVIGGAQFSGDPDNPIAHSHKGPVMAYLAKVDNAASASQTGLKWFKIWQDGFDTSSKTWGVDNLIKNNGWVYFHLPQCLAPGQYLLRVEVLALHSAYQQGQAQFYQSCAQINVSGSGSFSPSQTVSIPGVYSATDPSILINIYGSTGQPDNGGKAYNPPGPAPISC

SEQ ID NO: 41 - DNA, 1253 bp, Thielavia terrestris
atgaggacgacattcgccgccgcgttggcagccttcgctgcgcaggaagtggcaggccatgccatcttccaacagctctgggtggacggcaccgactatatacgtgctccccttttccttttgtgtttgcccatcctcgattgataacccgaggccatccaatgctgactcttacagcacggctcctcctgcgtccgcatgccgctgtcgaactcgcccgtcacgaacgtcggcagcagggacatgatctgcaacgccggcacgcgccccgtcagcgggaagtgccccgtcaaggccggcggcaccgtgacggttgagatgcaccaggtgggctgatttcctgagcgtcctattcctcccggaagcccctttcccatcctttgccctggctaacccctccgcccctcccagcaacccggggatcggtcgtgtaacaacgaagccatcggcggcgcccactggggaccggtgcaggtgtacctcagcaaggtggaggacgcgagcacggcggacgggtcgacgggctggttcaagatcttcgcggacacgtggtccaagaaggcgggcagctcggtgggggacgacgacaactggggcacgcgcgacctcaacgcgtgctgcggcaagatgcaggtcaagatcccggcggacatcccgtcgggcgactacctgctgcgggcggaggcgctggcgctgcacacggcgggccaggtgggcggcgcgcagttctacatgagctgctaccagatcaccgtgtcgggcggcggcagcgccagcccggccaccgtcaagttccccggcgcctacagcgccaacgacccgggcatccacatcaacatccacgcggccgtgtccaactacgtcgcgcccggcccggccgtctattccggcggcacgaccaaggtggccgggtccgggtgccaaggctgcgagaacacgtgcaaggtcggctcgtcgcccacggcgacggcgccgtcgggcaagagcggcgcgggttccgacggcggcgctgggaccgacggcgggtcttcgtcttcgagccccgacacgggcagcgcgtgcagcgtgcaggcctacgggcagtgcggcgggaacgggtactcgggttgcacccagtgcgcggtaagttcggggtcgtctgtcttttgtaggaacatccgagaggcttggctgacgaggcgttgttgtagcccggctatacttgcaaggcggtctctccgccgtactattcgcagtgcgccccttcttcttag

SEQ ID NO: 42 - protein, 334 aa, Thielavia terrestris
MRTTFAAALAAFAAQEVAGHAIFQQLWHGSSCVRMPLSNSPVTNVGSRDMICNAGTRPVSGKCPVKAGGTVTVEMHQQPGDRSCNNEAIGGAHWGPVQVYLSKVEDASTADGSTGWFKIFADTWSKKAGSSVGDDDNWGTRDLNACCGKMQVKIPADIPSGDYLLRAEALALHTAGQVGGAQFYMSCYQITVSGGGSASPATVKFPGAYSANDPGIHINIHAAVSNYVAPGPAVYSGGTTKVAGSGCQGCENTCKVGSSPTATAPSGKSGAGSDGGAGTDGGSSSSSPDTGSACSVQAYGQCGGNGYSGCTQCAPGYTCKAVSPPYYSQCAPSS

SEQ ID NO: 43 - DNA, 798 bp, Thielavia terrestris
atgaagctgagcgttgccatcgccgtgctggcgtcggctcttgccgaggctcactgtgagtgcatcgtctcactccagctactgcgaagcttgctgacgatggtccctagacaccttccccagcatcggaaacaccgctgactggcagtatgtgcggattacaacgaactaccagagcaacgggccggtgacggacgtcacctcggatcaaattcggtgctacgaacggaacccaggcacgggagcgcagggcatatacaacgtcaccgccggccagaccatcaactacaacgcgaaggcgtccatctcccacccggggcccatgtccttctacattgctaaggttcccgccggccaaaccgctgcgacctgggacggtaagggggctgtgtggaccaagatctaccaggacatgcccaagttcggcagcagcctgacctggcccaccatgggtaagaattctcaccctggaaatgaacgcacatttgcacagatctaacatggcctacaggcgccaagtctgtccccgtcaccatccctcgttgcctccagaacggcgattaccttctgcgagccgagcacatcgctctacacagcgcgagcagcgtcggtggcgcccagttctacctctcgtgcgcccagcttactgtcagcggcggcagtggcacctggaaccccaagaaccgggtctccttccccggcgcttacaaggcaacagacccgggcatcttgatcaacatctactaccccgtgccgaccagctactcgccgcccggcccgccggctgagacgtgctaa

SEQ ID NO: 44 - protein, 227 aa, Thielavia terrestris
MKLSVAIAVLASALAEAHYTFPSIGNTADWQYVRITTNYQSNGPVTDVTSDQIRCYERNPGTGAQGIYNVTAGQTINYNAKASISHPGPMSFYIAKVPAGQTAATWDGKGAVWTKIYQDMPKFGSSLTWPTMGAKSVPVTIPRCLQNGDYLLRAEHIALHSASSVGGAQFYLSCAQLTVSGGSGTWNPKNRVSFPGAYKATDPGILINIYYPVPTSYSPPGPPAETC

SEQ ID NO: 45 - DNA, 1107 bp, Thielavia terrestris
atgccttctttcgcctccaagactctcctttccaccctggcgggtgccgcatccgtggccgcccacgggcacgtgtcgaacatcgtcatcaacggggtctcgtaccagggttacgatccgacctccttcccttacatgcagaacccgcccatcgtggtcggctggactgccgccgacacggacaacggctttgttgccccggatgccttcgccagtggcgatatcatctgccacaagaacgccaccaacgccaagggccacgccgtggtcgccgcgggagacaagatcttcatccagtggaacacatggcccgagtcccaccacggccccgtcatcgactacctcgcgagctgcggcagcgcgtcctgcgagaccgtcgacaagaccaagctcgagttcttcaagatcgacgaggtcggcctggtcgacggcagctcggcgcccggtgtgtggggctccgaccagctcatcgccaacaacaactcgtggctcgtcgagatcccgcccaccatcgcgccgggcaactacgtcctgcgccacgagatcatcgcgctgcacagcgccgaaaacgccgacggcgcccagaactacccgcagtgcttcaacctgcagatcaccggcaccggcaccgccaccccctccggcgtccccggcacctcgctctacaccccgaccgacccgggcatcctcgtcaacatctacagcgccccgatcacctacaccgtcccggggccggccctcatctccggcgccgtcagcatcgcccagtcctcctccgccatcaccgcctccggcaccgccctgaccggctctgccaccgcacccgccgccgccgctgctaccacaacttccaccaccaacgccgcggctgctgctacctctgctgctgctgctgctggtacttccacaaccaccaccagcgccgcggccgtggtccagacctcctcctcctcctcctccgccccgtcctctgccgccgccgccgccaccaccaccgcggctgccagcgcccgcccgaccggctgctcctctggccgctccaggaagcagccgcgccgccacgcgcgggatatggtggttgcgcgaggggctgaggaggcaaactga

SEQ ID NO: 46 - protein, 368 aa, Thielavia terrestris
MPSFASKTLLSTLAGAASVAAHGHVSNIVINGVSYQGYDPTSFPYMQNPPIVVGWTAADTDNGFVAPDAFASGDIICHKNATNAKGHAVVAAGDKIFIQWNTWPESHHGPVIDYLASCGSASCETVDKTKLEFFKIDEVGLVDGSSAPGVWGSDQLIANNNSWLVEIPPTIAPGNYVLRHEIIALHSAENADGAQNYPQCFNLQITGTGTATPSGVPGTSLYTPTDPGILVNIYSAPITYTVPGPALISGAVSIAQSSSAITASGTALTGSATAPAAAAATTTSTTNAAAAATSAAAAAGTSTTTTSAAAVVQTSSSSSSAPSSAAAAATTTAAASARPTGCSSGRSRKQPRRHARDMVVARGAEEAN

SEQ ID NO: 47 - DNA, 993 bp, Thielavia terrestris
atgccgcccgcactccctcaactcctaaccacggtcctgaccgccctcaccctcggttccaccgccctcgcccactcacacctcgcgtacattatcgttaacggcaagctctaccagggcttcgacccgcgcccgcaccaggccaactacccttcccgggtcgggtggtccaccggcgccgtcgacgacggcttcgtcacgccggccaactactccaccccggacatcatttgccacatcgccggcaccagcccggccggccacgcgcccgtgcgcccgggcgaccgcatccacgtccagtggaacggctggccggtcggccacatcggtcccgtgctgtcgtacctcgcccgctgcgagtcggacacgggctgcacgggccagaacaagaccgcgctgcggtggaccaagatcgacgactccagcccgaccatgcagaacgtcgccggcgcgggcacccagggcgagggcacccccggcaagcgctgggccaccgacgtgctgatcgccgccaacaacagctggcaggtcgccgtgccggcggggctgccgaccggcgcgtacgtgctgcgcaacgagatcatcgcgctgcactacgcggcgaggaagaacggggcgcagaactatccgctctgcatgaacctgtgggtggacgccagtggtgataatagtagtgtggctgcaacgacggcggcggtgacggcggggggtctgcagatggatgcgtatgacgcgcgcgggttctacaaggagaacgatccgggcgtgctggtcaatgtcacggccgcgctgtcgtcgtatgtcgtgcccgggccgacggtggcggcgggcgccacgccggtgccgtacgcgcagcagagcccgagcgtgtcgacggcggcgggcacgcccgtcgtcgttacaaggactagcgagacggcgccgtacacgggcgccatgacgccgacggttgcggcgaggatgaaggggagggggtatgatcggcggggttag

SEQ ID NO: 48 - protein, 330 aa, Thielavia terrestris
MPPALPQLLTTVLTALTLGSTALAHSHLAYIIVNGKLYQGFDPRPHQANYPSRVGWSTGAVDDGFVTPANYSTPDIICHIAGTSPAGHAPVRPGDRIHVQWNGWPVGHIGPVLSYLARCESDTGCTGQNKTALRWTKIDDSSPTMQNVAGAGTQGEGTPGKRWATDVLIAANNSWQVAVPAGLPTGAYVLRNEIIALHYAARKNGAQNYPLCMNLWVDASGDNSSVAATTAAVTAGGLQMDAYDARGFYKENDPGVLVNVTAALSSYVVPGPTVAAGATPVPYAQQSPSVSTAAGTPVVVTRTSETAPYTGAMTPTVAARMKGRGYDRRG

SEQ ID NO: 49 - DNA, 1221 bp, Thielavia terrestris
atgaagacattcaccgccctcctggccgcagccggcctcgtcgccggccatggatatgtcgacaacgccaccattggcggccagttttatcaggtactctaccgcttcacccaaggtccgctggccacaactctataggtgtcataaattaacaagccaccgtcccgcagttctatcaggtgtgctcgctaccgaccatgtggtcccgtctcagcaagccactcacacgcccatgatcccctagccttacgtcgacccgtatttagcaaccttggcacgtagtatttattgtcccaaatattgagctgaactgcacctccctagaatcccgcggtgctaacattctttcagcccgacagggtctctcgatccatcccgggcaacggcccggtcacggacgtcactctcatcgacctgcagtgcaacgccaattccaccccggccaagctccacgccactgccgctgccggctcggacgtgattctccgctggacgctctggcctgagtcgcacgttggccccgtcatcacctacatggcccgctgccccgacacgggctgccaggactggatgccgggcacttcgtaggagcccatcttgcaccatatccatttcaaccggccacacgcactgacccatatgtctgtctacccctgcagtgcggtctggttcaagatcaaggagggcggccgcgacggcacttccaacacctgggccgacgtacgtgtaccccgtcccagagagccaaagcccccccttcaacaaagcaaacatctcaatagcccgagcctacgcactaacccctctccttccccctcgaaaacacagaccccgctgatgacggcgcccacctcgtacacgtacacgatcccctcctgcctgaagaagggctactacctggtccgccacgagatcatcgcgctgcacgccgcctacacctaccccggcgcgcagttctacccgggctgccaccagctcaacgtcacgggcggcgggtccaccgtaccgtcgagcggcctggtggcctttcccggggcgtacaagggcagtgaccccgggattacgtacgatgcgtataaaggtgggttggctggttggcccaggtcttggtgatgggggaatgtggtgatgaggtttattatttgggatcccgtggctaacgtaaccctgggtgtagcgcaaacgtaccagattcctgggccggcggtctttacttgctga

SEQ ID NO: 50 - protein, 236 aa, Thielavia terrestris
MKTFTALLAAAGLVAGHGYVDNATIGGQFYQNPAVLTFFQPDRVSRSIPGNGPVTDVTLIDLQCNANSTPAKLHATAAAGSDVILRWTLWPESHVGPVITYMARCPDTGCQDWMPGTSAVWFKIKEGGRDGTSNTWADTPLMTAPTSYTYTIPSCLKKGYYLVRHEIIALHAAYTYPGAQFYPGCHQLNVTGGGSTVPSSGLVAFPGAYKGSDPGITYDAYKAQTYQIPGPAVFTC

SEQ ID NO: 51 - DNA, 933 bp, Thielavia terrestris
atggccttgctgctcttggcaggcttggccattctggccgggccggctcatgcccacggcggcctcgccaactacacagtgggcaacacctggtatagggggtgcgtaaggggggcaccgacaacgcctgcttagtaactccaccatttcgagcgggctaacaccgggcgcagctacgaccccttcacgccggcggccgaccagatcggccagccgtggatgatccaacgcgcgtgggactcgatcgacccgatcttcagcgtcaacgacaaggcgctcgcctgcaacaccccggccacggcgccgacctcttacattcccatccgcgcgggcgagaacatcacggccgtgtactggtactggctgcacccggtgggccccatgacggcgtggctggcgcggtgcgacggcgactgccgcgacgccgacgtcaacgaggcgcgctggttcaagatctgggaggccggcctgctcagcgggccgaacctggccgagggcatgtggtaccagaaggcgttccagaactgggacggcagcccggacctgtggcccgtcacgatcccggccgggctgaagagcggcctgtacatgatccggcacgagatcttgtcgatccacgtcgaggataaaccgcagttttatcccgagtgtgcgcatctgaatgtgaccgggggtggggacctgctgccgcctgatgagtttttggtgaagttcccgggcgcttacaaagaagatagtgagtgaaacgcgaagcttcggtagccattgggttgcgctgatggaggttagacccgtcgatcaagatcaatatctactcggaccagtacgccaatacaacggtgagtgtaacaggtcgagcaaaaccaaacagatgccgatgactgatgatctcagaattacacaattcccggagggccgatatgggatgggtga

SEQ ID NO: 52 - protein, 250 aa, Thielavia terrestris
MALLLLAGLAILAGPAHAHGGLANYTVGNTWYRGYDPFTPAADQIGQPWMIQRAWDSIDPIFSVNDKALACNTPATAPTSYIPIRAGENITAVYWYWLHPVGPMTAWLARCDGDCRDADVNEARWFKIWEAGLLSGPNLAEGMWYQKAFQNWDGSPDLWPVTIPAGLKSGLYMIRHEILSIHVEDKPQFYPECAHLNVTGGGDLLPPDEFLVKFPGAYKEDNPSIKINIYSDQYANTTNYTIPGGPIWDG

SEQ ID NO: 53 - DNA, 1584 bp, Thielavia terrestris
atgatgccgtcccttgttcgcttctcaatgggtctggcgaccgccttcgcctcgctgtccacagcacataccgtcttcaccacgcttttcatcaacggcgtcgaccaaggggacgggacctgcatccgcatggccaagaagggcagcgtttgcacccatcccattgctggtggcctcgacagcccagacatggcttgtggtatgccctctgcgtttcccctgcgagagctttcctcgagctaacccaatgccgcgttgcccaggccgagacggacaacaagccgtggcattcacctgcccagccccggcgggctccaagttgagcttcgagttccgcatgtgggccgacgcctctcagcccggctctatcgacccatcccacctcggctcgacggcaatctacctcaaacaagtctccaacatcagctccgactcggctgccggccctggctggttcaagatctacgccgagggctacgacacagccgccaagaagtgggccacagagaagctcatcgacaacggcggcctgctgagcatcgagcttccgcccactctgccggcgggatactacctcgcccgcagcgagatcgtcaccatccagaacgtcaccaacgaccacgtcgacccgcagttctacgttggctgcgcacagctcttcgtccaggggcctccgaccacccccaccgtcccgccagacagactcgtctccatcccgggccacgtccatgcctccgacccggggctgaccttcaacatctggcgcgacgacccctccaagacggcctacaccgtcgtcggcccggcccccttctcccccaccgccgcccccacccccacctccaccaacaccaacgggcagcaacaacaacaacagcaacaggcgataaagcagacggacggcgtgatccccgccgactgccagctcaagaacgccaactggtgcggcgccgaggtgcccgcgtacgccgacgaggccggctgctgggcgtcgtcggccgactgcttcgcccagctggacgcctgctacacgtcggcgccgcccacgggcagccgcggctgccggctgtgggaggactggtgcaccggcattcagcagggctgccgcgcggggcggtggcgggggccgccgccctttcatggggagggggcagcagcggaggtgtgaacggttcggggacgggtggcggtggtggtggtggtggtggtggcactggctcttcttcggcttctgccccgacggagacggcctctgctggccgggggggcgcaagaatagctgccgtggccggctgcggaggcgggacaggagacatggttgaagaggttttcctcttttattgggacgcttgcagcggctggcgacggagccgtggtggtggttcgattcttgcgaggcttatccttcatgtccttcttccacttttgagaccgaggcgagcccctcgagtccatttacttctcttccacctgtacctcaacttctgttatccaggaaccagtggtttctataatcgcctgagcattaaactaggcatatggccaagcaaaatgtcgcctgatgtagcgcattacgtgaaataa

SEQ ID NO: 54 - protein, 478 aa, Thielavia terrestris
MMPSLVRFSMGLATAFASLSTAHTVFTTLFINGVDQGDGTCIRMAKKGSVCTHPIAGGLDSPDMACGRDGQQAVAFTCPAPAGSKLSFEFRMWADASQPGSIDPSHLGSTAIYLKQVSNISSDSAAGPGWFKIYAEGYDTAAKKWATEKLIDNGGLLSIELPPTLPAGYYLARSEIVTIQNVTNDHVDPQFYVGCAQLFVQGPPTTPTVPPDRLVSIPGHVHASDPGLTFNIWRDDPSKTAYTVVGPAPFSPTAAPTPTSTNTNGQQQQQQQQAIKQTDGVIPADCQLKNANWCGAEVPAYADEAGCWASSADCFAQLDACYTSAPPTGSRGCRLWEDWCTGIQQGCRAGRWRGPPPFHGEGAAAETASAGRGGARIAAVAGCGGGTGDMVEEVFLFYWDACSGWRRSRGGGSILARLILHVLLPLLRPRRAPRVHLLLFHLYLNFCYPGTSGFYNRLSIKLGIWPSKMSPDVAHYVK

SEQ ID NO: 55 - DNA, 868 bp, Thielavia terrestris
atgcagctcctcgtgggcttgctgcttgcagccgtggctgctcgagcacattgtatttctacccctttccgcgtgcctcccagcctcaaggcaagaagacgcacgcagcagctaacggaccctatcagacacatttcccagactcgtggtaaatgggcagcccgaggacaaggactggtcggttacgcgcatgaccaagaacgcgcagagcaagcagggagtccaggacccgaccagtcccgacattcgctgctacacgtcgcagacggcgcctaacgtggctacggtccctgccggagccaccgtccattacatatcgactcagcagatcaaccacccgggcccgacgcagtactacctcgccaaggtaccggcggggtcgtcggccaagacgtgggacgggtcaggggccgtctggttcaagatctcgaccaccatgccttacttggacaacaacaagcagcttgtctggccgaatcagagtaggaacaattcccgctccaatcttcgatttggccttgagctacggccgattgcatgggagagaccgttgactgacggggcaacccaaccttcatcagacacgtacacgacggtcaacacgaccatccccgccgatacgcccagtggggaatacctcctccgggtcgagcagatcgcgctgcacctggcctcgcagcccaacggggctcagttctacctggcctgctcgcagatccagattacgggcggcggcaacggcacgcccggcccgctagtcgcgttgccgggggcgtacaagagcaacgacccgggcattttggtcaacatctactctatgcagcccggcgattacaagccgcccgggccgccggtgtggagtggctga

SEQ ID NO: 56 - protein, 230 aa, Thielavia terrestris
MQLLVGLLLAAVAARAHYTFPRLVVNGQPEDKDWSVTRMTKNAQSKQGVQDPTSPDIRCYTSQTAPNVATVPAGATVHYISTQQINHPGPTQYYLAKVPAGSSAKTWDGSGAVWFKISTTMPYLDNNKQLVWPNQNTYTTVNTTIPADTPSGEYLLRVEQIALHLASQPNGAQFYLACSQIQITGGGNGTPGPLVALPGAYKSNDPGILVNIYSMQPGDYKPPGPPVWSG

SEQ ID NO: 57 - DNA, 1068 bp, Thielavia terrestris
atgaagctgtacctggcggcctttctaggcgccgtcgccaccccgggagcgttcgctcatcgtaggttccccgtctatctccctaggggtagcaccacgactaatttctcgtcgtccccctgtagaaatccacgggattctacttgtcaacggcaccgaaacgccggaatggaaatacgtccggtaatatctaccttgctctccttcttccacaaccagcctaacacatcatcagtgacgtggcctgggagggcgcctacgaaccggaaaaataccccaacaccgagttctttaagacgcccccgcagacggacatcaacaacccgaacatcacctgcggcaggaacgcgttcgactcggccagcaagactgagacggccgacatactggccggctcagaggtcggcttccgcgtctcgtgggacggcaacggcaagtacggcgtgttctggcatcccgggccggggcagatctacctctctcgtgctccgaacgacgacctggaggactaccgcggcgacggagactggttcaagatcgcaaccggcgccgccgtctccaataccgagtggctgctgtggaacaagcatgacgtgagccccaacattcctcgcccaatcgatccccaacctggtcaccatggcggcgtccgggatgcaaagagactaactccagaggaacctacctagttcaacttcaccatccccaagacgacgccgccgggcaagtacctgatgcgcatcgagcagttcatgccctccacggtcgaatacagccagtggtacgtcaactgcgcccacgtcaacatcatcggccccggcggaggcacgccgacgggctttgccaggtttcccggcacctacactgttgacgatcccggtaagccggacctaccggacacagaggcctcgggatagcttgctaaccttgtttgctctctctctttttctctcccgactaggcatcaaggtgccgttgaaccagatcgtcaacagcggagagttgccgcaggaccaactgaggctgctcgagtacaagcccccgggcccagcgctgtggactggttga

SEQ ID NO: 58 - protein, 257 aa, Thielavia terrestris
MKLYLAAFLGAVATPGAFAHQIHGILLVNGTETPEWKYVRDVAWEGAYEPEKYPNTEFFKTPPQTDINNPNITCGRNAFDSASKTETADILAGSEVGFRVSWDGNGKYGVFWHPGPGQIYLSRAPNDDLEDYRGDGDWFKIATGAAVSNTEWLLWNKHDFNFTIPKTTPPGKYLMRIEQFMPSTVEYSQWYVNCAHVNIIGPGGGTPTGFARFPGTYTVDDPGIKVPLNQIVNSGELPQDQLRLLEYKPPGPALWTG

SEQ ID NO: 59 - DNA, 871 bp, Thermoascus crustaceus
atggccttttcccagataatggctattaccggcgtttttcttgcctctgcttccctggtggctggccatggctttgttcagaatatcgtgattgatggtaaaaggtacctaactacctaccttactatctgatgtcatttacaagaaagggcacagacacaagcggcaaaaaaaagaaagaaagaaagaaagaaagaaagctgacaaaaattcaacaagttatggcgggtacatcgtgaaccaatatccatacatgtcagatcctccggaggtcgtcggctggtctaccaccgcaaccgacctcggattcgtggacggtaccggataccaaggacctgatatcatctgccacaggggcgccaagcctgcagccctgactgcccaagtggccgccggaggaaccgtcaagctggaatggactccatggcctgattctcaccacggcccggtgatcaactaccttgctccttgcaacggtgactgttccaccgtggacaagacccaattgaaattcttcaagatcgcccaggccggtctcatcgatgacaacagtcctcctggtatctgggcctcagacaatctgatagcggccaacaacagctggactgtcaccatcccaaccacaactgcacctggaaactatgttctaaggcatgagatcattgctctccactcagctgggaacaaggatggtgcgcagaactatccccagtgcatcaacctgaaggtcactggaaatggttctggcaatcctcctgctggtgctcttggaacggcactctacaaggatacagatccgggaattctgatcaatatctaccagaaactttccagctatgttattcctggtcctgctttgtacactggttag

SEQ ID NO: 60 - protein, 251 aa, Thermoascus crustaceus
MAFSQIMAITGVFLASASLVAGHGFVQNIVIDGKSYGGYIVNQYPYMSDPPEVVGWSTTATDLGFVDGTGYQGPDIICHRGAKPAALTAQVAAGGTVKLEWTPWPDSHHGPVINYLAPCNGDCSTVDKTQLKFFKIAQAGLIDDNSPPGIWASDNLIAANNSWTVTIPTTTAPGNYVLRHEIIALHSAGNKDGAQNYPQCINLKVTGNGSGNPPAGALGTALYKDTDPGILINIYQKLSSYVIPGPALYTG

SEQ ID NO: 61 - DNA, 1102 bp, Thermoascus crustaceus
atgtcattctcgaagatacttgctatcgctggggccattacctacgcatcttcagctgccgctcatggttatgtccagggaattgttgtcgatggcagctagtatgtcactctggatggaaccttcagcacgtactgtactaacaatcagcagctacgggggatatatggtgacccaatatccctacaccgctcaacctccggaactcatcgcctggtccactaaagcaaccgatcttgggtttgtggacggcagtggctatacttctcctgatatcatctgccataagggtgctgagcctggtgcccagagcgccaaagtggcagctggagggaccgttgagctgcagtggacggcatggcccgagtctcacaagggcccagttattgactacctcgccgcctgcgacggggactgctcatctgttgataagactgcactaaagttctttaagattgacgagagtggtctgattgacggcaacggtgctggaacatgggcctctgatacgttgatcaaaaataacaacagctggactgtcaccatcccaagcacaattgcttccggaaactacgtactaagacacgaaataattgcgctccattctgccggaaacaaagatggtgctcagaactatccccagtgtatcaacctcgaggtcactggtagtggcaccgaaaaccctgctggcactctcggaacagcgctttacacagacactgatcctggccttctggtcaacatctaccagggtctgtccaactattcaatccctggtcctgctctgtatagcggcaacagtgataacgctggttccctcaaccctaccaccacgccgtcaattcagaatgctgctgctgctccctccacttccacagcatctgttgtcactgattcttcgtcagccacccagactgctagtgtcgccgccacgactccagcctccacttcggctgttacagcctcaccagctcccgatactggaagcgacgtaaccaaatatctggattcgatgagctcggatgaggtcctcaccctggtgcgcgggaccctgtcttggctggtttctaacaagaaacatgcgcgggatctttctcactga

SEQ ID NO: 62 - protein, 349 aa, Thermoascus crustaceus
MSFSKILAIAGAITYASSAAAHGYVQGIVVDGSYYGGYMVTQYPYTAQPPELIAWSTKATDLGFVDGSGYTSPDIICHKGAEPGAQSAKVAAGGTVELQWTAWPESHKGPVIDYLAACDGDCSSVDKTALKFFKIDESGLIDGNGAGTWASDTLIKNNNSWTVTIPSTIASGNYVLRHEIIALHSAGNKDGAQNYPQCINLEVTGSGTENPAGTLGTALYTDTDPGLLVNIYQGLSNYSIPGPALYSGNSDNAGSLNPTTTPSIQNAAAAPSTSTASVVTDSSSATQTASVAATTPASTSAVTASPAPDTGSDVTKYLDSMSSDEVLTLVRGTLSWLVSNKKHARDLSH

SEQ ID NO: 63 - DNA, 1493 bp, Thermoascus crustaceus
atgttgtcattcattcccaccaagtcagctgcgctgacgactcttctacttcttggaacagctcatgctcacactttgatgaccaccatgtttgtggacggcgtcaaccagggagatggtgtctgcattcgcatgaacaatgacggcggaactgccaatacctatatccagcctatcacgagcaaggatatcgcctgcggtaagtacccagatgtcatcatactctgccataacatccgtcatatctactagaatcggagcaatgttaagtatttccaggcatccaaggcgaaatcggcgcctcccgagtctgcccagtcaaggcatcttccaccctaaccttccaattccgcgagcaacccaacaacccaaactcctcccctctcgatccatcgcacaaaggccccgccgcggtgtacctgaaaaaggtcgactccgccatcgcgagcaacaacgccgccggagacagctggttcaagatctgggagtccgtctacgacgagtccacgggcaaatggggcacgaccaagatgatcgagaacaacgggcacatctccgtcaaggtgcccgatgatatcgagggtggttactatcttgcccggacggagctgctggcgctacattctgcggatcagggggatccgcagttctatgttggctgtgcgcagctgtttatcgattcggatgggacggcgaaaccgcccactgtttctattggagaggggacgtacgatctgagcatgcctgccatgacgtataatatctgggagacaccgttggctctgccgtatccgatgtatgggcctcctgtctatacgcctggctctggttctggatcagtccgtgcgacgagctcttctgctgtccctactgcaaccgaatcctcttttgtagaggaaagagcaaaccccgtcacggcaaacagtgtttattctgcaaggggcaaattcaaaacctggattgataaactgtcatggcgcgggaaggtccgtgagaacgtcagacaagccgcgggaagaagaagcactctcgtccagactgtgggtctaaagccaaaaggctgcatcttcgtcaatggaaactggtgcggcttcgaggttcccgactacaacgatgcggagagctgctgggctgtatgttcccctccttagcctcttacatccctaagtactacatttgaaaacaacaaaaagaaatgtatatactaactacgtacgctctactctaggcctccgacaactgctggaaacagtccgacgcctgctggaacaagacccaacccacgggctacaataactgccagatctggcaggacaagaaatgcaaggtcatccaggattcctgtagcggacccaacccgcatggaccaccgaataagggcaaggatttgactccggagtggccgccactgaagggctcgatggatacgttctccaagcgtactatcggttaccgcgattggattgttagaaggagaggtgcatgagggtgta

SEQ ID NO: 64 - protein, 436 aa, Thermoascus crustaceus
MLSFIPTKSAALTTLLLLGTAHAHTLMTTMFVDGVNQGDGVCIRMNNDGGTANTYIQPITSKDIACGIQGEIGASRVCPVKASSTLTFQFREQPNNPNSSPLDPSHKGPAAVYLKKVDSAIASNNAAGDSWFKIWESVYDESTGKWGTTKMIENNGHISVKVPDDIEGGYYLARTELLALHSADQGDPQFYVGCAQLFIDSDGTAKPPTVSIGEGTYDLSMPAMTYNIWETPLALPYPMYGPPVYTPGSGSGSVRATSSSAVPTATESSFVEERANPVTANSVYSARGKFKTWIDKLSWRGKVRENVRQAAGRRSTLVQTVGLKPKGCIFVNGNWCGFEVPDYNDAESCWAASDNCWKQSDACWNKTQPTGYNNCQIWQDKKCKVIQDSCSGPNPHGPPNKGKDLTPEWPPLKGSMDTFSKRTIGYRDWIVRRRGA

SEQ ID NO: 65 - DNA, 1377 bp, Trichoderma reesei
atggcgccctcagttacactgccgttgaccacggccatcctggccattgcccggctcgtcgccgcccagcaaccgggtaccagcacccccgaggtccatcccaagttgacaacctacaagtgtacaaagtccggggggtgcgtggcccaggacacctcggtggtccttgactggaactaccgctggatgcacgacgcaaactacaactcgtgcaccgtcaacggcggcgtcaacaccacgctctgccctgacgaggcgacctgtggcaagaactgcttcatcgagggcgtcgactacgccgcctcgggcgtcacgacctcgggcagcagcctcaccatgaaccagtacatgcccagcagctctggcggctacagcagcgtctctcctcggctgtatctcctggactctgacggtgagtacgtgatgctgaagctcaacggccaggagctgagcttcgacgtcgacctctctgctctgccgtgtggagagaacggctcgctctacctgtctcagatggacgagaacgggggcgccaaccagtataacacggccggtgccaactacgggagcggctactgcgatgctcagtgccccgtccagacatggaggaacggcaccctcaacactagccaccagggcttctgctgcaacgagatggatatcctggagggcaactcgagggcgaatgccttgacccctcactcttgcacggccacggcctgcgactctgccggttgcggcttcaacccctatggcagcggctacaaaagctactacggccccggagataccgttgacacctccaagaccttcaccatcatcacccagttcaacacggacaacggctcgccctcgggcaaccttgtgagcatcacccgcaagtaccagcaaaacggcgtcgacatccccagcgcccagcccggcggcgacaccatctcgtcctgcccgtccgcctcagcctacggcggcctcgccaccatgggcaaggccctgagcagcggcatggtgctcgtgttcagcatttggaacgacaacagccagtacatgaactggctcgacagcggcaacgccggcccctgcagcagcaccgagggcaacccatccaacatcctggccaacaaccccaacacgcacgtcgtcttctccaacatccgctggggagacattgggtctactacgaactcgactgcgcccccgcccccgcctgcgtccagcacgacgttttcgactacacggaggagctcgacgacttcgagcagcccgagctgcacgcagactcactgggggcagtgcggtggcattgggtacagcgggtgcaagacgtgcacgtcgggcactacgtgccagtatagcaacgactactactcgcaatgcctt

SEQ ID NO: 66 - protein, 459 aa, Trichoderma reesei
MAPSVTLPLTTAILAIARLVAAQQPGTSTPEVHPKLTTYKCTKSGGCVAQDTSVVLDWNYRWMHDANYNSCTVNGGVNTTLCPDEATCGKNCFIEGVDYAASGVTTSGSSLTMNQYMPSSSGGYSSVSPRLYLLDSDGEYVMLKLNGQELSFDVDLSALPCGENGSLYLSQMDENGGANQYNTAGANYGSGYCDAQCPVQTWRNGTLNTSHQGFCCNEMDILEGNSRANALTPHSCTATACDSAGCGFNPYGSGYKSYYGPGDTVDTSKTFTIITQFNTDNGSPSGNLVSITRKYQQNGVDIPSAQPGGDTISSCPSASAYGGLATMGKALSSGMVLVFSIWNDNSQYMNWLDSGNAGPCSSTEGNPSNILANNPNTHVVFSNIRWGDIGSTTNSTAPPPPPASSTTFSTTRRSSTTSSSPSCTQTHWGQCGGIGYSGCKTCTSGTTCQYSNDYYSQCL

SEQ ID NO: 67 - DNA, 1254 bp, Trichoderma reesei
atgaacaagtccgtggctccattgctgcttgcagcgtccatactatatggcggcgccgtcgcacagcagactgtctggggccagtgtggaggtattggttggagcggacctacgaattgtgctcctggctcagcttgttcgaccctcaatccttattatgcgcaatgtattccgggagccactactatcaccacttcgacccggccaccatccggtccaaccaccaccaccagggctacctcaacaagctcatcaactccacccacgagctctggggtccgatttgccggcgttaacatcgcgggttttgactttggctgtaccacagatggcacttgcgttacctcgaaggtttatcctccgttgaagaacttcaccggctcaaacaactaccccgatggcatcggccagatgcagcacttcgtcaacgaggacgggatgactattttccgcttacctgtcggatggcagtacctcgtcaacaacaatttgggcggcaatcttgattccacgagcatttccaagtatgatcagcttgttcaggggtgcctgtctctgggcgcatactgcatcgtcgacatccacaattatgctcgatggaacggtgggatcattggtcagggcggccctactaatgctcaattcacgagcctttggtcgcagttggcatcaaagtacgcatctcagtcgagggtgtggttcggcatcatgaatgagccccacgacgtgaacatcaacacctgggctgccacggtccaagaggttgtaaccgcaatccgcaacgctggtgctacgtcgcaattcatctctttgcctggaaatgattggcaatctgctggggctttcatatccgatggcagtgcagccgccctgtctcaagtcacgaacccggatgggtcaacaacgaatctgatttttgacgtgcacaaatacttggactcagacaactccggtactcacgccgaatgtactacaaataacattgacggcgccttttctccgcttgccacttggctccgacagaacaatcgccaggctatcctgacagaaaccggtggtggcaacgttcagtcctgcatacaagacatgtgccagcaaatccaatatctcaaccagaactcagatgtctatcttggctatgttggttggggtgccggatcatttgatagcacgtatgtcctgacggaaacaccgactagcagtggtaactcatggacggacacatccttggtcagctcgtgtctcgcaagaaag

SEQ ID NO: 68 - protein, 418 aa, Trichoderma reesei
MNKSVAPLLLAASILYGGAVAQQTVWGQCGGIGWSGPTNCAPGSACSTLNPYYAQCIPGATTITTSTRPPSGPTTTTRATSTSSSTPPTSSGVRFAGVNIAGFDFGCTTDGTCVTSKVYPPLKNFTGSNNYPDGIGQMQHFVNEDGMTIFRLPVGWQYLVNNNLGGNLDSTSISKYDQLVQGCLSLGAYCIVDIHNYARWNGGIIGQGGPTNAQFTSLWSQLASKYASQSRVWFGIMNEPHDVNINTWAATVQEVVTAIRNAGATSQFISLPGNDWQSAGAFISDGSAAALSQVTNPDGSTTNLIFDVHKYLDSDNSGTHAECTTNNIDGAFSPLATWLRQNNRQAILTETGGGNVQSCIQDMCQQIQYLNQNSDVYLGYVGWGAGSFDSTYVLTETPTSSGNSWTDTSLVSSCLARK

SEQ ID NO: 69 - DNA, 702 bp, Trichoderma reesei
atgaagttccttcaagtcctccctgccctcataccggccgccctggcccaaaccagctgtgaccagtgggcaaccttcactggcaacggctacacagtcagcaacaacctttggggagcatcagccggctctggatttggctgcgtgacggcggtatcgctcagcggcggggcctcctggcacgcagactggcagtggtccggcggccagaacaacgtcaagtcgtaccagaactctcagattgccattccccagaagaggaccgtcaacagcatcagcagcatgcccaccactgccagctggagctacagcgggagcaacatccgcgctaatgttgcgtatgacttgttcaccgcagccaacccgaatcatgtcacgtactcgggagactacgaactcatgatctggcttggcaaatacggcgatattgggccgattgggtcctcacagggaacagtcaacgtcggtggccagagctggacgctctactatggctacaacggagccatgcaagtctattcctttgtggcccagaccaacactaccaactacagcggagatgtcaagaacttcttcaattatctccgagacaataaaggatacaacgctgcaggccaatatgttcttagctaccaatttggtaccgagcccttcacgggcagtggaactctgaacgtcgcatcctggaccgcatctatcaac

SEQ ID NO: 70 - protein, 234 aa, Trichoderma reesei
MKFLQVLPALIPAALAQTSCDQWATFTGNGYTVSNNLWGASAGSGFGCVTAVSLSGGASWHADWQWSGGQNNVKSYQNSQIAIPQKRTVNSISSMPTTASWSYSGSNIRANVAYDLFTAANPNHVTYSGDYELMIWLGKYGDIGPIGSSQGTVNVGGQSWTLYYGYNGAMQVYSFVAQTNTTNYSGDVKNFFNYLRDNKGYNAAGQYVLSYQFGTEPFTGSGTLNVASWTASIN

SEQ ID NO: 71 - DNA, 726 bp, Trichoderma reesei
atgaaggcaactctggttctcggctccctcattgtaggcgccgtttccgcgtacaaggccaccaccacgcgctactacgatgggcaggagggtgcttgcggatgcggctcgagctccggcgcattcccgtggcagctcggcatcggcaacggagtctacacggctgccggctcccaggctctcttcgacacggccggagcttcatggtgcggcgccggctgcggtaaatgctaccagctcacctcgacgggccaggcgccctgctccagctgcggcacgggcggtgctgctggccagagcatcatcgtcatggtgaccaacctgtgcccgaacaatgggaacgcgcagtggtgcccggtggtcggcggcaccaaccaatacggctacagctaccatttcgacatcatggcgcagaacgagatctttggagacaatgtcgtcgtcgactttgagcccattgcttgccccgggcaggctgcctctgactgggggacgtgcctctgcgtgggacagcaagagacggatcccacgcccgtcctcggcaacgacacgggctcaactcctcccgggagctcgccgccagcgacatcgtcgagtccgccgtctggcggcggccagcagacgctctatggccagtgtggaggtgccggctggacgggacctacgacgtgccaggccccagggacctgcaaggttcagaaccagtggtactcccagtgtcttcct

SEQ ID NO: 72 - protein, 242 aa, Trichoderma reesei
MKATLVLGSLIVGAVSAYKATTTRYYDGQEGACGCGSSSGAFPWQLGIGNGVYTAAGSQALFDTAGASWCGAGCGKCYQLTSTGQAPCSSCGTGGAAGQSIIVMVTNLCPNNGNAQWCPVVGGTNQYGYSYHFDIMAQNEIFGDNVVVDFEPIACPGQAASDWGTCLCVGQQETDPTPVLGNDTGSTPPGSSPPATSSSPPSGGGQQTLYGQCGGAGWTGPTTCQAPGTCKVQNQWYSQCLP

SEQ ID NO: 73 - DNA, 923 bp, Trichoderma reesei
atgcgttcctcccccctcctccgctccgccgttgtggccgccctgccggtgttggcccttgccgctgatggcaggtccacccgctactgggactgctgcaagccttcgtgcggctgggccaagaaggctcccgtgaaccagcctgtcttttcctgcaacgccaacttccagcgtatcacggacttcgacgccaagtccggctgcgagccgggcggtgtcgcctactcgtgcgccgaccagaccccatgggctgtgaacgacgacttcgcgctcggttttgctgccacctctattgccggcagcaatgaggcgggctggtgctgcgcctgctacgagctcaccttcacatccggtcctgttgctggcaagaagatggtcgtccagtccaccagcactggcggtgatcttggcagcaaccacttcgatctcaacatccccggcggcggcgtcggcatcttcgacggatgcactccccagttcggcggtctgcccggccagcgctacggcggcatctcgtcccgcaacgagtgcgatcggttccccgacgccctcaagcccggctgctactggcgcttcgactggttcaagaacgccgacaatccgagcttcagcttccgtcaggtccagtgcccagccgagctcgtcgctcgcaccggatgccgccgcaacgacgacggcaacttccctgccgtccagatcccctccagcagcaccagctctccggtcaaccagcctaccagcaccagcaccacgtccacctccaccacctcgagcccgccagtccagcctacgactcccagcggctgcactgctgagaggtgggctcagtgcggcggcaatggctggagcggctgcaccacctgcgtcgctggcagcacttgcacgaagattaatgactggtaccatcagtgcctgtagaattc

SEQ ID NO: 74 - protein, 305 aa, Trichoderma reesei
MRSSPLLRSAVVAALPVLALAADGRSTRYWDCCKPSCGWAKKAPVNQPVFSCNANFQRITDFDAKSGCEPGGVAYSCADQTPWAVNDDFALGFAATSIAGSNEAGWCCACYELTFTSGPVAGKKMVVQSTSTGGDLGSNHFDLNIPGGGVGIFDGCTPQFGGLPGQRYGGISSRNECDRFPDALKPGCYWRFDWFKNADNPSFSFRQVQCPAELVARTGCRRNDDGNFPAVQIPSSSTSSPVNQPTSTSTTSTSTTSSPPVQPTTPSGCTAERWAQCGGNGWSGCTTCVAGSTCTKINDWYHQCL

SEQ ID NO: 75 - DNA, 1188 bp, Myceliophthora thermophila
cgacttgaaacgccccaaatgaagtcctccatcctcgccagcgtcttcgccacgggcgccgtggctcaaagtggtccgtggcagcaatgtggtggcatcggatggcaaggatcgaccgactgtgtgtcgggctaccactgcgtctaccagaacgattggtacagccagtgcgtgcctggcgcggcgtcgacaacgctgcagacatcgaccacgtccaggcccaccgccaccagcaccgcccctccgtcgtccaccacctcgcctagcaagggcaagctgaagtggctcggcagcaacgagtcgggcgccgagttcggggagggcaattaccccggcctctggggcaagcacttcatcttcccgtcgacttcggcgattcagacgctcatcaatgatggatacaacatcttccggatcgacttctcgatggagcgtctggtgcccaaccagttgacgtcgtccttcgaccagggttacctccgcaacctgaccgaggtggtcaacttcgtgacgaacgcgggcaagtacgccgtcctggacccgcacaactacggccggtactacggcaacatcatcacggacacgaacgcgttccggaccttctggaccaacctggccaagcagttcgcctccaactcgctcgtcatcttcgacaccaacaacgagtacaacacgatggaccagaccctggtgctcaacctcaaccaggccgccatcgacggcatccgggccgccggcgcgacctcgcagtacatcttcgtcgagggcaacgcgtggagcggggcctggagctggaacacgaccaacaccaacatggccgccctgacggacccgcagaacaagatcgtgtacgagatgcaccagtacctcgactcggacagctcgggcacccacgccgagtgcgtcagcagcaccatcggcgcccagcgcgtcgtcggagccacccagtggctccgcgccaacggcaagctcggcgtcctcggcgagttcgccggcggcgccaacgccgtctgccagcaggccgtcaccggcctcctcgaccacctccaggacaacagcgacgtctggctgggtgccctctggtgggccgccggtccctggtggggcgactacatgtactcgttcgagcctccttcgggcaccggctatgtcaactacaactcgatcttgaagaagtacttgccgtaa

SEQ ID NO: 76 - protein, 389 aa, Myceliophthora thermophila
MKSSILASVFATGAVAQSGPWQQCGGIGWQGSTDCVSGYHCVYQNDWYSQCVPGAASTTLQTSTTSRPTATSTAPPSSTTSPSKGKLKWLGSNESGAEFGEGNYPGLWGKHFIFPSTSAIQTLINDGYNIFRIDFSMERLVPNQLTSSFDQGYLRNLTEVVNFVTNAGKYAVLDPHNYGRYYGNIITDTNAFRTFWTNLAKQFASNSLVIFDTNNEYNTMDQTLVLNLNQAAIDGIRAAGATSQYIFVEGNAWSGAWSWNTTNTNMAALTDPQNKIVYEMHQYLDSDSSGTHAECVSSTIGAQRVVGATQWLRANGKLGVLGEFAGGANAVCQQAVTGLLDHLQDNSDVWLGALWWAAGPWWGDYMYSFEPPSGTGYVNYNSILKKYLP

SEQ ID NO: 77 - DNA, 1232 bp, BASIDIOMYCETE CBS 495.95
ggatccacttagtaacggccgccagtgtgctggaaagcatgaagtctctcttcctgtcacttgtagcgaccgtcgcgctcagctcgccagtattctctgtcgcagtctgggggcaatgcggcggcattggcttcagcggaagcaccgtctgtgatgcaggcgccggctgtgtgaagctcaacgactattactctcaatgccaacccggcgctcccactgctacatccgcggcgccaagtagcaacgcaccgtccggcacttcgacggcctcggccccctcctccagcctttgctctggcagccgcacgccgttccagttcttcggtgtcaacgaatccggcgcggagttcggcaacctgaacatccccggtgttctgggcaccgactacacctggccgtcgccatccagcattgacttcttcatgggcaagggaatgaataccttccgtattccgttcctcatggagcgtcttgtcccccctgccactggcatcacaggacctctcgaccagacgtacttgggcggcctgcagacgattgtcaactacatcaccggcaaaggcggctttgctctcattgacccgcacaactttatgatctacaatggccagacgatctccagtaccagcgacttccagaagttctggcagaacctcgcaggagtgtttaaatcgaacagtcacgtcatcttcgatgttatgaacgagcctcacgatattcccgcccagaccgtgttccaactgaaccaagccgctgtcaatggcatccgtgcgagcggtgcgacgtcgcagctcattctggtcgagggcacaagctggactggagcctggacctggacgacctctggcaacagcgatgcattcggtgccattaaggatcccaacaacaacgtcgcgatccagatgcatcagtacctggatagcgatggctctggcacttcgcagacctgcgtgtctcccaccatcggtgccgagcggttgcaggctgcgactcaatggttgaagcagaacaacctcaagggcttcctgggcgagatcggcgccggctctaactccgcttgcatcagcgctgtgcagggtgcgttgtgttcgatgcagcaatctggtgtgtggctcggcgctctctggtgggctgcgggcccgtggtggggcgactactaccagtccatcgagccgccctctggcccggcggtgtccgcgatcctcccgcaggccctgctgccgttcgcgtaa

SEQ ID NO: 78 - protein, 397 aa, BASIDIOMYCETE CBS 495.95
MKSLFLSLVATVALSSPVFSVAVWGQCGGIGFSGSTVCDAGAGCVKLNDYYSQCQPGAPTATSAAPSSNAPSGTSTASAPSSSLCSGSRTPFQFFGVNESGAEFGNLNIPGVLGTDYTWPSPSSIDFFMGKGMNTFRIPFLMERLVPPATGITGPLDQTYLGGLQTIVNYITGKGGFALIDPHNFMIYNGQTISSTSDFQKFWQNLAGVFKSNSHVIFDVMNEPHDIPAQTVFQLNQAAVNGIRASGATSQLILVEGTSWTGAWTWTTSGNSDAFGAIKDPNNNVAIQMHQYLDSDGSGTSQTCVSPTIGAERLQAATQWLKQNNLKGFLGEIGAGSNSACISAVQGALCSMQQSGVWLGALWWAAGPWWGDYYQSIEPPSGPAVSAILPQALLPFA

SEQ ID NO: 79 - DNA, 1303 bp, BASIDIOMYCETE CBS 495.95
ggaaagcgtcagtatggtgaaatttgcgcttgtggcaactgtcggcgcaatcttgagcgcttctgcggccaatgcggcttctatctaccagcaatgtggaggcattggatggtctgggtccactgtttgcgacgccggtctcgcttgcgttatcctcaatgcgtactactttcagtgcttgacgcccgccgcgggccagacaacgacgggctcgggcgcaccggcgtcaacatcaacctctcactcaacggtcactacggggagctcacactcaacaaccgggacgacggcgacgaaaacaactaccactccgtcgaccaccacgaccctacccgccatctctgtgtctggtcgcgtctgctctggctccaggacgaagttcaagttcttcggtgtgaatgaaagcggcgccgaattcgggaacactgcttggccagggcagctcgggaaagactatacatggccttcgcctagcagcgtggactacttcatgggggctggattcaatacattccgtatcaccttcttgatggagcgtatgagccctccggctaccggactcactggcccattcaaccagacgtacctgtcgggcctcaccaccattgtcgactacatcacgaacaaaggaggatacgctcttattgacccccacaacttcatgcgttacaacaacggcataatcagcagcacatctgacttcgcgacttggtggagcaatttggccactgtattcaaatccacgaagaacgccatcttcgacatccagaacgagccgtacggaatcgatgcgcagaccgtatacgaactgaatcaagctgccatcaattcgatccgcgccgctggcgctacgtcacagttgattctggttgaaggaacgtcatacactggagcttggacgtgggtctcgtccggaaacggagctgctttcgcggccgttacggatccttacaacaacacggcaattgaaatgcaccaatacctcgacagcgacggttctgggacaaacgaagactgtgtctcctccaccattgggtcgcaacgtctccaagctgccactgcgtggctgcaacaaacaggactcaagggattcctcggagagacgggtgctgggtcgaattcccagtgcatcgacgccgtgttcgatgaactttgctatatgcaacagcaaggcggctcctggatcggtgcactctggtgggctgcgggtccctggtggggcacgtacatttactcgattgaacctccgagcggtgccgctatcccagaagtccttcctcagggtctcgctccattcctctag

SEQ ID NO: 80 - protein, 429 aa, BASIDIOMYCETE CBS 495.95
MVKFALVATVGAILSASAANAASIYQQCGGIGWSGSTVCDAGLACVILNAYYFQCLTPAAGQTTTGSGAPASTSTSHSTVTTGSSHSTTGTTATKTTTTPSTTTTLPAISVSGRVCSGSRTKFKFFGVNESGAEFGNTAWPGQLGKDYTWPSPSSVDYFMGAGFNTFRITFLMERMSPPATGLTGPFNQTYLSGLTTIVDYITNKGGYALIDPHNFMRYNNGIISSTSDFATWWSNLATVFKSTKNAIFDIQNEPYGIDAQTVYELNQAAINSIRAAGATSQLILVEGTSYTGAWTWVSSGNGAAFAAVTDPYNNTAIEMHQYLDSDGSGTNEDCVSSTIGSQRLQAATAWLQQTGLKGFLGETGAGSNSQCIDAVFDELCYMQQQGGSWIGALWWAAGPWWGTYIYSIEPPSGAAIPEVLPQGLAPFL

SEQ ID NO: 81 - DNA, 1580 bp, Thielavia terrestris
agccccccgttcaggcacacttggcatcagatcagcttagcagcgcctgcacagcatgaagctctcgcagtcggccgcgctggcggcactcaccgcgacggcgctcgccgccccctcgcccacgacgccgcaggcgccgaggcaggcttcagccggctgctcgtctgcggtcacgctcgacgccagcaccaacgtttggaagaagtacacgctgcaccccaacagctactaccgcaaggaggttgaggccgcggtggcgcagatctcggacccggacctcgccgccaaggccaagaaggtggccgacgtcggcaccttcctgtggctcgactcgatcgagaacatcggcaagctggagccggcgatccaggacgtgccctgcgagaacatcctgggcctggtcatctacgacctgccgggccgcgactgcgcggccaaggcgtccaacggcgagctcaaggtcggcgagatcgaccgctacaagaccgagtacatcgacagtgagtgctgccccccgggttcgagaagagcgtgggggaaagggaaagggttgactgactgacacggcgcactgcagagatcgtgtcgatcctcaaggcacaccccaacacggcgttcgcgctggtcatcgagccggactcgctgcccaacctggtgaccaacagcaacttggacacgtgctcgagcagcgcgtcgggctaccgcgaaggcgtggcttacgccctcaagaacctcaacctgcccaacgtgatcatgtacctcgacgccggccacggcggctggctcggctgggacgccaacctgcagcccggcgcgcaggagctagccaaggcgtacaagaacgccggctcgcccaagcagctccgcggcttctcgaccaacgtggccggctggaactcctggtgagcttttttccattccatttcttcttcctcttctctcttcgctcccactctgcagccccccctcccccaagcacccactggcgttccggcttgctgactcggcctccctttccccgggcaccagggatcaatcgcccggcgaattctcccaggcgtccgacgccaagtacaacaagtgccagaacgagaagatctacgtcagcaccttcggctccgcgctccagtcggccggcatgcccaaccacgccatcgtcgacacgggccgcaacggcgtcaccggcctgcgcaaggagtggggtgactggtgcaacgtcaacggtgcaggttcgttgtcttctttttctcctcttttgtttgcacgtcgtggtccttttcaagcagccgtgtttggttgggggagatggactccggctgatgttctgcttcctctctaggcttcggcgtgcgcccgacgagcaacacgggcctcgagctggccgacgcgttcgtgtgggtcaagcccggcggcgagtcggacggcaccagcgacagctcgtcgccgcgctacgacagcttctgcggcaaggacgacgccttcaagccctcgcccgaggccggcacctggaacgaggcctacttcgagatgctgctcaagaacgccgtgccgtcgttctaagacggtccagcatcatccgg

SEQ ID NO: 82 - protein, 396 aa, Thielavia terrestris
MKLSQSAALAALTATALAAPSPTTPQAPRQASAGCSSAVTLDASTNVWKKYTLHPNSYYRKEVEAAVAQISDPDLAAKAKKVADVGTFLWLDSIENIGKLEPAIQDVPCENILGLVIYDLPGRDCAAKASNGELKVGEIDRYKTEYIDKIVSILKAHPNTAFALVIEPDSLPNLVTNSNLDTCSSSASGYREGVAYALKNLNLPNVIMYLDAGHGGWLGWDANLQPGAQELAKAYKNAGSPKQLRGFSTNVAGWNSWDQSPGEFSQASDAKYNKCQNEKIYVSTFGSALQSAGMPNHAIVDTGRNGVTGLRKEWGDWCNVNGAGFGVRPTSNTGLELADAFVWVKPGGESDGTSDSSSPRYDSFCGKDDAFKPSPEAGTWNEAYFEMLLKNAVPSF

SEQ ID NO: 83 - DNA, 1203 bp, Thielavia terrestris
atgaagtacctcaacctcctcgcagctctcctcgccgtcgctcctctctccctcgctgcacccagcatcgaggccagacagtcgaacgtcaacccatacatcggcaagagcccgctcgttattaggtcgtacgcccaaaagcttgaggagaccgtcaggaccttccagcaacgtggcgaccagctcaacgctgcgaggacacggacggtgcagaacgttgcgactttcgcctggatctcggataccaatggtattggagccattcgacctctcatccaagatgctctcgcccagcaggctcgcactggacagaaggtcatcgtccaaatcgtcgtctacaacctcccagatcgcgactgctctgccaacgcctcgactggagagttcaccgtaggaaacgacggtctcaaccgatacaagaactttgtcaacaccatcgcccgcgagctctcgactgctgacgctgacaagctccactttgccctcctcctcgaacccgacgcacttgccaacctcgtcaccaacgcgaatgcccccaggtgccgaatcgccgctcccgcttacaaggagggtatcgcctacaccctcgccaccttgtccaagcccaacgtcgacgtctacatcgacgccgccaacggtggctggctcggctggaacgacaacctccgccccttcgccgaactcttcaaggaagtctacgacctcgcccgccgcatcaaccccaacgccaaggtccgcggcgtccccgtcaacgtctccaactacaaccagtaccgcgctgaagtccgcgagcccttcaccgagtggaaggacgcctgggacgagagccgctacgtcaacgtcctcaccccgcacctcaacgccgtcggcttctccgcgcacttcatcgttgaccagggacgcggtggcaagggcggtatcaggacggagtggggccagtggtgcaacgttaggaacgctgggttcggtatcaggcctactgcggatcagggcgtgctccagaacccgaatgtggatgcgattgtgtgggttaagccgggtggagagtcggatggcacgagtgatttgaactcgaacaggtatgatcctacgtgcaggagtccggtggcgcatgttcccgctcctgaggctggccagtggttcaacgagtatgttgttaacctcgttttgaacgctaacccccctcttgagcctacctggtaa

SEQ ID NO: 84 - protein, 400 aa, Thielavia terrestris
MKYLNLLAALLAVAPLSLAAPSIEARQSNVNPYIGKSPLVIRSYAQKLEETVRTFQQRGDQLNAARTRTVQNVATFAWISDTNGIGAIRPLIQDALAQQARTGQKVIVQIVVYNLPDRDCSANASTGEFTVGNDGLNRYKNFVNTIARELSTADADKLHFALLLEPDALANLVTNANAPRCRIAAPAYKEGIAYTLATLSKPNVDVYIDAANGGWLGWNDNLRPFAELFKEVYDLARRINPNAKVRGVPVNVSNYNQYRAEVREPFTEWKDAWDESRYVNVLTPHLNAVGFSAHFIVDQGRGGKGGIRTEWGQWCNVRNAGFGIRPTADQGVLQNPNVDAIVWVKPGGESDGTSDLNSNRYDPTCRSPVAHVPAPEAGQWFNEYVVNLVLNANPPLEPTW

SEQ ID NO: 85 - DNA, 1501 bp, Thielavia terrestris
gccgttgtcaagatgggccagaagacgctgcacggattcgccgccacggctttggccgttctcccctttgtgaaggctcagcagcccggcaacttcacgccggaggtgcacccgcaactgccaacgtggaagtgcacgaccgccggcggctgcgttcagcaggacacttcggtggtgctcgactggaactaccgttggatccacaatgccgacggcaccgcctcgtgcacgacgtccagcggggtcgaccacacgctgtgtccagatgaggcgacctgcgcgaagaactgcttcgtggaaggcgtcaactacacgagcagcggtgtcaccacatccggcagttcgctgacgatgaggcagtatttcaaggggagcaacgggcagaccaacagcgtttcgcctcgtctctacctgctcggctcggatggaaactacgtaatgctcaagctgctcggccaggagctgagcttcgatgtcgatctctccacgctcccctgcggcgagaacggcgcgctgtacctgtccgagatggacgcgaccggtggcaggaaccagtacaacaccggcggtgccaactacggctcgggctactgtgacgcccagtgtcccgtgcagacgtggatgaacggcacgctgaacaccaacgggcagggctactgctgcaacgagatggacatcctcgaggccaactcccgcgccaacgcgatgacacctcacccctgcgccaacggcagctgcgacaagagcgggtgcggactcaacccctacgccgagggctacaagagctactacggaccgggcctcacggttgacacgtcgaagcccttcaccatcattacccgcttcatcaccgacgacggcacgaccagcggcaccctcaaccagatccagcggatctatgtgcagaatggcaagacggtcgcgtcggctgcgtccggaggcgacatcatcacggcatccggctgcacctcggcccaggcgttcggcgggctggccaacatgggcgcggcgcttggacggggcatggtgctgaccttcagcatctggaacgacgctgggggctacatgaactggctcgacagcggcaacaacggcccgtgcagcagcaccgagggcaacccgtccaacatcctggccaactacccggacacccacgtggtcttctccaacatccgctggggagacatcggctcgacggtccaggtctcgggaggcggcaacggcggctcgaccaccaccacgtcgaccaccacgctgaggacctcgaccacgaccaccaccaccgccccgacggccactgccacgcactggggacaatgcggcggaatcggggtacgtcaaccgcctcctgcattctgttgaggaagttaactaacgtggcctacgcagtggactggaccgaccgtctgcgaatcgccgtacgcatgcaaggagctgaacccctggtactaccagtgcctctaaagtattgcagtgaagccatactccgtgctcggcatgg

SEQ ID NO: 86 - protein, 464 aa, Thielavia terrestris
MGQKTLHGFAATALAVLPFVKAQQPGNFTPEVHPQLPTWKCTTAGGCVQQDTSVVLDWNYRWIHNADGTASCTTSSGVDHTLCPDEATCAKNCFVEGVNYTSSGVTTSGSSLTMRQYFKGSNGQTNSVSPRLYLLGSDGNYVMLKLLGQELSFDVDLSTLPCGENGALYLSEMDATGGRNQYNTGGANYGSGYCDAQCPVQTWMNGTLNTNGQGYCCNEMDILEANSRANAMTPHPCANGSCDKSGCGLNPYAEGYKSYYGPGLTVDTSKPFTIITRFITDDGTTSGTLNQIQRIYVQNGKTVASAASGGDIITASGCTSAQAFGGLANMGAALGRGMVLTFSIWNDAGGYMNWLDSGNNGPCSSTEGNPSNILANYPDTHVVFSNIRWGDIGSTVQVSGGGNGGSTTTTSTTTLRTSTTTTTTAPTATATHWGQCGGIGWTGPTVCESPYACKELNPWYYQCL

SEQ ID NO: 87 - DNA, 1368 bp, Thielavia terrestris
accgatccgctcgaagatggcgcccaagtctacagttctggccgcctggctgctctcctcgctggccgcggcccagcagatcggcaaagccgtgcccgaggtccaccccaaactgacaacgcagaagtgcactctccgcggcgggtgcaagcctgtccgcacctcggtcgtgctcgactcgtccgcgcgctcgctgcacaaggtcggggaccccaacaccagctgcagcgtcggcggcgacctgtgctcggacgcgaagtcgtgcggcaagaactgcgcgctcgagggcgtcgactacgcggcccacggcgtggcgaccaagggcgacgccctcacgctgcaccagtggctcaagggggccgacggcacctacaggaccgtctcgccgcgcgtatacctcctgggcgaggacgggaagaactacgaggacttcaagctgctcaacgccgagctcagcttcgacgtcgacgtgtcccagctcgtctgcggcatgaacggcgccctgtacttctccgagatggagatggacggcggccgcagcccgctgaacccggcgggcgccacgtacggcacgggctactgcgacgcgcagtgccccaagttggactttatcaacggcgaggtatttcttctctcttctgtttttcttttccatcgctttttctgaccggaatccgccctcttagctcaacaccaaccacacgtacggggcgtgctgcaacgagatggacatctgggaggccaacgcgctggcgcaggcgctcacgccgcacccgtgcaacgcgacgcgggtgtacaagtgcgacacggcggacgagtgcgggcagccggtgggcgtgtgcgacgaatgggggtgctcgtacaacccgtccaacttcggggtcaaggactactacgggcgcaacctgacggtggacacgaaccgcaagttcacggtgacgacgcagttcgtgacgtccaacgggcgggcggacggcgagctgaccgagatccggcggctgtacgtgcaggacggcgtggtgatccagaaccacgcggtcacggcgggcggggcgacgtacgacagcatcacggacggcttctgcaacgcgacggccacctggacgcagcagcggggcgggctcgcgcgcatgggcgaggccatcggccgcggcatggtgctcatcttcagcctgtgggttgacaacggcggcttcatgaactggctcgacagcggcaacgccgggccctgcaacgccaccgagggcgacccggccctgatcctgcagcagcacccggacgccagcgtcaccttctccaacatccgatggggcgagatcggcagcacgtacaagagcgagtgcagccactagagtagagcttgtaatt

SEQ ID NO: 88 - protein, 423 aa, Thielavia terrestris
MAPKSTVLAAWLLSSLAAAQQIGKAVPEVHPKLTTQKCTLRGGCKPVRTSVVLDSSARSLHKVGDPNTSCSVGGDLCSDAKSCGKNCALEGVDYAAHGVATKGDALTLHQWLKGADGTYRTVSPRVYLLGEDGKNYEDFKLLNAELSFDVDVSQLVCGMNGALYFSEMEMDGGRSPLNPAGATYGTGYCDAQCPKLDFINGELNTNHTYGACCNEMDIWEANALAQALTPHPCNATRVYKCDTADECGQPVGVCDEWGCSYNPSNFGVKDYYGRNLTVDTNRKFTVTTQFVTSNGRADGELTEIRRLYVQDGVVIQNHAVTAGGATYDSITDGFCNATATWTQQRGGLARMGEAIGRGMVLIFSLWVDNGGFMNWLDSGNAGPCNATEGDPALILQQHPDASVTFSNIRWGEIGSTYKSECSH

SEQ ID NO: 89 - DNA, 1011 bp, Thielavia terrestris
atgaccctacggctccctgtcatcagcctgctggcctcgctggcagcaggcgccgtcgtcgtcccacgggcggagtttcacccccctctcccgacttggaaatgcacgacctccgggggctgcgtgcagcagaacaccagcgtcgtcctggaccgtgactcgaagtacgccgcacacagcgccggctcgcggacggaatcggattacgcggcaatgggagtgtccacttcgggcaatgccgtgacgctgtaccactacgtcaagaccaacggcaccctcgtccccgcttcgccgcgcatctacctcctgggcgcggacggcaagtacgtgcttatggacctcctcaaccaggagctgtcggtggacgtcgacttctcggcgctgccgtgcggcgagaacggggccttctacctgtccgagatggcggcggacgggcggggcgacgcgggggcgggcgacgggtactgcgacgcgcagtgccagggctactgctgcaacgagatggacatcctcgaggccaactcgatggcgacggccatgacgccgcacccgtgcaagggcaacaactgcgaccgcagcggctgcggctacaacccgtacgccagcggccagcgcggcttctacgggcccggcaagacggtcgacacgagcaagcccttcaccgtcgtcacgcagttcgccgccagcggcggcaagctgacccagatcacccgcaagtacatccagaacggccgggagatcggcggcggcggcaccatctccagctgcggctccgagtcttcgacgggcggcctgaccggcatgggcgaggcgctggggcgcggaatggtgctggccatgagcatctggaacgacgcggcccaggagatggcatggctcgatgccggcaacaacggcccttgcgccagtggccagggcagcccgtccgtcattcagtcgcagcatcccgacacccacgtcgtcttctccaacatcaggtggggcgacatcgggtctaccacgaagaactag

SEQ ID NO: 90 - protein, 336 aa, Thielavia terrestris
MTLRLPVISLLASLAAGAVVVPRAEFHPPLPTWKCTTSGGCVQQNTSVVLDRDSKYAAHSAGSRTESDYAAMGVSTSGNAVTLYHYVKTNGTLVPASPRIYLLGADGKYVLMDLLNQELSVDVDFSALPCGENGAFYLSEMAADGRGDAGAGDGYCDAQCQGYCCNEMDILEANSMATAMTPHPCKGNNCDRSGCGYNPYASGQRGFYGPGKTVDTSKPFTVVTQFAASGGKLTQITRKYIQNGREIGGGGTISSCGSESSTGGLTGMGEALGRGMVLAMSIWNDAAQEMAWLDAGNNGPCASGQGSPSVIQSQHPDTHVVFSNIRWGDIGSTTKN

SEQ ID NO: 91 - DNA, 1480 bp, Cladorrhinum foecundissimum
gatccgaattcctcctctcgttctttagtcacagaccagacatctgcccacgatggttcacaagttcgccctcctcaccggcctcgccgcctccctcgcatctgcccagcagatcggcaccgtcgtccccgagtctcaccccaagcttcccaccaagcgctgcactctcgccggtggctgccagaccgtcgacacctccatcgtcatcgacgccttccagcgtcccctccacaagatcggcgacccttccactccttgcgtcgtcggcggccctctctgccccgacgccaagtcctgcgctgagaactgcgcgctcgagggtgtcgactatgcctcctggggcatcaagaccgagggcgacgccctaactctcaaccagtggatgcccgacccggcgaaccctggccagtacaagacgactactccccgtacttaccttgttgctgaggacggcaagaactacgaggatgtgaagctcctggctaaggagatctcgtttgatgccgatgtcagcaaccttccctgcggcatgaacggtgctttctacttgtctgagatgttgatggatggtggacgtggcgacctcaaccctgctggtgccgagtatggtaccggttactgtgatgcgcagtgcttcaagttggatttcatcaacggcgaggccaacatcgaccaaaagcacggcgcctgctgcaacgaaatggacattttcgaatccaactcgcgcgccaagaccttcgtcccccacccctgcaacatcacgcaggtctacaagtgcgaaggcgaagacgagtgcggccagcccgtcggcgtgtgcgacaagtgggggtgcggcttcaacgagtacaaatggggcgtcgagtccttctacggccggggctcgcagttcgccatcgactcctccaagaagttcaccgtcaccacgcagttcctgaccgacaacggcaaggaggacggcgtcctcgtcgagatccgccgcttgtggcaccaggatggcaagctgatcaagaacaccgctatccaggttgaggagaactacagcacggactcggtgagcaccgagttctgcgagaagactgcttctttcaccatgcagcgcggtggtctcaaggcgatgggcgaggctatcggtcgtggtatggtgctggttttcagcatctgggcggatgattcgggttttatgaactggttggatgcggagggtaatggcccttgcagcgcgactgagggcgatccgaaggagattgtcaagaataagccggatgctagggttacgttctcaaacattaggattggtgaggttggtagcacgtatgctccgggtgggaagtgcggtgttaagagcagggttgctagggggcttactgcttcttaaggggggtgtgaagagaggaggaggtgttgttgggggttggagatgataattgggcgagatggtgtagagcgggttggttggatatgaatacgttgaattggatgt

SEQ ID NO: 92 - protein, 440 aa, Cladorrhinum foecundissimum
MVHKFALLTGLAASLASAQQIGTVVPESHPKLPTKRCTLAGGCQTVDTSIVIDAFQRPLHKIGDPSTPCVVGGPLCPDAKSCAENCALEGVDYASWGIKTEGDALTLNQWMPDPANPGQYKTTTPRTYLVAEDGKNYEDVKLLAKEISFDADVSNLPCGMNGAFYLSEMLMDGGRGDLNPAGAEYGTGYCDAQCFKLDFINGEANIDQKHGACCNEMDIFESNSRAKTFVPHPCNITQVYKCEGEDECGQPVGVCDKWGCGFNEYKWGVESFYGRGSQFAIDSSKKFTVTTQFLTDNGKEDGVLVEIRRLWHQDGKLIKNTAIQVEENYSTDSVSTEFCEKTASFTMQRGGLKAMGEAIGRGMVLVFSIWADDSGFMNWLDAEGNGPCSATEGDPKEIVKNKPDARVTFSNIRIGEVGSTYAPGGKCGVKSRVARGLTAS

SEQ ID NO: 93 - DNA, 1380 bp, Trichoderma reesei
atggcgccctcagttacactgccgttgaccacggccatcctggccattgcccggctcgtcgccgcccagcaaccgggtaccagcacccccgaggtccatcccaagttgacaacctacaagtgtacaaagtccggggggtgcgtggcccaggacacctcggtggtccttgactggaactaccgctggatgcacgacgcaaactacaactcgtgcaccgtcaacggcggcgtcaacaccacgctctgccctgacgaggcgacctgtggcaagaactgcttcatcgagggcgtcgactacgccgcctcgggcgtcacgacctcgggcagcagcctcaccatgaaccagtacatgcccagcagctctggcggctacagcagcgtctctcctcggctgtatctcctggactctgacggtgagtacgtgatgctgaagctcaacggccaggagctgagcttcgacgtcgacctctctgctctgccgtgtggagagaacggctcgctctacctgtctcagatggacgagaacgggggcgccaaccagtataacacggccggtgccaactacgggagcggctactgcgatgctcagtgccccgtccagacatggaggaacggcaccctcaacactagccaccagggcttctgctgcaacgagatggatatcctggagggcaactcgagggcgaatgccttgacccctcactcttgcacggccacggcctgcgactctgccggttgcggcttcaacccctatggcagcggctacaaaagctactacggccccggagataccgttgacacctccaagaccttcaccatcatcacccagttcaacacggacaacggctcgccctcgggcaaccttgtgagcatcacccgcaagtaccagcaaaacggcgtcgacatccccagcgcccagcccggcggcgacaccatctcgtcctgcccgtccgcctcagcctacggcggcctcgccaccatgggcaaggccctgagcagcggcatggtgctcgtgttcagcatttggaacgacaacagccagtacatgaactggctcgacagcggcaacgccggcccctgcagcagcaccgagggcaacccatccaacatcctggccaacaaccccaacacgcacgtcgtcttctccaacatccgctggggagacattgggtctactacgaactcgactgcgcccccgcccccgcctgcgtccagcacgacgttttcgactacacggaggagctcgacgacttcgagcagcccgagctgcacgcagactcactgggggcagtgcggtggcattgggtacagcgggtgcaagacgtgcacgtcgggcactacgtgccagtatagcaacgactactactcgcaatgcctttag

SEQ ID NO: 94 - protein, 459 aa, Trichoderma reesei; identical to SEQ ID NO: 66

SEQ ID NO: 95 - DNA, 1545 bp, Trichoderma reesei
atgtatcggaagttggccgtcatctcggccttcttggccacagctcgtgctcagtcggcctgcactctccaatcggagactcacccgcctctgacatggcagaaatgctcgtctggtggcacgtgcactcaacagacaggctccgtggtcatcgacgccaactggcgctggactcacgctacgaacagcagcacgaactgctacgatggcaacacttggagctcgaccctatgtcctgacaacgagacctgcgcgaagaactgctgtctggacggtgccgcctacgcgtccacgtacggagttaccacgagcggtaacagcctctccattggctttgtcacccagtctgcgcagaagaacgttggcgctcgcctttaccttatggcgagcgacacgacctaccaggaattcaccctgcttggcaacgagttctctttcgatgttgatgtttcgcagctgccgtgcggcttgaacggagctctctacttcgtgtccatggacgcggatggtggcgtgagcaagtatcccaccaacaccgctggcgccaagtacggcacggggtactgtgacagccagtgtccccgcgatctgaagttcatcaatggccaggccaacgttgagggctgggagccgtcatccaacaacgcgaacacgggcattggaggacacggaagctgctgctctgagatggatatctgggaggccaactccatctccgaggctcttaccccccacccttgcacgactgtcggccaggagatctgcgagggtgatgggtgcggcggaacttactccgataacagatatggcggcacttgcgatcccgatggctgcgactggaacccataccgcctgggcaacaccagcttctacggccctggctcaagctttaccctcgataccaccaagaaattgaccgttgtcacccagttcgagacgtcgggtgccatcaaccgatactatgtccagaatggcgtcactttccagcagcccaacgccgagcttggtagttactctggcaacgagctcaacgatgattactgcacagctgaggaggcagaattcggcggatcctctttctcagacaagggcggcctgactcagttcaagaaggctacctctggcggcatggttctggtcatgagtctgtgggatgattactacgccaacatgctgtggctggactccacctacccgacaaacgagacctcctccacacccggtgccgtgcgcggaagctgctccaccagctccggtgtccctgctcaggtcgaatctcagtctcccaacgccaaggtcaccttctccaacatcaagttcggacccattggcagcaccggcaaccctagcggcggcaaccctcccggcggaaacccgcctggcaccaccaccacccgccgcccagccactaccactggaagctctcccggacctacccagtctcactacggccagtgcggcggtattggctacagcggccccacggtctgcgccagcggcacaacttgccaggtcctgaacccttactactctcagtgcctgtaa

SEQ ID NO: 96 - protein, 514 aa, Trichoderma reesei
MYRKLAVISAFLATARAQSACTLQSETHPPLTWQKCSSGGTCTQQTGSVVIDANWRWTHATNSSTNCYDGNTWSSTLCPDNETCAKNCCLDGAAYASTYGVTTSGNSLSIGFVTQSAQKNVGARLYLMASDTTYQEFTLLGNEFSFDVDVSQLPCGLNGALYFVSMDADGGVSKYPTNTAGAKYGTGYCDSQCPRDLKFINGQANVEGWEPSSNNANTGIGGHGSCCSEMDIWEANSISEALTPHPCTTVGQEICEGDGCGGTYSDNRYGGTCDPDGCDWNPYRLGNTSFYGPGSSFTLDTTKKLTVVTQFETSGAINRYYVQNGVTFQQPNAELGSYSGNELNDDYCTAEEAEFGGSSFSDKGGLTQFKKATSGGMVLVMSLWDDYYANMLWLDSTYPTNETSSTPGAVRGSCSTSSGVPAQVESQSPNAKVTFSNIKFGPIGSTGNPSGGNPPGGNPPGTTTTRRPATTTGSSPGPTQSHYGQCGGIGYSGPTVCASGTTCQVLNPYYSQCL

SEQ ID NO: 97 - DNA, 1611 bp, Trichoderma reesei
atgattgtcggcattctcaccacgctggctacgctggccacactcgcagctagtgtgcctctagaggagcggcaagcttgctcaagcgtctggtaattatgtgaaccctctcaagagacccaaatactgagatatgtcaaggggccaatgtggtggccagaattggtcgggtccgacttgctgtgcttccggaagcacatgcgtctactccaacgactattactcccagtgtcttcccggcgctgcaagctcaagctcgtccacgcgcgccgcgtcgacgacttctcgagtatcccccacaacatcccggtcgagctccgcgacgcctccacctggttctactactaccagagtacctccagtcggatcgggaaccgctacgtattcaggcaacccttttgttggggtcactccttgggccaatgcatattacgcctctgaagttagcagcctcgctattcctagcttgactggagccatggccactgctgcagcagctgtcgcaaaggttccctcttttatgtggctgtaggtcctcccggaaccaaggcaatctgttactgaaggctcatcattcactgcagagatactcttgacaagacccctctcatggagcaaaccttggccgacatccgcaccgccaacaagaatggcggtaactatgccggacagtttgtggtgtatgacttgccggatcgcgattgcgctgcccttgcctcgaatggcgaatactctattgccgatggtggcgtcgccaaatataagaactatatcgacaccattcgtcaaattgtcgtggaatattccgatatccggaccctcctggttattggtatgagtttaaacacctgcctccccccccccttcccttcctttcccgccggcatcttgtcgttgtgctaactattgttccctcttccagagcctgactctcttgccaacctggtgaccaacctcggtactccaaagtgtgccaatgctcagtcagcctaccttgagtgcatcaactacgccgtcacacagctgaaccttccaaatgttgcgatgtatttggacgctggccatgcaggatggcttggctggccggcaaaccaagacccggccgctcagctatttgcaaatgtttacaagaatgcatcgtctccgagagctcttcgcggattggcaaccaatgtcgccaactacaacgggtggaacattaccagccccccatcgtacacgcaaggcaacgctgtctacaacgagaagctgtacatccacgctattggacgtcttcttgccaatcacggctggtccaacgccttcttcatcactgatcaaggtcgatcgggaaagcagcctaccggacagcaacagtggggagactggtgcaatgtgatcggcaccggatttggtattcgcccatccgcaaacactggggactcgttgctggattcgtttgtctgggtcaagccaggcggcgagtgtgacggcaccagcgacagcagtgcgccacgatttgactcccactgtgcgctcccagatgccttgcaaccggcgcctcaagctggtgcttggttccaagcctactttgtgcagcttctcacaaacgcaaacccatcgttcctgtaa

SEQ ID NO: 98 - protein, 471 aa, Trichoderma reesei
MIVGILTTLATLATLAASVPLEERQACSSVWGQCGGQNWSGPTCCASGSTCVYSNDYYSQCLPGAASSSSSTRAASTTSRVSPTTSRSSSATPPPGSTTTRVPPVGSGTATYSGNPFVGVTPWANAYYASEVSSLAIPSLTGAMATAAAAVAKVPSFMWLDTLDKTPLMEQTLADIRTANKNGGNYAGQFVVYDLPDRDCAALASNGEYSIADGGVAKYKNYIDTIRQIVVEYSDIRTLLVIEPDSLANLVTNLGTPKCANAQSAYLECINYAVTQLNLPNVAMYLDAGHAGWLGWPANQDPAAQLFANVYKNASSPRALRGLATNVANYNGWNITSPPSYTQGNAVYNEKLYIHAIGRLLANHGWSNAFFITDQGRSGKQPTGQQQWGDWCNVIGTGFGIRPSANTGDSLLDSFVWVKPGGECDGTSDSSAPRFDSHCALPDALQPAPQAGAWFQAYFVQLLTNANPSFL

SEQ ID NO: 99 - DNA, 2046 bp, Humicola insolens
gccgtgaccttgcgcgctttgggtggcggtggcgagtcgtggacggtgcttgctggtcgccggccttcccggcgatccgcgtgatgagagggccaccaacggcgggatgatgctccatggggaacttccccatggagaagagagagaaacttgcggagccgtgatctggggaaagatgctccgtgtctcgtctatataactcgagtctccccgagccctcaacaccaccagctctgatctcaccatccccatcgacaatcacgcaaacacagcagttgtcgggccattccttcagacacatcagtcaccctccttcaaaatgcgtaccgccaagttcgccaccctcgccgcccttgtggcctcggccgccgcccagcaggcgtgcagtctcaccaccgagaggcacccttccctctcttggaacaagtgcaccgccggcggccagtgccagaccgtccaggcttccatcactctcgactccaactggcgctggactcaccaggtgtctggctccaccaactgctacacgggcaacaagtgggatactagcatctgcactgatgccaagtcgtgcgctcagaactgctgcgtcgatggtgccgactacaccagcacctatggcatcaccaccaacggtgattccctgagcctcaagttcgtcaccaagggccagcactcgaccaacgtcggctcgcgtacctacctgatggacggcgaggacaagtatcagagtacgttctatcttcagccttctcgcgccttgaatcctggctaacgtttacacttcacagccttcgagctcctcggcaacgagttcaccttcgatgtcgatgtctccaacatcggctgcggtctcaacggcgccctgtacttcgtctccatggacgccgatggtggtctcagccgctatcctggcaacaaggctggtgccaagtacggtaccggctactgcgatgctcagtgcccccgtgacatcaagttcatcaacggcgaggccaacattgagggctggaccggctccaccaacgaccccaacgccggcgcgggccgctatggtacctgctgctctgagatggatatctgggaagccaacaacatggctactgccttcactcctcacccttgcaccatcattggccagagccgctgcgagggcgactcgtgcggtggcacctacagcaacgagcgctacgccggcgtctgcgaccccgatggctgcgacttcaactcgtaccgccagggcaacaagaccttctacggcaagggcatgaccgtcgacaccaccaagaagatcactgtcgtcacccagttcctcaaggatgccaacggcgatctcggcgagatcaagcgcttctacgtccaggatggcaagatcatccccaactccgagtccaccatccccggcgtcgagggcaattccatcacccaggactggtgcgaccgccagaaggttgcctttggcgacattgacgacttcaaccgcaagggcggcatgaagcagatgggcaaggccctcgccggccccatggtcctggtcatgtccatctgggatgaccacgcctccaacatgctctggctcgactcgaccttccctgtcgatgccgctggcaagcccggcgccgagcgcggtgcctgcccgaccacctcgggtgtccctgctgaggttgaggccgaggcccccaacagcaacgtcgtcttctccaacatccgcttcggccccatcggctcgaccgttgctggtctccccggcgcgggcaacggcggcaacaacggcggcaaccccccgccccccaccaccaccacctcctcggctccggccaccaccaccaccgccagcgctggccccaaggctggccgctggcagcagtgcggcggcatcggcttcactggcccgacccagtgcgaggagccctacatttgcaccaagctcaacgactggtactctcagtgcctgtaaattctgagtcgctgactcgacgatcacggccggtttttgcatgaaaggaaacaaacgaccgcgataaaaatggagggtaatgagatgtc

SEQ ID NO: 100 - protein, 525 aa, Humicola insolens
MRTAKFATLAALVASAAAQQACSLTTERHPSLSWNKCTAGGQCQTVQASITLDSNWRWTHQVSGSTNCYTGNKWDTSICTDAKSCAQNCCVDGADYTSTYGITTNGDSLSLKFVTKGQHSTNVGSRTYLMDGEDKYQTFELLGNEFTFDVDVSNIGCGLNGALYFVSMDADGGLSRYPGNKAGAKYGTGYCDAQCPRDIKFINGEANIEGWTGSTNDPNAGAGRYGTCCSEMDIWEANNMATAFTPHPCTIIGQSRCEGDSCGGTYSNERYAGVCDPDGCDFNSYRQGNKTFYGKGMTVDTTKKITVVTQFLKDANGDLGEIKRFYVQDGKIIPNSESTIPGVEGNSITQDWCDRQKVAFGDIDDFNRKGGMKQMGKALAGPMVLVMSIWDDHASNMLWLDSTFPVDAAGKPGAERGACPTTSGVPAEVEAEAPNSNVVFSNIRFGPIGSTVAGLPGAGNGGNNGGNPPPPTTTTSSAPATTTTASAGPKAGRWQQCGGIGFTGPTQCEEPYICTKLNDWYSQCL

SEQ ID NO: 101 - DNA, 1812 bp, Myceliophthora thermophila
atggccaagaagcttttcatcaccgccgcccttgcggctgccgtgttggcggcccccgtcattgaggagcgccagaactgcggcgctgtgtggtaagaaagcccggtctgagtttcccatgactttctcatcgagtaatggcataaggcccaccccttcgactgactgtgagaatcgatcaaatccaggactcaatgcggcggcaacgggtggcagggtcccacatgctgcgcctcgggctcgacctgcgttgcgcagaacgagtggtactctcagtgcctgcccaacaatcaggtgacgagttccaacactccgtcgtcgacttccacctcgcagcgcagcagcagcacctccagcagcagcaccaggagcggcagctcctcctcctccaccaccacgccccctcccgtctccagccccgtgactagcattcccggcggtgcgaccaccacggcgagctactctggcaaccccttctcgggcgtccggctcttcgccaacgactactacaggtccgaggtccacaatctcgccattcctagcatgaccggtactctggcggccaaggcttccgccgtcgccgaagtccctagcttccagtggctcgaccggaacgtcaccatcgacaccctgatggtccagactctgtcccagatccgggctgccaataatgccggtgccaatcctccctatgctggtgagttacatggcggcgacttgccttctcgtcccccacctttcttgacgggatcggttacctgacctggaggcaaaacaaaaccagcccaacttgtcgtctacgacctccccgaccgtgactgcgccgccgctgcgtccaacggcgagttttcgattgcaaacggcggcgccgccaactacaggagctacatcgacgctatccgcaagcacatcattgagtactcggacatccggatcatcctggttatcgagcccgactcgatggccaacatggtgaccaacatgaacgtggccaagtgcagcaacgccgcgtcgacgtaccacgagttgaccgtgtacgcgctcaagcagctgaacctgcccaacgtcgccatgtatctcgacgccggccacgccggctggctcggctggcccgccaacatccagcccgccgccgacctgtttgccggcatctacaatgacgccggcaagccggctgccgtccgcggcctggccactaacgtcgccaactacaacgcctggagtatcgcttcggccccgtcgtacacgtcccctaaccctaactacgacgagaagcactacatcgaggccttcagcccgctcctgaacgcggccggcttccccgcacgcttcattgtcgacactggccgcaacggcaaacaacctaccggtatggtttttttctttttttttctctgttcccctcccccttccccttcagttggcgtccacaaggtctcttagtcttgcttcttctcggaccaaccttcccccacccccaaaacgcaccgcccacaaccgttcgactctatactcttgggaatgggcgccgaaactgaccgttcgacaggccaacaacagtggggtgactggtgcaatgtcaagggcactggctttggcgtgcgcccgacggccaacacgggccacgacctggtcgatgcctttgtctgggtcaagcccggcggcgagtccgacggcacaagcgacaccagcgccgcccgctacgactaccactgcggcctgtccgatgccctgcagcctgctccggaggctggacagtggttccaggcctacttcgagcagctgctcaccaacgccaacccgcccttctaa

SEQ ID NO: 102 - protein, 482 aa, Myceliophthora thermophila
MAKKLFITAALAAAVLAAPVIEERQNCGAVWTQCGGNGWQGPTCCASGSTCVAQNEWYSQCLPNNQVTSSNTPSSTSTSQRSSSTSSSSTRSGSSSSSTTTPPPVSSPVTSIPGGATTTASYSGNPFSGVRLFANDYYRSEVHNLAIPSMTGTLAAKASAVAEVPSFQWLDRNVTIDTLMVQTLSQIRAANNAGANPPYAAQLVVYDLPDRDCAAAASNGEFSIANGGAANYRSYIDAIRKHIIEYSDIRIILVIEPDSMANMVTNMNVAKCSNAASTYHELTVYALKQLNLPNVAMYLDAGHAGWLGWPANIQPAADLFAGIYNDAGKPAAVRGLATNVANYNAWSIASAPSYTSPNPNYDEKHYIEAFSPLLNAAGFPARFIVDTGRNGKQPTGQQQWGDWCNVKGTGFGVRPTANTGHDLVDAFVWVKPGGESDGTSDTSAARYDYHCGLSDALQPAPEAGQWFQAYFEQLLTNANPPF

SEQ ID NO: 103 - DNA, 1802 bp, Myceliophthora thermophila
atggccaagaagcttttcatcaccgccgcgcttgcggctgccgtgttggcggcccccgtcattgaggagcgccagaactgcggcgctgtgtggtaagaaagcccggtccgagtctcccatgattttctcgtcgagtaatggcataagggccaccccttcgactgaccgtgagaatcgatcaaatccaggactcaatgcggcggtaacgggtggcaaggtcccacatgctgcgcctcgggctcgacctgcgttgcgcagaacgagtggtactctcagtgcctgcccaacagccaggtgacgagttccaccactccgtcgtcgacttccacctcgcagcgcagcaccagcacctccagcagcaccaccaggagcggcagctcctcctcctcctccaccacgcccccgcccgtctccagccccgtgaccagcattcccggcggtgcgacctccacggcgagctactctggcaaccccttctcgggcgtccggctcttcgccaacgactactacaggtccgaggtccacaatctcgccattcctagcatgactggtactctggcggccaaggcttccgccgtcgccgaagtccctagcttccagtggctcgaccggaacgtcaccatcgacaccctgatggtccagactctgtcccaggtccgggctctcaataaggccggtgccaatcctccctatgctggtgagttacatggcgacttgccttctcgtcccctacctttcttgacgggatcggttacctgacctggaggcaaaacaacaacagcccaactcgtcgtctacgacctccccgaccgtgactgtgccgccgctgcgtccaacggcgagttttcgattgcaaacggcggcgccgccaactacaggagctacatcgacgctatccgcaagcacatcattgagtactcggacatccggatcatcctggttatcgagcccgactcgatggccaacatggtgaccaacatgaacgtggccaagtgcagcaacgccgcgtcgacgtaccacgagttgaccgtgtacgcgctcaagcagctgaacctgcccaacgtcgccatgtatctcgacgccggccacgccggctggctcggctggcccgccaacatccagcccgccgccgagctgtttgccggcatctacaatgatgccggcaagccggctgccgtccgcggcctggccactaacgtcgccaactacaacgcctggagcatcgcttcggccccgtcgtacacgtcgcctaaccctaactacgacgagaagcactacatcgaggccttcagcccgctcttgaactcggccggcttccccgcacgcttcattgtcgacactggccgcaacggcaaacaacctaccggtatgtttttttttcttttgtctctgtcccccccttttctcccccttcagttggcgtccacaaggtctcttagtcctgcttcatctgtgaccaacctccccccccccggcaccgcccacaaccgtttgactctatactcttgggaatgggcgccgaaactgaccgttccacaggccaacaacagtggggtgactggtgcaatgtcaagggcaccggctttggcgtgcgcccgacggccaacacgggccacgagctggtcgatgcctttgtctgggtcaagcccggcggcgagtccgacggcacaagcgacaccagcgccgcccgctacgactaccactgcggcctgtccgatgccctgcagcctgcccccgaggctggacagtggttccaggcctacttcgagcagctgctcaccaacgccaacccgcccttctaa

SEQ ID NO: 104 - protein, 481 aa, Myceliophthora thermophila
MAKKLFITAALAAAVLAAPVIEERQNCGAVWTQCGGNGWQGPTCCASGSTCVAQNEWYSQCLPNSQVTSSTTPSSTSTSQRSTSTSSSTTRSGSSSSSSTTPPPVSSPVTSIPGGATSTASYSGNPFSGVRLFANDYYRSEVHNLAIPSMTGTLAAKASAVAEVPSFQWLDRNVTIDTLMVQTLSQVRALNKAGANPPYAAQLVVYDLPDRDCAAAASNGEFSIANGGAANYRSYIDAIRKHIIEYSDIRIILVIEPDSMANMVTNMNVAKCSNAASTYHELTVYALKQLNLPNVAMYLDAGHAGWLGWPANIQPAAELFAGIYNDAGKPAAVRGLATNVANYNAWSIASAPSYTSPNPNYDEKHYIEAFSPLLNSAGFPARFIVDTGRNGKQPTGQQQWGDWCNVKGTGFGVRPTANTGHELVDAFVWVKPGGESDGTSDTSAARYDYHCGLSDALQPAPEAGQWFQAYFEQLLTNANPP

SEQ ID NO: 105 - DNA, 1446 bp, Thielavia terrestris
atggctcagaagctccttctcgccgccgcccttgcggccagcgccctcgctgctcccgtcgtcgaggagcgccagaactgcggttccgtctggagccaatgcggcggcattggctggtccggcgcgacctgctgcgcttcgggcaatacctgcgttgagctgaacccgtactactcgcagtgcctgcccaacagccaggtgactacctcgaccagcaagaccacctccaccaccaccaggagcagcaccaccagccacagcagcggtcccaccagcacgagcaccaccaccaccagcagtcccgtggtcactaccccgccgagtacctccatccccggcggtgcctcgtcaacggccagctggtccggcaacccgttctcgggcgtgcagatgtgggccaacgactactacgcctccgaggtctcgtcgctggccatccccagcatgacgggcgccatggccaccaaggcggccgaggtggccaaggtgcccagcttccagtggcttgaccgcaacgtcaccatcgacacgctgttcgcccacacgctgtcgcagatccgcgcggccaaccagaaaggcgccaacccgccctacgcgggcatcttcgtggtctacgaccttccggaccgcgactgcgccgccgccgcgtccaacggcgagttctccatcgcgaacaacggggcggccaactacaagacgtacatcgacgcgatccggagcctcgtcatccagtactcagacatccgcatcatcttcgtcatcgagcccgactcgctggccaacatggtgaccaacctgaacgtggccaagtgcgccaacgccgagtcgacctacaaggagttgaccgtctacgcgctgcagcagctgaacctgcccaacgtggccatgtacctggacgccggccacgccggctggctcggctggcccgccaacatccagccggccgccaacctcttcgccgagatctacacgagcgccggcaagccggccgccgtgcgcggcctcgccaccaacgtggccaactacaacggctggagcctggccacgccgccctcgtacacccagggcgaccccaactacgacgagagccactacgtccaggccctcgccccgctgctcaccgccaacggcttccccgcccacttcatcaccgacaccggccgcaacggcaagcagccgaccggacaacggcaatggggagactggtgcaacgttatcggaactggcttcggcgtgcgcccgacgacaaacaccggcctcgacatcgaggacgccttcgtctgggtcaagcccggcggcgagtgcgacggcacgagcaacacgacctctccccgctacgactaccactgcggcctgtcggacgcgctgcagcctgctccggaggccggcacttggttccaggcctacttcgagcagctcctgaccaacgccaacccgcccttttaa

SEQ ID NO: 106 - protein, 481 aa, Thielavia terrestris
MAQKLLLAAALAASALAAPVVEERQNCGSVWSQCGGIGWSGATCCASGNTCVELNPYYSQCLPNSQVTTSTSKTTSTTTRSSTTSHSSGPTSTSTTTTSSPVVTTPPSTSIPGGASSTASWSGNPFSGVQMWANDYYASEVSSLAIPSMTGAMATKAAEVAKVPSFQWLDRNVTIDTLFAHTLSQIRAANQKGANPPYAGIFVVYDLPDRDCAAAASNGEFSIANNGAANYKTYIDAIRSLVIQYSDIRIIFVIEPDSLANMVTNLNVAKCANAESTYKELTVYALQQLNLPNVAMYLDAGHAGWLGWPANIQPAANLFAEIYTSAGKPAAVRGLATNVANYNGWSLATPPSYTQGDPNYDESHYVQALAPLLTANGFPAHFITDTGRNGKQPTGQRQWGDWCNVIGTGFGVRPTTNTGLDIEDAFVWVKPGGECDGTSNTTSPRYDYHCGLSDALQPAPEAGTWFQAYFEQLLTNANPPF

SEQ ID NO: 107 - DNA, 1593 bp, Chaetomium thermophilum
atgatgtacaagaagttcgccgctctcgccgccctcgtggctggcgccgccgcccagcaggcttgctccctcaccactgagacccaccccagactcacttggaagcgctgcacctctggcggcaactgctcgaccgtgaacggcgccgtcaccatcgatgccaactggcgctggactcacactgtttccggctcgaccaactgctacaccggcaacgagtgggatacctccatctgctctgatggcaagagctgcgcccagacctgctgcgtcgacggcgctgactactcttcgacctatggtatcaccaccagcggtgactccctgaacctcaagttcgtcaccaagcaccagcacggcaccaatgtcggctctcgtgtctacctgatggagaacgacaccaagtaccagatgttcgagctcctcggcaacgagttcaccttcgatgtcgatgtctctaacctgggctgcggtctcaacggcgccctctacttcgtctccatggacgctgatggtggtatgagcaagtactctggcaacaaggctggcgccaagtacggtaccggctactgcgatgctcagtgcccgcgcgaccttaagttcatcaacggcgaggccaacattgagaactggaccccttcgaccaatgatgccaacgccggtttcggccgctatggcagctgctgctctgagatggatatctgggatgccaacaacatggctactgccttcactcctcacccttgcaccattatcggccagagccgctgcgagggcaacagctgcggtggcacctacagctctgagcgctatgctggtgtttgcgatcctgatggctgcgacttcaacgcctaccgccagggcgacaagaccttctacggcaagggcatgaccgtcgacaccaccaagaagatgaccgtcgtcacccagttccacaagaactcggctggcgtcctcagcgagatcaagcgcttctacgttcaggacggcaagatcattgccaacgccgagtccaagatccccggcaaccccggcaactccatcacccaggagtggtgcgatgcccagaaggtcgccttcggtgacatcgatgacttcaaccgcaagggcggtatggctcagatgagcaaggccctcgagggccctatggtcctggtcatgtccgtctgggatgaccactacgccaacatgctctggctcgactcgacctaccccattgacaaggccggcacccccggcgccgagcgcggtgcttgcccgaccacctccggtgtccctgccgagattgaggcccaggtccccaacagcaacgttatcttctccaacatccgcttcggccccatcggctcgaccgtccctggcctcgacggcagcacccccagcaacccgaccgccaccgttgctcctcccacttctaccaccaccagcgtgagaagcagcactactcagatttccaccccgactagccagcccggcggctgcaccacccagaagtggggccagtgcggtggtatcggctacaccggctgcactaactgcgttgctggcactacctgcactgagctcaacccctggtacagccagtgcctgtaa

SEQ ID NO: 108 - protein, 530 aa, Chaetomium thermophilum
MMYKKFAALAALVAGAAAQQACSLTTETHPRLTWKRCTSGGNCSTVNGAVTIDANWRWTHTVSGSTNCYTGNEWDTSICSDGKSCAQTCCVDGADYSSTYGITTSGDSLNLKFVTKHQHGTNVGSRVYLMENDTKYQMFELLGNEFTFDVDVSNLGCGLNGALYFVSMDADGGMSKYSGNKAGAKYGTGYCDAQCPRDLKFINGEANIENWTPSTNDANAGFGRYGSCCSEMDIWDANNMATAFTPHPCTIIGQSRCEGNSCGGTYSSERYAGVCDPDGCDFNAYRQGDKTFYGKGMTVDTTKKMTVVTQFHKNSAGVLSEIKRFYVQDGKIIANAESKIPGNPGNSITQEWCDAQKVAFGDIDDFNRKGGMAQMSKALEGPMVLVMSVWDDHYANMLWLDSTYPIDKAGTPGAERGACPTTSGVPAEIEAQVPNSNVIFSNIRFGPIGSTVPGLDGSTPSNPTATVAPPTSTTTSVRSSTTQISTPTSQPGGCTTQKWGQCGGIGYTGCTNCVAGTTCTELNPWYSQCL

SEQ ID NO: 109 - DNA, 1434 bp, Chaetomium thermophilum
atggctaagcagctgctgctcactgccgctcttgcggccacttcgctggctgcccctctccttgaggagcgccagagctgctcctccgtctggggtcaatgcggtggcatcaattacaacggcccgacctgctgccagtccggcagtgtttgcacttacctgaatgactggtacagccagtgcattcccggtcaggctcagcccggcacgactagcaccacggctcggaccaccagcaccagcaccaccagcacttcgtcggtccgcccgaccacctcgaatacccctgtgacgactgctcccccgacgaccaccatcccgggcggcgcctcgagcacggccagctacaacggcaacccgttttcgggtgttcaactttgggccaacacctactactcgtccgaggtgcacactttggccatccccagcttgtctcctgagctggctgccaaggccgccaaggtcgctgaggttcccagcttccagtggctcgaccgcaatgtgactgttgacactctcttctccggcactcttgccgaaatccgcgccgccaaccagcgcggtgccaacccgccttatgccggcattttcgtggtttatgacttaccagaccgtgattgcgcggctgctgcttcgaacggcgagtggtctatcgccaacaatggtgccaacaactacaagcgctacatcgaccggatccgtgagctccttatccagtactccgatatccgcactattctggtcattgaacctgattccctggccaacatggtcaccaacatgaacgtccagaagtgctcgaacgctgcctccacttacaaggagcttactgtctatgccctcaaacagctcaatcttcctcacgttgccatgtacatggatgctggccacgctggctggcttggctggcccgccaacatccagcctgctgctgagctctttgctcaaatctaccgcgacgctggcaggcccgctgctgtccgcggtcttgcgaccaacgttgccaactacaatgcttggtcgatcgccagccctccgtcctacacctctcctaacccgaactacgacgagaagcactatattgaggcctttgctcctcttctccgcaaccagggcttcgacgcaaagttcatcgtcgacaccggccgtaacggcaagcagcccactggccagcttgaatggggtcactggtgcaatgtcaagggaactggcttcggtgtgcgccctactgctaacactgggcatgaacttgttgatgctttcgtgtgggtcaagcccggtggcgagtccgacggcaccagtgcggacaccagcgctgctcgttatgactatcactgcggcctttccgacgcactgactccggcgcctgaggctggccaatggttccaggcttatttcgaacagctgctcatcaatgccaaccctccgctctga

SEQ ID NO: 110 - protein, 477 aa, Chaetomium thermophilum
MAKQLLLTAALAATSLAAPLLEERQSCSSVWGQCGGINYNGPTCCQSGSVCTYLNDWYSQCIPGQAQPGTTSTTARTTSTSTTSTSSVRPTTSNTPVTTAPPTTTIPGGASSTASYNGNPFSGVQLWANTYYSSEVHTLAIPSLSPELAAKAAKVAEVPSFQWLDRNVTVDTLFSGTLAEIRAANQRGANPPYAGIFVVYDLPDRDCAAAASNGEWSIANNGANNYKRYIDRIRELLIQYSDIRTILVIEPDSLANMVTNMNVQKCSNAASTYKELTVYALKQLNLPHVAMYMDAGHAGWLGWPANIQPAAELFAQIYRDAGRPAAVRGLATNVANYNAWSIASPPSYTSPNPNYDEKHYIEAFAPLLRNQGFDAKFIVDTGRNGKQPTGQLEWGHWCNVKGTGFGVRPTANTGHELVDAFVWVKPGGESDGTSADTSAARYDYHCGLSDALTPAPEAGQWFQAYFEQLLINANPPL

SEQ ID NO: 111 - DNA, 2586 bp, Aspergillus oryzae
atgaagcttggttggatcgaggtggccgcattggcggctgcctcagtagtcagtgccaaggatgatctcgcgtactcccctcctttctacccttccccatgggcagatggtcagggtgaatgggcggaagtatacaaacgcgctgtagacatagtttcccagatgacgttgacagagaaagtcaacttaacgactggaacaggatggcaactagagaggtgtgttggacaaactggcagtgttcccagactcaacatccccagcttgtgtttgcaggatagtcctcttggtattcgtttctcggactacaattcagctttccctgcgggtgttaatgtcgctgccacctgggacaagacgctcgcctaccttcgtggtcaggcaatgggtgaggagttcagtgataagggtattgacgttcagctgggtcctgctgctggccctctcggtgctcatccggatggcggtagaaactgggaaggtttctcaccagatccagccctcaccggtgtactttttgcggagacgattaagggtattcaagatgctggtgtcattgcgacagctaagcattatatcatgaacgaacaagagcatttccgccaacaacccgaggctgcgggttacggattcaacgtaagcgacagtttgagttccaacgttgatgacaagactatgcatgaattgtacctctggcccttcgcggatgcagtacgcgctggagtcggtgctgtcatgtgctcttacaaccaaatcaacaacagctacggttgcgagaatagcgaaactctgaacaagcttttgaaggcggagcttggtttccaaggcttcgtcatgagtgattggaccgctcatcacagcggcgtaggcgctgctttagcaggtctggatatgtcgatgcccggtgatgttaccttcgatagtggtacgtctttctggggtgcaaacttgacggtcggtgtccttaacggtacaatcccccaatggcgtgttgatgacatggctgtccgtatcatggccgcttattacaaggttggccgcgacaccaaatacacccctcccaacttcagctcgtggaccagggacgaatatggtttcgcgcataaccatgtttcggaaggtgcttacgagagggtcaacgaattcgtggacgtgcaacgcgatcatgccgacctaatccgtcgcatcggcgcgcagagcactgttctgctgaagaacaagggtgccttgcccttgagccgcaaggaaaagctggtcgcccttctgggagaggatgcgggttccaactcgtggggcgctaacggctgtgatgaccgtggttgcgataacggtacccttgccatggcctggggtagcggtactgcgaatttcccatacctcgtgacaccagagcaggcgattcagaacgaagttcttcagggccgtggtaatgtcttcgccgtgaccgacagttgggcgctcgacaagatcgctgcggctgcccgccaggccagcgtatctctcgtgttcgtcaactccgactcaggagaaggctatcttagtgtggatggaaatgagggcgatcgtaacaacatcactctgtggaagaacggcgacaatgtggtcaagaccgcagcgaataactgtaacaacaccgttgtcatcatccactccgtcggaccagttttgatcgatgaatggtatgaccaccccaatgtcactggtattctctgggctggtctgccaggccaggagtctggtaactccattgccgatgtgctgtacggtcgtgtcaaccctggcgccaagtctcctttcacttggggcaagacccgggagtcgtatggttctcccttggtcaaggatgccaacaatggcaacggagcgccccagtctgatttcacccagggtgttttcatcgattaccgccatttcgataagttcaatgagacccctatctacgagtttggctacggcttgagctacaccaccttcgagctctccgacctccatgttcagcccctgaacgcgtcccgatacactcccaccagtggcatgactgaagctgcaaagaactttggtgaaattggcgatgcgtcggagtacgtgtatccggaggggctggaaaggatccatgagtttatctatccctggatcaactctaccgacctgaaggcatcgtctgacgattctaactacggctgggaagactccaagtatattcccgaaggcgccacggatgggtctgcccagccccgtttgcccgctagtggtggtgccggaggaaaccccggtctgtacgaggatcttttccgcgtctctgtgaaggtcaagaacacgggcaatgtcgccggtgatgaagttcctcagctgtacgtttccctaggcggcccgaatgagcccaaggtggtactgcgcaagtttgagcgtattcacttggccccttcgcaggaggccgtgtggacaacgacccttacccgtcgtgaccttgcaaactgggacgtttcggctcaggactggaccgtcactccttaccccaagacgatctacgttggaaactcctcacggaaactgccgctccaggcctcgctgcctaaggcccagtaa

SEQ ID NO: 112 - protein, 861 aa, Aspergillus oryzae
MKLGWIEVAALAAASVVSAKDDLAYSPPFYPSPWADGQGEWAEVYKRAVDIVSQMTLTEKVNLTTGTGWQLERCVGQTGSVPRLNIPSLCLQDSPLGIRFSDYNSAFPAGVNVAATWDKTLAYLRGQAMGEEFSDKGIDVQLGPAAGPLGAHPDGGRNWEGFSPDPALTGVLFAETIKGIQDAGVIATAKHYIMNEQEHFRQQPEAAGYGFNVSDSLSSNVDDKTMHELYLWPFADAVRAGVGAVMCSYNQINNSYGCENSETLNKLLKAELGFQGFVMSDWTAHHSGVGAALAGLDMSMPGDVTFDSGTSFWGANLTVGVLNGTIPQWRVDDMAVRIMAAYYKVGRDTKYTPPNFSSWTRDEYGFAHNHVSEGAYERVNEFVDVQRDHADLIRRIGAQSTVLLKNKGALPLSRKEKLVALLGEDAGSNSWGANGCDDRGCDNGTLAMAWGSGTANFPYLVTPEQAIQNEVLQGRGNVFAVTDSWALDKIAAAARQASVSLVFVNSDSGEGYLSVDGNEGDRNNITLWKNGDNVVKTAANNCNNTVVIIHSVGPVLIDEWYDHPNVTGILWAGLPGQESGNSIADVLYGRVNPGAKSPFTWGKTRESYGSPLVKDANNGNGAPQSDFTQGVFIDYRHFDKFNETPIYEFGYGLSYTTFELSDLHVQPLNASRYTPTSGMTEAAKNFGEIGDASEYVYPEGLERIHEFIYPWINSTDLKASSDDSNYGWEDSKYIPEGATDGSAQPRLPASGGAGGNPGLYEDLFRVSVKVKNTGNVAGDEVPQLYVSLGGPNEPKVVLRKFERIHLAPSQEAVWTTTLTRRDLANWDVSAQDWTVTPYPKTIYVGNSSRKLPLQASLPKAQ

SEQ ID NO: 113 - DNA, 3060 bp, Aspergillus fumigatus
atgagattcggttggctcgaggtggccgctctgacggccgcttctgtagccaatgcccaggtttgtgatgctttcccgtcattgtttcggatatagttgacaatagtcatggaaataatcaggaattggctttctctccaccattctacccttcgccttgggctgatggccagggagagtgggcagatgcccatcgacgcgccgtcgagatcgtttctcagatgacactggcggagaaggttaaccttacaacgggtactgggtgggttgcgacttttttgttgacagtgagctttcttcactgaccatctacacagatgggaaatggaccgatgcgtcggtcaaaccggcagcgttcccaggtaagcttgcaattctgcaacaacgtgcaagtgtagttgctaaaacgcggtggtgcagacttggtatcaactggggtctttgtggccaggattcccctttgggtatccgtttctgtgagctatacccgcggagtctttcagtccttgtattatgtgctgatgattgtctctgtatagctgacctcaactccgccttccctgctggtactaatgtcgccgcgacatgggacaagacactcgcctaccttcgtggcaaggccatgggtgaggaattcaacgacaagggcgtggacattttgctggggcctgctgctggtcctctcggcaaatacccggacggcggcagaatctgggaaggcttctctcctgatccggttctcactggtgtacttttcgccgaaactatcaagggtatccaagacgcgggtgtgattgctactgccaagcattacattctgaatgaacaggagcatttccgacaggttggcgaggcccagggatatggttacaacatcacggagacgatcagctccaacgtggatgacaagaccatgcacgagttgtacctttggtgagtagttgacactgcaaatgaggaccttgattgatttgactgacctggaatgcaggccctttgcagatgctgtgcgcggtaagattttccgtagacttgacctcgcgacgaagaaatcgctgacgaaccatcgtagctggcgttggcgctgtcatgtgttcctacaatcaaatcaacaacagctacggttgtcaaaacagtcaaactctcaacaagctcctcaaggctgagctgggcttccaaggcttcgtcatgagtgactggagcgctcaccacagcggtgtcggcgctgccctcgctgggttggatatgtcgatgcctggagacatttccttcgacgacggactctccttctggggcacgaacctaactgtcagtgttcttaacggcaccgttccagcctggcgtgtcgatgacatggctgttcgtatcatgaccgcgtactacaaggttggtcgtgaccgtcttcgtattccccctaacttcagctcctggacccgggatgagtacggctgggagcattctgctgtctccgagggagcctggaccaaggtgaacgacttcgtcaatgtgcagcgcagtcactctcagatcatccgtgagattggtgccgctagtacagtgctcttgaagaacacgggtgctcttcctttgaccggcaaggaggttaaagtgggtgttctcggtgaagacgctggttccaacccgtggggtgctaacggctgccccgaccgcggctgtgataacggcactcttgctatggcctggggtagtggtactgccaacttcccttaccttgtcacccccgagcaggctatccagcgagaggtcatcagcaacggcggcaatgtctttgctgtgactgataacggggctctcagccagatggcagatgttgcatctcaatccaggtgagtgcgggctcttagaaaaagaacgttctctgaatgaagttttttaaccattgcgaacagcgtgtctttggtgtttgtcaacgccgactctggagagggtttcatcagtgtcgacggcaacgagggtgaccgcaaaaatctcactctgtggaagaacggcgaggccgtcattgacactgttgtcagccactgcaacaacacgattgtggttattcacagtgttgggcccgtcttgatcgaccggtggtatgataaccccaacgtcactgccatcatctgggccggcttgcccggtcaggagagtggcaactccctggtcgacgtgctctatggccgcgtcaaccccagcgccaagaccccgttcacctggggcaagactcgggagtcttacggggctcccttgctcaccgagcctaacaatggcaatggtgctccccaggatgatttcaacgagggcgtcttcattgactaccgtcactttgacaagcgcaatgagacccccatttatgagtttggccatggcttgagctacaccacctttggttactctcaccttcgggttcaggccctcaatagttcgagttcggcatatgtcccgactagcggagagaccaagcctgcgccaacctatggtgagatcggtagtgccgccgactacctgtatcccgagggtctcaaaagaattaccaagtttatttacccttggctcaactcgaccgacctcgaggattcttctgacgacccgaactacggctgggaggactcggagtacattcccgaaggcgctagggatgggtctcctcaacccctcctgaaggctggcggcgctcctggtggtaaccctaccctttatcaggatcttgttagggtgtcggccaccataaccaacactggtaacgtcgccggttatgaagtccctcaattggtgagtgacccgcatgttccttgcgttgcaatttggctaactcgcttctagtatgtttcactgggcggaccgaacgagcctcgggtcgttctgcgcaagttcgaccgaatcttcctggctcctggggagcaaaaggtttggaccacgactcttaaccgtcgtgatctcgccaattgggatgtggaggctcaggactgggtcatcacaaagtaccccaagaaagtgcacgtcggcagctcctcgcgtaagctgcctctgagagcgcctctgccccgtgtctactag

SEQ ID NO: 114 - protein, 863 aa, Aspergillus fumigatus
MRFGWLEVAALTAASVANAQELAFSPPFYPSPWADGQGEWADAHRRAVEIVSQMTLAEKVNLTTGTGWEMDRCVGQTGSVPRLGINWGLCGQDSPLGIRFSDLNSAFPAGTNVAATWDKTLAYLRGKAMGEEFNDKGVDILLGPAAGPLGKYPDGGRIWEGFSPDPVLTGVLFAETIKGIQDAGVIATAKHYILNEQEHFRQVGEAQGYGYNITETISSNVDDKTMHELYLWPFADAVRAGVGAVMCSYNQINNSYGCQNSQTLNKLLKAELGFQGFVMSDWSAHHSGVGAALAGLDMSMPGDISFDDGLSFWGTNLTVSVLNGTVPAWRVDDMAVRIMTAYYKVGRDRLRIPPNFSSWTRDEYGWEHSAVSEGAWTKVNDFVNVQRSHSQIIREIGAASTVLLKNTGALPLTGKEVKVGVLGEDAGSNPWGANGCPDRGCDNGTLAMAWGSGTANFPYLVTPEQAIQREVISNGGNVFAVTDNGALSQMADVASQSSVSLVFVNADSGEGFISVDGNEGDRKNLTLWKNGEAVIDTVVSHCNNTIVVIHSVGPVLIDRWYDNPNVTAIIWAGLPGQESGNSLVDVLYGRVNPSAKTPFTWGKTRESYGAPLLTEPNNGNGAPQDDFNEGVFIDYRHFDKRNETPIYEFGHGLSYTTFGYSHLRVQALNSSSSAYVPTSGETKPAPTYGEIGSAADYLYPEGLKRITKFIYPWLNSTDLEDSSDDPNYGWEDSEYIPEGARDGSPQPLLKAGGAPGGNPTLYQDLVRVSATITNTGNVAGYEVPQLYVSLGGPNEPRVVLRKFDRIFLAPGEQKVWTTTLNRRDLANWDVEAQDWVITKYPKKVHVGSSSRKLPLRAPLPRVY

SEQ ID NO: 115 - DNA, 2800 bp, Penicillium brasilianum
tgaaaatgcagggttctacaatctttctggctttcgcctcatgggcgagccaggttgctgccattgcgcagcccatacagaagcacgaggtttgttttatcttgctcatggacgtgctttgacttgactaattgttttacatacagcccggatttctgcacgggccccaagccatagaatcgttctcagaaccgttctacccgtcgccctggatgaatcctcacgccgagggctgggaggccgcatatcagaaagctcaagattttgtctcgcaactcactatcttggagaaaataaatctgaccaccggtgttgggtaagtctctccgactgcttctgggtcacggtgcgacgagccactgactttttgaagctgggaaaatgggccgtgtgtaggaaacactggatcaattcctcgtctcggattcaaaggattttgtacccaggattcaccacagggtgttcggttcgcagattattcctccgctttcacatctagccaaatggccgccgcaacatttgaccgctcaattctttatcaacgaggccaagccatggcacaggaacacaaggctaagggtatcacaattcaattgggccctgttgccggccctctcggtcgcatccccgagggcggccgcaactgggaaggattctcccctgatcctgtcttgactggtatagccatggctgagacaattaagggcatgcaggatactggagtgattgcttgcgctaaacattatattggaaacgagcaggagcacttccgtcaagtgggtgaagctgcgggtcacggatacactatttccgatactatttcatctaatattgacgaccgtgctatgcatgagctatacttgtggccatttgctgatgccgttcgcgctggtgtgggttctttcatgtgctcatactctcagatcaacaactcctacggatgccaaaacagtcagaccctcaacaagctcctcaagagcgaattgggcttccaaggctttgtcatgagcgattggggtgcccatcactctggagtgtcatcggcgctagctggacttgatatgagcatgccgggtgataccgaatttgattctggcttgagcttctggggctctaacctcaccattgcaattctgaacggcacggttcccgaatggcgcctggatgacatggcgatgcgaattatggctgcatacttcaaagttggccttactattgaggatcaaccagatgtcaacttcaatgcctggacccatgacacctacggatataaatacgcttatagcaaggaagattacgagcaggtcaactggcatgtcgatgttcgcagcgaccacaataagctcattcgcgagactgccgcgaagggtacagttctgctgaagaacaactttcatgctctccctctgaagcagcccaggttcgtggccgtcgttggtcaggatgccgggccaaaccccaagggccctaacggctgcgcagaccgaggatgcgaccaaggcactctcgcaatgggatggggctcagggtctaccgaattcccttacctggtcactcctgacactgctattcagtcaaaggtcctcgaatacgggggtcgatacgagagtatttttgataactatgacgacaatgctatcttgtcgcttgtctcacagcctgatgcaacctgtatcgtttttgcaaatgccgattccggtgaaggctacatcactgtcgacaacaactggggtgaccgcaacaatctgaccctctggcaaaatgccgatcaagtgattagcactgtcagctcgcgatgcaacaacacaatcgttgttctccactctgtcggaccagtgttgctaaatggtatatatgagcacccgaacatcacagctattgtctgggcagggatgccaggcgaagaatctggcaatgctctcgtggatattctttggggcaatgttaaccctgccggtcgcactccgttcacctgggccaaaagtcgagaggactatggcactgatataatgtacgagcccaacaacggccagcgtgcgcctcagcaggatttcaccgagagcatctacctcgactaccgccatttcgacaaagctggtatcgagccaatttacgagtttggattcggcctctcctataccaccttcgaatactctgacctccgtgttgtgaagaagtatgttcaaccatacagtcccacgaccggcaccggtgctcaagcaccttccatcggacagccacctagccagaacctggatacctacaagttccctgctacatacaagtacatcaaaaccttcatttatccctacctgaacagcactgtctccctccgcgctgcttccaaggatcccgaatacggtcgtacagactttatcccaccccacgcgcgtgatggctcccctcaacctctcaaccccgctggagacccagtggccagtggtggaaacaacatgctctacgacgaactttacgaggtcactgcacagatcaaaaacactggcgacgtggccggcgacgaagtcgtccagctttacgtagatctcgggggtgacaacccgcctcgtcagttgagaaactttgacaggttttatctgctgcccggtcagagctcaacattccgggctacattgacgcgccgtgatttgagcaactgggatattgaggcgcagaactggcgagttacggaatcgcctaagagagtgtatgttggacggtcgagtcgggatttgccgctgagctcacaattggagtaatgatcatgtctaccaatagatgttgaatgtctggtgtggatatt

SEQ ID NO: 116 - protein, 878 aa, Penicillium brasilianum
MQGSTIFLAFASWASQVAAIAQPIQKHEPGFLHGPQAIESFSEPFYPSPWMNPHAEGWEAAYQKAQDFVSQLTILEKINLTTGVGWENGPCVGNTGSIPRLGFKGFCTQDSPQGVRFADYSSAFTSSQMAAATFDRSILYQRGQAMAQEHKAKGITIQLGPVAGPLGRIPEGGRNWEGFSPDPVLTGIAMAETIKGMQDTGVIACAKHYIGNEQEHFRQVGEAAGHGYTISDTISSNIDDRAMHELYLWPFADAVRAGVGSFMCSYSQINNSYGCQNSQTLNKLLKSELGFQGFVMSDWGAHHSGVSSALAGLDMSMPGDTEFDSGLSFWGSNLTIAILNGTVPEWRLDDMAMRIMAAYFKVGLTIEDQPDVNFNAWTHDTYGYKYAYSKEDYEQVNWHVDVRSDHNKLIRETAAKGTVLLKNNFHALPLKQPRFVAVVGQDAGPNPKGPNGCADRGCDQGTLAMGWGSGSTEFPYLVTPDTAIQSKVLEYGGRYESIFDNYDDNAILSLVSQPDATCIVFANADSGEGYITVDNNWGDRNNLTLWQNADQVISTVSSRCNNTIVVLHSVGPVLLNGIYEHPNITAIVWAGMPGEESGNALVDILWGNVNPAGRTPFTWAKSREDYGTDIMYEPNNGQRAPQQDFTESIYLDYRHFDKAGIEPIYEFGFGLSYTTFEYSDLRVVKKYVQPYSPTTGTGAQAPSIGQPPSQNLDTYKFPATYKYIKTFIYPYLNSTVSLRAASKDPEYGRTDFIPPHARDGSPQPLNPAGDPVASGGNNMLYDELYEVTAQIKNTGDVAGDEVVQLYVDLGGDNPPRQLRNFDRFYLLPGQSSTFRATLTRRDLSNWDIEAQNWRVTESPKRVYVGRSSRDLPLSSQLE

SEQ ID NO: 117 - DNA, 2583 bp, Aspergillus niger
atgaggttcactttgatcgaggcggtggctctgactgccgtctcgctggccagcgctgatgaattggcctactccccaccgtattacccatccccttgggccaatggccagggcgactgggcgcaggcataccagcgcgctgttgatattgtctcgcaaatgacattggatgagaaggtcaatctgaccacaggaactggatgggaattggaactatgtgttggtcagactggcggtgttccccgattgggagttccgggaatgtgtttacaggatagccctctgggcgttcgcgactccgactacaactctgctttccctgccggcatgaacgtggctgcaacctgggacaagaatctggcataccttcgcggcaaggctatgggtcaggaatttagtgacaagggtgccgatatccaattgggtccagctgccggccctctcggtagaagtcccgacggtggtcgtaactgggagggcttctccccagaccctgccctaagtggtgtgctctttgccgagaccatcaagggtatccaagatgctggtgtggttgcgacggctaagcactacattgcttacgagcaagagcatttccgtcaggcgcctgaagcccaaggttttggatttaatatttccgagagtggaagtgcgaacctcgatgataagactatgcacgagctgtacctctggcccttcgcggatgccatccgtgcaggtgctggcgctgtgatgtgctcctacaaccagatcaacaacagttatggctgccagaacagctacactctgaacaagctgctcaaggccgagctgggcttccagggctttgtcatgagtgattgggctgctcaccatgctggtgtgagtggtgctttggcaggattggatatgtctatgccaggagacgtcgactacgacagtggtacgtcttactggggtacaaacttgaccattagcgtgctcaacggaacggtgccccaatggcgtgttgatgacatggctgtccgcatcatggccgcctactacaaggtcggccgtgaccgtctgtggactcctcccaacttcagctcatggaccagagatgaatacggctacaagtactactacgtgtcggagggaccgtacgagaaggtcaaccagtacgtgaatgtgcaacgcaaccacagcgaactgattcgccgcattggagcggacagcacggtgctcctcaagaacgacggcgctctgcctttgactggtaaggagcgcctggtcgcgcttatcggagaagatgcgggctccaacccttatggtgccaacggctgcagtgaccgtggatgcgacaatggaacattggcgatgggctggggaagtggtactgccaacttcccatacctggtgacccccgagcaggccatctcaaacgaggtgcttaagcacaagaatggtgtattcaccgccaccgataactgggctatcgatcagattgaggcgcttgctaagaccgccagtgtctctcttgtctttgtcaacgccgactctggtgagggttacatcaatgtggacggaaacctgggtgaccgcaggaacctgaccctgtggaggaacggcgataatgtgatcaaggctgctgctagcaactgcaacaacacaatcgttgtcattcactctgtcggaccagtcttggttaacgagtggtacgacaaccccaatgttaccgctatcctctggggtggtttgcccggtcaggagtctggcaactctcttgccgacgtcctctatggccgtgtcaaccccggtgccaagtcgccctttacctggggcaagactcgtgaggcctaccaagactacttggtcaccgagcccaacaacggcaacggagcccctcaggaagactttgtcgagggcgtcttcattgactaccgtggatttgacaagcgcaacgagaccccgatctacgagttcggctatggtctgagctacaccactttcaactactcgaaccttgaggtgcaggtgctgagcgcccctgcatacgagcctgcttcgggtgagaccgaggcagcgccaaccttcggagaggttggaaatgcgtcggattacctctaccccagcggattgcagagaattaccaagttcatctacccctggctcaacggtaccgatctcgaggcatcttccggggatgctagctacgggcaggactcctccgactatcttcccgagggagccaccgatggctctgcgcaaccgatcctgcctgccggtggcggtcctggcggcaaccctcgcctgtacgacgagctcatccgcgtgtcagtgaccatcaagaacaccggcaaggttgctggtgatgaagttccccaactgtatgtttcccttggcggtcccaatgagcccaagatcgtgctgcgtcaattcgagcgcatcacgctgcagccgtcggaggagacgaagtggagcacgactctgacgcgccgtgaccttgcaaactggaatgttgagaagcaggactgggagattacgtcgtatcccaagatggtgtttgtcggaagctcctcgcggaagctgccgctccgggcgtctctgcctactgttcactaa

SEQ ID NO: 118 - protein, 860 aa, Aspergillus niger
MRFTLIEAVALTAVSLASADELAYSPPYYPSPWANGQGDWAQAYQRAVDIVSQMTLDEKVNLTTGTGWELELCVGQTGGVPRLGVPGMCLQDSPLGVRDSDYNSAFPAGMNVAATWDKNLAYLRGKAMGQEFSDKGADIQLGPAAGPLGRSPDGGRNWEGFSPDPALSGVLFAETIKGIQDAGVVATAKHYIAYEQEHFRQAPEAQGFGFNISESGSANLDDKTMHELYLWPFADAIRAGAGAVMCSYNQINNSYGCQNSYTLNKLLKAELGFQGFVMSDWAAHHAGVSGALAGLDMSMPGDVDYDSGTSYWGTNLTISVLNGTVPQWRVDDMAVRIMAAYYKVGRDRLWTPPNFSSWTRDEYGYKYYYVSEGPYEKVNQYVNVQRNHSELIRRIGADSTVLLKNDGALPLTGKERLVALIGEDAGSNPYGANGCSDRGCDNGTLAMGWGSGTANFPYLVTPEQAISNEVLKHKNGVFTATDNWAIDQIEALAKTASVSLVFVNADSGEGYINVDGNLGDRRNLTLWRNGDNVIKAAASNCNNTIVVIHSVGPVLVNEWYDNPNVTAILWGGLPGQESGNSLADVLYGRVNPGAKSPFTWGKTREAYQDYLVTEPNNGNGAPQEDFVEGVFIDYRGFDKRNETPIYEFGYGLSYTTFNYSNLEVQVLSAPAYEPASGETEAAPTFGEVGNASDYLYPSGLQRITKFIYPWLNGTDLEASSGDASYGQDSSDYLPEGATDGSAQPILPAGGGPGGNPRLYDELIRVSVTIKNTGKVAGDEVPQLYVSLGGPNEPKIVLRQFERITLQPSEETKWSTTLTRRDLANWNVEKQDWEITSYPKMVFVGSSSRKLPLRASLPTVH

SEQ ID NO: 119 - DNA, 2583 bp, Aspergillus aculeatus
atgaagctcagttggcttgaggcggctgccttgacggctgcttcagtcgtcagcgctgatgaactggcgttctctcctcctttctacccctctccgtgggccaatggccagggagagtgggcggaagcctaccagcgtgcagtggccattgtatcccagatgactctggatgagaaggtcaacctgaccaccggaactggatgggagctggagaagtgcgtcggtcagactggtggtgtcccaagactgaacatcggtggcatgtgtcttcaggacagtcccttgggaattcgtgatagtgactacaattcggctttccctgctggtgtcaacgttgctgcgacatgggacaagaaccttgcttatctacgtggtcaggctatgggtcaagagttcagtgacaaaggaattgatgttcaattgggaccggccgcgggtcccctcggcaggagccctgatggaggtcgcaactgggaaggtttctctccagacccggctcttactggtgtgctctttgcggagacgattaagggtattcaagacgctggtgtcgtggcgacagccaagcattacattctcaatgagcaagagcatttccgccaggtcgcagaggctgcgggctacggattcaatatctccgacacgatcagctctaacgttgatgacaagaccattcatgaaatgtacctctggcccttcgcggatgccgttcgcgccggcgttggcgccatcatgtgttcctacaaccagatcaacaacagctacggttgccagaacagttacactctgaacaagcttctgaaggccgagctcggcttccagggctttgtgatgtctgactggggtgctcaccacagtggtgttggctctgctttggccggcttggatatgtcaatgcctggcgatatcaccttcgattctgccactagtttctggggtaccaacctgaccattgctgtgctcaacggtaccgtcccgcagtggcgcgttgacgacatggctgtccgtatcatggctgcctactacaaggttggccgcgaccgcctgtaccagccgcctaacttcagctcctggactcgcgatgaatacggcttcaagtatttctacccccaggaagggccctatgagaaggtcaatcactttgtcaatgtgcagcgcaaccacagcgaggttattcgcaagttgggagcagacagtactgttctactgaagaacaacaatgccctgccgctgaccggaaaggagcgcaaagttgcgatcctgggtgaagatgctggatccaactcgtacggtgccaatggctgctctgaccgtggctgtgacaacggtactcttgctatggcttggggtagcggcactgccgaattcccatatctcgtgacccctgagcaggctattcaagccgaggtgctcaagcataagggcagcgtctacgccatcacggacaactgggcgctgagccaggtggagaccctcgctaaacaagccagtgtctctcttgtatttgtcaactcggacgcgggagagggctatatctccgtggacggaaacgagggcgaccgcaacaacctcaccctctggaagaacggcgacaacctcatcaaggctgctgcaaacaactgcaacaacaccatcgttgtcatccactccgttggacctgttttggttgacgagtggtatgaccaccccaacgttactgccatcctctgggcgggcttgcctggccaggagtctggcaactccttggctgacgtgctctacggccgcgtcaacccgggcgccaaatctccattcacctggggcaagacgagggaggcgtacggggattaccttgtccgtgagctcaacaacggcaacggagctccccaagatgatttctcggaaggtgttttcattgactaccgcggattcgacaagcgcaatgagaccccgatctacgagttcggacatggtctgagctacaccactttcaactactctggccttcacatccaggttctcaacgcttcctccaacgctcaagtagccactgagactggcgccgctcccaccttcggacaagtcggcaatgcctctgactacgtgtaccctgagggattgaccagaatcagcaagttcatctatccctggcttaattccacagacctgaaggcctcatctggcgacccgtactatggagtcgacaccgcggagcacgtgcccgagggtgctactgatggctctccgcagcccgttctgcctgccggtggtggctctggtggtaacccgcgcctctacgatgagttgatccgtgtttcggtgacagtcaagaacactggtcgtgttgccggtgatgctgtgcctcaattgtatgtttcccttggtggacccaatgagcccaaggttgtgttgcgcaaattcgaccgcctcaccctcaagccctccgaggagacggtgtggacgactaccctgacccgccgcgatctgtctaactgggacgttgcggctcaggactgggtcatcacttcttacccgaagaaggtccatgttggtagctcttcgcgtcagctgccccttcacgcggcgctcccgaaggtgcaatga

SEQ ID NO: 120 - protein, 860 aa, Aspergillus aculeatus
MKLSWLEAAALTAASVVSADELAFSPPFYPSPWANGQGEWAEAYQRAVAIVSQMTLDEKVNLTTGTGWELEKCVGQTGGVPRLNIGGMCLQDSPLGIRDSDYNSAFPAGVNVAATWDKNLAYLRGQAMGQEFSDKGIDVQLGPAAGPLGRSPDGGRNWEGFSPDPALTGVLFAETIKGIQDAGVVATAKHYILNEQEHFRQVAEAAGYGFNISDTISSNVDDKTIHEMYLWPFADAVRAGVGAIMCSYNQINNSYGCQNSYTLNKLLKAELGFQGFVMSDWGAHHSGVGSALAGLDMSMPGDITFDSATSFWGTNLTIAVLNGTVPQWRVDDMAVRIMAAYYKVGRDRLYQPPNFSSWTRDEYGFKYFYPQEGPYEKVNHFVNVQRNHSEVIRKLGADSTVLLKNNNALPLTGKERKVAILGEDAGSNSYGANGCSDRGCDNGTLAMAWGSGTAEFPYLVTPEQAIQAEVLKHKGSVYAITDNWALSQVETLAKQASVSLVFVNSDAGEGYISVDGNEGDRNNLTLWKNGDNLIKAAANNCNNTIVVIHSVGPVLVDEWYDHPNVTAILWAGLPGQESGNSLADVLYGRVNPGAKSPFTWGKTREAYGDYLVRELNNGNGAPQDDFSEGVFIDYRGFDKRNETPIYEFGHGLSYTTFNYSGLHIQVLNASSNAQVATETGAAPTFGQVGNASDYVYPEGLTRISKFIYPWLNSTDLKASSGDPYYGVDTAEHVPEGATDGSPQPVLPAGGGSGGNPRLYDELIRVSVTVKNTGRVAGDAVPQLYVSLGGPNEPKVVLRKFDRLTLKPSEETVWTTTLTRRDLSNWDVAAQDWVITSYPKKVHVGSSSRQLPLHAALPKVQ

SEQ ID NO: 121 - DNA, 3294 bp, Aspergillus oryzae
atgcgttcctcccccctcctccgctccgccgttgtggccgccctgccggtgttggcccttgccgctgatggcaggtccacccgctactgggactgctgcaagccttcgtgcggctgggccaagaaggctcccgtgaaccagcctgtcttttcctgcaacgccaacttccagcgtatcacggacttcgacgccaagtccggctgcgagccgggcggtgtcgcctactcgtgcgccgaccagaccccatgggctgtgaacgacgacttcgcgctcggttttgctgccacctctattgccggcagcaatgaggcgggctggtgctgcgcctgctacgagctcaccttcacatccggtcctgttgctggcaagaagatggtcgtccagtccaccagcactggcggtgatcttggcagcaaccacttcgatctcaacatccccggcggcggcgtcggcatcttcgacggatgcactccccagttcggtggtctgcccggccagcgctacggcggcatctcgtcccgcaacgagtgcgatcggttccccgacgccctcaagcccggctgctactggcgcttcgactggttcaagaacgccgacaatccgagcttcagcttccgtcaggtccagtgcccagccgagctcgtcgctcgcaccggatgccgccgcaacgacgacggcaacttccctgccgtccagatccccatgcgttcctcccccctcctccgctccgccgttgtggccgccctgccggtgttggcccttgccaaggatgatctcgcgtactcccctcctttctacccttccccatgggcagatggtcagggtgaatgggcggaagtatacaaacgcgctgtagacatagtttcccagatgacgttgacagagaaagtcaacttaacgactggaacaggatggcaactagagaggtgtgttggacaaactggcagtgttcccagactcaacatccccagcttgtgtttgcaggatagtcctcttggtattcgtttctcggactacaattcagctttccctgcgggtgttaatgtcgctgccacctgggacaagacgctcgcctaccttcgtggtcaggcaatgggtgaggagttcagtgataagggtattgacgttcagctgggtcctgctgctggccctctcggtgctcatccggatggcggtagaaactgggaaggtttctcaccagatccagccctcaccggtgtactttttgcggagacgattaagggtattcaagatgctggtgtcattgcgacagctaagcattatatcatgaacgaacaagagcatttccgccaacaacccgaggctgcgggttacggattcaacgtaagcgacagtttgagttccaacgttgatgacaagactatgcatgaattgtacctctggcccttcgcggatgcagtacgcgctggagtcggtgctgtcatgtgctcttacaaccaaatcaacaacagctacggttgcgagaatagcgaaactctgaacaagcttttgaaggcggagcttggtttccaaggcttcgtcatgagtgattggaccgctcatcacagcggcgtaggcgctgctttagcaggtctggatatgtcgatgcccggtgatgttaccttcgatagtggtacgtctttctggggtgcaaacttgacggtcggtgtccttaacggtacaatcccccaatggcgtgttgatgacatggctgtccgtatcatggccgcttattacaaggttggccgcgacaccaaatacacccctcccaacttcagctcgtggaccagggacgaatatggtttcgcgcataaccatgtttcggaaggtgcttacgagagggtcaacgaattcgtggacgtgcaacgcgatcatgccgacctaatccgtcgcatcggcgcgcagagcactgttctgctgaagaacaagggtgccttgcccttgagccgcaaggaaaagctggtcgcccttctgggagaggatgcgggttccaactcgtggggcgctaacggctgtgatgaccgtggttgcgataacggtacccttgccatggcctggggtagcggtactgcgaatttcccatacctcgtgacaccagagcaggcgattcagaacgaagttcttcagggccgtggtaatgtcttcgccgtgaccgacagttgggcgctcgacaagatcgctgcggctgcccgccaggccagcgtatctctcgtgttcgtcaactccgactcaggagaaggctatcttagtgtggatggaaatgagggcgatcgtaacaacatcactctgtggaagaacggcgacaatgtggtcaagaccgcagcgaataactgtaacaacaccgttgtcatcatccactccgtcggaccagttttgatcgatgaatggtatgaccaccccaatgtcactggtattctctgggctggtctgccaggccaggagtctggtaactccattgccgatgtgctgtacggtcgtgtcaaccctggcgccaagtctcctttcacttggggcaagacccgggagtcgtatggttctcccttggtcaaggatgccaacaatggcaacggagcgccccagtctgatttcacccagggtgttttcatcgattaccgccatttcgataagttcaatgagacccctatctacgagtttggctacggcttgagctacaccaccttcgagctctccgacctccatgttcagcccctgaacgcgtcccgatacactcccaccagtggcatgactgaagctgcaaagaactttggtgaaattggcgatgcgtcggagtacgtgtatccggaggggctggaaaggatccatgagtttatctatccctggatcaactctaccgacctgaaggcatcgtctgacgattctaactacggctgggaagactccaagtatattcccgaaggcgccacggatgggtctgcccagccccgtttgcccgctagtggtggtgccggaggaaaccccggtctgtacgaggatcttttccgcgtctctgtgaaggtcaagaacacgggcaatgtcgccggtgatgaagttcctcagctgtacgtttccctaggcggcccgaatgagcccaaggtggtactgcgcaagtttgagcgtattcacttggccccttcgcaggaggccgtgtggacaacgacccttacccgtcgtgaccttgcaaactgggacgtttcggctcaggactggaccgtcactccttaccccaagacgatctacgttggaaactcctcacggaaactgccgctccaggcctcgctgcctaaggcccagtaa

SEQ ID NO: 122 - protein, 1097 aa, Aspergillus oryzae
MRSSPLLRSAVVAALPVLALAADGRSTRYWDCCKPSCGWAKKAPVNQPVFSCNANFQRITDFDAKSGCEPGGVAYSCADQTPWAVNDDFALGFAATSIAGSNEAGWCCACYELTFTSGPVAGKKMVVQSTSTGGDLGSNHFDLNIPGGGVGIFDGCTPQFGGLPGQRYGGISSRNECDRFPDALKPGCYWRFDWFKNADNPSFSFRQVQCPAELVARTGCRRNDDGNFPAVQIPMRSSPLLRSAVVAALPVLALAKDDLAYSPPFYPSPWADGQGEWAEVYKRAVDIVSQMTLTEKVNLTTGTGWQLERCVGQTGSVPRLNIPSLCLQDSPLGIRFSDYNSAFPAGVNVAATWDKTLAYLRGQAMGEEFSDKGIDVQLGPAAGPLGAHPDGGRNWEGFSPDPALTGVLFAETIKGIQDAGVIATAKHYIMNEQEHFRQQPEAAGYGFNVSDSLSSNVDDKTMHELYLWPFADAVRAGVGAVMCSYNQINNSYGCENSETLNKLLKAELGFQGFVMSDWTAHHSGVGAALAGLDMSMPGDVTFDSGTSFWGANLTVGVLNGTIPQWRVDDMAVRIMAAYYKVGRDTKYTPPNFSSWTRDEYGFAHNHVSEGAYERVNEFVDVQRDHADLIRRIGAQSTVLLKNKGALPLSRKEKLVALLGEDAGSNSWGANGCDDRGCDNGTLAMAWGSGTANFPYLVTPEQAIQNEVLQGRGNVFAVTDSWALDKIAAAARQASVSLVFVNSDSGEGYLSVDGNEGDRNNITLWKNGDNVVKTAANNCNNTVVIIHSVGPVLIDEWYDHPNVTGILWAGLPGQESGNSIADVLYGRVNPGAKSPFTWGKTRESYGSPLVKDANNGNGAPQSDFTQGVFIDYRHFDKFNETPIYEFGYGLSYTTFELSDLHVQPLNASRYTPTSGMTEAAKNFGEIGDASEYVYPEGLERIHEFIYPWINSTDLKASSDDSNYGWEDSKYIPEGATDGSAQPRLPASGGAGGNPGLYEDLFRVSVKVKNTGNVAGDEVPQLYVSLGGPNEPKVVLRKFERIHLAPSQEAVWTTTLTRRDLANWDVSAQDWTVTPYPKTIYVGNSSRKLPLQASLPKAQ

SEQ ID NO: 123 - DNA, 3294 bp, Aspergillus oryzae
atgcgttcctcccccctcctccgctccgccgttgtggccgccctgccggtgttggcccttgccgctgatggcaggtccacccgctactgggactgctgcaagccttcgtgcggctgggccaagaaggctcccgtgaaccagcctgtcttttcctgcaacgccaacttccagcgtatcacggacttcgacgccaagtccggctgcgagccgggcggtgtcgcctactcgtgcgccgaccagaccccatgggctgtgaacgacgacttcgcgctcggttttgctgccacctctattgccggcagcaatgaggcgggctggtgctgcgcctgctacgagctcaccttcacatccggtcctgttgctggcaagaagatggtcgtccagtccaccagcactggcggtgatcttggcagcaaccacttcgatctcaacatccccggcggcggcgtcggcatcttcgacggatgcactccccagttcggtggtctgcccggccagcgctacggcggcatctcgtcccgcaacgagtgcgatcggttccccgacgccctcaagcccggctgctactggcgcttcgactggttcaagaacgccgacaatccgagcttcagcttccgtcaggtccagtgcccagccgagctcgtcgctcgcaccggatgccgccgcaacgacgacggcaacttccctgccgtccagatccccatgcgttcctcccccctcctccgctccgccgttgtggccgccctgccggtgttggcccttgccaaggatgatctcgcgtactcccctcctttctacccttccccatgggcagatggtcagggtgaatgggcggaagtatacaaacgcgctgtagacatagtttcccagatgacgttgacagagaaagtcaacttaacgactggaacaggatggcaactagagaggtgtgttggacaaactggcagtgttcccagactcaacatccccagcttgtgtttgcaggatagtcctcttggtattcgtttctcggactacaattcagctttccctgcgggtgttaatgtcgctgccacctgggacaagacgctcgcctaccttcgtggtcaggcaatgggtgaggagttcagtgataagggtattgacgttcagctgggtcctgctgctggccctctcggtgctcatccggatggcggtagaaactgggaaagtttctcaccagatccagccctcaccggtgtactttttgcggagacgattaagggtattcaagatgctggtgtcattgcgacagctaagcattatatcatgaacgaacaagagcatttccgccaacaacccgaggctgcgggttacggattcaacgtaagcgacagtttgagttccaacgttgatgacaagactatgcatgaattgtacctctggcccttcgcggatgcagtacgcgctggagtcggtgctgttatgtgctcttacaaccaaatcaacaacagctacggttgcgagaatagcgaaactctgaacaagcttttgaaggcggagcttggtttccaaggcttcgtcatgagtgattggaccgctcaacacagcggcgtaggcgctgctttagcaggtctggatatgtcgatgcccggtgatgttaccttcgatagtggtacgtctttctggggtgcaaacttgacggtcggtgtccttaacggtacaatcccccaatggcgtgttgatgacatggctgtccgtatcatggccgcttattacaaggttggccgcgacaccaaatacacccctcccaacttcagctcgtggaccagggacgaatatggtttcgcgcataaccatgtttcggaaggtgcttacgagagggtcaacgaattcgtggacgtgcaacgcgatcatgccgacctaatccgtcgcatcggcgcgcagagcactgttctgctgaagaacaagggtgccttgcccttgagccgcaaggaaaagctggtcgcccttctgggagaggatgcgggttccaactcgtggggcgctaacggctgtgatgaccgtggttgcgataacggtacccttgccatggcctggggtagcggtactgcgaatttcccatacctcgtgacaccagagcaggcgattcagaacgaagttcttcagggccgtggtaatgtcttcgccgtgaccgacagttgggcgctcgacaagatcgctgcggctgcccgccaggccagcgtatctctcgtgttcgtcaactccgactcaggagaaggctatcttagtgtggatggaaatgagggcgatcgtaacaacatcactctgtggaagaacggcgacaatgtggtcaagaccgcagcgaataactgtaacaacaccgttgtcatcatccactccgtcggaccagttttgatcgatgaatggtatgaccaccccaatgtcactggtattctctgggctggtctgccaggccaggagtctggtaactccattgccgatgtgctgtacggtcgtgtcaaccctggcgccaagtctcctttcacttggggcaagacccgggagtcgtatggttctcccttggtcaaggatgccaacaatggcaacggagcgccccagtctgatttcacccagggtgttttcatcgattaccgccatttcgataagttcaatgagacccctatctacgagtttggctacggcttgagctacaccaccttcgagctctccgacctccatgttcagcccctgaacgcgtcccgatacactcccaccagtggcatgactgaagctgcaaagaactttggtgaaattggcgatgcgtcggagtacgtgtatccggaggggctggaaaggatccatgagtttatctatccctggatcaactctaccgacctgaaggcatcgtctgacgattctaactacggctgggaagactccaagtatattcccgaaggcgccacggatgggtctgcccagccccgtttgcccgctagtggtggtgccggaggaaaccccggtctgtacgaggatcttttccgcgtctctgtgaaggtcaagaacacgggcaatgtcgccggtgatgaagttcctcagctgtacgtttccctaggcggcccgaatgagcccaaggtggtactgcgcaagtttgagcgtattcacttggccccttcgcaggaggccgtgtggacaacgacccttacccgtcgtgaccttgcaaactgggacgtttcggctcaggactggaccgtcactccttaccccaagacgatctacgttggaaactcctcacggaaactgccgctccaggcctcgctgcctaaggcccagtaa

SEQ ID NO: 124 - protein, 1097 aa, Aspergillus oryzae
MRSSPLLRSAVVAALPVLALAADGRSTRYWDCCKPSCGWAKKAPVNQPVFSCNANFQRITDFDAKSGCEPGGVAYSCADQTPWAVNDDFALGFAATSIAGSNEAGWCCACYELTFTSGPVAGKKMVVQSTSTGGDLGSNHFDLNIPGGGVGIFDGCTPQFGGLPGQRYGGISSRNECDRFPDALKPGCYWRFDWFKNADNPSFSFRQVQCPAELVARTGCRRNDDGNFPAVQIPMRSSPLLRSAVVAALPVLALAKDDLAYSPPFYPSPWADGQGEWAEVYKRAVDIVSQMTLTEKVNLTTGTGWQLERCVGQTGSVPRLNIPSLCLQDSPLGIRFSDYNSAFPAGVNVAATWDKTLAYLRGQAMGEEFSDKGIDVQLGPAAGPLGAHPDGGRNWESFSPDPALTGVLFAETIKGIQDAGVIATAKHYIMNEQEHFRQQPEAAGYGFNVSDSLSSNVDDKTMHELYLWPFADAVRAGVGAVMCSYNQINNSYGCENSETLNKLLKAELGFQGFVMSDWTAQHSGVGAALAGLDMSMPGDVTFDSGTSFWGANLTVGVLNGTIPQWRVDDMAVRIMAAYYKVGRDTKYTPPNFSSWTRDEYGFAHNHVSEGAYERVNEFVDVQRDHADLIRRIGAQSTVLLKNKGALPLSRKEKLVALLGEDAGSNSWGANGCDDRGCDNGTLAMAWGSGTANFPYLVTPEQAIQNEVLQGRGNVFAVTDSWALDKIAAAARQASVSLVFVNSDSGEGYLSVDGNEGDRNNITLWKNGDNVVKTAANNCNNTVVIIHSVGPVLIDEWYDHPNVTGILWAGLPGQESGNSIADVLYGRVNPGAKSPFTWGKTRESYGSPLVKDANNGNGAPQSDFTQGVFIDYRHFDKFNETPIYEFGYGLSYTTFELSDLHVQPLNASRYTPTSGMTEAAKNFGEIGDASEYVYPEGLERIHEFIYPWINSTDLKASSDDSNYGWEDSKYIPEGATDGSAQPRLPASGGAGGNPGLYEDLFRVSVKVKNTGNVAGDEVPQLYVSLGGPNEPKVVLRKFERIHLAPSQEAVWTTTLTRRDLANWDVSAQDWTVTPYPKTIYVGNSSRKLPLQASLPKAQ

SEQ ID NO: 125 - protein, 19 aa, Thielavia terrestris
  MISC_FEATURE (1)..(1): X=I,L,M, OR V
  misc_feature (3)..(6): Xaa can be any naturally occurring amino acid
  misc_feature (8)..(8): Xaa can be any naturally occurring amino acid
  MISC_FEATURE (10)..(10): X=I,L,M, OR V
  misc_feature (11)..(11): Xaa can be any naturally occurring amino acid
  misc_feature (13)..(13): Xaa can be any naturally occurring amino acid
  MISC_FEATURE (14)..(14): X=E OR Q
  misc_feature (15)..(18): Xaa can be any naturally occurring amino acid
  MISC_FEATURE (19)..(19): X=H,N, OR Q
XPXXXXGXYXXRXXXXXXX

SEQ ID NO: 126 - protein, 20 aa, Thielavia terrestris
  MISC_FEATURE (1)..(1): X=I,L,M, OR V
  misc_feature (3)..(7): Xaa can be any naturally occurring amino acid
  misc_feature (9)..(9): Xaa can be any naturally occurring amino acid
  MISC_FEATURE (11)..(11): X=I,L,M, OR V
  misc_feature (12)..(12): Xaa can be any naturally occurring amino acid
  misc_feature (14)..(14): Xaa can be any naturally occurring amino acid
  MISC_FEATURE (15)..(15): X=E OR Q
  misc_feature (16)..(19): Xaa can be any naturally occurring amino acid
  MISC_FEATURE (20)..(20): X=H,N, OR Q
XPXXXXXGXYXXRXXXXXXX

SEQ ID NO: 127 - protein, 9 aa, Thielavia terrestris
  misc_feature (2)..(2): Xaa can be any naturally occurring amino acid
  misc_feature (5)..(7): Xaa can be any naturally occurring amino acid
  MISC_FEATURE (8)..(8): X= Y OR W
  MISC_FEATURE (9)..(9): X= A,I,L,M OR V
HXGPXXXXX

SEQ ID NO: 128 - protein, 10 aa, Thielavia terrestris
  misc_feature (2)..(3): Xaa can be any naturally occurring amino acid
  misc_feature (6)..(8): Xaa can be any naturally occurring amino acid
  MISC_FEATURE (9)..(9): X= Y OR W
  MISC_FEATURE (10)..(10): X= Y OR W
HXXGPXXXXX

SEQ ID NO: 129 - protein, 11 aa, Thielavia terrestris
  MISC_FEATURE (1)..(1): X= E OR Q
  misc_feature (2)..(2): Xaa can be any naturally occurring amino acid
  misc_feature (4)..(5): Xaa can be any naturally occurring amino acid
  misc_feature (7)..(7): Xaa can be any naturally occurring amino acid
  MISC_FEATURE (8)..(8): X= E,H,Q OR N
  MISC_FEATURE (9)..(9): X=F,I,L, OR V
  misc_feature (10)..(10): Xaa can be any naturally occurring amino acid
  MISC_FEATURE (11)..(11): X=I,L,OR V
XXYXXCXXXXX

SEQ ID NO: 130 - protein, 9 aa, Thielavia terrestris
  misc_feature (2)..(2): Xaa can be any naturally occurring amino acid
  misc_feature (5)..(7): Xaa can be any naturally occurring amino acid
  MISC_FEATURE (8)..(8): X= Y OR W
  MISC_FEATURE (9)..(9): X= A,I,L,M OR V
HXGPXXXXX

SEQ ID NO: 131 - protein, 10 aa, Thielavia terrestris
  misc_feature (2)..(3): Xaa can be any naturally occurring amino acid
  misc_feature (6)..(8): Xaa can be any naturally occurring amino acid
  MISC_FEATURE (9)..(9): X= Y OR W
  MISC_FEATURE (10)..(10): X= A,I,L,M OR V
HXXGPXXXXX

SEQ ID NO: 132 - protein, 11 aa, Thielavia terrestris
  MISC_FEATURE (1)..(1): X= E OR Q
  misc_feature (2)..(2): Xaa can be any naturally occurring amino acid
  misc_feature (4)..(5): Xaa can be any naturally occurring amino acid
  misc_feature (7)..(7): Xaa can be any naturally occurring amino acid
  MISC_FEATURE (8)..(8): X= E,H,Q OR N
  MISC_FEATURE (9)..(9): X=F,I,L, OR V
  misc_feature (10)..(10): Xaa can be any naturally occurring amino acid
  MISC_FEATURE (11)..(11): X=I,L,OR V
XXYXXCXXXXX

SEQ ID NO: 133 - protein, 9 aa, Thielavia terrestris
  misc_feature (2)..(2): Xaa can be any naturally occurring amino acid
  misc_feature (5)..(7): Xaa can be any naturally occurring amino acid
  MISC_FEATURE (8)..(8): X= Y OR W
  MISC_FEATURE (9)..(9): X= A,I,L,M OR V
HXGPXXXXX

SEQ ID NO: 134 - protein, 10 aa, Thielavia terrestris
  misc_feature (2)..(3): Xaa can be any naturally occurring amino acid
  misc_feature (6)..(8): Xaa can be any naturally occurring amino acid
  MISC_FEATURE (9)..(9): X= Y OR W
  MISC_FEATURE (10)..(10): X= A,I,L,M OR V
HXXGPXXXXX

SEQ ID NO: 135 - protein, 11 aa, Thielavia terrestris
  MISC_FEATURE (1)..(1): X= E OR Q
  misc_feature (2)..(2): Xaa can be any naturally occurring amino acid
  misc_feature (4)..(5): Xaa can be any naturally occurring amino acid
  misc_feature (7)..(7): Xaa can be any naturally occurring amino acid
  MISC_FEATURE (8)..(8): X= E,H,Q OR N
  MISC_FEATURE (9)..(9): X=F,I,L, OR V
  misc_feature (10)..(10): Xaa can be any naturally occurring amino acid
  MISC_FEATURE (11)..(11): X=I,L,OR V
XXYXXCXXXXX

SEQ ID NO: 136 - protein, 9 aa, Thielavia terrestris
  misc_feature (2)..(2): Xaa can be any naturally occurring amino acid
  misc_feature (5)..(7): Xaa can be any naturally occurring amino acid
  MISC_FEATURE (8)..(8): X= Y OR W
  MISC_FEATURE (9)..(9): X= A,I,L,M OR V
HXGPXXXXX

SEQ ID NO: 137 - protein, 10 aa, Thielavia terrestris
  misc_feature (2)..(3): Xaa can be any naturally occurring amino acid
  misc_feature (6)..(8): Xaa can be any naturally occurring amino acid
  MISC_FEATURE (9)..(9): X= Y OR W
  MISC_FEATURE (10)..(10): X= A,I,L,M OR V
HXXGPXXXXX

SEQ ID NO: 138 - protein, 11 aa, Thielavia terrestris
  MISC_FEATURE (1)..(1): X= E OR Q
  misc_feature (2)..(2): Xaa can be any naturally occurring amino acid
  misc_feature (4)..(5): Xaa can be any naturally occurring amino acid
  misc_feature (7)..(7): Xaa can be any naturally occurring amino acid
  MISC_FEATURE (8)..(8): X= E,H,Q OR N
  MISC_FEATURE (9)..(9): X=F,I,L, OR V
  misc_feature (10)..(10): Xaa can be any naturally occurring amino acid
  MISC_FEATURE (11)..(11): X=I,L,OR V
XXYXXCXXXXX

SEQ ID NO: 139 - protein, 19 aa, Thielavia terrestris
  MISC_FEATURE (1)..(1): X=I,L,M OR V
  misc_feature (3)..(6): Xaa can be any naturally occurring amino acid
  misc_feature (8)..(8): Xaa can be any naturally occurring amino acid
  MISC_FEATURE (10)..(10): X=I,L,M OR V
  misc_feature (11)..(11): Xaa can be any naturally occurring amino acid
  misc_feature (13)..(13): Xaa can be any naturally occurring amino acid
  MISC_FEATURE (14)..(14): X= E OR Q
  misc_feature (15)..(17): Xaa can be any naturally occurring amino acid
  MISC_FEATURE (19)..(19): X= H,N, OR Q
XPXXXXGXYXXRXXXXXAX

SEQ ID NO: 140 - protein, 20 aa, Thielavia terrestris
  MISC_FEATURE (1)..(1): X=I,L,M OR V
  misc_feature (3)..(7): Xaa can be any naturally occurring amino acid
  misc_feature (9)..(9): Xaa can be any naturally occurring amino acid
  MISC_FEATURE (11)..(11): X=I,L,M OR V
  misc_feature (12)..(12): Xaa can be any naturally occurring amino acid
  misc_feature (14)..(14): Xaa can be any naturally occurring amino acid
  MISC_FEATURE (15)..(15): X=E OR Q
  misc_feature (16)..(18): Xaa can be any naturally occurring amino acid
  MISC_FEATURE (20)..(20): X= H,N, OR Q
XPXXXXXGXYXXRXXXXXAX

SEQ ID NO: 141 - DNA, 1035 bp, Aspergillus aculeatus
atgaagtatattcctctcgttattgcagttgctgccggcctggcacgtccggctactgcccactacatcttcagcaagctcgtgctgaacggagaggcatctgcggactggcaatacatccgcgagactactcgcagcatagtctatgagccgaccaagtacacctctaccttcgataacctaacacccagcgatagcgacttccgctgtaatctcggttccttcagcaatgctgcgaagaccgaggtcgctgaggttgcggcaggcgataccatcgcaatgaagctattctacgacaccagtattgcgcatcctggcccgggacaagtttatatgtccaaggcaccgaccggcaatgttcaggaataccaaggagacggggattggttcaaaatctgggaaaagaccctttgcaacacggatggtgatctgactacagaggcctggtgcacctggggcatgtcacagtttgaatttcaaatcccagctgcgaccccggcaggagagtacctagtgcgcgccgagcatataggcctgcatggcgctcaagcgaacgaggccgaattcttctacagctgtgcgcagatcaaggttacaggctcgggaactggatctcccagtctcacgtatcaaattcctggtctctataacgacactatgaccctgttcaatggcctcaatctttggactgattcagccgagaaggtgcagctggatttcctggagacgccaattggggacgacgtgtggagcggagcaggctcggggagcccatctgctgccacctcttcgaccagcggtgcaactcttgcagctcagggtacaactacctctgccgcgcatgctcaggcccagaccaccattaccaccagcaccagcaccatcacgtctctcgaatcagccagctcaaccgatctcgttgcgcagtatggtcagtgcggaggccttaactggtccggtccaaccgagtgtgagacaccttatacctgtgtgcagcagaacccttactaccatcaatgcgtgaattcgtgctga

SEQ ID NO: 142 - protein, 344 aa, Aspergillus aculeatus
MKYIPLVIAVAAGLARPATAHYIFSKLVLNGEASADWQYIRETTRSIVYEPTKYTSTFDNLTPSDSDFRCNLGSFSNAAKTEVAEVAAGDTIAMKLFYDTSIAHPGPGQVYMSKAPTGNVQEYQGDGDWFKIWEKTLCNTDGDLTTEAWCTWGMSQFEFQIPAATPAGEYLVRAEHIGLHGAQANEAEFFYSCAQIKVTGSGTGSPSLTYQIPGLYNDTMTLFNGLNLWTDSAEKVQLDFLETPIGDDVWSGAGSGSPSAATSSTSGATLAAQGTTTSAAHAQAQTTITTSTSTITSLESASSTDLVAQYGQCGGLNWSGPTECETPYTCVQQNPYYHQCVNSC

SEQ ID NO: 143 - DNA, 1170 bp, Aspergillus aculeatus
atgaagtcctctactttcggtatgctcgctctggcagcagcagccaagatggtcgatgcccacaccaccgtcttcgccgtctggatcaacggcgaggaccagggtctgggcaacagtgccagtggctacatccggtctccccccagcaacagccccgtcaaggacgtgacctcgaccgacatcacctgcaacgtcaacggcgaccaggcggcggctaagaccctctccgtcaagggcggcgacgtcgtcaccttcgagtggcaccacgacagccgggacgcctccgacgacatcatcgcctcctcccacaagggccccgtcatggtctacatggccccgaccaccgccggcagcagcggcaagaactgggtcaagatcgccgaggacggatactccgacggcacctgggccgtcgacaccctgatcgccaacagcggcaagcacaacatcaccgtccccgacgtccccgccggcgactacctcttccgcccggagatcatcgccctccacgaggccgagaacgagggcggcgcccagttctacatggagtgtgtccagttcaaggtcacctccgacggtgccaacactctgcccgacggtgtcagcctgcccggcgcctactccgccactgaccccggtatcctcttcaacatgtacggctccttcgacagctatcccatccccggtccctccgtctgggatggcactagctctggctcttcctcttcttcctcttcttcctcttccagctcttccgccgccgctgccgttgttgccacctcctcttcctcttcctctgcttccatcgaggccgtgaccaccaagggtgccgtcgccgccgtctccaccgccgccgccgtggctcctaccaccaccaccgctgcccccaccaccttcgccacggccgtcgcctccaccaagaaggccactgcctgccgcaacaagaccaagtcctcctccgctgccaccaccgccgccgccgtcgccgagaccacctcttccaccgctgccgccaccgctgctgcttcctctgcctcttccgcctccggcaccgccggcaagtacgagcgctgcggtggccagggctggaccggtgccaccacctgcgttgatggctggacctgcaagcagtggaacccttactactaccagtgcgttgagtctgcctag

SEQ ID NO: 144 - protein, 389 aa, Aspergillus aculeatus
MKSSTFGMLALAAAAKMVDAHTTVFAVWINGEDQGLGNSASGYIRSPPSNSPVKDVTSTDITCNVNGDQAAAKTLSVKGGDVVTFEWHHDSRDASDDIIASSHKGPVMVYMAPTTAGSSGKNWVKIAEDGYSDGTWAVDTLIANSGKHNITVPDVPAGDYLFRPEIIALHEAENEGGAQFYMECVQFKVTSDGANTLPDGVSLPGAYSATDPGILFNMYGSFDSYPIPGPSVWDGTSSGSSSSSSSSSSSSSAAAAVVATSSSSSSASIEAVTTKGAVAAVSTAAAVAPTTTTAAPTTFATAVASTKKATACRNKTKSSSAATTAAAVAETTSSTAAATAAASSASSASGTAGKYERCGGQGWTGATTCVDGWTCKQWNPYYYQCVESA

SEQ ID NO: 145 - DNA, 1221 bp, Aspergillus aculeatus
atgcgtcaggctcagtctttgtccctcttgacagctcttctgtctgccacgcgtgtggctggacacggtcacgtcactaacgttgtcgtcaacggtgtttactacgagggcttcgatatcaacagcttcccctacgagtccgatccccctaaggtggcggcttggaccactcctaacactggcaacggtttcatttcccccagcgactacggtaccgatgacattatttgccaccagaatgccaccaacgcccaggcccacattgttgttgcggctggtgacaagatcaacatccagtggaccgcgtggcccgattcccaccacggtcctgtccttgactacctcgctcgctgcgacggtgagtgtgagacggttgataagaccactcttgagtttttcaagatcgacggcgtcggtctcatcagtgacaccgaagtgcccggtacctggggagatgaccagctgatcgccaacaacaacagctggttggtcgagatccccccgaccattgctcctggcaactatgttcttcgccacgagcttatcgctctccacagcgccggcactgaagatggtgctcagaactacccccagtgtttcaacctccaggtcactggctccggtactgacgagcccgctggtaccctcggcaccaagctctacactgaggatgaggctggtatcgttgtgaacatctacacctctctgtcttcctatgccgtccccggccccacccagtacagcggcgccgtctctgtcagccaatccacttcggccattacctccaccggaactgctgttgtcggtagcggcagcgctgttgccacctctgccgccgcggctaccaccagcgctgctgcttcttctgccgctgctgctaccaccgctgctgccgttaccagcgccaatgccaacactcagattgcccagcccagcagcagctcttcttactcccagatcgccgtgcaggtgccctcctcctggaccacccttgtgaccgtcactcctcccgccgccgccgccaccacccctgctgccgtccctgagcctcagaccccctctgccagctctggagccaccactaccagcagcagcagcggcgccgcccagtctctctacggccagtgcggtggtatcaactggaccggagctacctcttgcgttgagggcgctacttgctaccagtacaacccttactactaccagtgcatctctgcctaa

SEQ ID NO: 146 - protein, 406 aa, Aspergillus aculeatus
MRQAQSLSLLTALLSATRVAGHGHVTNVVVNGVYYEGFDINSFPYESDPPKVAAWTTPNTGNGFISPSDYGTDDIICHQNATNAQAHIVVAAGDKINIQWTAWPDSHHGPVLDYLARCDGECETVDKTTLEFFKIDGVGLISDTEVPGTWGDDQLIANNNSWLVEIPPTIAPGNYVLRHELIALHSAGTEDGAQNYPQCFNLQVTGSGTDEPAGTLGTKLYTEDEAGIVVNIYTSLSSYAVPGPTQYSGAVSVSQSTSAITSTGTAVVGSGSAVATSAAAATTSAAASSAAAATTAAAVTSANANTQIAQPSSSSSYSQIAVQVPSSWTTLVTVTPPAAAATTPAAVPEPQTPSASSGATTTSSSSGAAQSLYGQCGGINWTGATSCVEGATCYQYNPYYYQCISA

SEQ ID NO: 147 - DNA, 1284 bp, Aspergillus aculeatus
atgtctctttccaagattgccactcttctgctgggctcggtctcgctggtcgctggtcatgggtatgtctcgagcatcgaggtggacggtaccacctatggagggtacttggtcgacacttattactacgaatccgacccgcccgagttaatcgcctggtccacaaatgccacggatgatggctatgtatcgccctccgactacgagagcgtgaacatcatctgccacaaggggtctgcgcccggcgcgttgtcggcccctgtcgcgcccggaggctgggtgcagatgacctggaacacctggcccaccgaccatcacggccctgtcatcacgtatatggccaattgccacggttcttgcgcagatgtggacaagaccaccctcgagttcttcaagatcgatgctggcggcttgatcgatgacacggacgtgcctggaacttgggcgaccgatgagctcattgaagatagctatagtcgcaacatcactatccccagcgatattgcccccgggtactatgttttgcgacacgagatcattgctctgcacagcgccgagaacctggacggagcccagaactacccccagtgcatcaatctggaagtcaccggcagcgagacagcaaccccgagtggcaccttgggcactgctctgtacaaggagaccgaccccggcatctatgttgacatctggaacacgttgagcacgtatactattcccggccccgcgctgtacactgctggtagcactgcgaccgcagccgctgctgccgataccaccactacttctgctggcaccaccgctgaggccaccaccgctgccgccgccgtgagtaccaccgcggacgctgttccgaccgagtcttcagctccttccgagaccagcgcgactaccgcgaaccctgctcggcccactgccggcagcgacatccgcttccagcccggtcaggtcaaggctggtgcttcagtcaacaactcggctactgagacttcctctggtgagtctgccacgacgaccacaacatcagtggccactgcggcttcgagcgcggattcgtcgacgacttctggggttttgagtggcgcctgcagccaggagggctactggtactgcaacgggggcactgcgttccagcgctgtgtcaacggggaatgggatgcgtcccagagtgtggctgcgggcacggtctgcaccgccggtatctcggagaccatcaccatttcagccgccgccacgcgccgggatgccatgcgtcgtcatctggcgcgtcccaagcgtcactga

SEQ ID NO: 148 - protein, 427 aa, Aspergillus aculeatus
MSLSKIATLLLGSVSLVAGHGYVSSIEVDGTTYGGYLVDTYYYESDPPELIAWSTNATDDGYVSPSDYESVNIICHKGSAPGALSAPVAPGGWVQMTWNTWPTDHHGPVITYMANCHGSCADVDKTTLEFFKIDAGGLIDDTDVPGTWATDELIEDSYSRNITIPSDIAPGYYVLRHEIIALHSAENLDGAQNYPQCINLEVTGSETATPSGTLGTALYKETDPGIYVDIWNTLSTYTIPGPALYTAGSTATAAAAADTTTTSAGTTAEATTAAAAVSTTADAVPTESSAPSETSATTANPARPTAGSDIRFQPGQVKAGASVNNSATETSSGESATTTTTSVATAASSADSSTTSGVLSGACSQEGYWYCNGGTAFQRCVNGEWDASQSVAAGTVCTAGISETITISAAATRRDAMRRHLARPKRH

SEQ ID NO: 149 - DNA, 804 bp, Aspergillus aculeatus
atgcttgtcaaactcatctcttttctttcagctgctaccagcgtagctgctcatggtcatgtgtcaaacattgtgatcaacggggtgtcctaccgcggatgggacatcaattcggacccttacaattccaaccctccggtggtggttgcatggcaaacacccaacacagctaatggcttcatctcccctgatgcatacgacacagatgatgttatttgccatctgagcgctacgaatgccagaggccacgcagtcgtcgctgctggcgacaagatcagcctccagtggacgacctggcctgacagtcaccatggccctgtcatcagctacctagccaactgcggctccagctgcgagacagtcgataagaccaccctcgagttcttcaagatcgatggtgttggcttggtggatgagagcaatccccctggtatctggggagacgatgagctcattgccaacaacaactcttggctggtagagattccagctagtatcgcgccaggatactatgtgctgcgtcacgagttgatcgctctgcatggagcagggagtgagaatggagcccagaattacatgcaatgtttcaaccttcaggttactgggactggcacggtccagccttccggggtcctgggcacggagctgtacaaacccacagacgctggaattcttgtcaatatctaccagtcgctctccacctatgttgttcctggcccgaccctgatcccccaggccgtttccctcgttcagtcgagctccaccattaccgcctcgggcacggcagtgacaaccacggcttga

SEQ ID NO: 150 - protein, 267 aa, Aspergillus aculeatus
MLVKLISFLSAATSVAAHGHVSNIVINGVSYRGWDINSDPYNSNPPVVVAWQTPNTANGFISPDAYDTDDVICHLSATNARGHAVVAAGDKISLQWTTWPDSHHGPVISYLANCGSSCETVDKTTLEFFKIDGVGLVDESNPPGIWGDDELIANNNSWLVEIPASIAPGYYVLRHELIALHGAGSENGAQNYMQCFNLQVTGTGTVQPSGVLGTELYKPTDAGILVNIYQSLSTYVVPGPTLIPQAVSLVQSSSTITASGTAVTTTA

SEQ ID NO: 151 - DNA, 822 bp, Aspergillus aculeatus
atgaagtatcttgcgatcttcgcggcagcagcagctggactggcccgcccgacagcagcgcactacatcttcagcaagctgattctggacggcgaagtctctgaggactggcagtatattcgtaaaaccacccgggagacatgctatttgccgaccaagttcaccgacaccttcgacaacttgactccgaacgaccaggatttccggtgcaatctcggctcgttcagcaacgccgccaagaccgaagtggccgaggtggaagcgggctccacgattggcatgcagcttttcgctggtagccacatgcgtcacccgggacctgcgcaagtcttcatgtctaaggccccgtccggcaacgtacagagctacgagggtgacggctcctggttcaagatctgggagcgtacactctgcgacaaaagtggcgatctgactggagatgcgtggtgtacatacggccagaccgagatcgagtttcaaatccccgaggcgaccccgacgggcgaatacctggtccgagcggagcacatcggtcttcaccgcgcacagagtaatcaagccgagttctactacagctgcgcccaggtcaaggtcacgggcaatggtaccggggtgccgagccagacatatcagatccctggcatgtacaatgaccgctcggagcttttcaacgggctgaacttgtggtcctactcggtggagaacgtcgaggcagccatgaagaattctatcgtgggtgatgaaatttggaatggaagttctgttccctctgagtcccatgtcccgaagtataagaagagtcatgcttgtcgtgtttattga

SEQ ID NO: 152 - protein, 273 aa, Aspergillus aculeatus
MKYLAIFAAAAAGLARPTAAHYIFSKLILDGEVSEDWQYIRKTTRETCYLPTKFTDTFDNLTPNDQDFRCNLGSFSNAAKTEVAEVEAGSTIGMQLFAGSHMRHPGPAQVFMSKAPSGNVQSYEGDGSWFKIWERTLCDKSGDLTGDAWCTYGQTEIEFQIPEATPTGEYLVRAEHIGLHRAQSNQAEFYYSCAQVKVTGNGTGVPSQTYQIPGMYNDRSELFNGLNLWSYSVENVEAAMKNSIVGDEIWNGSSVPSESHVPKYKKSHACRVY

SEQ ID NO: 153 - DNA, 969 bp, Aurantiporus alborubescens
atgcgaaccatcgccacgtttgttacgcttgtagcctcagttctccctgcggtcctcgcacacggaggtgtcctctcctattcsaacggggggaattggtactggggatggaagccttacaattcacctgacgggcagaccaccatccaacgcccgtgggcaacatacaatccgatcactgatgcgacggatcctaccattgcttgcaacaacgacgggacatctggagctctgcagttgactgcgacagtcgcggcgggatctgccatcacggcgtattggaaccaggtgtggccgcatgataaagggccgatgacgacatacctcgcacaatgccccggcagtacctgcacaggagtcaacgcgaagactctgaaatggttcaagatcgatcacgccgggttgctttctggtactgtctacagtggctcgtgggcatcaggcaagatgattgcacagaactcgacctggacaactaccattccagcgacggtgccttcagggaactatctgatacgtttcgagactattgccctgcactctttgccagcgcaattttaccctgagtgcgcacaaattcaaatcacgggcggaggttcccgtgctccaaccgctgcagagcttgttagcttccctggcgcgtacagcaacaatgatcctggtgtcaacattgacatctactccaatgccgcgcagagtgcaaccacatacgtaataccaggacctccattgtacggcggtgcttccggatctggtccatcttccgcgcctccatcaagtaccccaggtagttcgtccacttcccacggtcccacgtccgtcagcacgtccagcagtgctgcaccatcgacgacaggaaccgtgacgcagtacggtcagtgcggtggcattggttgggctggagctaccggctgtatctcaccattcaagtgcacggtcatcaacgattattactaccagtgcctctga

SEQ ID NO: 154 - protein, 322 aa, Aurantiporus alborubescens
MRTIATFVTLVASVLPAVLAHGGVLSYSNGGNWYWGWKPYNSPDGQTTIQRPWATYNPITDATDPTIACNNDGTSGALQLTATVAAGSAITAYWNQVWPHDKGPMTTYLAQCPGSTCTGVNAKTLKWFKIDHAGLLSGTVYSGSWASGKMIAQNSTWTTTIPATVPSGNYLIRFETIALHSLPAQFYPECAQIQITGGGSRAPTAAELVSFPGAYSNNDPGVNIDIYSNAAQSATTYVIPGPPLYGGASGSGPSSAPPSSTPGSSSTSHGPTSVSTSSSAAPSTTGTVTQYGQCGGIGWAGATGCISPFKCTVINDYYYQCL

SEQ ID NO: 155 - DNA, 705 bp, Aurantiporus alborubescens
atgaaggctatcttggctattttctcggcccttgctccacttgccgctgcgcattataccttccctgattttattgtcaacggaacaacaactgccgattgggtctacatccgagagaccgcgaaccactactcgaatggtcctgtaaccaacgtgaacgatccagaattccgatgctacgagctggacctgcaaaacacggcagcgagtaccctcaccgccacggtctctgcaggctccagcgtcggctttaaagctaacagcgccctttaccatcctggttatctcgatgtgtatatgtccaaagcgaccccagctgctaattcacccagtgctggaacggaccaaagctggttcaaggtctatgaatccgctccggtcttcgcgaatggggccctaagcttcccttcggagaacatccaatctttcacgttcacaatcccgaagtcccttcccagtggccaatatctcatccgtgtggaacacatcgctctccactccgccagtagctacggaggtgcacaattctacatcagctgcgctcaagtcaatgtcgtcaacggcgggaacggaaacccaggaccgttagtcaagattcccggcgtttacactgggaacgagcctggcatcctcatcaacatctacagcttcccaccgggtttcagtggctaccaatccccgggacctgctgtgtggcgtggttga

SEQ ID NO: 156 - protein, 234 aa, Aurantiporus alborubescens
MKAILAIFSALAPLAAAHYTFPDFIVNGTTTADWVYIRETANHYSNGPVTNVNDPEFRCYELDLQNTAASTLTATVSAGSSVGFKANSALYHPGYLDVYMSKATPAANSPSAGTDQSWFKVYESAPVFANGALSFPSENIQSFTFTIPKSLPSGQYLIRVEHIALHSASSYGGAQFYISCAQVNVVNGGNGNPGPLVKIPGVYTGNEPGILINIYSFPPGFSGYQSPGPAVWRG

SEQ ID NO: 157 - DNA, 702 bp, Trichophaea saccata
atgacgcccctgaaactccgcccccttctcctcctggtgctttccacgaccctcagcctcgtgcacgcgcactatcgcttctacgaactgatcgccaacggggccacccacgcttccttcgaatacatccgccaatgggtgcccatctacagcaactctcccgtaaccgacgtcaccagcgtcaacctccgctgcaacgtcaacgccactcccgccgccgaggtgatcaccgttgctgccggtagcaccgtcggcttcgtagcagacacaacagtaacgcaccccggtgcgttcaccgcgtacatggcgaaagcgcccgaagacatcacggaatgggatggcaacggggactggttcaagatctgggagaagggtccaacgagtataaccagtagcgggataacctgggacgtcacggatacccaatggaccttcaccatcccttccgcgacaccaaacggtcaatacctactccgcttcgagcacatagcgctccacgccgccagcaccgtggggggtgctcaattctacatgtcgtgcgcgcagatacaagtaacgaacggcggcaacgggagtcccgggcccaccatcaagttcccgggcggatacagcgccacagaccccggtatcctgatcaatatctattatcccatccccactagttacactattcctggtccaccggtttggaccggtaagtaa

SEQ ID NO: 158 - protein, 233 aa, Trichophaea saccata
MTPLKLRPLLLLVLSTTLSLVHAHYRFYELIANGATHASFEYIRQWVPIYSNSPVTDVTSVNLRCNVNATPAAEVITVAAGSTVGFVADTTVTHPGAFTAYMAKAPEDITEWDGNGDWFKIWEKGPTSITSSGITWDVTDTQWTFTIPSATPNGQYLLRFEHIALHAASTVGGAQFYMSCAQIQVTNGGNGSPGPTIKFPGGYSATDPGILINIYYPIPTSYTIPGPPVWTGK

SEQ ID NO: 159 - DNA, 714 bp, Trichophaea saccata
atgaaatgccttctctccctccttctcgccgcgacagcggtctccgctcacacgatcttccaagaaatcggcataaacggggtgatgcaagctcgctacgactacatgcggctgccgtcctacgacggtcccattacggacgtaacgagcacctacatggcgtgcaacggtggtcccaatccattggtccaaatctcgaacgacgtcgctttcgtaaaagccggcgacagcatcacgctgcaatgggcgcaaacgttgacgacagatttcaacacggggctgatcatcgatccatcgcacttgggtcctgtgatggtctacatggccaaagtaccctccgccaccggtccgatccccaacagcggctggttcaaaatctacgaagacggctacgacccgacaacaaagacatgggcggtaaccaagctcatcaacaacaagggaaaagtgaccgtcaccatcccatcgtgtctaccggcaggggactacttgctgcgcggtgaaatcattgccttgcacgcggctagtacctatccaggcgcacagttttacatggagtgtgcgcagttgcggcttaccagtggcggcactaagatgcctaccacgtataacattccggggatctattcgcccactgatccgggtgttacgttcaatctttacaatggattcacgagttataccattcctggcccaaggccgtttacatgctag

SEQ ID NO: 160 - protein, 237 aa, Trichophaea saccata
MKCLLSLLLAATAVSAHTIFQEIGINGVMQARYDYMRLPSYDGPITDVTSTYMACNGGPNPLVQISNDVAFVKAGDSITLQWAQTLTTDFNTGLIIDPSHLGPVMVYMAKVPSATGPIPNSGWFKIYEDGYDPTTKTWAVTKLINNKGKVTVTIPSCLPAGDYLLRGEIIALHAASTYPGAQFYMECAQLRLTSGGTKMPTTYNIPGIYSPTDPGVTFNLYNGFTSYTIPGPRPFTC

SEQ ID NO: 161 - DNA, 1455 bp, Penicillium thomii
atgtctctgtctaagatttctggattgatcctcggatctgctgccttggtggctggccacggttacgtgagcggaatcgtcgttgacgatacctactatggtggataccttgtcacccagtacccttatgagagtgacgccccagagctcattgcctggtcggagcaagagaccgatctgggttacatcgatggctctgagtatgccaactccaacatcatctgtcacaaggaggccaaacctggtgctttggaagcacccgttaaggctggtggctccgtcgagctccagtggaccacttggcctaccagccaccacggtcctgtcattacctacatggccaactgtaacggcgactgtgacgacgttgacaagactactttgcagttcttcaagattgaccagggtggtttgatcagcgataccaccgagcccggtacctgggcaactgacaacctcatcgccaacaacaatagccgtactgtcaccgtccccagcgacattgccgatggaaactacgtcctccgtcacgagatcattgccctccactccgccggggagaccaacggtgcccagaactacccccaatgtatcaacttgaaggtcactggcggcggtagcgctactccttctggtaccctgggtaccgccctgtacaagaacaccgaccccggtatcctgatcaacatctacacttccctcagcacctacgatatccccggcccaaccctgtacactgccggcgccgccgctgctaccgctgcctccacggctgcctcttccaccgccgctgccgttactactgccgacgccgtcactaccgccgctgccgtcaccagcagctctgcatccgtggaagttgtgcccacaactactcccagctcatcaatcgtcagtgccttcccaacctggagcccctcttctaccccacccttctccaactcttccaacggatggcgtccgtcattcagccgcggacctggtggcccccgcttcacatctgctcctgctcctcagttctccgctcctagcggcgctcagcagaagcagtctgccactgctacccccatcgtggctacccctgtcgtgatcaccatgaccgagaccagcacctcctgggtcaccgaaatggttactcttactgacaagtctgttgtgcagaccaccagcgctgtcccagtcgtcgtcgccgccaccactacccttaccgagggaagcgagcctgctcagacagcctcccccagcgttgtctccggctcctctagctccggctctagctcctcatctaccaccaccacctcaaagacctcaactggatccgactacgtctccagcgactggatgtcttacctcagctccttgagcgctgctgaggtcctccagatgctgcgccagaccttccgttggatggtcagcaacgacaaggtgcacgctcgtgatattaccatcaactag

SEQ ID NO: 162 - protein, 484 aa, Penicillium thomii
MSLSKISGLILGSAALVAGHGYVSGIVVDDTYYGGYLVTQYPYESDAPELIAWSEQETDLGYIDGSEYANSNIICHKEAKPGALEAPVKAGGSVELQWTTWPTSHHGPVITYMANCNGDCDDVDKTTLQFFKIDQGGLISDTTEPGTWATDNLIANNNSRTVTVPSDIADGNYVLRHEIIALHSAGETNGAQNYPQCINLKVTGGGSATPSGTLGTALYKNTDPGILINIYTSLSTYDIPGPTLYTAGAAAATAASTAASSTAAAVTTADAVTTAAAVTSSSASVEVVPTTTPSSSIVSAFPTWSPSSTPPFSNSSNGWRPSFSRGPGGPRFTSAPAPQFSAPSGAQQKQSATATPIVATPVVITMTETSTSWVTEMVTLTDKSVVQTTSAVPVVVAATTTLTEGSEPAQTASPSVVSGSSSSGSSSSSTTTTSKTSTGSDYVSSDWMSYLSSLSAAEVLQMLRQTFRWMVSNDKVHARDITIN

SEQ ID NO: 163 - DNA, 1021 bp, Talaromyces stipitatus
atgccttccactaaagttgctgctctatctgccgtcctggctttggcctccacggttgctggccatggctttgtgcaaaatattgtcattgacggtaaatcgtaagtgacttgcttttgtactatagagctagataaatacttatactaaataattcagctacactggctacctcgtgaaccagtatccttaccagtccaacccaccagctgttattgggtggtcaaccactgcaaccgacttgggatttgtcgatggatctggatacaccaacccggatatcatctgccacaaaaacgccaaacccggtcagctttctgctccggttgccgcaggaggcaaggttgagctcgaatggacaacatggcccgagagccatcacggccctgtcatcagctatctcgccaattgcaatggcgattgtactaccgtggataagacgaagctcgaatttgtcaaaatcgatcagcggggtctgatcgacgacagcaatcctcccggtacatgggccgccgaccagctcatcgccgccaacaacagctggactgtaactattcccgagagcatcgcgcctggaaactacgtccttcgccacgaaatcatcgctcttcactccgccaacaacgcaaccggagctcaaaactaccctcaatgcatcaacttgcaaatcactggcagcgggacggccaacccatctggtacccctggcgagaaactctataccccaactgacccaggtatcttggtcaacatctaccagtcattgtcgtcttatgttattcccggtccgactttgtggagtggtgctgcagcgcacgttgttgccactgcagccggttctgctactggggttgcttctgccaccgctactccgaccactcttgtgactgccgtttcatcgcctaccggtgctccttcagtggtgactcctgaggctccttcagtaacctcgttcgccccagtggtgactgttactgatgtcgttactgtgactaccgtcatcactactactatctcttag

SEQ ID NO: 164 - protein, 320 aa, Talaromyces stipitatus
MPSTKVAALSAVLALASTVAGHGFVQNIVIDGKSYTGYLVNQYPYQSNPPAVIGWSTTATDLGFVDGSGYTNPDIICHKNAKPGQLSAPVAAGGKVELEWTTWPESHHGPVISYLANCNGDCTTVDKTKLEFVKIDQRGLIDDSNPPGTWAADQLIAANNSWTVTIPESIAPGNYVLRHEIIALHSANNATGAQNYPQCINLQITGSGTANPSGTPGEKLYTPTDPGILVNIYQSLSSYVIPGPTLWSGAAAHVVATAAGSATGVASATATPTTLVTAVSSPTGAPSVVTPEAPSVTSFAPVVTVTDVVTVTTVITTTIS

(19) United States
(12) Patent Application Publication
(10) Pub. No.: US 2017/0260557 A1
(43) Pub. Date: Sep. 14, 2017

(54) Compositions Comprising A Polypeptide Having Cellulolytic Enhancing Activity And An Organic Compound And Uses Thereof

(71) Applicant: Novozymes, Inc., Davis, CA (US)

(72) Inventors: Jason Quinlan, Woodland, CA (US); Feng Xu, Davis, CA (US); Matthew Sweeney, Sacramento, CA (US); Katja Salomon Johansen, Gentofte (DK)

(73) Assignee: Novozymes A/S, Bagsvaerd (DK)

(21) Appl. No.: 15/465,990

(22) Filed: Mar. 22, 2017

Related U.S. Application Data
(62) Division of application No. 14/500,641, filed on Sep. 29, 2014, now Pat. No. 9,663,808.
Application No. 14/500,641 is a division of application No. 13/816,432, filed on Mar. 22, 2013, now Pat. No. 8,846,351.
(60) Provisional application No. 61/373,210, filed on Aug. 12, 2010.
(60) Provisional application No. 61/373,124, filed on Aug. 12, 2010.
(60) Provisional application No. 61/373,128, filed on Aug. 12, 2010.
(60) Provisional application No. 61/373,145, filed on Aug. 12, 2010.
(60) Provisional application No. 61/373,150, filed on Aug. 12, 2010.
(60) Provisional application No. 61/373,157, filed on Aug. 12, 2010.
(60) Provisional application No. 61/373,170, filed on Aug. 12, 2010.

(51) Int. Cl. C12P 19/14; C12P 7/40; C07K 14/37; C12P 7/22; C12P 17/06; C12P 19/02; C12P 17/02

(52) U.S. Cl. CPC C12P 19/14; C12P 19/02; C12P 7/40; C12P 17/02; C12P 7/22; C12P 17/06; C07K 14/37; C12P 2203/00

(57) ABSTRACT

The present invention relates to compositions comprising: a polypeptide having cellulolytic enhancing activity and an organic compound. The present invention also relates to methods of using the compositions.

### CROSS-REFERENCE TO RELATED APPLICATIONS

This application is a divisional application of U.S. application Ser. No. 14/500,641, filed Sep. 29, 2014, now U.S. Pat. No. 9,663,808, which is a divisional application of U.S. application Ser. No. 13/816,432, filed Feb. 11, 2013, now U.S. Pat. No. 8,846,351, which is a 35 U.S.C. 371 national application of PCT/US2011/046723, filed Aug. 5, 2011, which claims priority or the benefit under 35 U.S.C. 119 of U.S. Provisional Application Ser. No. 61/373,124, filed Aug. 12, 2010, U.S. Provisional Application Ser. No. 61/373,128, filed Aug. 12, 2010, U.S. Provisional Application Ser. No. 61/373,145, filed Aug. 12, 2010, U.S. Provisional Application Ser. No. 61/373,150, filed Aug. 12, 2010, U.S. Provisional Application Ser. No. 61/373,157, filed Aug. 12, 2010, U.S. Provisional Application Ser. No. 61/373,166, filed Aug. 12, 2010, U.S. Provisional Application Ser. No. 61/373,170, filed Aug. 12, 2010, and U.S. Provisional Application Ser. No. 61/373,210, filed Aug. 12, 2010, the contents of which are fully incorporated herein by reference.

### STATEMENT AS TO RIGHTS TO INVENTIONS MADE UNDER FEDERALLY SPONSORED RESEARCH AND DEVELOPMENT

This invention was made with Government support under Cooperative Agreement DE-FC36-08GO18080 awarded by the Department of Energy. The government has certain rights in this invention.

### REFERENCE TO A SEQUENCE LISTING

This application contains a Sequence Listing in computer readable form. The computer readable form is incorporated herein by reference.

### BACKGROUND OF THE INVENTION

### Field of the Invention

The present invention relates to compositions comprising a polypeptide having cellulolytic enhancing activity and an organic compound, and to methods of using the compositions.

### Description of the Related Art

Cellulose is a polymer of the simple sugar glucose covalently linked by beta-1,4-bonds. Many microorganisms produce enzymes that hydrolyze beta-linked glucans. These enzymes include endoglucanases, cellobiohydrolases, and beta-glucosidases. Endoglucanases digest the cellulose polymer at random locations, opening it to attack by cellobiohydrolases. Cellobiohydrolases sequentially release molecules of cellobiose from the ends of the cellulose polymer. Cellobiose is a water-soluble beta-1,4-linked dimer of glucose. Beta-glucosidases hydrolyze cellobiose to glucose.

The conversion of lignocellulosic feedstocks into ethanol has the advantages of the ready availability of large amounts of feedstock, the desirability of avoiding burning or land filling the materials, and the cleanliness of the ethanol fuel. Wood, agricultural residues, herbaceous crops, and municipal solid wastes have been considered as feedstocks for ethanol production. These materials primarily consist of cellulose, hemicellulose, and lignin. Once the lignocellulose is converted to fermentable sugars, e.g., glucose, the fermentable sugars are easily fermented by yeast into ethanol.

WO 2005/074647, WO 2008/148131, WO 2011/035027 disclose isolated GH61 polypeptides having cellulolytic enhancing activity and the polynucleotides thereof from Thielavia terrestris. WO 2005/074656 and WO 2010/065830 disclose isolated GH61 polypeptides having cellulolytic enhancing activity and the polynucleotides thereof from Thermoascus aurantiacus. WO 2007/089290 discloses an isolated GH61 polypeptide having cellulolytic enhancing activity and the polynucleotide thereof from Trichoderma reesei. WO 2009/085935, WO 2009/085859, WO 2009/085864, and WO 2009/085868 disclose isolated GH61 polypeptides having cellulolytic enhancing activity and the polynucleotides thereof from Myceliophthora thermophila. WO 2010/138754 discloses isolated GH61 polypeptides having cellulolytic enhancing activity and the polynucleotides thereof from Aspergillus fumigatus. WO 2011/005867 discloses isolated GH61 polypeptides having cellulolytic enhancing activity and the polynucleotides thereof from Penicillium pinophilum. WO 2011/039319 discloses isolated GH61 polypeptides having cellulolytic enhancing activity and the polynucleotides thereof from Thermoascus sp. WO 2011/041397 discloses isolated GH61 polypeptides having cellulolytic enhancing activity and the polynucleotides thereof from Penicillium sp. WO 2011/041504 discloses isolated GH61 polypeptides having cellulolytic enhancing activity and the polynucleotides thereof from Thermoascus crustaceous. WO 2008/151043 discloses methods of increasing the activity of a GH61 polypeptide having cellulolytic enhancing activity by adding a soluble activating divalent metal cation to a composition comprising the polypeptidelt would be advantageous in the art to improve the ability of polypeptides having cellulolytic enhancing activity to enhance enzymatic hydrolysis of lignocellulosic feedstocks.

The present invention relates to compositions comprising a polypeptide having cellulolytic enhancing activity and an organic compound, and to methods of using the compositions.

### SUMMARY OF THE INVENTION

The present invention relates to compositions comprising: (a) a polypeptide having cellulolytic enhancing activity; and (b) an organic compound, wherein the combination of the polypeptide having cellulolytic enhancing activity and the organic compound enhances hydrolysis of a cellulosic material by a cellulolytic enzyme.

The present invention also relates to methods for degrading or converting a cellulosic material, comprising: treating the cellulosic material with an enzyme composition in the presence of a polypeptide having cellulolytic enhancing activity and an organic compound, wherein the combination of the polypeptide having cellulolytic enhancing activity and the organic compound enhances hydrolysis of the cellulosic material by the enzyme composition.

The present invention also relates to methods for producing a fermentation product, comprising:

(a) saccharifying a cellulosic material with an enzyme composition in the presence of a polypeptide having cellulolytic enhancing activity and an organic compound, wherein the combination of the polypeptide having cellulolytic enhancing activity and the organic compound enhances hydrolysis of the cellulosic material by the enzyme composition;

(b) fermenting the saccharified cellulosic material with one or more (e.g., several) fermenting microorganisms to produce the fermentation product; and

(c) recovering the fermentation product from the fermentation.

The present invention also relates to methods of fermenting a cellulosic material, comprising: fermenting the cellulosic material with one or more (e.g., several) fermenting microorganisms, wherein the cellulosic material is saccharified with an enzyme composition in the presence of a polypeptide having cellulolytic enhancing activity and an organic compound, wherein the combination of the polypeptide having cellulolytic enhancing activity and the organic compound enhances hydrolysis of the cellulosic material by the enzyme composition.

In one aspect, the organic compound is any compound described herein, such as a compound with a pKa that is less than or equal to the pH of the solution referenced in the method or composition. In one aspect, the organic compound comprises a pKa less than or equal to about any of 8.0, 7.5, 7.0, 6.5, 6.0, 5.5, 5.0, 4.5, 3.5, 3.0, or 2.5. In one aspect, the organic compound comprises a pKa less than or about equal to about 5.0.

### BRIEF DESCRIPTION OF THE FIGURES

FIGS. 1A (methyl-4-hydroxybenzoate), 1B (3,5-dihydroxybenzoic acid), 1C (4-hydroxybenzoic acid), 1D (glutamate), 1E (tartrate), 1F (4-hydroxy-3-methoxycinnamic acid), 1G (methyl-3,4,5-trihydroxybenzoate), 1H (3,4-dihydroxybenzoic acid), 1I (ascorbate), 1J (4-methyl hexanoic acid), 1K (2-methyl hexanoic acid), 1L (pyrocatechol), 1M (pyrogallol), and 1N (dehydroascorbate) show (1) the effect of an organic compound on hydrolysis of AVICEL® by the Trichoderma reesei cellulase composition in the absence of a GH61 polypeptide (organic compound effect_{(no GH61)}, white bars), (2) the effect of an organic compound on hydrolysis of AVICEL® by a T. reesei cellulase composition in the presence of a GH61 polypeptide (organic compound effect_{(+GH61)}, grey bars), and (3) the effect of a GH61 polypeptide on hydrolysis of AVICEL® by the T. reesei cellulase composition in the presence of an organic compound (GH61 effect, black bars) for 3 and 7 days.

FIGS. 2A, 2B, 2C, and 2D show the influence of pH on the incubation of organic compounds and the Thermoascus aurantiacus GH61A polypeptide with PASC and Ca(II). FIG. 2A: PACE gel of the products of incubation of ascorbate and gallic acid with the T. aurantiacus GH61A polypeptide, PASC and Ca(II) at various pH values. Lane 1: glucose; lane 2: ascorbate, pH 4.0; lane 3: ascorbate, pH 4.5; lane 4: ascorbate, pH 5.5; lane 5: ascorbate, pH 5.6; lane 6: gallic acid, pH 3.9; lane 7: gallic acid, pH 4.5; lane 8: gallic acid, pH 5.5. FIG. 2B: PACE gel of the products of incubation of cysteine with the T. aurantiacus GH61A polypeptide, PASC and Ca(II) at various pH values. Lane 1: glucose; lane 2: pH 4.6; lane 3: pH 5.24; lane 4: blank; lane 5: pH 6.29; lane 6: pH 7.35; lane 7: blank; lane 8: pH 8.00; lane 9: pH 8.75; lane 11: DP 6 cellodextrin. FIGS. 2C and 2D show the densitometry analysis of the PACE gel shown in FIG. 2A for ascorbate (FIG. 2C) and gallic acid (FIG. 2D). Circles: degree of polymerization=4; squares: degree of polymerization=8; diamonds: degree of polymerization=9.

FIGS. 3A and 3B show (A) the results of a microcrystalline cellulose hydrolysis assay with the T. reesei cellulase composition in the presence of the T. aurantiacus GH61A polypeptide and chromatographed fractions of acid-pretreated xylan. Solid line: fractional hydrolysis with GH61; dashed line: mean fractional hydrolysis; and (B) an LC-MS chromatogram of a representative HPLC fraction of acid-pretreated xylan.

### DEFINITIONS

Acetylxylan esterase: The term “acetylxylan esterase” means a carboxylesterase (EC 3.1.1.72) that catalyzes the hydrolysis of acetyl groups from polymeric xylan, acetylated xylose, acetylated glucose, alpha-napthyl acetate, and p-nitrophenyl acetate. For purposes of the present invention, acetylxylan esterase activity is determined using 0.5 mM p-nitrophenylacetate as substrate in 50 mM sodium acetate pH 5.0 containing 0.01% TWEEN™ 20 (polyoxyethylene sorbitan monolaurate). One unit of acetylxylan esterase is defined as the amount of enzyme capable of releasing 1 μmole of p-nitrophenolate anion per minute at pH 5, 25° C.

Allelic variant: The term “allelic variant” means any of two or more alternative forms of a gene occupying the same chromosomal locus. Allelic variation arises naturally through mutation, and may result in polymorphism within populations. Gene mutations can be silent (no change in the encoded polypeptide) or may encode polypeptides having altered amino acid sequences. An allelic variant of a polypeptide is a polypeptide encoded by an allelic variant of a gene.

Alpha-L-arabinofuranosidase: The term “alpha-L-arabinofuranosidase” means an alpha-L-arabinofuranoside arabinofuranohydrolase (EC 3.2.1.55) that catalyzes the hydrolysis of terminal non-reducing alpha-L-arabinofuranoside residues in alpha-L-arabinosides. The enzyme acts on alpha-L-arabinofuranosides, alpha-L-arabinans containing (1,3)- and/or (1,5)-linkages, arabinoxylans, and arabinogalactans. Alpha-L-arabinofuranosidase is also known as arabinosidase, alpha-arabinosidase, alpha-L-arabinosidase, alpha-arabinofuranosidase, polysaccharide alpha-L-arabinofuranosidase, alpha-L-arabinofuranoside hydrolase, L-arabinosidase, or alpha-L-arabinanase. For purposes of the present invention, alpha-L-arabinofuranosidase activity is determined using 5 mg of medium viscosity wheat arabinoxylan (Megazyme International Ireland, Ltd., Bray, Co. Wicklow, Ireland) per ml of 100 mM sodium acetate pH 5 in a total volume of 200 μl for 30 minutes at 40° C. followed by arabinose analysis by AMINEX® HPX-87H column chromatography (Bio-Rad Laboratories, Inc., Hercules, Calif., USA).

Alpha-glucuronidase: The term “alpha-glucuronidase” means an alpha-D-glucosiduronate glucuronohydrolase (EC 3.2.1.139) that catalyzes the hydrolysis of an alpha-D-glucuronoside to D-glucuronate and an alcohol. For purposes of the present invention, alpha-glucuronidase activity is determined according to de Vries, 1998, J. Bacteriol. 180: 243-249. One unit of alpha-glucuronidase equals the amount of enzyme capable of releasing 1 μmole of glucuronic or 4-O-methylglucuronic acid per minute at pH 5, 40° C.

Beta-glucosidase: The term “beta-glucosidase” means a beta-D-glucoside glucohydrolase (E.C. 3.2.1.21) that catalyzes the hydrolysis of terminal non-reducing beta-D-glucose residues with the release of beta-D-glucose. For purposes of the present invention, beta-glucosidase activity is determined using p-nitrophenyl-beta-D-glucopyranoside as substrate according to the procedure of Venturi et al., 2002, Extracellular beta-D-glucosidase from Chaetomium thermophilum var. coprophilum: production, purification and some biochemical properties, J. Basic Microbiol. 42: 55-66. One unit of beta-glucosidase is defined as 1.0 μmole of p-nitrophenolate anion produced per minute at 25° C., pH 4.8 from 1 mM p-nitrophenyl-beta-D-glucopyranoside as substrate in 50 mM sodium citrate containing 0.01% TWEEN® 20.

Beta-xylosidase: The term “beta-xylosidase” means a beta-D-xyloside xylohydrolase (E.C. 3.2.1.37) that catalyzes the exo-hydrolysis of short beta (1-4)-xylooligosaccharides to remove successive D-xylose residues from non-reducing termini. For purposes of the present invention, one unit of beta-xylosidase is defined as 1.0 μmole of p-nitrophenolate anion produced per minute at 40° C., pH 5 from 1 mM p-nitrophenyl-beta-D-xyloside as substrate in 100 mM sodium citrate containing 0.01% TWEEN® 20.

Carbohydrate moiety: The term “carbohydrate moiety” as used herein refers to an optionally substituted 5 or 6-membered cyclic monosaccharide radical comprising an oxygen bridge between two carbon atoms. Non-limiting examples include an optionally substituted radical of glucose, sucrose, and fructose. In some aspects, the carbohydrate moiety is optionally substituted with a second carbohydrate moiety (to generate a disaccharide moiety, e.g., a lactose moiety). In some aspects, the carbohydrate moiety contains only one cyclic monosaccharide.

cDNA: The term “cDNA” means a DNA molecule that can be prepared by reverse transcription from a mature, spliced, mRNA molecule obtained from a eukaryotic cell. cDNA lacks intron sequences that may be present in the corresponding genomic DNA. The initial, primary RNA transcript is a precursor to mRNA that is processed through a series of steps, including splicing, before appearing as mature spliced mRNA.

Cellobiohydrolase: The term “cellobiohydrolase” means a 1,4-beta-D-glucan cellobiohydrolase (E.C. 3.2.1.91) that catalyzes the hydrolysis of 1,4-beta-D-glucosidic linkages in cellulose, cellooligosaccharides, or any beta-1,4-linked glucose containing polymer, releasing cellobiose from the reducing or non-reducing ends of the chain (Teeri, 1997, Crystalline cellulose degradation: New insight into the function of cellobiohydrolases, Trends in Biotechnology 15: 160-167; Teeri et al., 1998, Trichoderma reesei cellobiohydrolases: why so efficient on crystalline cellulose?, Biochem. Soc. Trans. 26: 173-178). For purposes of the present invention, cellobiohydrolase activity is determined according to the procedures described by Lever et al., 1972, Anal. Biochem. 47: 273-279; van Tilbeurgh et al., 1982, FEBS Letters, 149: 152-156; van Tilbeurgh and Claeyssens, 1985, FEBS Letters, 187: 283-288; and Tomme et al., 1988, Eur. J. Biochem. 170: 575-581. In the present invention, the Lever et al. method can be employed to assess hydrolysis of cellulose in corn stover, while the methods of van Tilbeurgh et al. and Tomme et al. can be used to determine the cellobiohydrolase activity on a fluorescent disaccharide derivative, 4-methylumbelliferyl-β-D-lactoside.

Cellulolytic enhancing activity: The term “cellulolytic enhancing activity” means a biological activity catalyzed by a GH61 polypeptide that enhances the hydrolysis of a cellulosic material by enzyme having cellulolytic activity. For purposes of the present invention, cellulolytic enhancing activity is determined by measuring the increase in reducing sugars or the increase of the total of cellobiose and glucose from the hydrolysis of a cellulosic material by cellulolytic enzyme under the following conditions: 1-50 mg of total protein/g of cellulose in PCS, wherein total protein is comprised of 50-99.5% w/w cellulolytic enzyme protein and 0.5-50% w/w protein of a GH61 polypeptide having cellulolytic enhancing activity for 1-7 days at 50° C. compared to a control hydrolysis with equal total protein loading without cellulolytic enhancing activity (1-50 mg of cellulolytic protein/g of cellulose in PCS). In a preferred aspect, a mixture of CELLUCLAST® 1.5L (Novozymes A/S, Bagsvaerd, Denmark) in the presence of 2-3% of total protein weight Aspergillus oryzae beta-glucosidase (recombinantly produced in Aspergillus oryzae according to WO 02/095014) or 2-3% of total protein weight Aspergillus fumigatus beta-glucosidase (recombinantly produced in Aspergillus oryzae as described in WO 2002/095014) of cellulase protein loading is used as the source of the cellulolytic activity.

The GH61 polypeptides having cellulolytic enhancing activity enhance the hydrolysis of a cellulosic material catalyzed by enzyme having cellulolytic activity by reducing the amount of cellulolytic enzyme required to reach the same degree of hydrolysis preferably at least 1.01-fold, more preferably at least 1.05-fold, more preferably at least 1.10-fold, more preferably at least 1.25-fold, more preferably at least 1.5-fold, more preferably at least 2-fold, more preferably at least 3-fold, more preferably at least 4-fold, more preferably at least 5-fold, even more preferably at least 10-fold, and most preferably at least 20-fold.

Cellulolytic enzyme or cellulase: The term “cellulolytic enzyme” or “cellulase” means one or more (e.g., several) enzymes that hydrolyze a cellulosic material. Such enzymes include endoglucanase(s), cellobiohydrolase(s), beta-glucosidase(s), or combinations thereof. The two basic approaches for measuring cellulolytic activity include: (1) measuring the total cellulolytic activity, and (2) measuring the individual cellulolytic activities (endoglucanases, cellobiohydrolases, and beta-glucosidases) as reviewed in Zhang et al., Outlook for cellulase improvement: Screening and selection strategies, 2006, Biotechnology Advances 24: 452-481. Total cellulolytic activity is usually measured using insoluble substrates, including Whatman No 1 filter paper, microcrystalline cellulose, bacterial cellulose, algal cellulose, cotton, pretreated lignocellulose, etc. The most common total cellulolytic activity assay is the filter paper assay using Whatman No 1 filter paper as the substrate. The assay was established by the International Union of Pure and Applied Chemistry (IUPAC) (Ghose, 1987, Measurement of cellulase activities, Pure Appl. Chem. 59: 257-68).

For purposes of the present invention, cellulolytic enzyme activity is determined by measuring the increase in hydrolysis of a cellulosic material by cellulolytic enzyme(s) under the following conditions: 1-20 mg of cellulolytic enzyme protein/g of cellulose in PCS for 3-7 days at 50° C. compared to a control hydrolysis without addition of cellulolytic enzyme protein. Typical conditions are 1 ml reactions, washed or unwashed PCS, 5% insoluble solids, 50 mM sodium acetate pH 5, 1 mM MnSO₄, 50° C., 72 hours, sugar analysis by AMINEX® HPX-87H column (Bio-Rad Laboratories, Inc., Hercules, Calif., USA).

Cellulosic material: The term “cellulosic material” means any material containing cellulose. The predominant polysaccharide in the primary cell wall of biomass is cellulose, the second most abundant is hemicellulose, and the third is pectin. The secondary cell wall, produced after the cell has stopped growing, also contains polysaccharides and is strengthened by polymeric lignin covalently cross-linked to hemicellulose. Cellulose is a homopolymer of anhydrocellobiose and thus a linear beta-(1-4)-D-glucan, while hemicelluloses include a variety of compounds, such as xylans, xyloglucans, arabinoxylans, and mannans in complex branched structures with a spectrum of substituents. Although generally polymorphous, cellulose is found in plant tissue primarily as an insoluble crystalline matrix of parallel glucan chains. Hemicelluloses usually hydrogen bond to cellulose, as well as to other hemicelluloses, which help stabilize the cell wall matrix.

Cellulose is generally found, for example, in the stems, leaves, hulls, husks, and cobs of plants or leaves, branches, and wood of trees. The cellulosic material can be, but is not limited to, agricultural residue, herbaceous material (including energy crops), municipal solid waste, pulp and paper mill residue, waste paper, and wood (including forestry residue) (see, for example, Wiselogel et al., 1995, in Handbook on Bioethanol (Charles E. Wyman, editor), pp. 105-118, Taylor & Francis, Washington D.C.; Wyman, 1994, Bioresource Technology 50: 3-16; Lynd, 1990, Applied Biochemistry and Biotechnology 24/25: 695-719; Mosier et al., 1999, Recent Progress in Bioconversion of Lignocellulosics, in Advances in Biochemical Engineering/Biotechnology, T. Scheper, managing editor, Volume 65, pp. 23-40, Springer-Verlag, New York). It is understood herein that the cellulose may be in the form of lignocellulose, a plant cell wall material containing lignin, cellulose, and hemicellulose in a mixed matrix. In a preferred aspect, the cellulosic material is any biomass material. In another preferred aspect, the cellulosic material is lignocellulose, which comprises cellulose, hemicelluloses, and lignin.

In one aspect, the cellulosic material is agricultural residue. In another aspect, the cellulosic material is herbaceous material (including energy crops). In another aspect, the cellulosic material is municipal solid waste. In another aspect, the cellulosic material is pulp and paper mill residue. In another aspect, the cellulosic material is waste paper. In another aspect, the cellulosic material is wood (including forestry residue).

In another aspect, the cellulosic material is arundo. In another aspect, the cellulosic material is bagasse. In another aspect, the cellulosic material is bamboo. In another aspect, the cellulosic material is corn cob. In another aspect, the cellulosic material is corn fiber. In another aspect, the cellulosic material is corn stover. In another aspect, the cellulosic material is miscanthus. In another aspect, the cellulosic material is orange peel. In another aspect, the cellulosic material is rice straw. In another aspect, the cellulosic material is switchgrass. In another aspect, the cellulosic material is wheat straw.

In another aspect, the cellulosic material is aspen. In another aspect, the cellulosic material is eucalyptus. In another aspect, the cellulosic material is fir. In another aspect, the cellulosic material is pine. In another aspect, the cellulosic material is poplar. In another aspect, the cellulosic material is spruce. In another aspect, the cellulosic material is willow.

In another aspect, the cellulosic material is algal cellulose. In another aspect, the cellulosic material is bacterial cellulose. In another aspect, the cellulosic material is cotton linter. In another aspect, the cellulosic material is filter paper. In another aspect, the cellulosic material is microcrystalline cellulose. In another aspect, the cellulosic material is phosphoric-acid treated cellulose.

In another aspect, the cellulosic material is an aquatic biomass. As used herein the term “aquatic biomass” means biomass produced in an aquatic environment by a photosynthesis process. The aquatic biomass can be algae, emergent plants, floating-leaf plants, or submerged plants.

The cellulosic material may be used as is or may be subjected to pretreatment, using conventional methods known in the art, as described herein. In a preferred aspect, the cellulosic material is pretreated.

Coding sequence: The term “coding sequence” means a polynucleotide, which directly specifies the amino acid sequence of a polypeptide. The boundaries of the coding sequence are generally determined by an open reading frame, which usually begins with the ATG start codon or alternative start codons such as GTG and TTG and ends with a stop codon such as TAA, TAG, and TGA. The coding sequence may be a DNA, cDNA, synthetic, or recombinant polynucleotide.

Control sequences: The term “control sequences” means all components necessary for the expression of a polynucleotide encoding a polypeptide. Each control sequence may be native or foreign to the polynucleotide encoding the polypeptide or native or foreign to each other. Such control sequences include, but are not limited to, a leader, polyadenylation sequence, propeptide sequence, promoter, signal peptide sequence, and transcription terminator. At a minimum, the control sequences include a promoter, and transcriptional and translational stop signals. The control sequences may be provided with linkers for the purpose of introducing specific restriction sites facilitating ligation of the control sequences with the coding region of the polynucleotide encoding a polypeptide.

Endoglucanase: The term “endoglucanase” means an endo-1,4-(1,3;1,4)-beta-D-glucan 4-glucanohydrolase (E.C. 3.2.1.4) that catalyzes endohydrolysis of 1,4-beta-D-glycosidic linkages in cellulose, cellulose derivatives (such as carboxymethyl cellulose and hydroxyethyl cellulose), lichenin, beta-1,4 bonds in mixed beta-1,3 glucans such as cereal beta-D-glucans or xyloglucans, and other plant material containing cellulosic components. Endoglucanase activity can be determined by measuring reduction in substrate viscosity or increase in reducing ends determined by a reducing sugar assay (Zhang et al., 2006, Biotechnology Advances 24: 452-481). For purposes of the present invention, endoglucanase activity is determined using carboxymethyl cellulose (CMC) as substrate according to the procedure of Ghose, 1987, Pure and Appl. Chem. 59: 257-268, at pH 5, 40° C.

Expression: The term “expression” includes any step involved in the production of the polypeptide including, but not limited to, transcription, post-transcriptional modification, translation, post-translational modification, and secretion.

Expression vector: The term “expression vector” means a linear or circular DNA molecule that comprises a polynucleotide encoding a polypeptide and is operably linked to additional nucleotides that provide for its expression.

Family 61 glycoside hydrolase: The term “Family 61 glycoside hydrolase” or “Family GH61” or “GH61” means a polypeptide falling into the glycoside hydrolase Family 61 according to Henrissat B., 1991, A classification of glycosyl hydrolases based on amino-acid sequence similarities, Biochem. J. 280: 309-316, and Henrissat B., and Bairoch A., 1996, Updating the sequence-based classification of glycosyl hydrolases, Biochem. J. 316: 695-696. The enzymes in this family were originally classified as a glycoside hydrolase family based on measurement of very weak endo-1,4-beta-D-glucanase activity in one family member. The structure and mode of action of these enzymes are non-canonical and they cannot be considered as bona fide glycosidases. However, they are kept in the CAZy classification on the basis of their capacity to enhance the breakdown of lignocellulose when used in conjunction with a cellulase or a mixture of cellulases.

Feruloyl esterase: The term “feruloyl esterase” means a 4-hydroxy-3-methoxycinnamoyl-sugar hydrolase (EC 3.1.1.73) that catalyzes the hydrolysis of 4-hydroxy-3-methoxycinnamoyl (feruloyl) groups from esterified sugar, which is usually arabinose in “natural” substrates, to produce ferulate (4-hydroxy-3-methoxycinnamate). Feruloyl esterase is also known as ferulic acid esterase, hydroxycinnamoyl esterase, FAE-III, cinnamoyl ester hydrolase, FAEA, cinnAE, FAE-I, or FAE-II. For purposes of the present invention, feruloyl esterase activity is determined using 0.5 mM p-nitrophenylferulate as substrate in 50 mM sodium acetate pH 5.0. One unit of feruloyl esterase equals the amount of enzyme capable of releasing 1 μmole of p-nitrophenolate anion per minute at pH 5, 25° C.

Flavonoid moiety: As used herein, the term “flavonoid moiety” refers to the structure:

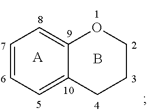

wherein ring B may optionally contain an annular double bond between positions 1 and 2, 2 and 3, and/or 3 and 4; and wherein rings A and B are optionally substituted at one or more (e.g., several) of positions 2, 3, 4, 5, 6, 7, and 8. The term “isoflavonoid moiety” refers to a flavonoid moiety wherein ring B contains no annular double bond between positions 1 and 2, 2 and 3, or 3 and 4. The term “flavylium ion moiety” refers to a flavonoid moiety wherein ring B contains a double bond between positions 1 and 2 and a double bond between positions 3 and 4. The term “anthocyanin moiety” refers to a flavylium ion containing an optionally substituted carbohydrate moiety and wherein position 2 is substituted with an optionally substituted phenyl moiety. The term “anthocyanidin moiety” refers to a flavylium ion moiety lacking an optionally substituted carbohydrate moiety and wherein position 2 is substituted with an optionally substituted phenyl moiety.

Hemicellulolytic enzyme or hemicellulase: The term “hemicellulolytic enzyme” or “hemicellulase” means one or more (e.g., several) enzymes that hydrolyze a hemicellulosic material. See, for example, Shallom, D. and Shoham, Y. Microbial hemicellulases. Current Opinion In Microbiology, 2003, 6(3): 219-228). Hemicellulases are key components in the degradation of plant biomass. Examples of hemicellulases include, but are not limited to, an acetylmannan esterase, an acetylxylan esterase, an arabinanase, an arabinofuranosidase, a coumaric acid esterase, a feruloyl esterase, a galactosidase, a glucuronidase, a glucuronoyl esterase, a mannanase, a mannosidase, a xylanase, and a xylosidase. The substrates of these enzymes, the hemicelluloses, are a heterogeneous group of branched and linear polysaccharides that are bound via hydrogen bonds to the cellulose microfibrils in the plant cell wall, crosslinking them into a robust network. Hemicelluloses are also covalently attached to lignin, forming together with cellulose a highly complex structure. The variable structure and organization of hemicelluloses require the concerted action of many enzymes for its complete degradation. The catalytic modules of hemicellulases are either glycoside hydrolases (GHs) that hydrolyze glycosidic bonds, or carbohydrate esterases (CEs), which hydrolyze ester linkages of acetate or ferulic acid side groups. These catalytic modules, based on homology of their primary sequence, can be assigned into GH and CE families marked by numbers. Some families, with an overall similar fold, can be further grouped into clans, marked alphabetically (e.g., GH-A). A most informative and updated classification of these and other carbohydrate active enzymes is available in the Carbohydrate-Active Enzymes (CAZy) database. Hemicellulolytic enzyme activities can be measured according to Ghose and Bisaria, 1987, Pure & Appl. Chem. 59: 1739-1752.

Host cell: The term “host cell” means any cell type that is susceptible to transformation, transfection, transduction, and the like with a nucleic acid construct or expression vector comprising a polynucleotide of the present invention. The term “host cell” encompasses any progeny of a parent cell that is not identical to the parent cell due to mutations that occur during replication.

Isolated or Purified: The term “isolated” or “purified” means a polypeptide or polynucleotide that is removed from at least one component with which it is naturally associated. For example, a polypeptide may be at least 1% pure, e.g., at least 5% pure, at least 10% pure, at least 20% pure, at least 40% pure, at least 60% pure, at least 80% pure, at least 90% pure, or at least 95% pure, as determined by SDS-PAGE, and a polynucleotide may be at least 1% pure, e.g., at least 5% pure, at least 10% pure, at least 20% pure, at least 40% pure, at least 60% pure, at least 80% pure, at least 90% pure, or at least 95% pure, as determined by agarose electrophoresis.

Lactone moiety: The term “lactone moiety” refers to an optionally substituted cyclic ester (e.g., an optionally substituted five or six-membered cyclic ester).

Mature polypeptide: The term “mature polypeptide” means a polypeptide in its final form following translation and any post-translational modifications, such as N-terminal processing, C-terminal truncation, glycosylation, phosphorylation, etc. It is known in the art that a host cell may produce a mixture of two of more different mature polypeptides (i.e., with a different C-terminal and/or N-terminal amino acid) expressed by the same polynucleotide. The mature polypeptide can be predicted using the SignalP program (Nielsen et al., 1997, Protein Engineering 10: 1-6).

Mature polypeptide coding sequence: The term “mature polypeptide coding sequence” is defined herein as a nucleotide sequence that encodes a mature polypeptide having biological activity. The mature polypeptide coding sequence can be predicted using the SignalP program (Nielsen et al., 1997, supra).

Nucleic acid construct: The term “nucleic acid construct” means a nucleic acid molecule, either single- or double-stranded, which is isolated from a naturally occurring gene or is modified to contain segments of nucleic acids in a manner that would not otherwise exist in nature or which is synthetic. The term nucleic acid construct is synonymous with the term “expression cassette” when the nucleic acid construct contains the control sequences required for expression of a coding sequence of the present invention.

Operably linked: The term “operably linked” means a configuration in which a control sequence is placed at an appropriate position relative to the coding sequence of a polynucleotide such that the control sequence directs the expression of the coding sequence.

Phenolic moiety: The term “phenolic moiety” refers to an optionally substituted six-membered aromatic ring bonded to one or more (e.g., several) hydroxyl groups. As used herein, a phenolic moiety includes both fused and non-fused ring systems.

Polypeptide fragment: The term “fragment” means a polypeptide having one or more (e.g., several) amino acids deleted from the amino and/or carboxyl terminus of a mature polypeptide; wherein the fragment has biological activity.

Pretreated corn stover: The term “PCS” or “Pretreated Corn Stover” means a cellulosic material derived from corn stover by treatment with heat and dilute sulfuric acid, alkaline pretreatment, or neutral pretreatment.

Sequence Identity: The relatedness between two amino acid sequences or between two nucleotide sequences is described by the parameter “sequence identity”.

For purposes of the present invention, the degree of sequence identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 3.0.0, 5.0.0, or later. The optional parameters used are gap open penalty of 10, gap extension penalty of 0.5, and the EBLOSUM62 (EMBOSS version of BLOSUM62) substitution matrix. The output of Needle labeled “longest identity” (obtained using the −nobrief option) is used as the percent identity and is calculated as follows:

(Identical Residues×100)/(Length of Alignment−Total Number of Gaps in Alignment)

For purposes of the present invention, the degree of sequence identity between two deoxyribonucleotide sequences is determined using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 3.0.0 or later. The optional parameters used are gap open penalty of 10, gap extension penalty of 0.5, and the EDNAFULL (EMBOSS version of NCBI NUC4.4) substitution matrix. The output of Needle labeled “longest identity” (obtained using the −nobrief option) is used as the percent identity and is calculated as follows:

(Identical Deoxyribonucleotides×100)/(Length of Alignment−Total Number of Gaps in Alignment)

Subsequence: The term “subsequence” means a polynucleotide having one or more (e.g., several) nucleotides deleted from the 5′ and/or 3′ end of a mature polypeptide coding sequence; wherein the subsequence encodes a fragment having biological activity.

Substituted: The term “substituted” refers to the replacement of one or more (e.g., several) hydrogen atoms of a moiety with a monovalent or divalent radical. “Optionally substituted” indicates that the moiety may be substituted or unsubstituted. A moiety lacking the terms “optionally substituted” and “substituted” is intended an unsubstituted moiety (e.g., “phenyl” is intended an unsubstituted phenyl unless indicated as a substituted phenyl or an optionally substituted phenyl).

Variant: The term “variant” means a polypeptide having cellulolytic enhancing activity comprising an alteration, i.e., a substitution, insertion, and/or deletion of one or more (e.g., several) amino acid residues at one or more (e.g., several) positions. A substitution means a replacement of an amino acid occupying a position with a different amino acid; a deletion means removal of an amino acid occupying a position; and an insertion means adding one or more (e.g., several) amino acids, e.g., 1-5 amino acids, adjacent to an amino acid occupying a position.

Xylan-containing material: The term “xylan-containing material” means any material comprising a plant cell wall polysaccharide containing a backbone of beta-(1-4)-linked xylose residues. Xylans of terrestrial plants are heteropolymers possessing a beta-(1-4)-D-xylopyranose backbone, which is branched by short carbohydrate chains. They comprise D-glucuronic acid or its 4-O-methyl ether, L-arabinose, and/or various oligosaccharides, composed of D-xylose, L-arabinose, D- or L-galactose, and D-glucose. Xylan-type polysaccharides can be divided into homoxylans and heteroxylans, which include glucuronoxylans, (arabino)glucuronoxylans, (glucurono)arabinoxylans, arabinoxylans, and complex heteroxylans. See, for example, Ebringerova et al., 2005, Adv. Polym. Sci. 186: 1-67.

In the methods of the present invention, any material containing xylan may be used. In a preferred aspect, the xylan-containing material is lignocellulose.

Xylan degrading activity or xylanolytic activity: The term “xylan degrading activity” or “xylanolytic activity” means a biological activity that hydrolyzes xylan-containing material. The two basic approaches for measuring xylanolytic activity include: (1) measuring the total xylanolytic activity, and (2) measuring the individual xylanolytic activities (e.g., endoxylanases, beta-xylosidases, arabinofuranosidases, alpha-glucuronidases, acetylxylan esterases, feruloyl esterases, and alpha-glucuronyl esterases). Recent progress in assays of xylanolytic enzymes was summarized in several publications including Biely and Puchard, Recent progress in the assays of xylanolytic enzymes, 2006, Journal of the Science of Food and Agriculture 86(11): 1636-1647; Spanikova and Biely, 2006, Glucuronoyl esterase—Novel carbohydrate esterase produced by Schizophyllum commune, FEBS Letters 580(19): 4597-4601; Herrmann, Vrsanska, Jurickova, Hirsch, Biely, and Kubicek, 1997, The beta-D-xylosidase of Trichoderma reesei is a multifunctional beta-D-xylan xylohydrolase, Biochemical Journal 321: 375-381.

Total xylan degrading activity can be measured by determining the reducing sugars formed from various types of xylan, including, for example, oat spelt, beechwood, and larchwood xylans, or by photometric determination of dyed xylan fragments released from various covalently dyed xylans. The most common total xylanolytic activity assay is based on production of reducing sugars from polymeric 4-O-methyl glucuronoxylan as described in Bailey, Biely, Poutanen, 1992, Interlaboratory testing of methods for assay of xylanase activity, Journal of Biotechnology 23(3): 257-270. Xylanase activity can also be determined with 0.2% AZCL-arabinoxylan as substrate in 0.01% TRITON® X-100 (4-(1,1,3,3-tetramethylbutyl)phenyl-polyethylene glycol) and 200 mM sodium phosphate buffer pH 6 at 37° C. One unit of xylanase activity is defined as 1.0 μmole of azurine produced per minute at 37° C., pH 6 from 0.2% AZCL-arabinoxylan as substrate in 200 mM sodium phosphate pH 6 buffer.

For purposes of the present invention, xylan degrading activity is determined by measuring the increase in hydrolysis of birchwood xylan (Sigma Chemical Co., Inc., St. Louis, Mo., USA) by xylan-degrading enzyme(s) under the following typical conditions: 1 ml reactions, 5 mg/ml substrate (total solids), 5 mg of xylanolytic protein/g of substrate, 50 mM sodium acetate pH 5, 50° C., 24 hours, sugar analysis using p-hydroxybenzoic acid hydrazide (PHBAH) assay as described by Lever, 1972, A new reaction for colorimetric determination of carbohydrates, Anal. Biochem 47: 273-279.

Xylanase: The term “xylanase” means a 1,4-beta-D-xylan-xylohydrolase (E.C. 3.2.1.8) that catalyzes the endohydrolysis of 1,4-beta-D-xylosidic linkages in xylans. For purposes of the present invention, xylanase activity is determined with 0.2% AZCL-arabinoxylan as substrate in 0.01% TRITON® X-100 and 200 mM sodium phosphate buffer pH 6 at 37° C. One unit of xylanase activity is defined as 1.0 μmole of azurine produced per minute at 37° C., pH 6 from 0.2% AZCL-arabinoxylan as substrate in 200 mM sodium phosphate pH 6 buffer.

As used herein and in the appended claims, the singular forms “a,” “or,” and “the” include plural referents unless the context clearly dictates otherwise. It is understood that the aspects of the invention described herein include “consisting” and/or “consisting essentially of” aspects.

### DETAILED DESCRIPTION OF THE INVENTION

The present invention relates to compositions comprising: (a) a polypeptide having cellulolytic enhancing activity; and (b) an organic compound, wherein the combination of the polypeptide having cellulolytic enhancing activity and the organic compound enhances hydrolysis of a cellulosic material by a cellulolytic enzyme. In one aspect, the compositions further comprise (c) one or more (e.g., several) enzymes selected from the group consisting of a cellulase, a hemicellulase, an esterase, an expansin, a laccase, a ligninolytic enzyme, a pectinase, a peroxidase, a protease, and a swollenin.

The present invention also relates to methods for degrading or converting a cellulosic material, comprising: treating the cellulosic material with an enzyme composition in the presence of a polypeptide having cellulolytic enhancing activity and an organic compound, wherein the combination of the polypeptide having cellulolytic enhancing activity and the organic compound enhances hydrolysis of the cellulosic material by the enzyme composition. In one aspect, the method above further comprises recovering the degraded or converted cellulosic material. Soluble products of degradation or conversion of the cellulosic material can be separated from the insoluble cellulosic material using technology well known in the art such as, for example, centrifugation, filtration, and gravity settling.

The present invention also relates to methods for producing a fermentation product, comprising:

(a) saccharifying a cellulosic material with an enzyme composition in the presence of a polypeptide having cellulolytic enhancing activity and an organic compound, wherein the combination of the polypeptide having cellulolytic enhancing activity and the organic compound enhances hydrolysis of the cellulosic material by the enzyme composition;

(b) fermenting the saccharified cellulosic material with one or more (e.g., several) fermenting microorganisms to produce the fermentation product; and

(c) recovering the fermentation product from the fermentation.

The present invention also relates to methods of fermenting a cellulosic material, comprising: fermenting the cellulosic material with one or more (e.g., several) fermenting microorganisms, wherein the cellulosic material is saccharified with an enzyme composition in the presence of a polypeptide having cellulolytic enhancing activity and an organic compound, wherein the combination of the polypeptide having cellulolytic enhancing activity and the organic compound enhances hydrolysis of the cellulosic material by the enzyme composition. In one aspect, the fermenting of the cellulosic material produces a fermentation product. In another aspect, the method further comprises recovering the fermentation product from the fermentation.

### Organic Compounds and Compositions

In the methods and compositions of the present invention, the organic compound may be any compound described herein, such as a compound with a pKa that is less than or about equal to the pH used in the methods or compositions. In one aspect, the organic compound comprises a pKa that is less than the pH of the composition. In another aspect, the organic compound comprises a pKa less than or equal to about any of 8.0, 7.5, 7.0, 6.5, 6.0, 5.5, 5.0, 4.5, 3.5, 3.0, or 2.5. In another aspect, the organic compound comprises a pKa less than or about equal to about 5.0.

In another aspect, the pH of a composition comprising the organic compound (e.g., a composition of any of the methods described herein) is greater than or equal to the lowest pKa of the organic compound. In another aspect, the composition comprising the organic compound has a pH of greater than or about any of 3.0, 3.5, 4.0, 4.5, 5.0, 5.5, 6.0, 6.5, 7.0, 7.5, or 8.0. In another aspect, the pH of the composition is greater than or about equal to about 5.0.

In another aspect of the methods and compositions, greater than or about 95%, 90%, 85%, 80%, 75%, 70%, 65%, 60%, 55%, 50%, 45%, 40%, 35%, 30%, 25%, 20%, 15%, 10%, 5%, 2.5%, or 1% of the organic compound is in proton dissociated form. In another aspect, the proton dissociated form of the organic compound is neutral with respect to the proton dissociated moiety.

In another aspect, the proton dissociated form of the organic compound is anionic with respect to the proton dissociated moiety. In another aspect, less than or about 1%, 2.5%, 5%, 10%, 15%, 20%, 25%, 30%, 35%, 40%, 45%, 50%, 55%, 60%, 65%, 70%, 75%, 80%, 85%, 90%, or 95% of the organic compound is in proton associated form. In another aspect, the proton associated form of the organic compound is neutral with respect to the proton associated moiety. In another aspect, the proton associated form of the organic compound is cationic with respect to the proton associated moiety.

In another aspect of the methods and compositions, the organic compound comprises an acidic moiety, such as a carboxylic acid moiety. In another aspect, the organic compound comprises a lactone moiety. In some aspect, the organic compound comprises a phenolic moiety.

In another aspect, the organic compound is a flavonoid. In another aspect, the organic compound comprises a isoflavonoid moiety. In another aspect, the organic compound comprises a flavylium ion, such as an anthocyanidin moiety or an anthocyanin moiety.

In another aspect of the methods and compositions, the organic compound is selected from the group consisting of:

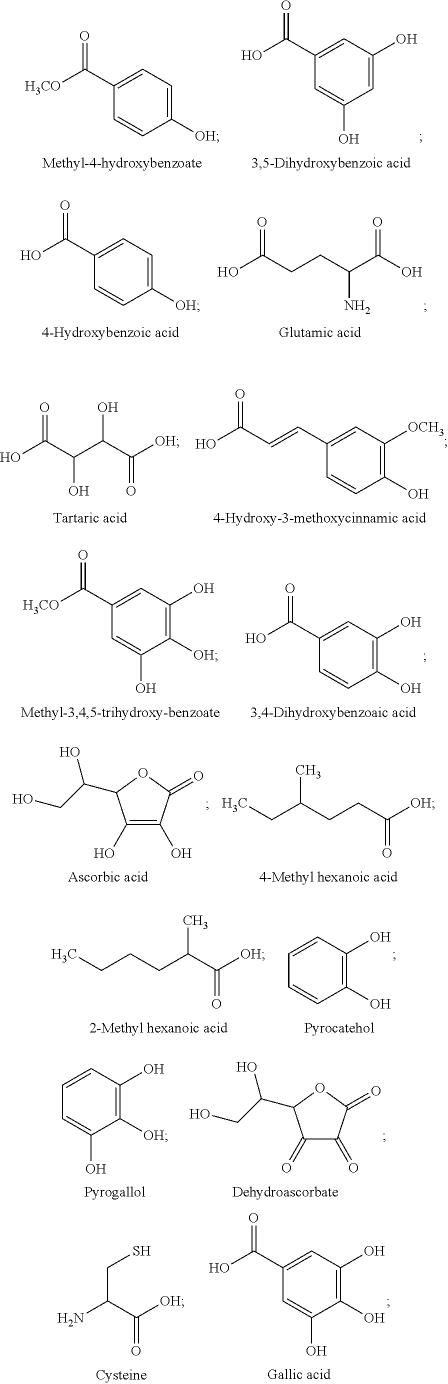

or a salt or solvate thereof.

In another aspect, the organic compound of the methods and compositions, is a compound from Table 1:

TABLE 1

| | | | GH61 | GH61 |
|---|---|---|---|---|
| | | | effect-day 1 | effect-day 1 |
| Organic Compound | ~pKa1 | ~pKa2 | (stand. dev.) | (stand. dev.) |
| glutamic acid | 2.13 | 4.31 | 0.989 | 1.07 |
| | | | (0.0188) | (0.0234) |
| fumaric acid | 3.03 | 4.54 | 0.989 | 1.02 |
| | | | (0.00776) | (0.0275) |
| trans-aconitic acid | 2.8 | 4.46 | 0.982 | 1.00 |
| | | | (0.0157) | (0.0279) |
| methylmalonic acid | 3.07 | 5.76 | 0.977 | 1.00 |
| | | | (0.00604) | (0.0304) |
| oxalic acid | 1.23 | 4.19 | 0.969 | 1.02 |
| | | | (0.0182) | (0.0487) |
| sorbic acid | 4.75 | | 1.01 | 0.973 |
| | | | (0.0322) | (0.0220) |
| maleic acid | 1.93 | 6.58 | 1.01 | 0.980 |
| | | | (0.00681) | (0.0219) |
| tartrate | 3.22 | 4.82 | 1.01 | 1.11 |
| | | | (0.0369) | (0.0140) |
| caproic acid | 4.85 | | 0.967 | 1.00 |
| | | | (0.0129) | (0.0172) |
| 3,5-dihydroxybenzoic | 4.04 | | 1.01 | 1.07 |
| acid | | | (0.0386) | (0.0224) |
| 4-hydroxy-3- | 4.08 | 8.45 | 1.00 | 1.00 |
| methoxybenzoic acid | | | (0.0423) | (0.0258) |
| 4-hydroxybenzoic | 4.58 | | 1.01 | 1.03 |
| acid | | | (0.0193) | (0.0276) |
| 2,6-dimethoxyphenol | 9.97 | | 1.05 | 1.17 |
| | | | (0.0142) | (0.0225) |
| abietic acid | 7.6 | | 0.992 | 1.02 |
| | | | (0.0236) | (0.0200) |
| 4-hydroxy-3- | 4.27 | 8.83 | 0.960 | 1.03 |
| methoxycinnamic | | | (0.0136) | (0.0273) |
| acid | | | | |
| 3,5-di-tert-butyl-4- | 4.77 | | 0.991 | 1.00 |
| hydroxybenzoic acid | | | (0.0197) | (0.0102) |
| 2-aminophenol | 4.86 | 9.99 | 1.12 | 1.09 |
| | | | (0.0220) | (0.0365) |
| 3,4-dihydroxybenzoic | 4.38 | 8.74 | 0.967 | 1.06 |
| acid | | | (0.0210) | (0.017141) |
| butyric acid | 4.82 | | 1.00 | 0.990 |
| | | | (0.0288) | (0.00914) |
| dehydroascorbate | 3.9 | | 1.05 | 1.08 |
| | | | (0.0104) | (0.00817) |
| pyrogallol | 5.1 | | 1.228 | 1.27 |
| | | | (0.0177) | (0.00827) |
| epicatechin | 8.16 | 9.4 | 1.26 | 1.684 |
| | | | (0.0168) | (0.0285) |
| morin | 3.5 | | 1.13 | 1.17 |
| | | | (0.0644) | (0.125) |
| naringenin | 6.9 | | 1.01 | 1. |
| | | | (0.0179) | (0.0292) |
| quercitin | 7 | | 1.02 | 1.11 |
| | | | (0.0271) | (0.0382) |

In one aspect, the organic compound is produced by acid-pretreatment of cellulosic, lignocellulosic or hemicellulosic material, or the monosaccharides thereof, generating one or more (e.g., several) organic acids of low pKa. In another aspect, potential sources of low pKa organic acids are added to cellulosic feedstock prior to pretreatment as described.

The effective amount of the organic compound can depend on one or more (e.g., several) factors including, but not limited to, the mixture of component cellulolytic enzymes, the cellulosic substrate, the concentration of cellulosic substrate, the pretreatment(s) of the cellulosic substrate, non-cellulosic components (e.g., native or degraded lignin or hemicellulose), non-cellulase components, temperature, and reaction time.

The organic compound is preferably present in an amount that is not limiting with regard to the GH61 polypeptide having cellulolytic enhancing activity, cellulolytic enzyme(s), and cellulose. In one aspect, the compound is present in an amount that is not limiting with regard to the GH61 polypeptide having cellulolytic enhancing activity. In another aspect, the compound is present in an amount that is not limiting with regard to the cellulolytic enzyme(s). In another aspect, the compound is present in an amount that is not limiting with regard to the cellulose. In another aspect, the compound is present in an amount that is not limiting with regard to the GH61 polypeptide having cellulolytic enhancing activity and the cellulolytic enzyme(s). In another aspect, the compound is present in an amount that is not limiting with regard to the GH61 polypeptide having cellulolytic enhancing activity and the cellulose. In another aspect, the compound is present in an amount that is not limiting with regard to the cellulolytic enzyme(s) and the cellulose. In another aspect, the compound is present in an amount that is not limiting with regard to the GH61 polypeptide having cellulolytic enhancing activity, the cellulolytic enzyme(s), and the cellulose.

In one aspect, an effective amount of the organic compound to cellulosic material as a molar ratio to glucosyl units of cellulose is about 10⁻⁶ to about 10, e.g., about 10⁻⁶ to about 7.5, about 10⁻⁶ to about 5, about 10⁻⁶ to about 2.5, about 10⁻⁶ to about 1, about 10⁻⁵ to about 1, about 10⁻⁵ to about 10⁻¹, about 10⁻⁴ to about 10⁻¹, about 10⁻³ to about 10⁻¹, or about 10⁻³ to about 10⁻². In another aspect, an effective amount of the organic compound to cellulosic material as a molar ratio to glucosyl units of cellulose is about 10⁻⁶ to about 10. In another aspect, an effective amount of the organic compound to cellulosic material as a molar ratio to glucosyl units of cellulose is about 10⁻⁶ to about 7.5. In another aspect, an effective amount of the organic compound to cellulosic material as a molar ratio to glucosyl units of cellulose is about 10⁻⁶ to about 5. In another aspect, an effective amount of the organic compound to cellulosic material as a molar ratio to glucosyl units of cellulose is about 10⁻⁶ to about 2.5. In another aspect, an effective amount of the organic compound to cellulosic material as a molar ratio to glucosyl units of cellulose is about 10⁻⁶ to about 1. In another aspect, an effective amount of the organic compound to cellulosic material as a molar ratio to glucosyl units of cellulose is about 10⁻⁵ to about 1. In another aspect, an effective amount of the organic compound to cellulosic material as a molar ratio to glucosyl units of cellulose is about 10⁻⁵ to about 10⁻¹. In another aspect, an effective amount of the organic compound to cellulosic material as a molar ratio to glucosyl units of cellulose is about 10⁻⁴ to about 10⁻¹. In another aspect, an effective amount of the organic compound to cellulosic material as a molar ratio to glucosyl units of cellulose is about 10⁻³ to about 10⁻¹. In another aspect, an effective amount of the organic compound to cellulosic material as a molar ratio to glucosyl units of cellulose is about 10⁻³ to about 10⁻².

In another aspect, an effective amount of the organic compound to cellulose is about 10⁻⁶ to about 10 g per g of cellulose, e.g., about 10⁻⁶ to about 7.5, about 10⁻⁶ to about 5, about 10⁻⁶ to about 2.5, about 10⁻⁶ to about 1, about 10⁻⁵ to about 1, about 10⁻⁵ to about 10⁻¹, about 10⁻⁴ to about 10⁻¹, about 10⁻³ to about 10⁻¹, or about 10⁻³ to about 10⁻² g per g of cellulose. In another aspect, an effective amount of the organic compound to cellulose is about 10⁻⁶ to about 10 g per g of cellulose. In another aspect, an effective amount of the organic compound to cellulose is about 10⁻⁶ to about 10 g per g of cellulose. In another aspect, an effective amount of the organic compound to cellulose is about 10⁻⁶ to about 7.5 g per g of cellulose. In another aspect, an effective amount of the organic compound to cellulose is about 10⁻⁶ to about 5 g per g of cellulose. In another aspect, an effective amount of the organic compound to cellulose is about 10⁻⁶ to about 2.5 g per g of cellulose. In another aspect, an effective amount of the organic compound to cellulose is about 10⁻⁶ to about 1 g per g of cellulose. In another aspect, an effective amount of the organic compound to cellulose is about 10⁻⁶ to about 1 g per g of cellulose. In another aspect, an effective amount of the organic compound to cellulose is about 10⁻⁶ to about 10⁻¹ g per g of cellulose. In another aspect, an effective amount of the organic compound to cellulose is about 10⁻⁴ to about 10⁻¹ g per g of cellulose. In another aspect, an effective amount of the organic compound to cellulose is about 10⁻³ to about 10⁻¹ g per g of cellulose. In another aspect, an effective amount of the organic compound to cellulose is about 10⁻³ to about 10⁻² g per g of cellulose.

In another aspect, an effective amount of the organic compound is about 0.1 μM to about 1 M, e.g., about 0.5 μM to about 0.75 M, about 0.75 μM to about 0.5 M, about 1 μM to about 0.25 M, about 1 μM to about 0.1 M, about 5 μM to about 50 mM, about 10 μM to about 25 mM, about 50 μM to about 25 mM, about 10 μM to about 10 mM, about 5 μM to about 5 mM, or about 0.1 mM to about 1 mM. In another aspect, an effective amount of the organic compound is about 0.1 μM to about 1 M. In another aspect, an effective amount of the organic compound is about 0.5 μM to about 0.75 M. In another aspect, an effective amount of the organic compound is about 0.75 μM to about 0.5 M. In another aspect, an effective amount of the organic compound is about 1 μM to about 0.25 M. In another aspect, an effective amount of the organic compound is about 1 μM to about 0.1 M. In another aspect, an effective amount of the organic compound is about 5 μM to about 50 mM. In another aspect, an effective amount of the organic compound is about 10 μM to about 25 mM. In another aspect, an effective amount of the organic compound is about 50 μM to about 25 mM. In another aspect, an effective amount of the organic compound is about 10 μM to about 10 mM. In another aspect, an effective amount of the organic compound is about 5 μM to about 5 mM. In another aspect, an effective amount of the organic compound is about 0.1 mM to about 1 mM.

In another aspect, one or more (e.g., several) organic compounds are used in any of the methods of the present invention.

In another aspect of the present invention, the organic compound(s) may be recycled from a completed saccharification or completed saccharification and fermentation to a new saccharification. The organic compound(s) can be recovered using standard methods in the art, e.g., filtration/centrifugation pre- or post-distillation, to remove residual solids, cellular debris, etc. and then recirculated to the new saccharification.

### Polypeptides Having Cellulolytic Enhancing Activity and Polynucleotides Thereof

In the methods of the present invention, any GH61 polypeptide having cellulolytic enhancing activity can be used.

In a first aspect, the polypeptide having cellulolytic enhancing activity comprises the following motifs:

[ILMV]-P-X(4,5)-G-X-Y-[ILMV]-X-R-X-[EQ]-X(4)-[HNQ] (SEQ ID NO: 125 or SEQ ID NO: 126) and [FW]-[TF]-K-[AIV],

wherein X is any amino acid, X(4,5) is any amino acid at 4 or 5 contiguous positions, and X(4) is any amino acid at 4 contiguous positions.

The isolated polypeptide comprising the above-noted motifs may further comprise:

H-X(1,2)-G-P-X(3)-[YW]-[AILMV] (SEQ ID NO: 127 or SEQ ID NO: 128),

[EQ]-X-Y-X(2)-C-X-[EHQN]-[FILV]-X-[ILV] (SEQ ID NO: 129), or

H-X(1,2)-G-P-X(3)-[YW]-[AILMV] (SEQ ID NO: 130 or SEQ ID NO: 131) and [EQ]-X-Y-X(2)-C-X-[EHQN]-[FILV]-X-[ILV] (SEQ ID NO: 132),

wherein X is any amino acid, X(1,2) is any amino acid at 1 position or 2 contiguous positions, X(3) is any amino acid at 3 contiguous positions, and X(2) is any amino acid at 2 contiguous positions. In the above motifs, the accepted IUPAC single letter amino acid abbreviation is employed.

In a preferred embodiment, the isolated GH61 polypeptide having cellulolytic enhancing activity further comprises H-X(1,2)-G-P-X(3)-[YW]-[AILMV] (SEQ ID NO: 133 or SEQ ID NO: 134). In another preferred embodiment, the isolated GH61 polypeptide having cellulolytic enhancing activity further comprises [EQ]-X-Y-X(2)-C-X-[EHQN]-[FILV]-X-[ILV] (SEQ ID NO: 135). In another preferred embodiment, the isolated GH61 polypeptide having cellulolytic enhancing activity further comprises H-X(1,2)-G-P-X(3)-[YW]-[AILMV] (SEQ ID NO: 136 or SEQ ID NO: 137) and [EQ]-X-Y-X(2)-C-X-[EHQN]-[FILV]-X-[ILV] (SEQ ID NO: 138).

In a second aspect, isolated polypeptides having cellulolytic enhancing activity, comprise the following motif:

[ILMV]-P-X(4,5)-G-X-Y-[ILMV]-X-R-X-[EQ]-X(3)-A-[HNQ] (SEQ ID NO: 139 or SEQ ID NO: 140),

wherein X is any amino acid, X(4,5) is any amino acid at 4 or 5 contiguous positions, and X(3) is any amino acid at 3 contiguous positions. In the above motif, the accepted IUPAC single letter amino acid abbreviation is employed.

In a third aspect, the polypeptide having cellulolytic enhancing activity comprises an amino acid sequence that has a degree of identity to the mature polypeptide of SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 42, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 142, SEQ ID NO: 144, SEQ ID NO: 146, SEQ ID NO: 148, SEQ ID NO: 150, SEQ ID NO: 152, SEQ ID NO: 154, SEQ ID NO: 156, SEQ ID NO: 158, SEQ ID NO: 160, SEQ ID NO: 162, or SEQ ID NO: 164 of preferably at least 60%, more preferably at least 65%, more preferably at least 70%, more preferably at least 75%, more preferably at least 80%, more preferably at least 85%, even more preferably at least 90%, most preferably at least 91%, at least 92%, at least 93%, at least 94%, or at least 95%, or at least 100% and even most preferably at least 96%, at least 97%, at least 98%, at least 99%, or at least 100%.

In a preferred aspect, the mature polypeptide is amino acids 20 to 326 of SEQ ID NO: 2, amino acids 18 to 239 of SEQ ID NO: 4, amino acids 20 to 258 of SEQ ID NO: 6, amino acids 19 to 226 of SEQ ID NO: 8, amino acids 20 to 304 of SEQ ID NO: 10, amino acids 23 to 250 of SEQ ID NO: 12, amino acids 22 to 249 of SEQ ID NO: 14, amino acids 20 to 249 of SEQ ID NO: 16, amino acids 18 to 232 of SEQ ID NO: 18, amino acids 16 to 235 of SEQ ID NO: 20, amino acids 19 to 323 of SEQ ID NO: 22, amino acids 16 to 310 of SEQ ID NO: 24, amino acids 20 to 246 of SEQ ID NO: 26, amino acids 22 to 354 of SEQ ID NO: 28, amino acids 22 to 250 of SEQ ID NO: 30, or amino acids 22 to 322 of SEQ ID NO: 32, amino acids 24 to 444 of SEQ ID NO: 34, amino acids 26 to 253 of SEQ ID NO: 36, amino acids 20 to 223 of SEQ ID NO: 38, amino acids 18 to 246 of SEQ ID NO: 40, amino acids 20 to 334 of SEQ ID NO: 42, amino acids 18 to 227 of SEQ ID NO: 44, amino acids 22 to 368 of SEQ ID NO: 46, amino acids 25 to 330 of SEQ ID NO: 48, amino acids 17 to 236 of SEQ ID NO: 50, amino acids 17 to 250 of SEQ ID NO: 52, amino acids 23 to 478 of SEQ ID NO: 54, amino acids 17 to 230 of SEQ ID NO: 56, amino acids 20 to 257 of SEQ ID NO: 58, amino acids 23 to 251 of SEQ ID NO: 60, amino acids 19 to 349 of SEQ ID NO: 62, amino acids 24 to 436 of SEQ ID NO: 64, amino acids 21 to 344 of SEQ ID NO: 142, amino acids 21 to 389 of SEQ ID NO: 144, amino acids 22 to 406 of SEQ ID NO: 146, amino acids 20 to 427 of SEQ ID NO: 148, amino acids 18 to 267 of SEQ ID NO: 150, amino acids 21 to 273 of SEQ ID NO: 152, amino acids 21 to 322 of SEQ ID NO: 154, amino acids 18 to 234 of SEQ ID NO: 156, amino acids 24 to 233 of SEQ ID NO: 158, amino acids 17 to 237 of SEQ ID NO: 160, amino acids 20 to 484 of SEQ ID NO: 162, or amino acids 22 to 320 of SEQ ID NO: 164.

A polypeptide having cellulolytic enhancing activity preferably comprises or consists of the amino acid sequence of SEQ ID NO: 2 or an allelic variant thereof; or a fragment thereof that has cellulolytic enhancing activity. In a preferred aspect, the polypeptide comprises or consists of the amino acid sequence of SEQ ID NO: 2. In another preferred aspect, the polypeptide comprises or consists of the mature polypeptide of SEQ ID NO: 2. In another preferred aspect, the polypeptide comprises or consists of amino acids 20 to 326 of SEQ ID NO: 2, or an allelic variant thereof; or a fragment thereof that has cellulolytic enhancing activity. In another preferred aspect, the polypeptide comprises or consists of amino acids 20 to 326 of SEQ ID NO: 2.

A polypeptide having cellulolytic enhancing activity preferably comprises or consists of the amino acid sequence of SEQ ID NO: 4 or an allelic variant thereof; or a fragment thereof that has cellulolytic enhancing activity. In a preferred aspect, the polypeptide comprises or consists of the amino acid sequence of SEQ ID NO: 4. In another preferred aspect, the polypeptide comprises or consists of the mature polypeptide of SEQ ID NO: 4. In another preferred aspect, the polypeptide comprises or consists of amino acids 18 to 239 of SEQ ID NO: 4, or an allelic variant thereof; or a fragment thereof that has cellulolytic enhancing activity. In another preferred aspect, the polypeptide comprises or consists of amino acids 18 to 239 of SEQ ID NO: 4.

A polypeptide having cellulolytic enhancing activity preferably comprises or consists of the amino acid sequence of SEQ ID NO: 6 or an allelic variant thereof; or a fragment thereof that has cellulolytic enhancing activity. In a preferred aspect, the polypeptide comprises or consists of the amino acid sequence of SEQ ID NO: 6. In another preferred aspect, the polypeptide comprises or consists of the mature polypeptide of SEQ ID NO: 6. In another preferred aspect, the polypeptide comprises or consists of amino acids 20 to 258 of SEQ ID NO: 6, or an allelic variant thereof; or a fragment thereof that has cellulolytic enhancing activity. In another preferred aspect, the polypeptide comprises or consists of amino acids 20 to 258 of SEQ ID NO: 6.

A polypeptide having cellulolytic enhancing activity preferably comprises or consists of the amino acid sequence of SEQ ID NO: 8 or an allelic variant thereof; or a fragment thereof that has cellulolytic enhancing activity. In a preferred aspect, the polypeptide comprises or consists of the amino acid sequence of SEQ ID NO: 8. In another preferred aspect, the polypeptide comprises or consists of the mature polypeptide of SEQ ID NO: 8. In another preferred aspect, the polypeptide comprises or consists of amino acids 19 to 226 of SEQ ID NO: 8, or an allelic variant thereof; or a fragment thereof that has cellulolytic enhancing activity. In another preferred aspect, the polypeptide comprises or consists of amino acids 19 to 226 of SEQ ID NO: 8.

A polypeptide having cellulolytic enhancing activity preferably comprises or consists of the amino acid sequence of SEQ ID NO: 10 or an allelic variant thereof; or a fragment thereof that has cellulolytic enhancing activity. In a preferred aspect, the polypeptide comprises or consists of the amino acid sequence of SEQ ID NO: 10. In another preferred aspect, the polypeptide comprises or consists of the mature polypeptide of SEQ ID NO: 10. In another preferred aspect, the polypeptide comprises or consists of amino acids 20 to 304 of SEQ ID NO: 10, or an allelic variant thereof; or a fragment thereof that has cellulolytic enhancing activity. In another preferred aspect, the polypeptide comprises or consists of amino acids 20 to 304 of SEQ ID NO: 10.

A polypeptide having cellulolytic enhancing activity preferably comprises or consists of the amino acid sequence of SEQ ID NO: 12 or an allelic variant thereof; or a fragment thereof having cellulolytic enhancing activity. In a preferred aspect, the polypeptide comprises or consists of the amino acid sequence of SEQ ID NO: 12. In another preferred aspect, the polypeptide comprises or consists of the mature polypeptide of SEQ ID NO: 12. In another preferred aspect, the polypeptide comprises or consists of amino acids 16 to 317 of SEQ ID NO: 12, or an allelic variant thereof; or a fragment thereof having cellulolytic enhancing activity. In another preferred aspect, the polypeptide comprises or consists of amino acids 16 to 317 of SEQ ID NO: 12.

A polypeptide having cellulolytic enhancing activity preferably comprises or consists of the amino acid sequence of SEQ ID NO: 14 or an allelic variant thereof; or a fragment thereof that has cellulolytic enhancing activity. In a preferred aspect, the polypeptide comprises or consists of the amino acid sequence of SEQ ID NO: 14. In another preferred aspect, the polypeptide comprises or consists of the mature polypeptide of SEQ ID NO: 14. In another preferred aspect, the polypeptide comprises or consists of amino acids 23 to 250 of SEQ ID NO: 14, or an allelic variant thereof; or a fragment thereof that has cellulolytic enhancing activity. In another preferred aspect, the polypeptide comprises or consists of amino acids 23 to 250 of SEQ ID NO: 14.

A polypeptide having cellulolytic enhancing activity preferably comprises or consists of the amino acid sequence of SEQ ID NO: 16 or an allelic variant thereof; or a fragment thereof that has cellulolytic enhancing activity. In a preferred aspect, the polypeptide comprises or consists of the amino acid sequence of SEQ ID NO: 16. In another preferred aspect, the polypeptide comprises or consists of the mature polypeptide of SEQ ID NO: 16. In another preferred aspect, the polypeptide comprises or consists of amino acids 20 to 249 of SEQ ID NO: 16, or an allelic variant thereof; or a fragment thereof that has cellulolytic enhancing activity. In another preferred aspect, the polypeptide comprises or consists of amino acids 20 to 249 of SEQ ID NO: 16.

A polypeptide having cellulolytic enhancing activity preferably comprises or consists of the amino acid sequence of SEQ ID NO: 18 or an allelic variant thereof; or a fragment thereof that has cellulolytic enhancing activity. In a preferred aspect, the polypeptide comprises or consists of the amino acid sequence of SEQ ID NO: 18. In another preferred aspect, the polypeptide comprises or consists of the mature polypeptide of SEQ ID NO: 18. In another preferred aspect, the polypeptide comprises or consists of amino acids 18 to 232 of SEQ ID NO: 18, or an allelic variant thereof; or a fragment thereof that has cellulolytic enhancing activity. In another preferred aspect, the polypeptide comprises or consists of amino acids 18 to 232 of SEQ ID NO: 18.

A polypeptide having cellulolytic enhancing activity preferably comprises or consists of the amino acid sequence of SEQ ID NO: 20 or an allelic variant thereof; or a fragment thereof that has cellulolytic enhancing activity. In a preferred aspect, the polypeptide comprises or consists of the amino acid sequence of SEQ ID NO: 20. In another preferred aspect, the polypeptide comprises or consists of the mature polypeptide of SEQ ID NO: 20. In another preferred aspect, the polypeptide comprises or consists of amino acids 16 to 235 of SEQ ID NO: 20, or an allelic variant thereof; or a fragment thereof that has cellulolytic enhancing activity. In another preferred aspect, the polypeptide comprises or consists of amino acids 16 to 235 of SEQ ID NO: 20.

A polypeptide having cellulolytic enhancing activity preferably comprises or consists of the amino acid sequence of SEQ ID NO: 22 or an allelic variant thereof; or a fragment thereof that has cellulolytic enhancing activity. In a preferred aspect, the polypeptide comprises or consists of the amino acid sequence of SEQ ID NO: 22. In another preferred aspect, the polypeptide comprises or consists of the mature polypeptide of SEQ ID NO: 22. In another preferred aspect, the polypeptide comprises or consists of amino acids 19 to 323 of SEQ ID NO: 22, or an allelic variant thereof; or a fragment thereof that has cellulolytic enhancing activity. In another preferred aspect, the polypeptide comprises or consists of amino acids 19 to 323 of SEQ ID NO: 22.

A polypeptide having cellulolytic enhancing activity preferably comprises or consists of the amino acid sequence of SEQ ID NO: 24 or an allelic variant thereof; or a fragment thereof that has cellulolytic enhancing activity. In a preferred aspect, the polypeptide comprises or consists of the amino acid sequence of SEQ ID NO: 24. In another preferred aspect, the polypeptide comprises or consists of the mature polypeptide of SEQ ID NO: 24. In another preferred aspect, the polypeptide comprises or consists of amino acids 16 to 310 of SEQ ID NO: 24, or an allelic variant thereof; or a fragment thereof that has cellulolytic enhancing activity. In another preferred aspect, the polypeptide comprises or consists of amino acids 16 to 310 of SEQ ID NO: 24.

A polypeptide having cellulolytic enhancing activity preferably comprises or consists of the amino acid sequence of SEQ ID NO: 26 or an allelic variant thereof; or a fragment thereof that has cellulolytic enhancing activity. In a preferred aspect, the polypeptide comprises or consists of the amino acid sequence of SEQ ID NO: 26. In another preferred aspect, the polypeptide comprises or consists of the mature polypeptide of SEQ ID NO: 26. In another preferred aspect, the polypeptide comprises or consists of amino acids 20 to 246 of SEQ ID NO: 26, or an allelic variant thereof; or a fragment thereof that has cellulolytic enhancing activity. In another preferred aspect, the polypeptide comprises or consists of amino acids 20 to 246 of SEQ ID NO: 26.

A polypeptide having cellulolytic enhancing activity preferably comprises or consists of the amino acid sequence of SEQ ID NO: 28 or an allelic variant thereof; or a fragment thereof that has cellulolytic enhancing activity. In a preferred aspect, the polypeptide comprises or consists of the amino acid sequence of SEQ ID NO: 28. In another preferred aspect, the polypeptide comprises or consists of the mature polypeptide of SEQ ID NO: 28. In another preferred aspect, the polypeptide comprises or consists of amino acids 22 to 354 of SEQ ID NO: 28, or an allelic variant thereof; or a fragment thereof that has cellulolytic enhancing activity. In another preferred aspect, the polypeptide comprises or consists of amino acids 22 to 354 of SEQ ID NO: 28.

A polypeptide having cellulolytic enhancing activity preferably comprises or consists of the amino acid sequence of SEQ ID NO: 30 or an allelic variant thereof; or a fragment thereof that has cellulolytic enhancing activity. In a preferred aspect, the polypeptide comprises or consists of the amino acid sequence of SEQ ID NO: 30. In another preferred aspect, the polypeptide comprises or consists of the mature polypeptide of SEQ ID NO: 30. In another preferred aspect, the polypeptide comprises or consists of amino acids 22 to 250 of SEQ ID NO: 30, or an allelic variant thereof; or a fragment thereof that has cellulolytic enhancing activity. In another preferred aspect, the polypeptide comprises or consists of amino acids 22 to 250 of SEQ ID NO: 30.

A polypeptide having cellulolytic enhancing activity preferably comprises or consists of the amino acid sequence of SEQ ID NO: 32 or an allelic variant thereof; or a fragment thereof that has cellulolytic enhancing activity. In a preferred aspect, the polypeptide comprises or consists of the amino acid sequence of SEQ ID NO: 32. In another preferred aspect, the polypeptide comprises or consists of the mature polypeptide of SEQ ID NO: 32. In another preferred aspect, the polypeptide comprises or consists of amino acids 22 to 322 of SEQ ID NO: 32, or an allelic variant thereof; or a fragment thereof that has cellulolytic enhancing activity. In another preferred aspect, the polypeptide comprises or consists of amino acids 22 to 322 of SEQ ID NO: 32.

A polypeptide having cellulolytic enhancing activity preferably comprises or consists of the amino acid sequence of SEQ ID NO: 34 or an allelic variant thereof; or a fragment thereof that has cellulolytic enhancing activity. In a preferred aspect, the polypeptide comprises or consists of the amino acid sequence of SEQ ID NO: 34. In another preferred aspect, the polypeptide comprises or consists of the mature polypeptide of SEQ ID NO: 34. In another preferred aspect, the polypeptide comprises or consists of amino acids 24 to 444 of SEQ ID NO: 34, or an allelic variant thereof; or a fragment thereof that has cellulolytic enhancing activity. In another preferred aspect, the polypeptide comprises or consists of amino acids 24 to 444 of SEQ ID NO: 34.

A polypeptide having cellulolytic enhancing activity preferably comprises or consists of the amino acid sequence of SEQ ID NO: 36 or an allelic variant thereof; or a fragment thereof that has cellulolytic enhancing activity. In a preferred aspect, the polypeptide comprises or consists of the amino acid sequence of SEQ ID NO: 36. In another preferred aspect, the polypeptide comprises or consists of the mature polypeptide of SEQ ID NO: 36. In another preferred aspect, the polypeptide comprises or consists of amino acids 26 to 253 of SEQ ID NO: 36, or an allelic variant thereof; or a fragment thereof that has cellulolytic enhancing activity. In another preferred aspect, the polypeptide comprises or consists of amino acids 26 to 253 of SEQ ID NO: 36.

A polypeptide having cellulolytic enhancing activity preferably comprises or consists of the amino acid sequence of SEQ ID NO: 38 or an allelic variant thereof; or a fragment thereof that has cellulolytic enhancing activity. In a preferred aspect, the polypeptide comprises or consists of the amino acid sequence of SEQ ID NO: 38. In another preferred aspect, the polypeptide comprises or consists of the mature polypeptide of SEQ ID NO: 38. In another preferred aspect, the polypeptide comprises or consists of amino acids 20 to 223 of SEQ ID NO: 38, or an allelic variant thereof; or a fragment thereof that has cellulolytic enhancing activity. In another preferred aspect, the polypeptide comprises or consists of amino acids 20 to 223 of SEQ ID NO: 38.

A polypeptide having cellulolytic enhancing activity preferably comprises or consists of the amino acid sequence of SEQ ID NO: 40 or an allelic variant thereof; or a fragment thereof that has cellulolytic enhancing activity. In a preferred aspect, the polypeptide comprises or consists of the amino acid sequence of SEQ ID NO: 40. In another preferred aspect, the polypeptide comprises or consists of the mature polypeptide of SEQ ID NO: 40. In another preferred aspect, the polypeptide comprises or consists of amino acids 18 to 246 of SEQ ID NO: 40, or an allelic variant thereof; or a fragment thereof that has cellulolytic enhancing activity. In another preferred aspect, the polypeptide comprises or consists of amino acids 18 to 246 of SEQ ID NO: 40.

A polypeptide having cellulolytic enhancing activity preferably comprises or consists of the amino acid sequence of SEQ ID NO: 42 or an allelic variant thereof; or a fragment thereof that has cellulolytic enhancing activity. In a preferred aspect, the polypeptide comprises or consists of the amino acid sequence of SEQ ID NO: 42. In another preferred aspect, the polypeptide comprises or consists of the mature polypeptide of SEQ ID NO: 42. In another preferred aspect, the polypeptide comprises or consists of amino acids 20 to 334 of SEQ ID NO: 42, or an allelic variant thereof; or a fragment thereof that has cellulolytic enhancing activity. In another preferred aspect, the polypeptide comprises or consists of amino acids 20 to 334 of SEQ ID NO: 42.

A polypeptide having cellulolytic enhancing activity preferably comprises or consists of the amino acid sequence of SEQ ID NO: 44 or an allelic variant thereof; or a fragment thereof that has cellulolytic enhancing activity. In a preferred aspect, the polypeptide comprises or consists of the amino acid sequence of SEQ ID NO: 44. In another preferred aspect, the polypeptide comprises or consists of the mature polypeptide of SEQ ID NO: 44. In another preferred aspect, the polypeptide comprises or consists of amino acids 18 to 227 of SEQ ID NO: 44, or an allelic variant thereof; or a fragment thereof that has cellulolytic enhancing activity. In another preferred aspect, the polypeptide comprises or consists of amino acids 18 to 227 of SEQ ID NO: 44.

A polypeptide having cellulolytic enhancing activity preferably comprises or consists of the amino acid sequence of SEQ ID NO: 46 or an allelic variant thereof; or a fragment thereof that has cellulolytic enhancing activity. In a preferred aspect, the polypeptide comprises or consists of the amino acid sequence of SEQ ID NO: 46. In another preferred aspect, the polypeptide comprises or consists of the mature polypeptide of SEQ ID NO: 46. In another preferred aspect, the polypeptide comprises or consists of amino acids 22 to 368 of SEQ ID NO: 46, or an allelic variant thereof; or a fragment thereof that has cellulolytic enhancing activity. In another preferred aspect, the polypeptide comprises or consists of amino acids 22 to 368 of SEQ ID NO: 46.

A polypeptide having cellulolytic enhancing activity preferably comprises or consists of the amino acid sequence of SEQ ID NO: 48 or an allelic variant thereof; or a fragment thereof that has cellulolytic enhancing activity. In a preferred aspect, the polypeptide comprises or consists of the amino acid sequence of SEQ ID NO: 48. In another preferred aspect, the polypeptide comprises or consists of the mature polypeptide of SEQ ID NO: 48. In another preferred aspect, the polypeptide comprises or consists of amino acids 25 to 330 of SEQ ID NO: 48, or an allelic variant thereof; or a fragment thereof that has cellulolytic enhancing activity. In another preferred aspect, the polypeptide comprises or consists of amino acids 25 to 330 of SEQ ID NO: 48.

A polypeptide having cellulolytic enhancing activity preferably comprises or consists of the amino acid sequence of SEQ ID NO: 50 or an allelic variant thereof; or a fragment thereof that has cellulolytic enhancing activity. In a preferred aspect, the polypeptide comprises or consists of the amino acid sequence of SEQ ID NO: 50. In another preferred aspect, the polypeptide comprises or consists of the mature polypeptide of SEQ ID NO: 50. In another preferred aspect, the polypeptide comprises or consists of amino acids 17 to 236 of SEQ ID NO: 50, or an allelic variant thereof; or a fragment thereof that has cellulolytic enhancing activity. In another preferred aspect, the polypeptide comprises or consists of amino acids 17 to 236 of SEQ ID NO: 50.

A polypeptide having cellulolytic enhancing activity preferably comprises or consists of the amino acid sequence of SEQ ID NO: 52 or an allelic variant thereof; or a fragment thereof that has cellulolytic enhancing activity. In a preferred aspect, the polypeptide comprises or consists of the amino acid sequence of SEQ ID NO: 52. In another preferred aspect, the polypeptide comprises or consists of the mature polypeptide of SEQ ID NO: 52. In another preferred aspect, the polypeptide comprises or consists of amino acids 19 to 250 of SEQ ID NO: 52, or an allelic variant thereof; or a fragment thereof that has cellulolytic enhancing activity. In another preferred aspect, the polypeptide comprises or consists of amino acids 19 to 250 of SEQ ID NO: 52.

A polypeptide having cellulolytic enhancing activity preferably comprises or consists of the amino acid sequence of SEQ ID NO: 54 or an allelic variant thereof; or a fragment thereof that has cellulolytic enhancing activity. In a preferred aspect, the polypeptide comprises or consists of the amino acid sequence of SEQ ID NO: 54. In another preferred aspect, the polypeptide comprises or consists of the mature polypeptide of SEQ ID NO: 54. In another preferred aspect, the polypeptide comprises or consists of amino acids 23 to 478 of SEQ ID NO: 54, or an allelic variant thereof; or a fragment thereof that has cellulolytic enhancing activity. In another preferred aspect, the polypeptide comprises or consists of amino acids 23 to 478 of SEQ ID NO: 54.

A polypeptide having cellulolytic enhancing activity preferably comprises or consists of the amino acid sequence of SEQ ID NO: 56 or an allelic variant thereof; or a fragment thereof that has cellulolytic enhancing activity. In a preferred aspect, the polypeptide comprises or consists of the amino acid sequence of SEQ ID NO: 56. In another preferred aspect, the polypeptide comprises or consists of the mature polypeptide of SEQ ID NO: 56. In another preferred aspect, the polypeptide comprises or consists of amino acids 17 to 230 of SEQ ID NO: 56, or an allelic variant thereof; or a fragment thereof that has cellulolytic enhancing activity. In another preferred aspect, the polypeptide comprises or consists of amino acids 17 to 230 of SEQ ID NO: 56.

A polypeptide having cellulolytic enhancing activity preferably comprises or consists of the amino acid sequence of SEQ ID NO: 58 or an allelic variant thereof; or a fragment thereof that has cellulolytic enhancing activity. In a preferred aspect, the polypeptide comprises or consists of the amino acid sequence of SEQ ID NO: 58. In another preferred aspect, the polypeptide comprises or consists of the mature polypeptide of SEQ ID NO: 58. In another preferred aspect, the polypeptide comprises or consists of amino acids 20 to 257 of SEQ ID NO: 58, or an allelic variant thereof; or a fragment thereof that has cellulolytic enhancing activity. In another preferred aspect, the polypeptide comprises or consists of amino acids 20 to 257 of SEQ ID NO: 58.

A polypeptide having cellulolytic enhancing activity preferably comprises or consists of the amino acid sequence of SEQ ID NO: 60 or an allelic variant thereof; or a fragment thereof that has cellulolytic enhancing activity. In a preferred aspect, the polypeptide comprises or consists of the amino acid sequence of SEQ ID NO: 60. In another preferred aspect, the polypeptide comprises or consists of the mature polypeptide of SEQ ID NO: 60. In another preferred aspect, the polypeptide comprises or consists of amino acids 23 to 251 of SEQ ID NO: 60, or an allelic variant thereof; or a fragment thereof that has cellulolytic enhancing activity. In another preferred aspect, the polypeptide comprises or consists of amino acids 23 to 251 of SEQ ID NO: 60.

A polypeptide having cellulolytic enhancing activity preferably comprises or consists of the amino acid sequence of SEQ ID NO: 62 or an allelic variant thereof; or a fragment thereof that has cellulolytic enhancing activity. In a preferred aspect, the polypeptide comprises or consists of the amino acid sequence of SEQ ID NO: 62. In another preferred aspect, the polypeptide comprises or consists of the mature polypeptide of SEQ ID NO: 62. In another preferred aspect, the polypeptide comprises or consists of amino acids 19 to 349 of SEQ ID NO: 62, or an allelic variant thereof; or a fragment thereof that has cellulolytic enhancing activity. In another preferred aspect, the polypeptide comprises or consists of amino acids 19 to 349 of SEQ ID NO: 62.

A polypeptide having cellulolytic enhancing activity preferably comprises or consists of the amino acid sequence of SEQ ID NO: 64 or an allelic variant thereof; or a fragment thereof that has cellulolytic enhancing activity. In a preferred aspect, the polypeptide comprises or consists of the amino acid sequence of SEQ ID NO: 64. In another preferred aspect, the polypeptide comprises or consists of the mature polypeptide of SEQ ID NO: 64. In another preferred aspect, the polypeptide comprises or consists of amino acids 24 to 436 of SEQ ID NO: 64, or an allelic variant thereof; or a fragment thereof that has cellulolytic enhancing activity. In another preferred aspect, the polypeptide comprises or consists of amino acids 24 to 436 of SEQ ID NO: 64.

A polypeptide having cellulolytic enhancing activity preferably comprises or consists of the amino acid sequence of SEQ ID NO: 142 or an allelic variant thereof; or a fragment thereof that has cellulolytic enhancing activity. In a preferred aspect, the polypeptide comprises or consists of the amino acid sequence of SEQ ID NO: 142. In another preferred aspect, the polypeptide comprises or consists of the mature polypeptide of SEQ ID NO: 142. In another preferred aspect, the polypeptide comprises or consists of amino acids 21 to 344 of SEQ ID NO: 142, or an allelic variant thereof; or a fragment thereof that has cellulolytic enhancing activity. In another preferred aspect, the polypeptide comprises or consists of amino acids 21 to 344 of SEQ ID NO: 142.

A polypeptide having cellulolytic enhancing activity preferably comprises or consists of the amino acid sequence of SEQ ID NO: 144 or an allelic variant thereof; or a fragment thereof that has cellulolytic enhancing activity. In a preferred aspect, the polypeptide comprises or consists of the amino acid sequence of SEQ ID NO: 144. In another preferred aspect, the polypeptide comprises or consists of the mature polypeptide of SEQ ID NO: 144. In another preferred aspect, the polypeptide comprises or consists of amino acids 21 to 389 of SEQ ID NO: 144, or an allelic variant thereof; or a fragment thereof that has cellulolytic enhancing activity. In another preferred aspect, the polypeptide comprises or consists of amino acids 21 to 389 of SEQ ID NO: 144.

A polypeptide having cellulolytic enhancing activity preferably comprises or consists of the amino acid sequence of SEQ ID NO: 146 or an allelic variant thereof; or a fragment thereof that has cellulolytic enhancing activity. In a preferred aspect, the polypeptide comprises or consists of the amino acid sequence of SEQ ID NO: 146. In another preferred aspect, the polypeptide comprises or consists of the mature polypeptide of SEQ ID NO: 146. In another preferred aspect, the polypeptide comprises or consists of amino acids 22 to 406 of SEQ ID NO: 146, or an allelic variant thereof; or a fragment thereof that has cellulolytic enhancing activity. In another preferred aspect, the polypeptide comprises or consists of amino acids 22 to 406 of SEQ ID NO: 146.

A polypeptide having cellulolytic enhancing activity preferably comprises or consists of the amino acid sequence of SEQ ID NO: 148 or an allelic variant thereof; or a fragment thereof that has cellulolytic enhancing activity. In a preferred aspect, the polypeptide comprises or consists of the amino acid sequence of SEQ ID NO: 148. In another preferred aspect, the polypeptide comprises or consists of the mature polypeptide of SEQ ID NO: 148. In another preferred aspect, the polypeptide comprises or consists of amino acids 20 to 427 of SEQ ID NO: 148, or an allelic variant thereof; or a fragment thereof that has cellulolytic enhancing activity. In another preferred aspect, the polypeptide comprises or consists of amino acids 20 to 427 of SEQ ID NO: 148.

A polypeptide having cellulolytic enhancing activity preferably comprises or consists of the amino acid sequence of SEQ ID NO: 150 or an allelic variant thereof; or a fragment thereof that has cellulolytic enhancing activity. In a preferred aspect, the polypeptide comprises or consists of the amino acid sequence of SEQ ID NO: 150. In another preferred aspect, the polypeptide comprises or consists of the mature polypeptide of SEQ ID NO: 150. In another preferred aspect, the polypeptide comprises or consists of amino acids 18 to 267 of SEQ ID NO: 150, or an allelic variant thereof; or a fragment thereof that has cellulolytic enhancing activity. In another preferred aspect, the polypeptide comprises or consists of amino acids 18 to 267 of SEQ ID NO: 150.

A polypeptide having cellulolytic enhancing activity preferably comprises or consists of the amino acid sequence of SEQ ID NO: 152 or an allelic variant thereof; or a fragment thereof that has cellulolytic enhancing activity. In a preferred aspect, the polypeptide comprises or consists of the amino acid sequence of SEQ ID NO: 152. In another preferred aspect, the polypeptide comprises or consists of the mature polypeptide of SEQ ID NO: 152. In another preferred aspect, the polypeptide comprises or consists of amino acids 21 to 273 of SEQ ID NO: 152, or an allelic variant thereof; or a fragment thereof that has cellulolytic enhancing activity. In another preferred aspect, the polypeptide comprises or consists of amino acids 21 to 273 of SEQ ID NO: 152.

A polypeptide having cellulolytic enhancing activity preferably comprises or consists of the amino acid sequence of SEQ ID NO: 154 or an allelic variant thereof; or a fragment thereof that has cellulolytic enhancing activity. In a preferred aspect, the polypeptide comprises or consists of the amino acid sequence of SEQ ID NO: 154. In another preferred aspect, the polypeptide comprises or consists of the mature polypeptide of SEQ ID NO: 154. In another preferred aspect, the polypeptide comprises or consists of amino acids 21 to 322 of SEQ ID NO: 154, or an allelic variant thereof; or a fragment thereof that has cellulolytic enhancing activity. In another preferred aspect, the polypeptide comprises or consists of amino acids 21 to 322 of SEQ ID NO: 154.

A polypeptide having cellulolytic enhancing activity preferably comprises or consists of the amino acid sequence of SEQ ID NO: 156 or an allelic variant thereof; or a fragment thereof that has cellulolytic enhancing activity. In a preferred aspect, the polypeptide comprises or consists of the amino acid sequence of SEQ ID NO: 156. In another preferred aspect, the polypeptide comprises or consists of the mature polypeptide of SEQ ID NO: 156. In another preferred aspect, the polypeptide comprises or consists of amino acids 18 to 234 of SEQ ID NO: 156, or an allelic variant thereof; or a fragment thereof that has cellulolytic enhancing activity. In another preferred aspect, the polypeptide comprises or consists of amino acids 18 to 234 of SEQ ID NO: 156.

A polypeptide having cellulolytic enhancing activity preferably comprises or consists of the amino acid sequence of SEQ ID NO: 158 or an allelic variant thereof; or a fragment thereof that has cellulolytic enhancing activity. In a preferred aspect, the polypeptide comprises or consists of the amino acid sequence of SEQ ID NO: 158. In another preferred aspect, the polypeptide comprises or consists of the mature polypeptide of SEQ ID NO: 158. In another preferred aspect, the polypeptide comprises or consists of amino acids 24 to 233 of SEQ ID NO: 158, or an allelic variant thereof; or a fragment thereof that has cellulolytic enhancing activity. In another preferred aspect, the polypeptide comprises or consists of amino acids 24 to 233 of SEQ ID NO: 158.

A polypeptide having cellulolytic enhancing activity preferably comprises or consists of the amino acid sequence of SEQ ID NO: 160 or an allelic variant thereof; or a fragment thereof that has cellulolytic enhancing activity. In a preferred aspect, the polypeptide comprises or consists of the amino acid sequence of SEQ ID NO: 160. In another preferred aspect, the polypeptide comprises or consists of the mature polypeptide of SEQ ID NO: 160. In another preferred aspect, the polypeptide comprises or consists of amino acids 17 to 237 of SEQ ID NO: 160, or an allelic variant thereof; or a fragment thereof that has cellulolytic enhancing activity. In another preferred aspect, the polypeptide comprises or consists of amino acids 17 to 237 of SEQ ID NO: 160.

A polypeptide having cellulolytic enhancing activity preferably comprises or consists of the amino acid sequence of SEQ ID NO: 162 or an allelic variant thereof; or a fragment thereof that has cellulolytic enhancing activity. In a preferred aspect, the polypeptide comprises or consists of the amino acid sequence of SEQ ID NO: 162. In another preferred aspect, the polypeptide comprises or consists of the mature polypeptide of SEQ ID NO: 162. In another preferred aspect, the polypeptide comprises or consists of amino acids 20 to 484 of SEQ ID NO: 162, or an allelic variant thereof; or a fragment thereof that has cellulolytic enhancing activity. In another preferred aspect, the polypeptide comprises or consists of amino acids 20 to 484 of SEQ ID NO: 162.

A polypeptide having cellulolytic enhancing activity preferably comprises or consists of the amino acid sequence of SEQ ID NO: 164 or an allelic variant thereof; or a fragment thereof that has cellulolytic enhancing activity. In a preferred aspect, the polypeptide comprises or consists of the amino acid sequence of SEQ ID NO: 164. In another preferred aspect, the polypeptide comprises or consists of the mature polypeptide of SEQ ID NO: 164. In another preferred aspect, the polypeptide comprises or consists of amino acids 22 to 320 of SEQ ID NO: 164, or an allelic variant thereof; or a fragment thereof that has cellulolytic enhancing activity. In another preferred aspect, the polypeptide comprises or consists of amino acids 22 to 320 of SEQ ID NO: 164.

Preferably, a fragment of the mature polypeptide of SEQ ID NO: 2 contains at least 277 amino acid residues, more preferably at least 287 amino acid residues, and most preferably at least 297 amino acid residues. Preferably, a fragment of the mature polypeptide of SEQ ID NO: 4 contains at least 185 amino acid residues, more preferably at least 195 amino acid residues, and most preferably at least 205 amino acid residues. Preferably, a fragment of the mature polypeptide of SEQ ID NO: 6 contains at least 200 amino acid residues, more preferably at least 212 amino acid residues, and most preferably at least 224 amino acid residues. Preferably, a fragment of the mature polypeptide of SEQ ID NO: 8 contains at least 175 amino acid residues, more preferably at least 185 amino acid residues, and most preferably at least 195 amino acid residues. Preferably, a fragment of the mature polypeptide of SEQ ID NO: 10 contains at least 240 amino acid residues, more preferably at least 255 amino acid residues, and most preferably at least 270 amino acid residues. Preferably, a fragment of the mature polypeptide of SEQ ID NO: 12 contains at least 255 amino acid residues, more preferably at least 270 amino acid residues, and most preferably at least 285 amino acid residues. Preferably, a fragment of the mature polypeptide of SEQ ID NO: 14 contains at least 175 amino acid residues, more preferably at least 190 amino acid residues, and most preferably at least 205 amino acid residues. Preferably, a fragment of the mature polypeptide of SEQ ID NO: 16 contains at least 200 amino acid residues, more preferably at least 210 amino acid residues, and most preferably at least 220 amino acid residues. Preferably, a fragment of the mature polypeptide of SEQ ID NO: 18 contains at least 185 amino acid residues, more preferably at least 195 amino acid residues, and most preferably at least 205 amino acid residues. Preferably, a fragment of the mature polypeptide of SEQ ID NO: 20 contains at least 190 amino acid residues, more preferably at least 200 amino acid residues, and most preferably at least 210 amino acid residues. Preferably, a fragment of the mature polypeptide of SEQ ID NO: 22 contains at least 260 amino acid residues, more preferably at least 275 amino acid residues, and most preferably at least 290 amino acid residues. Preferably, a fragment of the mature polypeptide of SEQ ID NO: 24 contains at least 250 amino acid residues, more preferably at least 265 amino acid residues, and most preferably at least 280 amino acid residues. Preferably, a fragment of the mature polypeptide of SEQ ID NO: 26 contains at least 195 amino acid residues, more preferably at least 205 amino acid residues, and most preferably at least 214 amino acid residues. Preferably, a fragment of the mature polypeptide of SEQ ID NO: 28 contains at least 285 amino acid residues, more preferably at least 300 amino acid residues, and most preferably at least 315 amino acid residues. Preferably, a fragment of the mature polypeptide of SEQ ID NO: 30 contains at least 200 amino acid residues, more preferably at least 210 amino acid residues, and most preferably at least 220 amino acid residues. Preferably, a fragment of the mature polypeptide of SEQ ID NO: 32 contains at least 255 amino acid residues, more preferably at least 270 amino acid residues, and most preferably at least 285 amino acid residues. Preferably, a fragment of the mature polypeptide of SEQ ID NO: 34 contains at least 360 amino acid residues, more preferably at least 380 amino acid residues, and most preferably at least 400 amino acid residues. Preferably, a fragment of the mature polypeptide of SEQ ID NO: 36 contains at least 200 amino acid residues, more preferably at least 210 amino acid residues, and most preferably at least 220 amino acid residues. Preferably, a fragment of the mature polypeptide of SEQ ID NO: 38 contains at least 170 amino acid residues, more preferably at least 180 amino acid residues, and most preferably at least 190 amino acid residues. Preferably, a fragment of the mature polypeptide of SEQ ID NO: 40 contains at least 190 amino acid residues, more preferably at least 200 amino acid residues, and most preferably at least 210 amino acid residues. Preferably, a fragment of the mature polypeptide of SEQ ID NO: 42 contains at least 265 amino acid residues, more preferably at least 280 amino acid residues, and most preferably at least 295 amino acid residues. Preferably, a fragment of the mature polypeptide of SEQ ID NO: 44 contains at least 180 amino acid residues, more preferably at least 190 amino acid residues, and most preferably at least 200 amino acid residues. Preferably, a fragment of the mature polypeptide of SEQ ID NO: 46 contains at least 320 amino acid residues, more preferably at least 335 amino acid residues, and most preferably at least 350 amino acid residues. Preferably, a fragment of the mature polypeptide of SEQ ID NO: 48 contains at least 255 amino acid residues, more preferably at least 270 amino acid residues, and most preferably at least 285 amino acid residues. Preferably, a fragment of the mature polypeptide of SEQ ID NO: 50 contains at least 190 amino acid residues, more preferably at least 200 amino acid residues, and most preferably at least 210 amino acid residues. Preferably, a fragment of the mature polypeptide of SEQ ID NO: 52 contains at least 200 amino acid residues, more preferably at least 210 amino acid residues, and most preferably at least 220 amino acid residues. Preferably, a fragment of the mature polypeptide of SEQ ID NO: 54 contains at least 380 amino acid residues, more preferably at least 400 amino acid residues, and most preferably at least 420 amino acid residues. Preferably, a fragment of the mature polypeptide of SEQ ID NO: 56 contains at least 180 amino acid residues, more preferably at least 190 amino acid residues, and most preferably at least 200 amino acid residues. Preferably, a fragment of the mature polypeptide of SEQ ID NO: 58 contains at least 210 amino acid residues, more preferably at least 220 amino acid residues, and most preferably at least 230 amino acid residues. Preferably, a fragment of the mature polypeptide of SEQ ID NO: 60 contains at least 190 amino acid residues, more preferably at least 200 amino acid residues, and most preferably at least 210 amino acid residues. Preferably, a fragment of the mature polypeptide of SEQ ID NO: 62 contains at least 270 amino acid residues, more preferably at least 290 amino acid residues, and most preferably at least 310 amino acid residues. Preferably, a fragment of the mature polypeptide of SEQ ID NO: 64 contains at least 340 amino acid residues, more preferably at least 360 amino acid residues, and most preferably at least 380 amino acid residues. Preferably, a fragment of the mature polypeptide of SEQ ID NO: 142 contains at least 280 amino acid residues, more preferably at least 295 amino acid residues, and most preferably at least 310 amino acid residues. Preferably, a fragment of the mature polypeptide of SEQ ID NO: 144 contains at least 310 amino acid residues, more preferably at least 330 amino acid residues, and most preferably at least 350 amino acid residues. Preferably, a fragment of the mature polypeptide of SEQ ID NO: 146 contains at least 320 amino acid residues, more preferably at least 340 amino acid residues, and most preferably at least 360 amino acid residues. Preferably, a fragment of the mature polypeptide of SEQ ID NO: 148 contains at least 350 amino acid residues, more preferably at least 370 amino acid residues, and most preferably at least 390 amino acid residues. Preferably, a fragment of the mature polypeptide of SEQ ID NO: 150 contains at least 220 amino acid residues, more preferably at least 230 amino acid residues, and most preferably at least 240 amino acid residues. Preferably, a fragment of the mature polypeptide of SEQ ID NO: 152 contains at least 220 amino acid residues, more preferably at least 230 amino acid residues, and most preferably at least 240 amino acid residues. Preferably, a fragment of the mature polypeptide of SEQ ID NO: 154 contains at least 255 amino acid residues, more preferably at least 270 amino acid residues, and most preferably at least 285 amino acid residues. Preferably, a fragment of the mature polypeptide of SEQ ID NO: 156 contains at least 185 amino acid residues, more preferably at least 195 amino acid residues, and most preferably at least 205 amino acid residues. Preferably, a fragment of the mature polypeptide of SEQ ID NO: 158 contains at least 180 amino acid residues, more preferably at least 190 amino acid residues, and most preferably at least 200 amino acid residues. Preferably, a fragment of the mature polypeptide of SEQ ID NO: 160 contains at least 190 amino acid residues, more preferably at least 200 amino acid residues, and most preferably at least 210 amino acid residues. Preferably, a fragment of the mature polypeptide of SEQ ID NO: 162 contains at least 385 amino acid residues, more preferably at least 410 amino acid residues, and most preferably at least 435 amino acid residues. Preferably, a fragment of the mature polypeptide of SEQ ID NO: 164 contains at least 255 amino acid residues, more preferably at least 270 amino acid residues, and most preferably at least 285 amino acid residues.

Preferably, a subsequence of the mature polypeptide coding sequence of SEQ ID NO: 1 contains at least 831 nucleotides, more preferably at least 861 nucleotides, and most preferably at least 891 nucleotides. Preferably, a subsequence of the mature polypeptide coding sequence of SEQ ID NO: 3 contains at least 555 nucleotides, more preferably at least 585 nucleotides, and most preferably at least 615 nucleotides. Preferably, a subsequence of the mature polypeptide coding sequence of SEQ ID NO: 5 contains at least 600 nucleotides, more preferably at least 636 nucleotides, and most preferably at least 672 nucleotides. Preferably, a subsequence of the mature polypeptide coding sequence of SEQ ID NO: 7 contains at least 525 nucleotides, more preferably at least 555 nucleotides, and most preferably at least 585 nucleotides. Preferably, a subsequence of the mature polypeptide coding sequence of SEQ ID NO: 9 contains at least 720 nucleotides, more preferably at least 765 nucleotides, and most preferably at least 810 nucleotides. Preferably, a subsequence of the mature polypeptide coding sequence of SEQ ID NO: 11 contains at least 765 nucleotides, more preferably at least 810 nucleotides, and most preferably at least 855 nucleotides. Preferably, a subsequence of the mature polypeptide coding sequence of nucleotides 67 to 796 of SEQ ID NO: 13 contains at least 525 nucleotides, more preferably at least 570 nucleotides, and most preferably at least 615 nucleotides. Preferably, a subsequence of the mature polypeptide coding sequence of SEQ ID NO: 15 contains at least 600 nucleotides, more preferably at least 630 nucleotides, and most preferably at least 660 nucleotides. Preferably, a subsequence of the mature polypeptide coding sequence of SEQ ID NO: 17 contains at least 555 nucleotides, more preferably at least 585 nucleotides, and most preferably at least 615 nucleotides. Preferably, a subsequence of the mature polypeptide coding sequence of SEQ ID NO: 19 contains at least 570 nucleotides, more preferably at least 600 nucleotides, and most preferably at least 630 nucleotides. Preferably, a subsequence of the mature polypeptide coding sequence of SEQ ID NO: 21 contains at least 780 nucleotides, more preferably at least 825 nucleotides, and most preferably at least 870 nucleotides. Preferably, a subsequence of the mature polypeptide coding sequence of SEQ ID NO: 23 contains at least 750 nucleotides, more preferably at least 795 nucleotides, and most preferably at least 840 nucleotides. Preferably, a subsequence of the mature polypeptide coding sequence of SEQ ID NO: 25 contains at least 585 nucleotides, more preferably at least 615 nucleotides, and most preferably at least 645 nucleotides. Preferably, a subsequence of the mature polypeptide coding sequence of SEQ ID NO: 27 contains at least 855 nucleotides, more preferably at least 900 nucleotides, and most preferably at least 945 nucleotides. Preferably, a subsequence of the mature polypeptide coding sequence of SEQ ID NO: 29 contains at least 600 nucleotides, more preferably at least 630 nucleotides, and most preferably at least 660 nucleotides. Preferably, a subsequence of the mature polypeptide coding sequence of SEQ ID NO: 31 contains at least 765 nucleotides, more preferably at least 810 nucleotides, and most preferably at least 855 nucleotides. Preferably, a subsequence of the mature polypeptide coding sequence of SEQ ID NO: 33 contains at least 1180 nucleotides, more preferably at least 1140 nucleotides, and most preferably at least 1200 nucleotides. Preferably, a subsequence of the mature polypeptide coding sequence of SEQ ID NO: 35 contains at least 600 nucleotides, more preferably at least 630 nucleotides, and most preferably at least 660 nucleotides. Preferably, a subsequence of the mature polypeptide coding sequence of SEQ ID NO: 37 contains at least 170 amino acid residues, more preferably at least 180 amino acid residues, and most preferably at least 190 nucleotides. Preferably, a subsequence of the mature polypeptide coding sequence of SEQ ID NO: 39 contains at least 570 nucleotides, more preferably at least 600 nucleotides, and most preferably at least 630 nucleotides. Preferably, a subsequence of the mature polypeptide coding sequence of SEQ ID NO: 41 contains at least 795 nucleotides, more preferably at least 840 nucleotides, and most preferably at least 885 nucleotides. Preferably, a subsequence of the mature polypeptide coding sequence of SEQ ID NO: 43 contains at least 540 nucleotides, more preferably at least 570 nucleotides, and most preferably at least 600 nucleotides. Preferably, a subsequence of the mature polypeptide coding sequence of SEQ ID NO: 45 contains at least 960 nucleotides, more preferably at least 1005 nucleotides, and most preferably at least 1050 nucleotides. Preferably, a subsequence of the mature polypeptide coding sequence of SEQ ID NO: 47 contains at least 765 nucleotides, more preferably at least 810 nucleotides, and most preferably at least 855 nucleotides. Preferably, a subsequence of the mature polypeptide coding sequence of SEQ ID NO: 49 contains at least 570 nucleotides, more preferably at least 600 nucleotides, and most preferably at least 630 nucleotides. Preferably, a subsequence of the mature polypeptide coding sequence of SEQ ID NO: 51 contains at least 600 nucleotides, more preferably at least 630 nucleotides, and most preferably at least 660 nucleotides. Preferably, a subsequence of the mature polypeptide coding sequence of SEQ ID NO: 53 contains at least 1140 nucleotides, more preferably at least 1200 nucleotides, and most preferably at least 1260 nucleotides. Preferably, a subsequence of the mature polypeptide coding sequence of SEQ ID NO: 55 contains at least 540 nucleotides, more preferably at least 570 nucleotides, and most preferably at least 600 nucleotides. Preferably, a subsequence of the mature polypeptide coding sequence of SEQ ID NO: 57 contains at least 630 nucleotides, more preferably at least 690 nucleotides, and most preferably at least 720 nucleotides. Preferably, a subsequence of the mature polypeptide coding sequence of SEQ ID NO: 59 contains at least 570 nucleotides, more preferably at least 600 nucleotides, and most preferably at least 630 nucleotides. Preferably, a subsequence of the mature polypeptide coding sequence of SEQ ID NO: 61 contains at least 810 nucleotides, more preferably at least 870 nucleotides, and most preferably at least 930 nucleotides. Preferably, a subsequence of the mature polypeptide coding sequence of SEQ ID NO: 63 contains at least 1020 nucleotides, more preferably at least 1080 nucleotides, and most preferably at least 1140 nucleotides. Preferably, a subsequence of the mature polypeptide coding sequence of SEQ ID NO: 141 contains at least 840 nucleotides, more preferably at least 885 nucleotides, and most preferably at least 930 nucleotides. Preferably, a subsequence of the mature polypeptide coding sequence of SEQ ID NO: 143 contains at least 930 nucleotides, more preferably at least 960 nucleotides, and most preferably at least 1050 nucleotides. Preferably, a subsequence of the mature polypeptide coding sequence of SEQ ID NO: 145 contains at least 960 nucleotides, more preferably at least 1020 nucleotides, and most preferably at least 1080 nucleotides. Preferably, a subsequence of the mature polypeptide coding sequence of SEQ ID NO: 147 contains at least 1050 nucleotides, more preferably at least 1110 nucleotides, and most preferably at least 1170 nucleotides. Preferably, a subsequence of the mature polypeptide coding sequence of SEQ ID NO: 149 contains at least 660 nucleotides, more preferably at least 690 nucleotides, and most preferably at least 720 nucleotides. Preferably, a subsequence of the mature polypeptide coding sequence of SEQ ID NO: 151 contains at least 660 nucleotides, more preferably at least 690 nucleotides, and most preferably at least 720 nucleotides. Preferably, a subsequence of the mature polypeptide coding sequence of SEQ ID NO: 153 contains at least 765 nucleotides, more preferably at least 810 nucleotides, and most preferably at least 855 nucleotides. Preferably, a subsequence of the mature polypeptide coding sequence of SEQ ID NO: 155 contains at least 555 nucleotides, more preferably at least 585 nucleotides, and most preferably at least 615 nucleotides. Preferably, a subsequence of the mature polypeptide coding sequence of SEQ ID NO: 157 contains at least 540 nucleotides, more preferably at least 570 nucleotides, and most preferably at least 600 nucleotides. Preferably, a subsequence of the mature polypeptide coding sequence of SEQ ID NO: 159 contains at least 570 nucleotides, more preferably at least 600 nucleotides, and most preferably at least 630 nucleotides. Preferably, a subsequence of the mature polypeptide coding sequence of SEQ ID NO: 161 contains at least 1155 nucleotides, more preferably at least 1230 nucleotides, and most preferably at least 1305 nucleotides. Preferably, a subsequence of the mature polypeptide coding sequence of SEQ ID NO: 163 contains at least 765 nucleotides, more preferably at least 810 nucleotides, and most preferably at least 855 nucleotides.

In a fourth aspect, the polypeptide having cellulolytic enhancing activity is encoded by a polynucleotide that hybridizes under at least very low stringency conditions, preferably at least low stringency conditions, more preferably at least medium stringency conditions, more preferably at least medium-high stringency conditions, even more preferably at least high stringency conditions, and most preferably at least very high stringency conditions with (i) the mature polypeptide coding sequence of SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 141, SEQ ID NO: 143, SEQ ID NO: 145, SEQ ID NO: 147, SEQ ID NO: 149, SEQ ID NO: 151, SEQ ID NO: 153, SEQ ID NO: 155, SEQ ID NO: 157, SEQ ID NO: 159, SEQ ID NO: 161, or SEQ ID NO: 163, (ii) the genomic DNA sequence of the mature polypeptide coding sequence of SEQ ID NO: 7, SEQ ID NO: 9, SEQ ID NO: 11, SEQ ID NO: 15, SEQ ID NO: 155, SEQ ID NO: 157, or SEQ ID NO: 159, or the cDNA sequence of the mature polypeptide coding sequence of SEQ ID NO: 1, SEQ ID NO: 3, SEQ ID NO: 5, SEQ ID NO: 13, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 141, SEQ ID NO: 143, SEQ ID NO: 145, SEQ ID NO: 147, SEQ ID NO: 149, SEQ ID NO: 151, SEQ ID NO: 153, SEQ ID NO: 161, or SEQ ID NO: 163, (iii) a subsequence of (i) or (ii), or (iv) a full-length complementary strand of (i), (ii), or (iii) (J. Sambrook, E. F. Fritsch, and T. Maniatus, 1989, supra). A subsequence of the mature polypeptide coding sequence of SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 141, SEQ ID NO: 143, SEQ ID NO: 145, SEQ ID NO: 147, SEQ ID NO: 149, SEQ ID NO: 151, SEQ ID NO: 153, SEQ ID NO: 155, SEQ ID NO: 157, SEQ ID NO: 159, SEQ ID NO: 161, or SEQ ID NO: 163 contains at least 100 contiguous nucleotides or preferably at least 200 contiguous nucleotides. Moreover, the subsequence may encode a polypeptide fragment that has cellulolytic enhancing activity. In a preferred aspect, the mature polypeptide coding sequence is nucleotides 388 to 1332 of SEQ ID NO: 1, nucleotides 98 to 821 of SEQ ID NO: 3, nucleotides 126 to 978 of SEQ ID NO: 5, nucleotides 55 to 678 of SEQ ID NO: 7, nucleotides 58 to 912 of SEQ ID NO: 9, nucleotides 46 to 951 of SEQ ID NO: 11, nucleotides 67 to 796 of SEQ ID NO: 13, nucleotides 77 to 766 of SEQ ID NO: 15, nucleotides 52 to 921 of SEQ ID NO: 17, nucleotides 46 to 851 of SEQ ID NO: 19, nucleotides 55 to 1239 of SEQ ID NO: 21, nucleotides 46 to 1250 of SEQ ID NO: 23, nucleotides 58 to 811 of SEQ ID NO: 25, nucleotides 64 to 1112 of SEQ ID NO: 27, nucleotides 64 to 859 of SEQ ID NO: 29, nucleotides 64 to 1018 of SEQ ID NO: 31, nucleotides 70 to 1483 of SEQ ID NO: 33, nucleotides 76 to 832 of SEQ ID NO: 35, nucleotides 58 to 974 of SEQ ID NO: 37, nucleotides 52 to 875 of SEQ ID NO: 39, nucleotides 58 to 1250 of SEQ ID NO: 41, nucleotides 52 to 795 of SEQ ID NO: 43, nucleotides 64 to 1104 of SEQ ID NO: 45, nucleotides 73 to 990 of SEQ ID NO: 47, nucleotides 49 to 1218 of SEQ ID NO: 49, nucleotides 55 to 930 of SEQ ID NO: 51, nucleotides 67 to 1581 of SEQ ID NO: 53, nucleotides 49 to 865 of SEQ ID NO: 55, nucleotides 58 to 1065 of SEQ ID NO: 57, nucleotides 67 to 868 of SEQ ID NO: 59, nucleotides 55 to 1099 of SEQ ID NO: 61, nucleotides 70 to 1483 of SEQ ID NO: 63, nucleotides 61 to 1032 of SEQ ID NO: 141, nucleotides 61 to 1167 of SEQ ID NO: 143, nucleotides 64 to 1218 of SEQ ID NO: 145, nucleotides 58 to 1281 of SEQ ID NO: 147, nucleotides 52 to 801 of SEQ ID NO: 149, nucleotides 61 to 819 of SEQ ID NO: 151, nucleotides 61 to 966 of SEQ ID NO: 153, nucleotides 52 to 702 of SEQ ID NO: 155, nucleotides 70 to 699 of SEQ ID NO: 157, nucleotides 49 to 711 of SEQ ID NO: 159, nucleotides 76 to 1452 of SEQ ID NO: 161, or nucleotides 64 to 1018 of SEQ ID NO: 163.

The nucleotide sequence of SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 141, SEQ ID NO: 143, SEQ ID NO: 145, SEQ ID NO: 147, SEQ ID NO: 149, SEQ ID NO: 151, SEQ ID NO: 153, SEQ ID NO: 155, SEQ ID NO: 157, SEQ ID NO: 159, SEQ ID NO: 161, or SEQ ID NO: 163, or a subsequence thereof; as well as the amino acid sequence of SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 42, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 142, SEQ ID NO: 144, SEQ ID NO: 146, SEQ ID NO: 148, SEQ ID NO: 150, SEQ ID NO: 152, SEQ ID NO: 154, SEQ ID NO: 156, SEQ ID NO: 158, SEQ ID NO: 160, SEQ ID NO: 162, or SEQ ID NO: 164, or a fragment thereof, may be used to design a nucleic acid probe to identify and clone DNA encoding polypeptides having cellulolytic enhancing activity from strains of different genera or species according to methods well known in the art. In particular, such probes can be used for hybridization with the genomic DNA or cDNA of the genus or species of interest, following standard Southern blotting procedures, in order to identify and isolate the corresponding gene therein. Such probes can be considerably shorter than the entire sequence, but should be at least 14, preferably at least 25, more preferably at least 35, and most preferably at least 70 nucleotides in length. It is, however, preferred that the nucleic acid probe is at least 100 nucleotides in length. For example, the nucleic acid probe may be at least 200 nucleotides, preferably at least 300 nucleotides, more preferably at least 400 nucleotides, or most preferably at least 500 nucleotides in length. Even longer probes may be used, e.g., nucleic acid probes that are preferably at least 600 nucleotides, more preferably at least 700 nucleotides, even more preferably at least 800 nucleotides, or most preferably at least 900 nucleotides in length. Both DNA and RNA probes can be used. The probes are typically labeled for detecting the corresponding gene (for example, with ³²P, ³H, ³⁵S, biotin, or avidin). Such probes are encompassed by the present invention.

A genomic DNA or cDNA library prepared from such other strains may, therefore, be screened for DNA that hybridizes with the probes described above and encodes a polypeptide having cellulolytic enhancing activity. Genomic or other DNA from such other strains may be separated by agarose or polyacrylamide gel electrophoresis, or other separation techniques. DNA from the libraries or the separated DNA may be transferred to and immobilized on nitrocellulose or other suitable carrier material. In order to identify a clone or DNA that is homologous with SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 141, SEQ ID NO: 143, SEQ ID NO: 145, SEQ ID NO: 147, SEQ ID NO: 149, SEQ ID NO: 151, SEQ ID NO: 153, SEQ ID NO: 155, SEQ ID NO: 157, SEQ ID NO: 159, SEQ ID NO: 161, or SEQ ID NO: 163, or a subsequence thereof, the carrier material is preferably used in a Southern blot.

For purposes of the present invention, hybridization indicates that the nucleotide sequence hybridizes to a labeled nucleic acid probe corresponding to the mature polypeptide coding sequence of SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 141, SEQ ID NO: 143, SEQ ID NO: 145, SEQ ID NO: 147, SEQ ID NO: 149, SEQ ID NO: 151, SEQ ID NO: 153, SEQ ID NO: 155, SEQ ID NO: 157, SEQ ID NO: 159, SEQ ID NO: 161, or SEQ ID NO: 163; the genomic DNA sequence of the mature polypeptide coding sequence of SEQ ID NO: 7, SEQ ID NO: 9, SEQ ID NO: 11, SEQ ID NO: 15, SEQ ID NO: 155, SEQ ID NO: 157, or SEQ ID NO: 159, or the cDNA sequence of the mature polypeptide coding sequence of SEQ ID NO: 1, SEQ ID NO: 3, SEQ ID NO: 5, SEQ ID NO: 13, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 141, SEQ ID NO: 143, SEQ ID NO: 145, SEQ ID NO: 147, SEQ ID NO: 149, SEQ ID NO: 151, SEQ ID NO: 153, SEQ ID NO: 161, or SEQ ID NO: 163; the full-length complementary strand thereof; or a subsequence thereof, under very low to very high stringency conditions, as described supra.

In a preferred aspect, the nucleic acid probe is the mature polypeptide coding sequence of SEQ ID NO: 1. In another preferred aspect, the nucleic acid probe is nucleotides 388 to 1332 of SEQ ID NO: 1. In another preferred aspect, the nucleic acid probe is a polynucleotide sequence that encodes the polypeptide of SEQ ID NO: 2, or a subsequence thereof. In another preferred aspect, the nucleic acid probe is SEQ ID NO: 1. In another preferred aspect, the nucleic acid probe is the polynucleotide sequence contained in plasmid pEJG120 which is contained in E. coli NRRL B-30699, wherein the polynucleotide sequence thereof encodes a polypeptide having cellulolytic enhancing activity. In another preferred aspect, the nucleic acid probe is the mature polypeptide coding sequence contained in plasmid pEJG120 which is contained in E. coli NRRL B-30699.

In another preferred aspect, the nucleic acid probe is the mature polypeptide coding sequence of SEQ ID NO: 3. In another preferred aspect, the nucleic acid probe is nucleotides 98 to 821 of SEQ ID NO: 3. In another preferred aspect, the nucleic acid probe is a polynucleotide sequence that encodes the polypeptide of SEQ ID NO: 4, or a subsequence thereof. In another preferred aspect, the nucleic acid probe is SEQ ID NO: 3. In another preferred aspect, the nucleic acid probe is the polynucleotide sequence contained in plasmid pTter61C which is contained in E. coli NRRL B-30813, wherein the polynucleotide sequence thereof encodes a polypeptide having cellulolytic enhancing activity. In another preferred aspect, the nucleic acid probe is the mature polypeptide coding sequence contained in plasmid pTter61C which is contained in E. coli NRRL B-30813.

In another preferred aspect, the nucleic acid probe is the mature polypeptide coding sequence of SEQ ID NO: 5. In another preferred aspect, the nucleic acid probe is nucleotides 126 to 978 of SEQ ID NO: 5. In another preferred aspect, the nucleic acid probe is a polynucleotide sequence that encodes the polypeptide of SEQ ID NO: 6, or a subsequence thereof. In another preferred aspect, the nucleic acid probe is SEQ ID NO: 5. In another preferred aspect, the nucleic acid probe is the polynucleotide sequence contained in plasmid pTter61D which is contained in E. coli NRRL B-30812, wherein the polynucleotide sequence thereof encodes a polypeptide having cellulolytic enhancing activity. In another preferred aspect, the nucleic acid probe is the mature polypeptide coding sequence contained in plasmid pTter61D which is contained in E. coli NRRL B-30812.

In another preferred aspect, the nucleic acid probe is the mature polypeptide coding sequence of SEQ ID NO: 7. In another preferred aspect, the nucleic acid probe is nucleotides 55 to 678 of SEQ ID NO: 7. In another preferred aspect, the nucleic acid probe is a polynucleotide sequence that encodes the polypeptide of SEQ ID NO: 8, or a subsequence thereof. In another preferred aspect, the nucleic acid probe is SEQ ID NO: 7. In another preferred aspect, the nucleic acid probe is the polynucleotide sequence contained in plasmid pTter61E which is contained in E. coli NRRL B-30814, wherein the polynucleotide sequence thereof encodes a polypeptide having cellulolytic enhancing activity. In another preferred aspect, the nucleic acid probe is the mature polypeptide coding sequence contained in plasmid pTter61E which is contained in E. coli NRRL B-30814.

In another preferred aspect, the nucleic acid probe is the mature polypeptide coding sequence of SEQ ID NO: 9. In another preferred aspect, the nucleic acid probe is nucleotides 58 to 912 of SEQ ID NO: 9 In another preferred aspect, the nucleic acid probe is a polynucleotide sequence that encodes the polypeptide of SEQ ID NO: 10, or a subsequence thereof. In another preferred aspect, the nucleic acid probe is SEQ ID NO: 9. In another preferred aspect, the nucleic acid probe is the polynucleotide sequence contained in plasmid pTter61G which is contained in E. coli NRRL B-30811, wherein the polynucleotide sequence thereof encodes a polypeptide having cellulolytic enhancing activity. In another preferred aspect, the nucleic acid probe is the mature polypeptide coding sequence contained in plasmid pTter61G which is contained in E. coli NRRL B-30811.

In another preferred aspect, the nucleic acid probe is the mature polypeptide coding sequence of SEQ ID NO: 11. In another preferred aspect, the nucleic acid probe is nucleotides 46 to 951 of SEQ ID NO: 11. In another preferred aspect, the nucleic acid probe is a polynucleotide sequence that encodes the polypeptide of SEQ ID NO: 12, or a subsequence thereof. In another preferred aspect, the nucleic acid probe is SEQ ID NO: 11. In another preferred aspect, the nucleic acid probe is the polynucleotide sequence contained in plasmid pTter61F which is contained in E. coli NRRL B-50044, wherein the polynucleotide sequence thereof encodes a polypeptide having cellulolytic enhancing activity. In another preferred aspect, the nucleic acid probe is the mature polypeptide coding region contained in plasmid pTter61F which is contained in E. coli NRRL B-50044.

In another preferred aspect, the nucleic acid probe is the mature polypeptide coding sequence of SEQ ID NO: 13. In another preferred aspect, the nucleic acid probe is nucleotides 67 to 796 of SEQ ID NO: 13. In another preferred aspect, the nucleic acid probe is a polynucleotide sequence that encodes the polypeptide of SEQ ID NO: 14, or a subsequence thereof. In another preferred aspect, the nucleic acid probe is SEQ ID NO: 13. In another preferred aspect, the nucleic acid probe is the polynucleotide sequence contained in plasmid pDZA2-7 which is contained in E. coli NRRL B-30704, wherein the polynucleotide sequence thereof encodes a polypeptide having cellulolytic enhancing activity. In another preferred aspect, the nucleic acid probe is the mature polypeptide coding sequence contained in plasmid pDZA2-7 which is contained in E. coli NRRL B-30704.

In another preferred aspect, the nucleic acid probe is the mature polypeptide coding sequence of SEQ ID NO: 15. In another preferred aspect, the nucleic acid probe is nucleotides 77 to 766 of SEQ ID NO: 15. In another preferred aspect, the nucleic acid probe is a polynucleotide sequence that encodes the polypeptide of SEQ ID NO: 16, or a subsequence thereof. In another preferred aspect, the nucleic acid probe is SEQ ID NO: 15. In another preferred aspect, the nucleic acid probe is the polynucleotide sequence contained in plasmid pTr3337 which is contained in E. coli NRRL B-30878, wherein the polynucleotide sequence thereof encodes a polypeptide having cellulolytic enhancing activity. In another preferred aspect, the nucleic acid probe is the mature polypeptide coding sequence contained in plasmid pTr3337 which is contained in E. coli NRRL B-30878.

In another preferred aspect, the nucleic acid probe is the mature polypeptide coding sequence of SEQ ID NO: 17. In another preferred aspect, the nucleic acid probe is nucleotides 52 to 921 of SEQ ID NO: 17. In another preferred aspect, the nucleic acid probe is a polynucleotide sequence that encodes the polypeptide of SEQ ID NO: 18, or a subsequence thereof. In another preferred aspect, the nucleic acid probe is SEQ ID NO: 17. In another preferred aspect, the nucleic acid probe is the polynucleotide sequence contained in plasmid pSMai190 which is contained in E. coli NRRL B-50084, wherein the polynucleotide sequence thereof encodes a polypeptide having cellulolytic enhancing activity. In another preferred aspect, the nucleic acid probe is the mature polypeptide coding sequence contained in plasmid pSMai190 which is contained in E. coli NRRL B-50084.

In another preferred aspect, the nucleic acid probe is the mature polypeptide coding sequence of SEQ ID NO: 19. In another preferred aspect, the nucleic acid probe is nucleotides 46 to 851 of SEQ ID NO: 19. In another preferred aspect, the nucleic acid probe is a polynucleotide sequence that encodes the polypeptide of SEQ ID NO: 20, or a subsequence thereof. In another preferred aspect, the nucleic acid probe is SEQ ID NO: 19. In another preferred aspect, the nucleic acid probe is the polynucleotide sequence contained in plasmid pSMai192 which is contained in E. coli NRRL B-50086, wherein the polynucleotide sequence thereof encodes a polypeptide having cellulolytic enhancing activity. In another preferred aspect, the nucleic acid probe is the mature polypeptide coding sequence contained in plasmid pSMai192 which is contained in E. coli NRRL B-50086.

In another preferred aspect, the nucleic acid probe is the mature polypeptide coding sequence of SEQ ID NO: 21. In another preferred aspect, the nucleic acid probe is nucleotides 55 to 1239 of SEQ ID NO: 21. In another preferred aspect, the nucleic acid probe is a polynucleotide sequence that encodes the polypeptide of SEQ ID NO: 22, or a subsequence thereof. In another preferred aspect, the nucleic acid probe is SEQ ID NO: 21. In another preferred aspect, the nucleic acid probe is the polynucleotide sequence contained in plasmid pSMai191 which is contained in E. coli NRRL B-50085, wherein the polynucleotide sequence thereof encodes a polypeptide having cellulolytic enhancing activity. In another preferred aspect, the nucleic acid probe is the mature polypeptide coding sequence contained in plasmid pSMai191 which is contained in E. coli NRRL B-50085.

In another preferred aspect, the nucleic acid probe is the mature polypeptide coding sequence of SEQ ID NO: 23. In another preferred aspect, the nucleic acid probe is nucleotides 46 to 1250 of SEQ ID NO: 23. In another preferred aspect, the nucleic acid probe is a polynucleotide sequence that encodes the polypeptide of SEQ ID NO: 24, or a subsequence thereof. In another preferred aspect, the nucleic acid probe is SEQ ID NO: 23. In another preferred aspect, the nucleic acid probe is the polynucleotide sequence contained in plasmid pSMai193 which is contained in E. coli NRRL B-50087, wherein the polynucleotide sequence thereof encodes a polypeptide having cellulolytic enhancing activity. In another preferred aspect, the nucleic acid probe is the mature polypeptide coding sequence contained in plasmid pSMai193 which is contained in E. coli NRRL B-50087.

In another preferred aspect, the nucleic acid probe is the mature polypeptide coding sequence of SEQ ID NO: 25. In another preferred aspect, the nucleic acid probe is nucleotides 58 to 811 of SEQ ID NO: 25. In another preferred aspect, the nucleic acid probe is a polynucleotide sequence that encodes the polypeptide of SEQ ID NO: 26, or a subsequence thereof. In another preferred aspect, the nucleic acid probe is SEQ ID NO: 25. In another preferred aspect, the nucleic acid probe is the polynucleotide sequence contained in plasmid pSMai187 which is contained in E. coli NRRL B-50083, wherein the polynucleotide sequence thereof encodes a polypeptide having cellulolytic enhancing activity. In another preferred aspect, the nucleic acid probe is the mature polypeptide coding sequence contained in plasmid pSMai187 which is contained in E. coli NRRL B-50083.

In another preferred aspect, the nucleic acid probe is the mature polypeptide coding sequence of SEQ ID NO: 27. In another preferred aspect, the nucleic acid probe is nucleotides 64 to 1112 of SEQ ID NO: 27. In another preferred aspect, the nucleic acid probe is a polynucleotide sequence that encodes the polypeptide of SEQ ID NO: 28, or a subsequence thereof. In another preferred aspect, the nucleic acid probe is SEQ ID NO: 27. In another preferred aspect, the nucleic acid probe is the polynucleotide sequence contained in plasmid pXYZ1473 which is contained in E. coli DSM 22075, wherein the polynucleotide sequence thereof encodes a polypeptide having cellulolytic enhancing activity. In another preferred aspect, the nucleic acid probe is the mature polypeptide coding sequence contained in plasmid pXYZ1473 which is contained in E. coli DSM 22075.

In another preferred aspect, the nucleic acid probe is the mature polypeptide coding sequence of SEQ ID NO: 29. In another preferred aspect, the nucleic acid probe is nucleotides 64 to 859 of SEQ ID NO: 29. In another preferred aspect, the nucleic acid probe is a polynucleotide sequence that encodes the polypeptide of SEQ ID NO: 30, or a subsequence thereof. In another preferred aspect, the nucleic acid probe is SEQ ID NO: 29.

In another preferred aspect, the nucleic acid probe is the mature polypeptide coding sequence of SEQ ID NO: 31. In another preferred aspect, the nucleic acid probe is nucleotides 64 to 1018 of SEQ ID NO: 31. In another preferred aspect, the nucleic acid probe is a polynucleotide sequence that encodes the polypeptide of SEQ ID NO: 32, or a subsequence thereof. In another preferred aspect, the nucleic acid probe is SEQ ID NO: 31. In another preferred aspect, the nucleic acid probe is the polynucleotide sequence contained in plasmid pGEM-T-Ppin7 which is contained in E. coli DSM 22711, wherein the polynucleotide sequence thereof encodes a polypeptide having cellulolytic enhancing activity. In another preferred aspect, the nucleic acid probe is the mature polypeptide coding sequence contained in plasmid pGEM-T-Ppin7 which is contained in E. coli DSM 22711.

In another preferred aspect, the nucleic acid probe is the mature polypeptide coding sequence of SEQ ID NO: 33. In another preferred aspect, the nucleic acid probe is nucleotides 70 to 1483 of SEQ ID NO: 33. In another preferred aspect, the nucleic acid probe is a polynucleotide sequence that encodes the polypeptide of SEQ ID NO: 34, or a subsequence thereof. In another preferred aspect, the nucleic acid probe is SEQ ID NO: 33. In another preferred aspect, the nucleic acid probe is the polynucleotide sequence contained in plasmid pXYZ1483 which is contained in E. coli DSM 22600, wherein the polynucleotide sequence thereof encodes a polypeptide having cellulolytic enhancing activity. In another preferred aspect, the nucleic acid probe is the mature polypeptide coding sequence contained in plasmid pXYZ1483 which is contained in E. coli DSM 22600.

In another preferred aspect, the nucleic acid probe is the mature polypeptide coding sequence of SEQ ID NO: 35. In another preferred aspect, the nucleic acid probe is nucleotides 76 to 832 of SEQ ID NO: 35. In another preferred aspect, the nucleic acid probe is a polynucleotide sequence that encodes the polypeptide of SEQ ID NO: 36, or a subsequence thereof. In another preferred aspect, the nucleic acid probe is SEQ ID NO: 35. In another preferred aspect, the nucleic acid probe is the polynucleotide sequence contained in plasmid pGEM-T-GH61D23Y4 which is contained in E. coli DSM 22882, wherein the polynucleotide sequence thereof encodes a polypeptide having cellulolytic enhancing activity. In another preferred aspect, the nucleic acid probe is the mature polypeptide coding sequence contained in plasmid pGEM-T-GH61D23Y4 which is contained in E. coli DSM 22882.

In another preferred aspect, the nucleic acid probe is the mature polypeptide coding sequence of SEQ ID NO: 37. In another preferred aspect, the nucleic acid probe is nucleotides 58 to 974 of SEQ ID NO: 37. In another preferred aspect, the nucleic acid probe is a polynucleotide sequence that encodes the polypeptide of SEQ ID NO: 38, or a subsequence thereof. In another preferred aspect, the nucleic acid probe is SEQ ID NO: 37. In another preferred aspect, the nucleic acid probe is the polynucleotide sequence contained in plasmid pSMai213 which is contained in E. coli NRRL B-50300, wherein the polynucleotide sequence thereof encodes a polypeptide having cellulolytic enhancing activity. In another preferred aspect, the nucleic acid probe is the mature polypeptide coding sequence contained in plasmid pSMai213 which is contained in E. coli NRRL B-50300.

In another preferred aspect, the nucleic acid probe is the mature polypeptide coding sequence of SEQ ID NO: 39. In another preferred aspect, the nucleic acid probe is nucleotides 52 to 875 of SEQ ID NO: 39. In another preferred aspect, the nucleic acid probe is a polynucleotide sequence that encodes the polypeptide of SEQ ID NO: 40, or a subsequence thereof. In another preferred aspect, the nucleic acid probe is SEQ ID NO: 39. In another preferred aspect, the nucleic acid probe is the polynucleotide sequence contained in plasmid pSMai216 which is contained in E. coli NRRL B-50301, wherein the polynucleotide sequence thereof encodes a polypeptide having cellulolytic enhancing activity. In another preferred aspect, the nucleic acid probe is the mature polypeptide coding sequence contained in plasmid pSMai216 which is contained in E. coli NRRL B-50301.

In another preferred aspect, the nucleic acid probe is the mature polypeptide coding sequence of SEQ ID NO: 41. In another preferred aspect, the nucleic acid probe is nucleotides 58 to 1250 of SEQ ID NO: 41. In another preferred aspect, the nucleic acid probe is a polynucleotide sequence that encodes the polypeptide of SEQ ID NO: 42, or a subsequence thereof. In another preferred aspect, the nucleic acid probe is SEQ ID NO: 41. In another preferred aspect, the nucleic acid probe is the polynucleotide sequence contained in plasmid p pSMai217 which is contained in E. coli NRRL B-50302, wherein the polynucleotide sequence thereof encodes a polypeptide having cellulolytic enhancing activity. In another preferred aspect, the nucleic acid probe is the mature polypeptide coding sequence contained in plasmid pSMai217 which is contained in E. coli NRRL B-50302.

In another preferred aspect, the nucleic acid probe is the mature polypeptide coding sequence of SEQ ID NO: 43. In another preferred aspect, the nucleic acid probe is nucleotides 52 to 795 of SEQ ID NO: 43. In another preferred aspect, the nucleic acid probe is a polynucleotide sequence that encodes the polypeptide of SEQ ID NO: 44, or a subsequence thereof. In another preferred aspect, the nucleic acid probe is SEQ ID NO: 43. In another preferred aspect, the nucleic acid probe is the polynucleotide sequence contained in plasmid pSMai218 which is contained in E. coli NRRL B-50303, wherein the polynucleotide sequence thereof encodes a polypeptide having cellulolytic enhancing activity. In another preferred aspect, the nucleic acid probe is the mature polypeptide coding sequence contained in plasmid pSMai218 which is contained in E. coli NRRL B-50303.

In another preferred aspect, the nucleic acid probe is the mature polypeptide coding sequence of SEQ ID NO: 45. In another preferred aspect, the nucleic acid probe is nucleotides 64 to 1104 of SEQ ID NO: 45. In another preferred aspect, the nucleic acid probe is a polynucleotide sequence that encodes the polypeptide of SEQ ID NO: 46, or a subsequence thereof. In another preferred aspect, the nucleic acid probe is SEQ ID NO: 45. In another preferred aspect, the nucleic acid probe is the polynucleotide sequence contained in plasmid pAG68 which is contained in E. coli NRRL B-50320, wherein the polynucleotide sequence thereof encodes a polypeptide having cellulolytic enhancing activity. In another preferred aspect, the nucleic acid probe is the mature polypeptide coding sequence contained in plasmid pAG68 which is contained in E. coli NRRL B-50320.

In another preferred aspect, the nucleic acid probe is the mature polypeptide coding sequence of SEQ ID NO: 47. In another preferred aspect, the nucleic acid probe is nucleotides 73 to 990 of SEQ ID NO: 47. In another preferred aspect, the nucleic acid probe is a polynucleotide sequence that encodes the polypeptide of SEQ ID NO: 48, or a subsequence thereof. In another preferred aspect, the nucleic acid probe is SEQ ID NO: 47. In another preferred aspect, the nucleic acid probe is the polynucleotide sequence contained in plasmid pAG69 which is contained in E. coli NRRL B-50321, wherein the polynucleotide sequence thereof encodes a polypeptide having cellulolytic enhancing activity. In another preferred aspect, the nucleic acid probe is the mature polypeptide coding sequence contained in plasmid pAG69 which is contained in E. coli NRRL B-50321.

In another preferred aspect, the nucleic acid probe is the mature polypeptide coding sequence of SEQ ID NO: 49. In another preferred aspect, the nucleic acid probe is nucleotides 49 to 1218 of SEQ ID NO: 49. In another preferred aspect, the nucleic acid probe is a polynucleotide sequence that encodes the polypeptide of SEQ ID NO: 50, or a subsequence thereof. In another preferred aspect, the nucleic acid probe is SEQ ID NO: 49. In another preferred aspect, the nucleic acid probe is the polynucleotide sequence contained in plasmid pAG75 which is contained in E. coli NRRL B-50322, wherein the polynucleotide sequence thereof encodes a polypeptide having cellulolytic enhancing activity. In another preferred aspect, the nucleic acid probe is the mature polypeptide coding sequence contained in plasmid pAG75 which is contained in E. coli NRRL B-50322.

In another preferred aspect, the nucleic acid probe is the mature polypeptide coding sequence of SEQ ID NO: 51. In another preferred aspect, the nucleic acid probe is nucleotides 55 to 930 of SEQ ID NO: 51. In another preferred aspect, the nucleic acid probe is a polynucleotide sequence that encodes the polypeptide of SEQ ID NO: 52, or a subsequence thereof. In another preferred aspect, the nucleic acid probe is SEQ ID NO: 51. In another preferred aspect, the nucleic acid probe is the polynucleotide sequence contained in plasmid pAG76 which is contained in E. coli NRRL B-50323, wherein the polynucleotide sequence thereof encodes a polypeptide having cellulolytic enhancing activity. In another preferred aspect, the nucleic acid probe is the mature polypeptide coding sequence contained in plasmid pAG76 which is contained in E. coli NRRL B-50323.

In another preferred aspect, the nucleic acid probe is the mature polypeptide coding sequence of SEQ ID NO: 53. In another preferred aspect, the nucleic acid probe is nucleotides 67 to 1581 of SEQ ID NO: 53. In another preferred aspect, the nucleic acid probe is a polynucleotide sequence that encodes the polypeptide of SEQ ID NO: 54, or a subsequence thereof. In another preferred aspect, the nucleic acid probe is SEQ ID NO: 53. In another preferred aspect, the nucleic acid probe is the polynucleotide sequence contained in plasmid pAG77 which is contained in E. coli NRRL B-50324, wherein the polynucleotide sequence thereof encodes a polypeptide having cellulolytic enhancing activity. In another preferred aspect, the nucleic acid probe is the mature polypeptide coding sequence contained in plasmid pAG77 which is contained in E. coli NRRL B-50324.

In another preferred aspect, the nucleic acid probe is the mature polypeptide coding sequence of SEQ ID NO: 55. In another preferred aspect, the nucleic acid probe is nucleotides 49 to 865 of SEQ ID NO: 55. In another preferred aspect, the nucleic acid probe is a polynucleotide sequence that encodes the polypeptide of SEQ ID NO: 56, or a subsequence thereof. In another preferred aspect, the nucleic acid probe is SEQ ID NO: 55. In another preferred aspect, the nucleic acid probe is the polynucleotide sequence contained in plasmid pAG78 which is contained in E. coli NRRL B-50325, wherein the polynucleotide sequence thereof encodes a polypeptide having cellulolytic enhancing activity. In another preferred aspect, the nucleic acid probe is the mature polypeptide coding sequence contained in plasmid pAG78 which is contained in E. coli NRRL B-50325.

In another preferred aspect, the nucleic acid probe is the mature polypeptide coding sequence of SEQ ID NO: 57. In another preferred aspect, the nucleic acid probe is nucleotides 58 to 1065 of SEQ ID NO: 57. In another preferred aspect, the nucleic acid probe is a polynucleotide sequence that encodes the polypeptide of SEQ ID NO: 58, or a subsequence thereof. In another preferred aspect, the nucleic acid probe is SEQ ID NO: 57. In another preferred aspect, the nucleic acid probe is the polynucleotide sequence contained in plasmid p pAG79 which is contained in E. coli NRRL B-50326, wherein the polynucleotide sequence thereof encodes a polypeptide having cellulolytic enhancing activity. In another preferred aspect, the nucleic acid probe is the mature polypeptide coding sequence contained in plasmid pAG79 which is contained in E. coli NRRL B-50326.

In another preferred aspect, the nucleic acid probe is the mature polypeptide coding sequence of SEQ ID NO: 59. In another preferred aspect, the nucleic acid probe is nucleotides 67 to 868 of SEQ ID NO: 59. In another preferred aspect, the nucleic acid probe is a polynucleotide sequence that encodes the polypeptide of SEQ ID NO: 60, or a subsequence thereof. In another preferred aspect, the nucleic acid probe is SEQ ID NO: 59. In another preferred aspect, the nucleic acid probe is the polynucleotide sequence contained in plasmid pGEM-T-GH61a51486 which is contained in E. coli DSM 22656, wherein the polynucleotide sequence thereof encodes a polypeptide having cellulolytic enhancing activity. In another preferred aspect, the nucleic acid probe is the mature polypeptide coding sequence contained in plasmid pGEM-T-GH61a51486 which is contained in E. coli DSM 22656.

In another preferred aspect, the nucleic acid probe is the mature polypeptide coding sequence of SEQ ID NO: 61. In another preferred aspect, the nucleic acid probe is nucleotides 55 to 1099 of SEQ ID NO: 61. In another preferred aspect, the nucleic acid probe is a polynucleotide sequence that encodes the polypeptide of SEQ ID NO: 62, or a subsequence thereof. In another preferred aspect, the nucleic acid probe is SEQ ID NO: 61. In another preferred aspect, the nucleic acid probe is the polynucleotide sequence contained in plasmid pGEM-T-GH61DYF which is contained in E. coli DSM 22654, wherein the polynucleotide sequence thereof encodes a polypeptide having cellulolytic enhancing activity. In another preferred aspect, the nucleic acid probe is the mature polypeptide coding sequence contained in plasmid pGEM-T-GH61DYF which is contained in E. coli DSM 22654.

In another preferred aspect, the nucleic acid probe is the mature polypeptide coding sequence of SEQ ID NO: 63. In another preferred aspect, the nucleic acid probe is nucleotides 70 to 1483 of SEQ ID NO: 63. In another preferred aspect, the nucleic acid probe is a polynucleotide sequence that encodes the polypeptide of SEQ ID NO: 64, or a subsequence thereof. In another preferred aspect, the nucleic acid probe is SEQ ID NO: 63. In another preferred aspect, the nucleic acid probe is the polynucleotide sequence contained in plasmid pGEM-T-GH61D14YH which is contained in E. coli DSM 22657, wherein the polynucleotide sequence thereof encodes a polypeptide having cellulolytic enhancing activity. In another preferred aspect, the nucleic acid probe is the mature polypeptide coding sequence contained in plasmid pGEM-T-GH61D14YH which is contained in E. coli DSM 22657.

In another preferred aspect, the nucleic acid probe is the mature polypeptide coding sequence of SEQ ID NO: 141. In another preferred aspect, the nucleic acid probe is nucleotides 61 to 1032 of SEQ ID NO: 141. In another preferred aspect, the nucleic acid probe is a polynucleotide sequence that encodes the polypeptide of SEQ ID NO: 141, or a subsequence thereof. In another preferred aspect, the nucleic acid probe is SEQ ID NO: 141.

In another preferred aspect, the nucleic acid probe is the mature polypeptide coding sequence of SEQ ID NO: 143. In another preferred aspect, the nucleic acid probe is nucleotides 61 to 1167 of SEQ ID NO: 143. In another preferred aspect, the nucleic acid probe is a polynucleotide sequence that encodes the polypeptide of SEQ ID NO: 143, or a subsequence thereof. In another preferred aspect, the nucleic acid probe is SEQ ID NO: 143.

In another preferred aspect, the nucleic acid probe is the mature polypeptide coding sequence of SEQ ID NO: 145. In another preferred aspect, the nucleic acid probe is nucleotides 64 to 1218 of SEQ ID NO: 145. In another preferred aspect, the nucleic acid probe is a polynucleotide sequence that encodes the polypeptide of SEQ ID NO: 145, or a subsequence thereof. In another preferred aspect, the nucleic acid probe is SEQ ID NO: 145.

In another preferred aspect, the nucleic acid probe is the mature polypeptide coding sequence of SEQ ID NO: 147. In another preferred aspect, the nucleic acid probe is nucleotides 58 to 1281 of SEQ ID NO: 147. In another preferred aspect, the nucleic acid probe is a polynucleotide sequence that encodes the polypeptide of SEQ ID NO: 147, or a subsequence thereof. In another preferred aspect, the nucleic acid probe is SEQ ID NO: 147.

In another preferred aspect, the nucleic acid probe is the mature polypeptide coding sequence of SEQ ID NO: 149. In another preferred aspect, the nucleic acid probe is nucleotides 52 to 801 of SEQ ID NO: 149. In another preferred aspect, the nucleic acid probe is a polynucleotide sequence that encodes the polypeptide of SEQ ID NO: 149, or a subsequence thereof. In another preferred aspect, the nucleic acid probe is SEQ ID NO: 149.

In another preferred aspect, the nucleic acid probe is the mature polypeptide coding sequence of SEQ ID NO: 151. In another preferred aspect, the nucleic acid probe is nucleotides 61 to 819 of SEQ ID NO: 151. In another preferred aspect, the nucleic acid probe is a polynucleotide sequence that encodes the polypeptide of SEQ ID NO: 151, or a subsequence thereof. In another preferred aspect, the nucleic acid probe is SEQ ID NO: 151.

In another preferred aspect, the nucleic acid probe is the mature polypeptide coding sequence of SEQ ID NO: 153. In another preferred aspect, the nucleic acid probe is nucleotides 61 to 966 of SEQ ID NO: 153. In another preferred aspect, the nucleic acid probe is a polynucleotide sequence that encodes the polypeptide of SEQ ID NO: 153, or a subsequence thereof. In another preferred aspect, the nucleic acid probe is SEQ ID NO: 153.

In another preferred aspect, the nucleic acid probe is the mature polypeptide coding sequence of SEQ ID NO: 155. In another preferred aspect, the nucleic acid probe is nucleotides 52 to 702 of SEQ ID NO: 155. In another preferred aspect, the nucleic acid probe is a polynucleotide sequence that encodes the polypeptide of SEQ ID NO: 155, or a subsequence thereof. In another preferred aspect, the nucleic acid probe is SEQ ID NO: 155.

In another preferred aspect, the nucleic acid probe is the mature polypeptide coding sequence of SEQ ID NO: 157. In another preferred aspect, the nucleic acid probe is nucleotides 70 to 699 of SEQ ID NO: 157. In another preferred aspect, the nucleic acid probe is a polynucleotide sequence that encodes the polypeptide of SEQ ID NO: 157, or a subsequence thereof. In another preferred aspect, the nucleic acid probe is SEQ ID NO: 157.

In another preferred aspect, the nucleic acid probe is the mature polypeptide coding sequence of SEQ ID NO: 159. In another preferred aspect, the nucleic acid probe is nucleotides 49 to 711 of SEQ ID NO: 159. In another preferred aspect, the nucleic acid probe is a polynucleotide sequence that encodes the polypeptide of SEQ ID NO: 159, or a subsequence thereof. In another preferred aspect, the nucleic acid probe is SEQ ID NO: 159.

In another preferred aspect, the nucleic acid probe is the mature polypeptide coding sequence of SEQ ID NO: 161. In another preferred aspect, the nucleic acid probe is nucleotides 76 to 1452 of SEQ ID NO: 161. In another preferred aspect, the nucleic acid probe is a polynucleotide sequence that encodes the polypeptide of SEQ ID NO: 161, or a subsequence thereof. In another preferred aspect, the nucleic acid probe is SEQ ID NO: 161.

In another preferred aspect, the nucleic acid probe is the mature polypeptide coding sequence of SEQ ID NO: 163. In another preferred aspect, the nucleic acid probe is nucleotides 64 to 1018 of SEQ ID NO: 163. In another preferred aspect, the nucleic acid probe is a polynucleotide sequence that encodes the polypeptide of SEQ ID NO: 163, or a subsequence thereof. In another preferred aspect, the nucleic acid probe is SEQ ID NO: 163.

For long probes of at least 100 nucleotides in length, very low to very high stringency conditions are defined as prehybridization and hybridization at 42° C. in 5×SSPE, 0.3% SDS, 200 micrograms/ml sheared and denatured salmon sperm DNA, and either 25% formamide for very low and low stringencies, 35% formamide for medium and medium-high stringencies, or 50% formamide for high and very high stringencies, following standard Southern blotting procedures for 12 to 24 hours optimally. The carrier material is finally washed three times each for 15 minutes using 2×SSC, 0.2% SDS at 45° C. (very low stringency), at 50° C. (low stringency), at 55° C. (medium stringency), at 60° C. (medium-high stringency), at 65° C. (high stringency), and at 70° C. (very high stringency).

For short probes of about 15 nucleotides to about 70 nucleotides in length, stringency conditions are defined as prehybridization and hybridization at about 5° C. to about 10° C. below the calculated Tₘ using the calculation according to Bolton and McCarthy (1962, Proc. Natl. Acad. Sci. USA 48:1390) in 0.9 M NaCl, 0.09 M Tris-HCl pH 7.6, 6 mM EDTA, 0.5% NP-40, 1×Denhardt's solution, 1 mM sodium pyrophosphate, 1 mM sodium monobasic phosphate, 0.1 mM ATP, and 0.2 mg of yeast RNA per ml following standard Southern blotting procedures for 12 to 24 hours optimally. The carrier material is finally washed once in 6×SCC plus 0.1% SDS for 15 minutes and twice each for 15 minutes using 6×SSC at 5° C. to 10° C. below the calculated Tₘ.

In a fifth aspect, the polypeptide having cellulolytic enhancing activity is encoded by a polynucleotide comprising or consisting of a nucleotide sequence that has a degree of identity to the mature polypeptide coding sequence of SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 141, SEQ ID NO: 143, SEQ ID NO: 145, SEQ ID NO: 147, SEQ ID NO: 149, SEQ ID NO: 151, SEQ ID NO: 153, SEQ ID NO: 155, SEQ ID NO: 157, SEQ ID NO: 159, SEQ ID NO: 161, or SEQ ID NO: 163 of preferably at least 60%, more preferably at least 65%, more preferably at least 70%, more preferably at least 75%, more preferably at least 80%, more preferably at least 85%, even more preferably at least 90%, most preferably at least 91%, at least 92%, at least 93%, at least 94%, or at least 95%, and even most preferably at least 96%, at least 97%, at least 98%, at least 99%, or at least 100%.

In a sixth aspect, the polypeptide having cellulolytic enhancing activity is an artificial variant comprising a substitution, deletion, and/or insertion of one or more (e.g., several) amino acids of the mature polypeptide of SEQ ID NO: 2, SEQ ID NO: 4, SEQ ID NO: 6, SEQ ID NO: 8, SEQ ID NO: 10, SEQ ID NO: 12, or SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 42, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 142, SEQ ID NO: 144, SEQ ID NO: 146, SEQ ID NO: 148, SEQ ID NO: 150, SEQ ID NO: 152, SEQ ID NO: 154, SEQ ID NO: 156, SEQ ID NO: 158, SEQ ID NO: 160, SEQ ID NO: 162, or SEQ ID NO: 164; or a homologous sequence thereof. Preferably, amino acid changes are of a minor nature, that is conservative amino acid substitutions or insertions that do not significantly affect the folding and/or activity of the protein; small deletions, typically of one to about 30 amino acids; small amino- or carboxyl-terminal extensions, such as an amino-terminal methionine residue; a small linker peptide of up to about 20-25 residues; or a small extension that facilitates purification by changing net charge or another function, such as a poly-histidine tract, an antigenic epitope or a binding domain.

Examples of conservative substitutions are within the group of basic amino acids (arginine, lysine and histidine), acidic amino acids (glutamic acid and aspartic acid), polar amino acids (glutamine and asparagine), hydrophobic amino acids (leucine, isoleucine and valine), aromatic amino acids (phenylalanine, tryptophan and tyrosine), and small amino acids (glycine, alanine, serine, threonine and methionine). Amino acid substitutions that do not generally alter specific activity are known in the art and are described, for example, by H. Neurath and R. L. Hill, 1979, In, The Proteins, Academic Press, New York. The most commonly occurring exchanges are Ala/Ser, Val/Ile, Asp/Glu, Thr/Ser, Ala/Gly, Ala/Thr, Ser/Asn, Ala/Val, Ser/Gly, Tyr/Phe, Ala/Pro, Lys/Arg, Asp/Asn, Leu/Ile, Leu/Val, Ala/Glu, and Asp/Gly.

Alternatively, the amino acid changes are of such a nature that the physico-chemical properties of the polypeptides are altered. For example, amino acid changes may improve the thermal stability of the polypeptide, alter the substrate specificity, change the pH optimum, and the like.

Essential amino acids in a parent polypeptide can be identified according to procedures known in the art, such as site-directed mutagenesis or alanine-scanning mutagenesis (Cunningham and Wells, 1989, Science 244: 1081-1085). In the latter technique, single alanine mutations are introduced at every residue in the molecule, and the resultant mutant molecules are tested for cellulolytic enhancing activity to identify amino acid residues that are critical to the activity of the molecule. See also, Hilton et al., 1996, J. Biol. Chem. 271: 4699-4708. The active site of the enzyme or other biological interaction can also be determined by physical analysis of structure, as determined by such techniques as nuclear magnetic resonance, crystallography, electron diffraction, or photoaffinity labeling, in conjunction with mutation of putative contact site amino acids. See, for example, de Vos et al., 1992, Science 255: 306-312; Smith et al., 1992, J. Mol. Biol. 224: 899-904; Wlodaver et al., 1992, FEBS Lett. 309: 59-64. The identities of essential amino acids can also be inferred from analysis of identities with polypeptides that are related to the parent polypeptide.

Single or multiple amino acid substitutions, deletions, and/or insertions can be made and tested using known methods of mutagenesis, recombination, and/or shuffling, followed by a relevant screening procedure, such as those disclosed by Reidhaar-Olson and Sauer, 1988, Science 241: 53-57; Bowie and Sauer, 1989, Proc. Natl. Acad. Sci. USA 86: 2152-2156; WO 95/17413; or WO 95/22625. Other methods that can be used include error-prone PCR, phage display (e.g., Lowman et al., 1991, Biochemistry 30: 10832-10837; U.S. Pat. No. 5,223,409; WO 92/06204), and region-directed mutagenesis (Derbyshire et al., 1986, Gene 46: 145; Ner et al., 1988, DNA 7: 127).

Mutagenesis/shuffling methods can be combined with high-throughput, automated screening methods to detect activity of cloned, mutagenized polypeptides expressed by host cells (Ness et al., 1999, Nature Biotechnology 17: 893-896). Mutagenized DNA molecules that encode active polypeptides can be recovered from the host cells and rapidly sequenced using standard methods in the art. These methods allow the rapid determination of the importance of individual amino acid residues in a polypeptide.

The total number of amino acid substitutions, deletions and/or insertions of the mature polypeptide of SEQ ID NO: 2, SEQ ID NO: 4, SEQ ID NO: 6, SEQ ID NO: 8, SEQ ID NO: 10, SEQ ID NO: 12, or SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 42, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 142, SEQ ID NO: 144, SEQ ID NO: 146, SEQ ID NO: 148, SEQ ID NO: 150, SEQ ID NO: 152, SEQ ID NO: 154, SEQ ID NO: 156, SEQ ID NO: 158, SEQ ID NO: 160, SEQ ID NO: 162, or SEQ ID NO: 164, is not more than 10, e.g., 1, 2, 3, 4, 5, 6, 7, 8 or 9.

A polypeptide having cellulolytic enhancing activity may be obtained from microorganisms of any genus. For purposes of the present invention, the term “obtained from” as used herein in connection with a given source shall mean that the polypeptide encoded by a polynucleotide is produced by the source or by a strain in which the polynucleotide from the source has been inserted. In one aspect, the polypeptide obtained from a given source is secreted extracellularly.

A polypeptide having cellulolytic enhancing activity may be a bacterial polypeptide. For example, the polypeptide may be a gram positive bacterial polypeptide such as a Bacillus, Streptococcus, Streptomyces, Staphylococcus, Enterococcus, Lactobacillus, Lactococcus, Clostridium, Geobacillus, or Oceanobacillus polypeptide having cellulolytic enhancing activity, or a Gram negative bacterial polypeptide such as an E. coli, Pseudomonas, Salmonella, Campylobacter, Helicobacter, Flavobacterium, Fusobacterium, Ilyobacter, Neisseria, or Ureaplasma polypeptide having cellulolytic enhancing activity.

In one aspect, the polypeptide is a Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, or Bacillus thuringiensis polypeptide having cellulolytic enhancing activity.

In another aspect, the polypeptide is a Streptococcus equisimilis, Streptococcus pyogenes, Streptococcus uberis, or Streptococcus equi subsp. Zooepidemicus polypeptide having cellulolytic enhancing activity.

In another aspect, the polypeptide is a Streptomyces achromogenes, Streptomyces avermitilis, Streptomyces coelicolor, Streptomyces griseus, or Streptomyces lividans polypeptide having cellulolytic enhancing activity.

The polypeptide having cellulolytic enhancing activity may also be a fungal polypeptide, and more preferably a yeast polypeptide such as a Candida, Kluyveromyces, Pichia, Saccharomyces, Schizosaccharomyces, or Yarrowia polypeptide having cellulolytic enhancing activity; or more preferably a filamentous fungal polypeptide such as aan Acremonium, Agaricus, Alternaria, Aspergillus, Aureobasidium, Botryosphaeria, Ceriporiopsis, Chaetomidium, Chrysosporium, Claviceps, Cochliobolus, Coprinopsis, Coptotermes, Corynascus, Cryphonectria, Cryptococcus, Diplodia, Exidia, Filibasidium, Fusarium, Gibberella, Holomastigotoides, Humicola, Irpex, Lentinula, Leptospaeria, Magnaporthe, Melanocarpus, Meripilus, Mucor, Myceliophthora, Neocallimastix, Neurospora, Paecilomyces, Penicillium, Phanerochaete, Piromyces, Poitrasia, Pseudoplectania, Pseudotrichonympha, Rhizomucor, Schizophyllum, Scytalidium, Talaromyces, Thermoascus, Thielavia, Tolypocladium, Trichoderma, Trichophaea, Verticillium, Volvariella, or Xylaria polypeptide having cellulolytic enhancing activity.

In another aspect, the polypeptide is a Saccharomyces car/sbergensis, Saccharomyces cerevisiae, Saccharomyces diastaticus, Saccharomyces douglasii, Saccharomyces kluyveri, Saccharomyces norbensis, or Saccharomyces oviformis polypeptide having cellulolytic enhancing activity.

In another aspect, the polypeptide is an Acremonium cellulolyticus, Aspergillus aculeatus, Aspergillus awamori, Aspergillus fumigatus, Aspergillus foetidus, Aspergillus japonicus, Aspergillus nidulans, Aspergillus niger, Aspergillus oryzae, Chrysosporium keratinophilum, Chrysosporium lucknowense, Chrysosporium tropicum, Chrysosporium merdarium, Chrysosporium inops, Chrysosporium pannicola, Chrysosporium queenslandicum, Chrysosporium zonatum, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola grisea, Humicola insolens, Humicola lanuginosa, Irpex lacteus, Mucor miehei, Myceliophthora thermophila, Neurospora crassa, Penicillium funiculosum, Penicillium pinophilum, Penicillium purpurogenum, Phanerochaete chrysosporium, Thielavia achromatica, Thielavia albomyces, Thielavia albopilosa, Thielavia australeinsis, Thielavia fimeti, Thielavia microspora, Thielavia ovispora, Thielavia peruviana, Thielavia spededonium, Thielavia setosa, Thielavia subthermophila, Thielavia terrestris, Trichoderma harzianum, Trichoderma koningii, Trichoderma longibrachiatum, Trichoderma reesei, Trichoderma viride, or Trichophaea saccata polypeptide having cellulolytic enhancing activity.

It will be understood that for the aforementioned species the invention encompasses both the perfect and imperfect states, and other taxonomic equivalents, e.g., anamorphs, regardless of the species name by which they are known. Those skilled in the art will readily recognize the identity of appropriate equivalents.

Strains of these species are readily accessible to the public in a number of culture collections, such as the American Type Culture Collection (ATCC), Deutsche Sammlung von Mikroorganismen und Zellkulturen GmbH (DSM), Centraalbureau Voor Schimmelcultures (CBS), and Agricultural Research Service Patent Culture Collection, Northern Regional Research Center (NRRL).

Furthermore, polypeptides having cellulolytic enhancing activity may be identified and obtained from other sources including microorganisms isolated from nature (e.g., soil, composts, water, etc.) using the above-mentioned probes. Techniques for isolating microorganisms from natural habitats are well known in the art. The polynucleotide may then be obtained by similarly screening a genomic DNA or cDNA library of such a microorganism. Once a polynucleotide encoding a polypeptide has been detected with the probe(s), the polynucleotide can be isolated or cloned by utilizing techniques that are well known to those of ordinary skill in the art (see, e.g., Sambrook et al., 1989, supra)

Polynucleotides comprising nucleotide sequences that encode polypeptide having cellulolytic enhancing activity can be isolated and utilized to express the polypeptide having cellulolytic enhancing activity for evaluation in the methods of the present invention.

The techniques used to isolate or clone a polynucleotide encoding a polypeptide are known in the art and include isolation from genomic DNA, preparation from cDNA, or a combination thereof. The cloning of the polynucleotides from such genomic DNA can be effected, e.g., by using the well known polymerase chain reaction (PCR) or antibody screening of expression libraries to detect cloned DNA fragments with shared structural features. See, e.g., Innis et al., 1990, PCR: A Guide to Methods and Application, Academic Press, New York. Other nucleic acid amplification procedures such as ligase chain reaction (LCR), ligation activated transcription (LAT) and polynucleotide-based amplification (NASBA) may be used. The polynucleotides may be cloned from a strain or a related organism and thus, for example, may be an allelic or species variant of the polypeptide encoding region of the polynucleotide.

The polynucleotides comprise nucleotide sequences that have a degree of identity to the mature polypeptide coding sequence of SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 141, SEQ ID NO: 143, SEQ ID NO: 145, SEQ ID NO: 147, SEQ ID NO: 149, SEQ ID NO: 151, SEQ ID NO: 153, SEQ ID NO: 155, SEQ ID NO: 157, SEQ ID NO: 159, SEQ ID NO: 161, or SEQ ID NO: 163 of preferably at least 60%, more preferably at least 65%, more preferably at least 70%, more preferably at least 75%, more preferably at least 80%, more preferably at least 85%, even more preferably at least 90%, most preferably at least 91%, at least 92%, at least 93%, at least 94%, or at least 95%, and even most preferably at least 96%, at least 97%, at least 98%, or at least 99%, which encode a polypeptide having cellulolytic enhancing activity.

The polynucleotide may also be a polynucleotide encoding a polypeptide having cellulolytic enhancing activity that hybridizes under at least very low stringency conditions, preferably at least low stringency conditions, more preferably at least medium stringency conditions, more preferably at least medium-high stringency conditions, even more preferably at least high stringency conditions, and most preferably at least very high stringency conditions with (i) the mature polypeptide coding sequence of SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 141, SEQ ID NO: 143, SEQ ID NO: 145, SEQ ID NO: 147, SEQ ID NO: 149, SEQ ID NO: 151, SEQ ID NO: 153, SEQ ID NO: 155, SEQ ID NO: 157, SEQ ID NO: 159, SEQ ID NO: 161, or SEQ ID NO: 163, (ii) the genomic DNA sequence of the mature polypeptide coding sequence of SEQ ID NO: 7, SEQ ID NO: 9, SEQ ID NO: 11, SEQ ID NO: 15, SEQ ID NO: 155, SEQ ID NO: 157, or SEQ ID NO: 159 or the cDNA sequence of the mature polypeptide coding sequence of SEQ ID NO: 1, SEQ ID NO: 3, SEQ ID NO: 5, SEQ ID NO: 13, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 141, SEQ ID NO: 143, SEQ ID NO: 145, SEQ ID NO: 147, SEQ ID NO: 149, SEQ ID NO: 151, SEQ ID NO: 153, SEQ ID NO: 161, or SEQ ID NO: 163, or (iii) a full-length complementary strand of (i) or (ii); or allelic variants and subsequences thereof (Sambrook et al., 1989, supra), as defined herein.

As described earlier, the techniques used to isolate or clone a polynucleotide encoding a polypeptide are known in the art and include isolation from genomic DNA, preparation from cDNA, or a combination thereof.

### Enzyme Compositions

The enzyme compositions can comprise any protein that is useful in degrading or converting a cellulosic material.

In one aspect, the enzyme composition comprises or further comprises one or more (e.g., several) proteins selected from the group consisting of a cellulase, a hemicellulase, an esterase, an expansin, a laccase, a ligninolytic enzyme, a pectinase, a peroxidase, a protease, and a swollenin. In another aspect, the cellulase is preferably one or more (e.g., several) enzymes selected from the group consisting of an endoglucanase, a cellobiohydrolase, and a beta-glucosidase. In another aspect, the hemicellulase is preferably one or more (e.g., several) enzymes selected from the group consisting of an acetylmannan esterase, an acetylxylan esterase, an arabinanase, an arabinofuranosidase, a coumaric acid esterase, a feruloyl esterase, a galactosidase, a glucuronidase, a glucuronoyl esterase, a mannanase, a mannosidase, a xylanase, and a xylosidase.

In another aspect, the enzyme composition comprises one or more (e.g., several) cellulolytic enzymes. In another aspect, the enzyme composition comprises or further comprises one or more (e.g., several) hemicellulolytic enzymes. In another aspect, the enzyme composition comprises one or more (e.g., several) cellulolytic enzymes and one or more (e.g., several) hemicellulolytic enzymes. In another aspect, the enzyme composition comprises one or more (e.g., several) enzymes selected from the group of cellulolytic enzymes and hemicellulolytic enzymes. In another aspect, the enzyme composition comprises an endoglucanase. In another aspect, the enzyme composition comprises a cellobiohydrolase. In another aspect, the enzyme composition comprises a beta-glucosidase. In another aspect, the enzyme composition comprises an endoglucanase and a cellobiohydrolase. In another aspect, the enzyme composition comprises an endoglucanase and a beta-glucosidase. In another aspect, the enzyme composition comprises a cellobiohydrolase and a beta-glucosidase. In another aspect, the enzyme composition comprises an endoglucanase, a cellobiohydrolase, and a beta-glucosidase.

In another aspect, the enzyme composition comprises an acetylmannan esterase. In another aspect, the enzyme composition comprises an acetylxylan esterase. In another aspect, the enzyme composition comprises an arabinanase (e.g., alpha-L-arabinanase). In another aspect, the enzyme composition comprises an arabinofuranosidase (e.g., alpha-L-arabinofuranosidase). In another aspect, the enzyme composition comprises a coumaric acid esterase. In another aspect, the enzyme composition comprises a feruloyl esterase. In another aspect, the enzyme composition comprises a galactosidase (e.g., alpha-galactosidase and/or beta-galactosidase). In another aspect, the enzyme composition comprises a glucuronidase (e.g., alpha-D-glucuronidase). In another aspect, the enzyme composition comprises a glucuronoyl esterase. In another aspect, the enzyme composition comprises a mannanase. In another aspect, the enzyme composition comprises a mannosidase (e.g., beta-mannosidase). In another aspect, the enzyme composition comprises a xylanase. In a preferred aspect, the xylanase is a Family 10 xylanase. In another aspect, the enzyme composition comprises a xylosidase (e.g., beta-xylosidase).

In another aspect, the enzyme composition comprises an esterase. In another aspect, the enzyme composition comprises an expansin. In another aspect, the enzyme composition comprises a laccase. In another aspect, the enzyme composition comprises a ligninolytic enzyme. In a preferred aspect, the ligninolytic enzyme is a manganese peroxidase. In another preferred aspect, the ligninolytic enzyme is a lignin peroxidase. In another preferred aspect, the ligninolytic enzyme is a H₂O₂-producing enzyme. In another aspect, the enzyme composition comprises a pectinase. In another aspect, the enzyme composition comprises a peroxidase. In another aspect, the enzyme composition comprises a protease. In another aspect, the enzyme composition comprises a swollenin

In the methods of the present invention, the enzyme(s) can be added prior to or during fermentation, e.g., during saccharification or during or after propagation of the fermenting microorganism(s).

One or more (e.g., several) components of the enzyme composition may be wild-type proteins, recombinant proteins, or a combination of wild-type proteins and recombinant proteins. For example, one or more (e.g., several) components may be native proteins of a cell, which is used as a host cell to express recombinantly one or more (e.g., several) other components of the enzyme composition. One or more (e.g., several) components of the enzyme composition may be produced as monocomponents, which are then combined to form the enzyme composition. The enzyme composition may be a combination of multicomponent and monocomponent protein preparations.

The enzymes used in the methods of the present invention may be in any form suitable for use, such as, for example, a crude fermentation broth with or without cells removed, a cell lysate with or without cellular debris, a semi-purified or purified enzyme preparation, or a host cell as a source of the enzymes. The enzyme composition may be a dry powder or granulate, a non-dusting granulate, a liquid, a stabilized liquid, or a stabilized protected enzyme. Liquid enzyme preparations may, for instance, be stabilized by adding stabilizers such as a sugar, a sugar alcohol or another polyol, and/or lactic acid or another organic acid according to established processes.

The enzymes can be derived or obtained from any suitable origin, including, bacterial, fungal, yeast, plant, or mammalian origin. The term “obtained” means herein that the enzyme may have been isolated from an organism that naturally produces the enzyme as a native enzyme. The term “obtained” also means herein that the enzyme may have been produced recombinantly in a host organism employing methods described herein, wherein the recombinantly produced enzyme is either native or foreign to the host organism or has a modified amino acid sequence, e.g., having one or more (e.g., several) amino acids that are deleted, inserted and/or substituted, i.e., a recombinantly produced enzyme that is a mutant and/or a fragment of a native amino acid sequence or an enzyme produced by nucleic acid shuffling processes known in the art.

Encompassed within the meaning of a native enzyme are natural variants and within the meaning of a foreign enzyme are variants obtained recombinantly, such as by site-directed mutagenesis or shuffling.

The polypeptide having enzyme activity may be a bacterial polypeptide. For example, the polypeptide may be a gram positive bacterial polypeptide such as a Bacillus, Streptococcus, Streptomyces, Staphylococcus, Enterococcus, Lactobacillus, Lactococcus, Clostridium, Geobacillus, or Oceanobacillus polypeptide having enzyme activity, or a Gram negative bacterial polypeptide such as an E. coli, Pseudomonas, Salmonella, Campylobacter, Helicobacter, Flavobacterium, Fusobacterium, Ilyobacter, Neisseria, or Ureaplasma polypeptide having enzyme activity.

In a preferred aspect, the polypeptide is a Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, or Bacillus thuringiensis polypeptide having enzyme activity.

In another preferred aspect, the polypeptide is a Streptococcus equisimilis, Streptococcus pyogenes, Streptococcus uberis, or Streptococcus equi subsp. Zooepidemicus polypeptide having enzyme activity.

In another preferred aspect, the polypeptide is a Streptomyces achromogenes, Streptomyces avermitilis, Streptomyces coelicolor, Streptomyces griseus, or Streptomyces lividans polypeptide having enzyme activity.

The polypeptide having enzyme activity may also be a fungal polypeptide, and more preferably a yeast polypeptide such as a Candida, Kluyveromyces, Pichia, Saccharomyces, Schizosaccharomyces, or Yarrowia polypeptide having enzyme activity; or more preferably a filamentous fungal polypeptide such as an Acremonium, Agaricus, Alternaria, Aspergillus, Aureobasidium, Botryosphaeria, Ceriporiopsis, Chaetomidium, Chrysosporium, Claviceps, Cochliobolus, Coprinopsis, Coptotermes, Corynascus, Cryphonectria, Cryptococcus, Diplodia, Exidia, Filibasidium, Fusarium, Gibberella, Holomastigotoides, Humicola, Irpex, Lentinula, Leptospaeria, Magnaporthe, Melanocarpus, Meripilus, Mucor, Myceliophthora, Neocallimastix, Neurospora, Paecilomyces, Penicillium, Phanerochaete, Piromyces, Poitrasia, Pseudoplectania, Pseudotrichonympha, Rhizomucor, Schizophyllum, Scytalidium, Talaromyces, Thermoascus, Thielavia, Tolypocladium, Trichoderma, Trichophaea, Verticillium, Volvariella, or Xylaria polypeptide having enzyme activity.

In a preferred aspect, the polypeptide is a Saccharomyces car/sbergensis, Saccharomyces cerevisiae, Saccharomyces diastaticus, Saccharomyces douglasii, Saccharomyces kluyveri, Saccharomyces norbensis, or Saccharomyces oviformis polypeptide having enzyme activity.

In another preferred aspect, the polypeptide is an Acremonium cellulolyticus, Aspergillus aculeatus, Aspergillus awamori, Aspergillus fumigatus, Aspergillus foetidus, Aspergillus japonicus, Aspergillus nidulans, Aspergillus niger, Aspergillus oryzae, Chrysosporium keratinophilum, Chrysosporium lucknowense, Chrysosporium tropicum, Chrysosporium merdarium, Chrysosporium inops, Chrysosporium pannicola, Chrysosporium queenslandicum, Chrysosporium zonatum, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola grisea, Humicola insolens, Humicola lanuginosa, Irpex lacteus, Mucor miehei, Myceliophthora thermophila, Neurospora crassa, Penicillium funiculosum, Penicillium purpurogenum, Phanerochaete chrysosporium, Thielavia achromatica, Thielavia albomyces, Thielavia albopilosa, Thielavia australeinsis, Thielavia fimeti, Thielavia microspora, Thielavia ovispora, Thielavia peruviana, Thielavia spededonium, Thielavia setosa, Thielavia subthermophila, Thielavia terrestris, Trichoderma harzianum, Trichoderma koningii, Trichoderma longibrachiatum, Trichoderma reesei, Trichoderma viride, or Trichophaea saccata polypeptide having enzyme activity.

Chemically modified or protein engineered mutants of the polypeptides having enzyme activity may also be used.

One or more (e.g., several) components of the enzyme composition may be a recombinant component, i.e., produced by cloning of a DNA sequence encoding the single component and subsequent cell transformed with the DNA sequence and expressed in a host (see, for example, WO 91/17243 and WO 91/17244). The host is preferably a heterologous host (enzyme is foreign to host), but the host may under certain conditions also be a homologous host (enzyme is native to host). Monocomponent cellulolytic enzymes may also be prepared by purifying such a protein from a fermentation broth.

In one aspect, the one or more (e.g., several) cellulolytic enzymes comprise a commercial cellulolytic enzyme preparation. Examples of commercial cellulolytic enzyme preparations suitable for use in the present invention include, for example, CELLIC™ CTec (Novozymes A/S), CELLIC™ CTec2 (Novozymes A/S), CELLUCLAST™ (Novozymes A/S), NOVOZYM™ 188 (Novozymes A/S), CELLUZYME™ (Novozymes A/S), CEREFLO™ (Novozymes A/S), and ULTRAFLO™ (Novozymes A/S), ACCELERASE™ (Genencor Int.), LAMINEX™ (Genencor Int.), SPEZYME™ CP (Genencor Int.), FILTRASE® NL (DSM); METHAPLUS® S/L 100 (DSM), ROHAMENT™ 7069 W (Röhm GmbH), FIBREZYME® LDI (Dyadic International, Inc.), FIBREZYME® LBR (Dyadic International, Inc.), or VISCOSTAR® 150L (Dyadic International, Inc.). The cellulase enzymes are added in amounts effective from about 0.001 to about 5.0 wt % of solids, more preferably from about 0.025 to about 4.0 wt % of solids, and most preferably from about 0.005 to about 2.0 wt % of solids. The cellulase enzymes are added in amounts effective from about 0.001 to about 5.0 wt % of solids, more preferably from about 0.025 to about 4.0 wt % of solids, and most preferably from about 0.005 to about 2.0 wt % of solids.

Examples of bacterial endoglucanases that can be used in the methods of the present invention, include, but are not limited to, an Acidothermus cellulolyticus endoglucanase (WO 91/05039; WO 93/15186; U.S. Pat. No. 5,275,944; WO 96/02551; U.S. Pat. No. 5,536,655, WO 00/70031, WO 05/093050); Thermobifida fusca endoglucanase III (WO 05/093050); and Thermobifida fusca endoglucanase V (WO 05/093050).

Examples of fungal endoglucanases that can be used in the present invention include, but are not limited to, a Trichoderma reesei endoglucanase I (Penttila et al., 1986, Gene 45: 253-263; Trichoderma reesei Cel7B endoglucanase I; GENBANK™ accession no. M15665; SEQ ID NO: 66); Trichoderma reesei endoglucanase II (Saloheimo, et al., 1988, Gene 63:11-22; Trichoderma reesei Cel5A endoglucanase II; GENBANK™ accession no. M19373; SEQ ID NO: 68); Trichoderma reesei endoglucanase Ill (Okada et al., 1988, Appl. Environ. Microbiol. 64: 555-563; GENBANK™ accession no. AB003694; SEQ ID NO: 70); Trichoderma reesei endoglucanase V (Saloheimo et al., 1994, Molecular Microbiology 13: 219-228; GENBANK™ accession no. Z33381; SEQ ID NO: 72); Aspergillus aculeatus endoglucanase (Ooi et al., 1990, Nucleic Acids Research 18: 5884); Aspergillus kawachii endoglucanase (Sakamoto et al., 1995, Current Genetics 27: 435-439); Erwinia carotovara endoglucanase (Saarilahti et al., 1990, Gene 90: 9-14); Fusarium oxysporum endoglucanase (GENBANK™ accession no. L29381); Humicola grisea var. thermoidea endoglucanase (GENBANK™ accession no. AB003107); Melanocarpus albomyces endoglucanase (GENBANK™ accession no. MAL515703); Neurospora crassa endoglucanase (GENBANK™ accession no. XM_324477); Humicola insolens endoglucanase V (SEQ ID NO: 74); Myceliophthora thermophila CBS 117.65 endoglucanase (SEQ ID NO: 76); basidiomycete CBS 495.95 endoglucanase (SEQ ID NO: 78); basidiomycete CBS 494.95 endoglucanase (SEQ ID NO: 80); Thielavia terrestris NRRL 8126 CEL6B endoglucanase (SEQ ID NO: 82); Thielavia terrestris NRRL 8126 CEL6C endoglucanase (SEQ ID NO: 84); Thielavia terrestris NRRL 8126 CEL7C endoglucanase (SEQ ID NO: 86); Thielavia terrestris NRRL 8126 CEL7E endoglucanase (SEQ ID NO: 88); Thielavia terrestris NRRL 8126 CEL7F endoglucanase (SEQ ID NO: 90); Cladorrhinum foecundissimum ATCC 62373 CEL7A endoglucanase (SEQ ID NO: 92); and Trichoderma reesei strain No. VTT-D-80133 endoglucanase (SEQ ID NO: 94; GENBANK™ accession no. M15665). The endoglucanases of SEQ ID NO: 66, SEQ ID NO: 68, SEQ ID NO: 70, SEQ ID NO: 72, SEQ ID NO: 74, SEQ ID NO: 76, SEQ ID NO: 78, SEQ ID NO: 80, SEQ ID NO: 82, SEQ ID NO: 84, SEQ ID NO: 86, SEQ ID NO: 88, SEQ ID NO: 90, SEQ ID NO: 92, and SEQ ID NO: 94 described above are encoded by the mature polypeptide coding sequence of SEQ ID NO: 65, SEQ ID NO: 67, SEQ ID NO: 69, SEQ ID NO: 71, SEQ ID NO: 73, SEQ ID NO: 75, SEQ ID NO: 77, SEQ ID NO: 79, SEQ ID NO: 81, SEQ ID NO: 83, SEQ ID NO: 85, SEQ ID NO: 87, SEQ ID NO: 89, SEQ ID NO: 91, and SEQ ID NO: 93, respectively.

Examples of cellobiohydrolases useful in the present invention include, but are not limited to, Trichoderma reesei cellobiohydrolase I (SEQ ID NO: 96); Trichoderma reesei cellobiohydrolase II (SEQ ID NO: 98); Humicola insolens cellobiohydrolase I (SEQ ID NO: 100); Myceliophthora thermophila cellobiohydrolase II (SEQ ID NO: 102 and SEQ ID NO: 104); Thielavia terrestris cellobiohydrolase II (CEL6A) (SEQ ID NO: 106); Chaetomium thermophilum cellobiohydrolase I (SEQ ID NO: 108); and Chaetomium thermophilum cellobiohydrolase II (SEQ ID NO: 110). The cellobiohydrolases of SEQ ID NO: 96, SEQ ID NO: 98, SEQ ID NO: 100, SEQ ID NO: 102, SEQ ID NO: 104, SEQ ID NO: 106, SEQ ID NO: 108, SEQ ID NO: 110, and SEQ ID NO: 112 described above are encoded by the mature polypeptide coding sequence of SEQ ID NO: 97, SEQ ID NO: 99, SEQ ID NO: 101, SEQ ID NO: 103, SEQ ID NO: 105, SEQ ID NO: 107, and SEQ ID NO: 109, respectively.

Examples of beta-glucosidases useful in the present invention include, but are not limited to, Aspergillus oryzae beta-glucosidase (SEQ ID NO: 112); Aspergillus fumigatus beta-glucosidase (SEQ ID NO: 114); Penicillium brasilianum IBT 20888 beta-glucosidase (SEQ ID NO: 116); Aspergillus niger beta-glucosidase (SEQ ID NO: 118); and Aspergillus aculeatus beta-glucosidase (SEQ ID NO: 120). The beta-glucosidases of SEQ ID NO: 112, SEQ ID NO: 114, SEQ ID NO: 116, SEQ ID NO: 118, and SEQ ID NO: 120 described above are encoded by the mature polypeptide coding sequence of SEQ ID NO: 111, SEQ ID NO: 113, SEQ ID NO: 115, SEQ ID NO: 117, and SEQ ID NO: 119, respectively.

Examples of other beta-glucosidases useful in the present invention include a Aspergillus oryzae beta-glucosidase variant fusion protein of SEQ ID NO: 122 or the Aspergillus oryzae beta-glucosidase fusion protein of SEQ ID NO: 124. The beta-glucosidase fusion proteins of SEQ ID NO: 122 and SEQ ID NO: 124 are encoded by SEQ ID NO: 121 and SEQ ID NO: 123, respectively.

The Aspergillus oryzae polypeptide having beta-glucosidase activity can be obtained according to WO 2002/095014. The Aspergillus fumigatus polypeptide having beta-glucosidase activity can be obtained according to WO 2005/047499. The Penicillium brasilianum polypeptide having beta-glucosidase activity can be obtained according to WO 2007/019442. The Aspergillus niger polypeptide having beta-glucosidase activity can be obtained according to Dan et al., 2000, J. Biol. Chem. 275: 4973-4980. The Aspergillus aculeatus polypeptide having beta-glucosidase activity can be obtained according to Kawaguchi et al., 1996, Gene 173: 287-288.

Other useful endoglucanases, cellobiohydrolases, and beta-glucosidases are disclosed in numerous Glycosyl Hydrolase families using the classification according to Henrissat B., 1991, A classification of glycosyl hydrolases based on amino-acid sequence similarities, Biochem. J. 280: 309-316, and Henrissat B., and Bairoch A., 1996, Updating the sequence-based classification of glycosyl hydrolases, Biochem. J. 316: 695-696.

Other cellulolytic enzymes that may be useful in the present invention are described in EP 495,257, EP 531,315, EP 531,372, WO 89/09259, WO 94/07998, WO 95/24471, WO 96/11262, WO 96/29397, WO 96/034108, WO 97/14804, WO 98/08940, WO 98/012307, WO 98/13465, WO 98/015619, WO 98/015633, WO 98/028411, WO 99/06574, WO 99/10481, WO 99/025846, WO 99/025847, WO 99/031255, WO 2000/009707, WO 2002/050245, WO 2002/0076792, WO 2002/101078, WO 2003/027306, WO 2003/052054, WO 2003/052055, WO 2003/052056, WO 2003/052057, WO 2003/052118, WO 2004/016760, WO 2004/043980, WO 2004/048592, WO 2005/001065, WO 2005/028636, WO 2005/093050, WO 2005/093073, WO 2006/074005, WO 2006/117432, WO 2007/071818, WO 2007/071820, WO 2008/008070, WO 2008/008793, U.S. Pat. No. 4,435,307, U.S. Pat. No. 5,457,046, U.S. Pat. No. 5,648,263, U.S. Pat. No. 5,686,593, U.S. Pat. No. 5,691,178, U.S. Pat. No. 5,763,254, and U.S. Pat. No. 5,776,757.

In one aspect, the one or more (e.g., several) hemicellulolytic enzymes comprise a commercial hemicellulolytic enzyme preparation. Examples of commercial hemicellulolytic enzyme preparations suitable for use in the present invention include, for example, SHEARZYME™ (Novozymes A/S), CELLIC™ HTec (Novozymes A/S), CELLIC™ HTec2 (Novozymes A/S), VISCOZYME® (Novozymes A/S), ULTRAFLO® (Novozymes A/S), PULPZYME® HC (Novozymes A/S), MULTI FECT® Xylanase (Genencor), ACCELLERASE® XY (Genencor), ACCELLERASE® XC (Genencor), ECOPULP® TX-200A (AB Enzymes), HSP 6000 Xylanase (DSM), DEPOL™ 333P (Biocatalysts Limit, Wales, UK), DEPOL™ 740L. (Biocatalysts Limit, Wales, UK), and DEPOL™ 762P (Biocatalysts Limit, Wales, UK).

Examples of xylanases useful in the methods of the present invention include, but are not limited to, Aspergillus aculeatus xylanase (GeneSeqP:AAR63790; WO 94/21785), Aspergillus fumigatus xylanases (WO 2006/078256), and Thielavia terrestris NRRL 8126 xylanases (WO 2009/079210).

Examples of beta-xylosidases useful in the methods of the present invention include, but are not limited to, Trichoderma reesei beta-xylosidase (UniProtKB/TrEMBL accession number Q92458), Talaromyces emersonii (SwissProt accession number Q8X212), and Neurospora crassa (SwissProt accession number Q7SOW4).

Examples of acetylxylan esterases useful in the methods of the present invention include, but are not limited to, Hypocrea jecorina acetylxylan esterase (WO 2005/001036), Neurospora crassa acetylxylan esterase (UniProt accession number q7s259), Thielavia terrestris NRRL 8126 acetylxylan esterase (WO 2009/042846), Chaetomium globosum acetylxylan esterase (Uniprot accession number Q2GWX4), Chaetomium gracile acetylxylan esterase (GeneSeqP accession number AAB82124), Phaeosphaeria nodorum acetylxylan esterase (Uniprot accession number QOUHJ 1), and Humicola insolens DSM 1800 acetylxylan esterase (WO 2009/073709).

Examples of ferulic acid esterases useful in the methods of the present invention include, but are not limited to, Humicola insolens DSM 1800 feruloyl esterase (WO 2009/076122), Neurospora crassa feruloyl esterase (UniProt accession number Q9HGR3), and Neosartorya fischeri feruloyl esterase (UniProt Accession number A1D9T4).

Examples of arabinofuranosidases useful in the methods of the present invention include, but are not limited to, Humicola insolens DSM 1800 arabinofuranosidase (WO 2009/073383) and Aspergillus niger arabinofuranosidase (GeneSeqP accession number AAR94170).

Examples of alpha-glucuronidases useful in the methods of the present invention include, but are not limited to, Aspergillus clavatus alpha-glucuronidase (UniProt accession number alcc12), Trichoderma reesei alpha-glucuronidase (Uniprot accession number Q99024), Talaromyces emersonii alpha-glucuronidase (UniProt accession number Q8X211), Aspergillus niger alpha-glucuronidase (Uniprot accession number Q96WX9), Aspergillus terreus alpha-glucuronidase (SwissProt accession number QOCJP9), and Aspergillus fumigatus alpha-glucuronidase (SwissProt accession number Q4WW45).

The enzymes and proteins used in the methods of the present invention may be produced by fermentation of the above-noted microbial strains on a nutrient medium containing suitable carbon and nitrogen sources and inorganic salts, using procedures known in the art (see, e.g., Bennett, J. W. and LaSure, L. (eds.), More Gene Manipulations in Fungi, Academic Press, C A, 1991). Suitable media are available from commercial suppliers or may be prepared according to published compositions (e.g., in catalogues of the American Type Culture Collection). Temperature ranges and other conditions suitable for growth and enzyme production are known in the art (see, e.g., Bailey, J. E., and Ollis, D. F., Biochemical Engineering Fundamentals, McGraw-Hill Book Company, N Y, 1986).

The fermentation can be any method of cultivation of a cell resulting in the expression or isolation of an enzyme. Fermentation may, therefore, be understood as comprising shake flask cultivation, or small- or large-scale fermentation (including continuous, batch, fed-batch, or solid state fermentations) in laboratory or industrial fermentors performed in a suitable medium and under conditions allowing the enzyme to be expressed or isolated. The resulting enzymes produced by the methods described above may be recovered from the fermentation medium and purified by conventional procedures.

### Nucleic Acid Constructs

An isolated polynucleotide encoding a polypeptide, e.g., a polypeptide having cellulolytic enhancing activity, a cellulolytic enzyme, a hemicellulolytic enzyme, etc., may be manipulated in a variety of ways to provide for expression of the polypeptide by constructing a nucleic acid construct comprising an isolated polynucleotide encoding the polypeptide operably linked to one or more (e.g., several) control sequences that direct the expression of the coding sequence in a suitable host cell under conditions compatible with the control sequences. Manipulation of the polynucleotide's sequence prior to its insertion into a vector may be desirable or necessary depending on the expression vector. The techniques for modifying polynucleotide sequences utilizing recombinant DNA methods are well known in the art.

The control sequence may be a promoter sequence, a polynucleotide that is recognized by a host cell for expression of a polynucleotide encoding a polypeptide. The promoter sequence contains transcriptional control sequences that mediate the expression of the polypeptide. The promoter may be any polynucleotide that shows transcriptional activity in the host cell of choice including mutant, truncated, and hybrid promoters, and may be obtained from genes encoding extracellular or intracellular polypeptides either homologous or heterologous to the host cell.

Examples of suitable promoters for directing the transcription of the nucleic acid constructs in the present invention in a bacterial host cell are the promoters obtained from the Bacillus amyloliquefaciens alpha-amylase gene (amyQ), Bacillus licheniformis alpha-amylase gene (amyL), Bacillus licheniformis penicillinase gene (penP), Bacillus stearothermophilus maltogenic amylase gene (amyM), Bacillus subtilis levansucrase gene (sacB), Bacillus subtilis xylA and xylB genes, E. coli lac operon, Streptomyces coelicolor agarase gene (dagA), and prokaryotic beta-lactamase gene (Villa-Kamaroff et al., 1978, Proc. Natl. Acad. Sci. USA 75: 3727-3731), as well as the tac promoter (DeBoer et al., 1983, Proc. Natl. Acad. Sci. USA 80: 21-25). Further promoters are described in “Useful proteins from recombinant bacteria” in Gilbert et al., 1980, Scientific American, 242: 74-94; and in Sambrook et al., 1989, supra.

Examples of suitable promoters for directing the transcription of the nucleic acid constructs in the present invention in a filamentous fungal host cell are promoters obtained from the genes for Aspergillus nidulans acetamidase, Aspergillus niger neutral alpha-amylase, Aspergillus niger acid stable alpha-amylase, Aspergillus niger or Aspergillus awamori glucoamylase (glaA), Aspergillus oryzae TAKA amylase, Aspergillus oryzae alkaline protease, Aspergillus oryzae triose phosphate isomerase, Fusarium oxysporum trypsin-like protease (WO 96/00787), Fusarium venenatum amyloglucosidase (WO 00/56900), Fusarium venenatum Daria (WO 00/56900), Fusarium venenatum Quinn (WO 00/56900), Rhizomucor miehei lipase, Rhizomucor miehei aspartic proteinase, Trichoderma reesei beta-glucosidase, Trichoderma reesei cellobiohydrolase I, Trichoderma reesei cellobiohydrolase II, Trichoderma reesei endoglucanase I, Trichoderma reesei endoglucanase II, Trichoderma reesei endoglucanase Ill, Trichoderma reesei endoglucanase V, Trichoderma reesei xylanase I, Trichoderma reesei xylanase II, Trichoderma reesei beta-xylosidase, as well as the NA2-tpi promoter (a modified promoter from a gene encoding a neutral alpha-amylase in Aspergilli in which the untranslated leader has been replaced by an untranslated leader from a gene encoding triose phosphate isomerase in Aspergilli; non-limiting examples include modified promoters from the gene encoding neutral alpha-amylase in Aspergillus niger in which the untranslated leader has been replaced by an untranslated leader from the gene encoding triose phosphate isomerase in Aspergillus nidulans or Aspergillus oryzae); and mutant, truncated, and hybrid promoters thereof.

In a yeast host, useful promoters are obtained from the genes for Saccharomyces cerevisiae enolase (ENO-1), Saccharomyces cerevisiae galactokinase (GAL1), Saccharomyces cerevisiae alcohol dehydrogenase/glyceraldehyde-3-phosphate dehydrogenase (ADH1, ADH2/GAP), Saccharomyces cerevisiae triose phosphate isomerase (TPI), Saccharomyces cerevisiae metallothionein (CUP1), and Saccharomyces cerevisiae 3-phosphoglycerate kinase. Other useful promoters for yeast host cells are described by Romanos et al., 1992, Yeast 8: 423-488.

The control sequence may also be a suitable transcription terminator sequence, which is recognized by a host cell to terminate transcription. The terminator sequence is operably linked to the 3′-terminus of the polynucleotide encoding the polypeptide. Any terminator that is functional in the host cell of choice may be used in the present invention.

Preferred terminators for filamentous fungal host cells are obtained from the genes for Aspergillus nidulans anthranilate synthase, Aspergillus niger glucoamylase, Aspergillus niger alpha-glucosidase, Aspergillus oryzae TAKA amylase, and Fusarium oxysporum trypsin-like protease.

Preferred terminators for yeast host cells are obtained from the genes for Saccharomyces cerevisiae enolase, Saccharomyces cerevisiae cytochrome C (CYC1), and Saccharomyces cerevisiae glyceraldehyde-3-phosphate dehydrogenase. Other useful terminators for yeast host cells are described by Romanos et al., 1992, supra.

The control sequence may also be a suitable leader sequence, when transcribed is a nontranslated region of an mRNA that is important for translation by the host cell. The leader sequence is operably linked to the 5′-terminus of the polynucleotide encoding the polypeptide. Any leader sequence that is functional in the host cell of choice may be used.

Preferred leaders for filamentous fungal host cells are obtained from the genes for Aspergillus oryzae TAKA amylase and Aspergillus nidulans triose phosphate isomerase.

Suitable leaders for yeast host cells are obtained from the genes for Saccharomyces cerevisiae enolase (ENO-1), Saccharomyces cerevisiae 3-phosphoglycerate kinase, Saccharomyces cerevisiae alpha-factor, and Saccharomyces cerevisiae alcohol dehydrogenase/glyceraldehyde-3-phosphate dehydrogenase (ADH2/GAP).

The control sequence may also be a polyadenylation sequence, a sequence operably linked to the 3′-terminus of the polynucleotide and, when transcribed, is recognized by the host cell as a signal to add polyadenosine residues to transcribed mRNA. Any polyadenylation sequence that is functional in the host cell of choice may be used.

Preferred polyadenylation sequences for filamentous fungal host cells are obtained from the genes for Aspergillus oryzae TAKA amylase, Aspergillus niger glucoamylase, Aspergillus nidulans anthranilate synthase, Fusarium oxysporum trypsin-like protease, and Aspergillus niger alpha-glucosidase.

Useful polyadenylation sequences for yeast host cells are described by Guo and Sherman, 1995, Mol. Cellular Biol. 15: 5983-5990.

The control sequence may also be a signal peptide coding region that encodes a signal peptide linked to the N-terminus of a polypeptide and directs the polypeptide into the cell's secretory pathway. The 5′-end of the coding sequence of the polynucleotide may inherently contain a signal peptide coding sequence naturally linked in translation reading frame with the segment of the coding sequence that encodes the polypeptide. Alternatively, the 5′-end of the coding sequence may contain a signal peptide coding sequence that is foreign to the coding sequence. The foreign signal peptide coding sequence may be required where the coding sequence does not naturally contain a signal peptide coding sequence. Alternatively, the foreign signal peptide coding sequence may simply replace the natural signal peptide coding sequence in order to enhance secretion of the polypeptide. However, any signal peptide coding sequence that directs the expressed polypeptide into the secretory pathway of a host cell of choice may be used.

Effective signal peptide coding sequences for bacterial host cells are the signal peptide coding sequences obtained from the genes for Bacillus NCIB 11837 maltogenic amylase, Bacillus licheniformis subtilisin, Bacillus licheniformis beta-lactamase, Bacillus stearothermophilus alpha-amylase, Bacillus stearothermophilus neutral proteases (nprT, nprS, nprM), and Bacillus subtilis prsA. Further signal peptides are described by Simonen and Palva, 1993, Microbiological Reviews 57: 109-137.

Effective signal peptide coding sequences for filamentous fungal host cells are the signal peptide coding sequences obtained from the genes for Aspergillus niger neutral amylase, Aspergillus niger glucoamylase, Aspergillus oryzae TAKA amylase, Humicola insolens cellulase, Humicola insolens endoglucanase V, Humicola lanuginosa lipase, and Rhizomucor miehei aspartic proteinase.

Useful signal peptides for yeast host cells are obtained from the genes for Saccharomyces cerevisiae alpha-factor and Saccharomyces cerevisiae invertase. Other useful signal peptide coding sequences are described by Romanos et al., 1992, supra.

The control sequence may also be a propeptide coding sequence that encodes a propeptide positioned at the N-terminus of a polypeptide. The resultant polypeptide is known as a proenzyme or propolypeptide (or a zymogen in some cases). A propolypeptide is generally inactive and can be converted to an active polypeptide by catalytic or autocatalytic cleavage of the propeptide from the propolypeptide. The propeptide coding sequence may be obtained from the genes for Bacillus subtilis alkaline protease (aprE), Bacillus subtilis neutral protease (nprT), Myceliophthora thermophila laccase (WO 95/33836), Rhizomucor miehei aspartic proteinase, and Saccharomyces cerevisiae alpha-factor.

Where both signal peptide and propeptide sequences are present at the N-terminus of a polypeptide, the propeptide sequence is positioned next to the N-terminus of a polypeptide and the signal peptide sequence is positioned next to the N-terminus of the propeptide sequence.

It may also be desirable to add regulatory sequences that allow the regulation of the expression of the polypeptide relative to the growth of the host cell. Examples of regulatory systems are those that cause the expression of the gene to be turned on or off in response to a chemical or physical stimulus, including the presence of a regulatory compound. Regulatory systems in prokaryotic systems include the lac, tac, and trp operator systems. In yeast, the ADH2 system or GAL1 system may be used. In filamentous fungi, the Aspergillus niger glucoamylase promoter, Aspergillus oryzae TAKA alpha-amylase promoter, and Aspergillus oryzae glucoamylase promoter may be used. Other examples of regulatory sequences are those that allow for gene amplification. In eukaryotic systems, these regulatory sequences include the dihydrofolate reductase gene that is amplified in the presence of methotrexate, and the metallothionein genes that are amplified with heavy metals. In these cases, the polynucleotide encoding the polypeptide would be operably linked with the regulatory sequence.

### Expression Vectors

The various nucleotide and control sequences may be joined together to produce a recombinant expression vector that may include one or more (e.g., several) convenient restriction sites to allow for insertion or substitution of a polynucleotide encoding a polypeptide, e.g., a polypeptide having cellulolytic enhancing activity, a cellulolytic enzyme, a hemicellulolytic enzyme, etc., at such sites. Alternatively, the polynucleotide may be expressed by inserting the polynucleotide or a nucleic acid construct comprising the sequence into an appropriate vector for expression. In creating the expression vector, the coding sequence is located in the vector so that the coding sequence is operably linked with the appropriate control sequences for expression.

The recombinant expression vector may be any vector (e.g., a plasmid or virus) that can be conveniently subjected to recombinant DNA procedures and can bring about expression of the polynucleotide. The choice of the vector will typically depend on the compatibility of the vector with the host cell into which the vector is to be introduced. The vector may be a linear or closed circular plasmid.

The vector may be an autonomously replicating vector, i.e., a vector that exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one that, when introduced into the host cell, is integrated into the genome and replicated together with the chromosome(s) into which it has been integrated. Furthermore, a single vector or plasmid or two or more vectors or plasmids that together contain the total DNA to be introduced into the genome of the host cell, or a transposon, may be used.

The vector preferably contains one or more (e.g., several) selectable markers that permit easy selection of transformed, transfected, transduced, or the like cells. A selectable marker is a gene the product of which provides for biocide or viral resistance, resistance to heavy metals, prototrophy to auxotrophs, and the like.

Examples of bacterial selectable markers are the dal genes from Bacillus subtilis or Bacillus licheniformis, or markers that confer antibiotic resistance such as ampicillin, chloramphenicol, kanamycin, or tetracycline resistance. Suitable markers for yeast host cells are ADE2, HIS3, LEU2, LYS2, MET3, TRP1, and URA3. Selectable markers for use in a filamentous fungal host cell include, but are not limited to, amdS (acetamidase), argB (ornithine carbamoyltransferase), bar (phosphinothricin acetyltransferase), hph (hygromycin phosphotransferase), niaD (nitrate reductase), pyrG (orotidine-5′-phosphate decarboxylase), sC (sulfate adenyltransferase), and trpC (anthranilate synthase), as well as equivalents thereof. Preferred for use in an Aspergillus cell are the amdS and pyrG genes of Aspergillus nidulans or Aspergillus oryzae and the bar gene of Streptomyces hygroscopicus.

The vector preferably contains an element(s) that permits integration of the vector into the host cell's genome or autonomous replication of the vector in the cell independent of the genome.

For integration into the host cell genome, the vector may rely on the polynucleotide's sequence encoding the polypeptide or any other element of the vector for integration into the genome by homologous or non-homologous recombination. Alternatively, the vector may contain additional polynucleotides for directing integration by homologous recombination into the genome of the host cell at a precise location(s) in the chromosome(s). To increase the likelihood of integration at a precise location, the integrational elements should contain a sufficient number of nucleic acids, such as 100 to 10,000 base pairs, 400 to 10,000 base pairs, and 800 to 10,000 base pairs, which have a high degree of sequence identity to the corresponding target sequence to enhance the probability of homologous recombination. The integrational elements may be any sequence that is homologous with the target sequence in the genome of the host cell. Furthermore, the integrational elements may be non-encoding or encoding polynucleotides. On the other hand, the vector may be integrated into the genome of the host cell by non-homologous recombination.

For autonomous replication, the vector may further comprise an origin of replication enabling the vector to replicate autonomously in the host cell in question. The origin of replication may be any plasmid replicator mediating autonomous replication that functions in a cell. The term “origin of replication” or “plasmid replicator” means a polynucleotide that enables a plasmid or vector to replicate in vivo.

Examples of bacterial origins of replication are the origins of replication of plasmids pBR322, pUC19, pACYC177, and pACYC184 permitting replication in E. coli, and pUB110, pE194, pTA1060, and pAMβ1 permitting replication in Bacillus.

Examples of origins of replication for use in a yeast host cell are the 2 micron origin of replication, ARS1, ARS4, the combination of ARS1 and CEN3, and the combination of ARS4 and CEN6.

Examples of origins of replication useful in a filamentous fungal cell are AMA1 and ANS1 (Gems et al., 1991, Gene 98: 61-67; Cullen et al., 1987, Nucleic Acids Res. 15: 9163-9175; WO 00/24883). Isolation of the AMA1 gene and construction of plasmids or vectors comprising the gene can be accomplished according to the methods disclosed in WO 00/24883.

More than one copy of a polynucleotide may be inserted into a host cell to increase production of a polypeptide. An increase in the copy number of the polynucleotide can be obtained by integrating at least one additional copy of the sequence into the host cell genome or by including an amplifiable selectable marker gene with the polynucleotide where cells containing amplified copies of the selectable marker gene, and thereby additional copies of the polynucleotide, can be selected for by cultivating the cells in the presence of the appropriate selectable agent.

The procedures used to ligate the elements described above to construct the recombinant expression vectors are well known to one skilled in the art (see, e.g., Sambrook et al., 1989, supra).

### Host Cells

Recombinant host cells comprising a polynucleotide encoding a polypeptide, e.g., a polypeptide having cellulolytic enhancing activity, a cellulolytic enzyme, a hemicellulolytic enzyme, etc., can be advantageously used in the recombinant production of the polypeptide. A construct or vector comprising such a polynucleotide is introduced into a host cell so that the vector is maintained as a chromosomal integrant or as a self-replicating extra-chromosomal vector as described earlier. The term “host cell” encompasses any progeny of a parent cell that is not identical to the parent cell due to mutations that occur during replication. The choice of a host cell will to a large extent depend upon the gene encoding the polypeptide and its source. The host cell may be any cell useful in the recombinant production of a polypeptide, e.g., a prokaryote or a eukaryote.

The prokaryotic host cell may be any gram-positive or gram-negative bacterium. Gram-positive bacteria include, but not limited to, Bacillus, Clostridium, Enterococcus, Geobacillus, Lactobacillus, Lactococcus, Oceanobacillus, Staphylococcus, Streptococcus, and Streptomyces. Gram-negative bacteria include, but not limited to, Campylobacter, E. coli, Flavobacterium, Fusobacterium, Helicobacter, Ilyobacter, Neisseria, Pseudomonas, Salmonella, and Ureaplasma.

The bacterial host cell may be any Bacillus cell including, but not limited to, Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, and Bacillus thuringiensis cells.

The bacterial host cell may also be any Streptococcus cell including, but not limited to, Streptococcus equisimilis, Streptococcus pyogenes, Streptococcus uberis, and Streptococcus equi subsp. Zooepidemicus cells.

The bacterial host cell may also be any Streptomyces cell including, but not limited to, Streptomyces achromogenes, Streptomyces avermitilis, Streptomyces coelicolor, Streptomyces griseus, and Streptomyces lividans cells.

The introduction of DNA into a Bacillus cell may, for instance, be effected by protoplast transformation (see, e.g., Chang and Cohen, 1979, Mol. Gen. Genet. 168: 111-115), by using competent cells (see, e.g., Young and Spizizen, 1961, J. Bacteriol. 81: 823-829, or Dubnau and Davidoff-Abelson, 1971, J. Mol. Biol. 56: 209-221), by electroporation (see, e.g., Shigekawa and Dower, 1988, Biotechniques 6: 742-751), or by conjugation (see, e.g., Koehler and Thorne, 1987, J. Bacteriol. 169: 5271-5278). The introduction of DNA into an E. coli cell may, for instance, be effected by protoplast transformation (see, e.g., Hanahan, 1983, J. Mol. Biol. 166: 557-580) or electroporation (see, e.g., Dower et al., 1988, Nucleic Acids Res. 16: 6127-6145). The introduction of DNA into a Streptomyces cell may, for instance, be effected by protoplast transformation and electroporation (see, e.g., Gong et al., 2004, Folia Microbiol. (Praha) 49: 399-405), by conjugation (see, e.g., Mazodier et al., 1989, J. Bacteriol. 171: 3583-3585), or by transduction (see, e.g., Burke et al., 2001, Proc. Natil. Acad. Sci. USA 98: 6289-6294). The introduction of DNA into a Pseudomonas cell may, for instance, be effected by electroporation (see, e.g., Choi et al., 2006, J. Microbiol. Methods 64: 391-397) or by conjugation (see, e.g., Pinedo and Smets, 2005, Appl. Environ. Microbiol. 71: 51-57). The introduction of DNA into a Streptococcus cell may, for instance, be effected by natural competence (see, e.g., Perry and Kuramitsu, 1981, Infect. Immun. 32: 1295-1297), by protoplast transformation (see, e.g., Catt and Jollick, 1991, Microbios 68: 189-207), by electroporation (see, e.g., Buckley et al., 1999, Appl. Environ. Microbiol. 65: 3800-3804) or by conjugation (see, e.g., Clewell, 1981, Microbiol. Rev. 45: 409-436). However, any method known in the art for introducing DNA into a host cell can be used.

The host cell may also be a eukaryote, such as a mammalian, insect, plant, or fungal cell.

The host cell may be a fungal cell. “Fungi” as used herein includes the phyla Ascomycota, Basidiomycota, Chytridiomycota, and Zygomycota (as defined by Hawksworth et al., In, Ainsworth and Bisby's Dictionary of The Fungi, 8th edition, 1995, CAB International, University Press, Cambridge, UK) as well as the Oomycota (as cited in Hawksworth et al., 1995, supra, page 171) and all mitosporic fungi (Hawksworth et al., 1995, supra).

The fungal host cell may be a yeast cell. “Yeast” as used herein includes ascosporogenous yeast (Endomycetales), basidiosporogenous yeast, and yeast belonging to the Fungi Imperfecti (Blastomycetes). Since the classification of yeast may change in the future, for the purposes of this invention, yeast shall be defined as described in Biology and Activities of Yeast (Skinner, F. A., Passmore, S. M., and Davenport, R. R., eds, Soc. App. Bacteriol. Symposium Series No. 9, 1980).

The yeast host cell may be a Candida, Hansenula, Kluyveromyces, Pichia, Saccharomyces, Schizosaccharomyces, or Yarrowia cell such as a Kluyveromyces lactis, Saccharomyces carlsbergensis, Saccharomyces cerevisiae, Saccharomyces diastaticus, Saccharomyces douglasii, Saccharomyces kluyveri, Saccharomyces norbensis, Saccharomyces oviformis, or Yarrowia lipolytica cell.

The fungal host cell may be a filamentous fungal cell. “Filamentous fungi” include all filamentous forms of the subdivision Eumycota and Oomycota (as defined by Hawksworth et al., 1995, supra). The filamentous fungi are generally characterized by a mycelial wall composed of chitin, cellulose, glucan, chitosan, mannan, and other complex polysaccharides. Vegetative growth is by hyphal elongation and carbon catabolism is obligately aerobic. In contrast, vegetative growth by yeasts such as Saccharomyces cerevisiae is by budding of a unicellular thallus and carbon catabolism may be fermentative.

The filamentous fungal host cell may be an Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, or Trichoderma cell.

For example, the filamentous fungal host cell may be an Aspergillus awamori, Aspergillus foetidus, Aspergillus fumigatus, Aspergillus japonicus, Aspergillus nidulans, Aspergillus niger, Aspergillus oryzae, Bjerkandera adusta, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Coprinus cinereus, Coriolus hirsutus, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Thielavia terrestris, Trametes villosa, Trametes versicolor, Trichoderma harzianum, Trichoderma koningii, Trichoderma longibrachiatum, Trichoderma reesei, or Trichoderma viride cell.

Fungal cells may be transformed by a process involving protoplast formation, transformation of the protoplasts, and regeneration of the cell wall in a manner known per se. Suitable procedures for transformation of Aspergillus and Trichoderma host cells are described in EP 238023, Yelton et al., 1984, Proc. Natl. Acad. Sci. USA 81: 1470-1474, and Christensen et al., 1988, Bio/Technology 6: 1419-1422. Suitable methods for transforming Fusarium species are described by Malardier et al., 1989, Gene 78: 147-156, and WO 96/00787. Yeast may be transformed using the procedures described by Becker and Guarente, In Abelson, J. N. and Simon, M. I., editors, Guide to Yeast Genetics and Molecular Biology, Methods in Enzymology, Volume 194, pp 182-187, Academic Press, Inc., New York; Ito et al., 1983, J. Bacteriol. 153: 163; and Hinnen et al., 1978, Proc. Natl. Acad. Sci. USA 75: 1920.

### Methods of Production

Methods for producing a polypeptide, e.g., a polypeptide having cellulolytic enhancing activity, a cellulolytic enzyme, a hemicellulolytic enzyme, etc., comprise (a) cultivating a cell, which in its wild-type form is capable of producing the polypeptide, under conditions conducive for production of the polypeptide; and (b) recovering the polypeptide.

Alternatively, methods for producing a polypeptide, e.g., a polypeptide having cellulolytic enhancing activity, a cellulolytic enzyme, a hemicellulolytic enzyme, etc., comprise (a) cultivating a recombinant host cell under conditions conducive for production of the polypeptide; and (b) recovering the polypeptide.

In the production methods, the cells are cultivated in a nutrient medium suitable for production of the polypeptide using methods well known in the art. For example, the cell may be cultivated by shake flask cultivation, and small-scale or large-scale fermentation (including continuous, batch, fed-batch, or solid state fermentations) in laboratory or industrial fermentors performed in a suitable medium and under conditions allowing the polypeptide to be expressed and/or isolated. The cultivation takes place in a suitable nutrient medium comprising carbon and nitrogen sources and inorganic salts, using procedures known in the art. Suitable media are available from commercial suppliers or may be prepared according to published compositions (e.g., in catalogues of the American Type Culture Collection). If the polypeptide is secreted into the nutrient medium, the polypeptide can be recovered directly from the medium. If the polypeptide is not secreted, it can be recovered from cell lysates.

The polypeptide may be detected using methods known in the art that are specific for the polypeptides. These detection methods may include use of specific antibodies, formation of an enzyme product, or disappearance of an enzyme substrate. For example, an enzyme assay may be used to determine the activity of the polypeptide. The polypeptides having cellulolytic enhancing activity are detected using the methods described herein.

The resulting broth may be used as is or the polypeptide may be recovered using methods known in the art. For example, the polypeptide may be recovered from the nutrient medium by conventional procedures including, but not limited to, centrifugation, filtration, extraction, spray-drying, evaporation, or precipitation.

The polypeptides may be purified by a variety of procedures known in the art including, but not limited to, chromatography (e.g., ion exchange, affinity, hydrophobic, chromatofocusing, and size exclusion), electrophoretic procedures (e.g., preparative isoelectric focusing), differential solubility (e.g., ammonium sulfate precipitation), SDS-PAGE, or extraction (see, e.g., Protein Purification, J.-C. Janson and Lars Ryden, editors, VCH Publishers, New York, 1989) to obtain substantially pure polypeptides.

In an alternative aspect, the polypeptide is not recovered, but rather a host cell expressing a polypeptide is used as a source of the polypeptide.

### Methods for Processing Cellulosic Material

The compositions and methods of the present invention can be used to saccharify a cellulosic material to fermentable sugars and convert the fermentable sugars to many useful substances, e.g., fuel, potable ethanol, and/or fermentation products (e.g., acids, alcohols, ketones, gases, and the like). The production of a desired fermentation product from cellulosic material typically involves pretreatment, enzymatic hydrolysis (saccharification), and fermentation.

The present invention also relates to methods for degrading or converting a cellulosic material, comprising: treating the cellulosic material with an enzyme composition in the presence of a polypeptide having cellulolytic enhancing activity and an organic compound. In one aspect, the method above further comprises recovering the degraded or converted cellulosic material. Soluble products of degradation or conversion of the cellulosic material can be separated from the insoluble cellulosic material using technology well known in the art such as, for example, centrifugation, filtration, and gravity settling.

The present invention also relates to methods for producing a fermentation product, comprising: (a) saccharifying a cellulosic material with an enzyme composition in the presence of a polypeptide having cellulolytic enhancing activity and an organic compound; (b) fermenting the saccharified cellulosic material with one or more (e.g., several) fermenting microorganisms to produce the fermentation product; and (c) recovering the fermentation product from the fermentation.

The present invention also relates to methods of fermenting a cellulosic material, comprising: fermenting the cellulosic material with one or more (e.g., several) fermenting microorganisms, wherein the cellulosic material is saccharified with an enzyme composition in the presence of a polypeptide having cellulolytic enhancing activity and an organic compound. In one aspect, the fermenting of the cellulosic material produces a fermentation product. In another aspect, the method further comprises recovering the fermentation product from the fermentation.

In one aspect, the organic compound is recovered following saccharification or fermentation and recycled back to a new saccharification reaction. Recycling of the organic compound can be accomplished using processes conventional in the art.

The processing of cellulosic material according to the present invention can be accomplished using processes conventional in the art. Moreover, the methods of the present invention can be implemented using any conventional biomass processing apparatus configured to operate in accordance with the invention.

Hydrolysis (saccharification) and fermentation, separate or simultaneous, include, but are not limited to, separate hydrolysis and fermentation (SHF); simultaneous saccharification and fermentation (SSF); simultaneous saccharification and cofermentation (SSCF); hybrid hydrolysis and fermentation (HHF); separate hydrolysis and co-fermentation (SHCF); hybrid hydrolysis and co-fermentation (HHCF); and direct microbial conversion (DMC), also sometimes called consolidated bioprocessing (CBP). SHF uses separate process steps to first enzymatically hydrolyze cellulosic material to fermentable sugars, e.g., glucose, cellobiose, cellotriose, and pentose monomers, and then ferment the fermentable sugars to ethanol. In SSF, the enzymatic hydrolysis of cellulosic material and the fermentation of sugars to ethanol are combined in one step (Philippidis, G. P., 1996, Cellulose bioconversion technology, in Handbook on Bioethanol: Production and Utilization, Wyman, C. E., ed., Taylor & Francis, Washington, D.C., 179-212). SSCF involves the cofermentation of multiple sugars (Sheehan, J., and Himmel, M., 1999, Enzymes, energy and the environment: A strategic perspective on the U.S. Department of Energy's research and development activities for bioethanol, Biotechnol. Prog. 15: 817-827). HHF involves a separate hydrolysis step, and in addition a simultaneous saccharification and hydrolysis step, which can be carried out in the same reactor. The steps in an HHF process can be carried out at different temperatures, i.e., high temperature enzymatic saccharification followed by SSF at a lower temperature that the fermentation strain can tolerate. DMC combines all three processes (enzyme production, hydrolysis, and fermentation) in one or more (e.g., several) steps where the same organism is used to produce the enzymes for conversion of the cellulosic material to fermentable sugars and to convert the fermentable sugars into a final product (Lynd, L. R., Weimer, P. J., van Zyl, W. H., and Pretorius, I. S., 2002, Microbial cellulose utilization: Fundamentals and biotechnology, Microbiol. Mol. Biol. Reviews 66: 506-577). It is understood herein that any method known in the art comprising pretreatment, enzymatic hydrolysis (saccharification), fermentation, or a combination thereof, can be used in the practicing the methods of the present invention.

A conventional apparatus can include a fed-batch stirred reactor, a batch stirred reactor, a continuous flow stirred reactor with ultrafiltration, and/or a continuous plug-flow column reactor (Fernanda de Castilhos Corazza, Flavio Faria de Moraes, Gisella Maria Zanin and Ivo Neitzel, 2003, Optimal control in fed-batch reactor for the cellobiose hydrolysis, Acta Scientiarum. Technology 25: 33-38; Gusakov, A. V., and Sinitsyn, A. P., 1985, Kinetics of the enzymatic hydrolysis of cellulose: 1. A mathematical model for a batch reactor process, Enz. Microb. Technol. 7: 346-352), an attrition reactor (Ryu, S. K., and Lee, J. M., 1983, Bioconversion of waste cellulose by using an attrition bioreactor, Biotechnol. Bioeng. 25: 53-65), or a reactor with intensive stirring induced by an electromagnetic field (Gusakov, A. V., Sinitsyn, A. P., Davydkin, I. Y., Davydkin, V. Y., Protas, O. V., 1996, Enhancement of enzymatic cellulose hydrolysis using a novel type of bioreactor with intensive stirring induced by electromagnetic field, Appl. Biochem. Biotechnol. 56: 141-153). Additional reactor types include: fluidized bed, upflow blanket, immobilized, and extruder type reactors for hydrolysis and/or fermentation.

Pretreatment.

In practicing the methods of the present invention, any pretreatment process known in the art can be used to disrupt plant cell wall components of cellulosic material (Chandra et al., 2007, Substrate pretreatment: The key to effective enzymatic hydrolysis of lignocellulosics? Adv. Biochem. Engin./Biotechnol. 108: 67-93; Galbe and Zacchi, 2007, Pretreatment of lignocellulosic materials for efficient bioethanol production, Adv. Biochem. Engin./Biotechnol. 108: 41-65; Hendriks and Zeeman, 2009, Pretreatments to enhance the digestibility of lignocellulosic biomass, Bioresource Technol. 100: 10-18; Mosier et al., 2005, Features of promising technologies for pretreatment of lignocellulosic biomass, Bioresource Technol. 96: 673-686; Taherzadeh and Karimi, 2008, Pretreatment of lignocellulosic wastes to improve ethanol and biogas production: A review, Int. J. of Mol. Sci. 9: 1621-1651; Yang and Wyman, 2008, Pretreatment: the key to unlocking low-cost cellulosic ethanol, Biofuels Bioproducts and Biorefining-Biofpr. 2: 26-40).

The cellulosic material can also be subjected to particle size reduction, pre-soaking, wetting, washing, or conditioning prior to pretreatment using methods known in the art.

Conventional pretreatments include, but are not limited to, steam pretreatment (with or without explosion), dilute acid pretreatment, hot water pretreatment, alkaline pretreatment, lime pretreatment, wet oxidation, wet explosion, ammonia fiber explosion, organosolv pretreatment, and biological pretreatment. Additional pretreatments include ammonia percolation, ultrasound, electroporation, microwave, supercritical CO₂, supercritical H₂O, ozone, and gamma irradiation pretreatments.

The cellulosic material can be pretreated before hydrolysis and/or fermentation. Pretreatment is preferably performed prior to the hydrolysis. Alternatively, the pretreatment can be carried out simultaneously with enzyme hydrolysis to release fermentable sugars, such as glucose, xylose, and/or cellobiose. In most cases the pretreatment step itself results in some conversion of biomass to fermentable sugars (even in absence of enzymes).

Steam Pretreatment: In steam pretreatment, cellulosic material is heated to disrupt the plant cell wall components, including lignin, hemicellulose, and cellulose to make the cellulose and other fractions, e.g., hemicellulose, accessible to enzymes. Cellulosic material is passed to or through a reaction vessel where steam is injected to increase the temperature to the required temperature and pressure and is retained therein for the desired reaction time. Steam pretreatment is preferably done at 140-230° C., more preferably 160-200° C., and most preferably 170-190° C., where the optimal temperature range depends on any addition of a chemical catalyst. Residence time for the steam pretreatment is preferably 1-15 minutes, more preferably 3-12 minutes, and most preferably 4-10 minutes, where the optimal residence time depends on temperature range and any addition of a chemical catalyst. Steam pretreatment allows for relatively high solids loadings, so that cellulosic material is generally only moist during the pretreatment. The steam pretreatment is often combined with an explosive discharge of the material after the pretreatment, which is known as steam explosion, that is, rapid flashing to atmospheric pressure and turbulent flow of the material to increase the accessible surface area by fragmentation (Duff and Murray, 1996, Bioresource Technology 855: 1-33; Galbe and Zacchi, 2002, Appl. Microbiol. Biotechnol. 59: 618-628; U.S. Patent Application No. 20020164730). During steam pretreatment, hemicellulose acetyl groups are cleaved and the resulting acid autocatalyzes partial hydrolysis of the hemicellulose to monosaccharides and oligosaccharides. Lignin is removed to only a limited extent.

A catalyst such as H₂SO₄ or SO₂ (typically 0.3 to 3% w/w) is often added prior to steam pretreatment, which decreases the time and temperature, increases the recovery, and improves enzymatic hydrolysis (Ballesteros et al., 2006, Appl. Biochem. Biotechnol. 129-132: 496-508; Varga et al., 2004, Appl. Biochem. Biotechnol. 113-116: 509-523; Sassner et al., 2006, Enzyme Microb. Technol. 39: 756-762).

Chemical Pretreatment: The term “chemical treatment” refers to any chemical pretreatment that promotes the separation and/or release of cellulose, hemicellulose, and/or lignin. Examples of suitable chemical pretreatment processes include, for example, dilute acid pretreatment, lime pretreatment, wet oxidation, ammonia fiber/freeze explosion (AFEX), ammonia percolation (APR), and organosolv pretreatments.

In dilute acid pretreatment, cellulosic material is mixed with dilute acid, typically H₂SO₄, and water to form a slurry, heated by steam to the desired temperature, and after a residence time flashed to atmospheric pressure. The dilute acid pretreatment can be performed with a number of reactor designs, e.g., plug-flow reactors, counter-current reactors, or continuous counter-current shrinking bed reactors (Duff and Murray, 1996, supra; Schell et al., 2004, Bioresource Technol. 91: 179-188; Lee et al., 1999, Adv. Biochem. Eng. Biotechnol. 65: 93-115).

Several methods of pretreatment under alkaline conditions can also be used. These alkaline pretreatments include, but are not limited to, lime pretreatment, wet oxidation, ammonia percolation (APR), and ammonia fiber/freeze explosion (AFEX).

Lime pretreatment is performed with calcium carbonate, sodium hydroxide, or ammonia at low temperatures of 85-150° C. and residence times from 1 hour to several days (Wyman et al., 2005, Bioresource Technol. 96: 1959-1966; Mosier et al., 2005, Bioresource Technol. 96: 673-686). WO 2006/110891, WO 2006/11899, WO 2006/11900, and WO 2006/110901 disclose pretreatment methods using ammonia.

Wet oxidation is a thermal pretreatment performed typically at 180-200° C. for 5-15 minutes with addition of an oxidative agent such as hydrogen peroxide or over-pressure of oxygen (Schmidt and Thomsen, 1998, Bioresource Technol. 64: 139-151; Palonen et al., 2004, Appl. Biochem. Biotechnol. 117: 1-17; Varga et al., 2004, Biotechnol. Bioeng. 88: 567-574; Martin et al., 2006, J. Chem. Technol. Biotechnol. 81: 1669-1677). The pretreatment is performed at preferably 1-40% dry matter, more preferably 2-30% dry matter, and most preferably 5-20% dry matter, and often the initial pH is increased by the addition of alkali such as sodium carbonate.

A modification of the wet oxidation pretreatment method, known as wet explosion (combination of wet oxidation and steam explosion), can handle dry matter up to 30%. In wet explosion, the oxidizing agent is introduced during pretreatment after a certain residence time. The pretreatment is then ended by flashing to atmospheric pressure (WO 2006/032282).

Ammonia fiber explosion (AFEX) involves treating cellulosic material with liquid or gaseous ammonia at moderate temperatures such as 90-100° C. and high pressure such as 17-20 bar for 5-10 minutes, where the dry matter content can be as high as 60% (Gollapalli et al., 2002, Appl. Biochem. Biotechnol. 98: 23-35; Chundawat et al., 2007, Biotechnol. Bioeng. 96: 219-231; Alizadeh et al., 2005, Appl. Biochem. Biotechnol. 121: 1133-1141; Teymouri et al., 2005, Bioresource Technol. 96: 2014-2018). AFEX pretreatment results in the depolymerization of cellulose and partial hydrolysis of hemicellulose. Lignin-carbohydrate complexes are cleaved.

Organosolv pretreatment delignifies cellulosic material by extraction using aqueous ethanol (40-60% ethanol) at 160-200° C. for 30-60 minutes (Pan et al., 2005, Biotechnol. Bioeng. 90: 473-481; Pan et al., 2006, Biotechnol. Bioeng. 94: 851-861; Kurabi et al., 2005, Appl. Biochem. Biotechnol. 121: 219-230). Sulphuric acid is usually added as a catalyst. In organosolv pretreatment, the majority of hemicellulose is removed.

Other examples of suitable pretreatment methods are described by Schell et al., 2003, Appl. Biochem. and Biotechnol. Vol. 105-108, p. 69-85, and Mosier et al., 2005, Bioresource Technology 96: 673-686, and U.S. Published Application 2002/0164730.

In one aspect, the chemical pretreatment is preferably carried out as an acid treatment, and more preferably as a continuous dilute and/or mild acid treatment. The acid is typically sulfuric acid, but other acids can also be used, such as acetic acid, citric acid, nitric acid, phosphoric acid, tartaric acid, succinic acid, hydrogen chloride, or mixtures thereof. Mild acid treatment is conducted in the pH range of preferably 1-5, more preferably 1-4, and most preferably 1-3. In one aspect, the acid concentration is in the range from preferably 0.01 to 20 wt % acid, more preferably 0.05 to 10 wt % acid, even more preferably 0.1 to 5 wt % acid, and most preferably 0.2 to 2.0 wt % acid. The acid is contacted with cellulosic material and held at a temperature in the range of preferably 160-220° C., and more preferably 165-195° C., for periods ranging from seconds to minutes to, e.g., 1 second to 60 minutes.

In another aspect, pretreatment is carried out as an ammonia fiber explosion step (AFEX pretreatment step).

In another aspect, pretreatment takes place in an aqueous slurry. In preferred aspects, cellulosic material is present during pretreatment in amounts preferably between 10-80 wt %, more preferably between 20-70 wt %, and most preferably between 30-60 wt %, such as around 50 wt %. The pretreated cellulosic material can be unwashed or washed using any method known in the art, e.g., washed with water.

Mechanical Pretreatment: The term “mechanical pretreatment” refers to various types of grinding or milling (e.g., dry milling, wet milling, or vibratory ball milling).

Physical Pretreatment: The term “physical pretreatment” refers to any pretreatment that promotes the separation and/or release of cellulose, hemicellulose, and/or lignin from cellulosic material. For example, physical pretreatment can involve irradiation (e.g., microwave irradiation), steaming/steam explosion, hydrothermolysis, and combinations thereof.

Physical pretreatment can involve high pressure and/or high temperature (steam explosion). In one aspect, high pressure means pressure in the range of preferably about 300 to about 600 psi, more preferably about 350 to about 550 psi, and most preferably about 400 to about 500 psi, such as around 450 psi. In another aspect, high temperature means temperatures in the range of about 100 to about 300° C., preferably about 140 to about 235° C. In a preferred aspect, mechanical pretreatment is performed in a batch-process, steam gun hydrolyzer system that uses high pressure and high temperature as defined above, e.g., a Sunds Hydrolyzer available from Sunds Defibrator AB, Sweden.

Combined Physical and Chemical Pretreatment: Cellulosic material can be pretreated both physically and chemically. For instance, the pretreatment step can involve dilute or mild acid treatment and high temperature and/or pressure treatment. The physical and chemical pretreatments can be carried out sequentially or simultaneously, as desired. A mechanical pretreatment can also be included.

Accordingly, in a preferred aspect, cellulosic material is subjected to mechanical, chemical, or physical pretreatment, or any combination thereof, to promote the separation and/or release of cellulose, hemicellulose, and/or lignin.

Biological Pretreatment: The term “biological pretreatment” refers to any biological pretreatment that promotes the separation and/or release of cellulose, hemicellulose, and/or lignin from cellulosic material. Biological pretreatment techniques can involve applying lignin-solubilizing microorganisms (see, for example, Hsu, T.-A., 1996, Pretreatment of biomass, in Handbook on Bioethanol: Production and Utilization, Wyman, C. E., ed., Taylor & Francis, Washington, D.C., 179-212; Ghosh and Singh, 1993, Physicochemical and biological treatments for enzymatic/microbial conversion of cellulosic biomass, Adv. Appl. Microbiol. 39: 295-333; McMillan, J. D., 1994, Pretreating lignocellulosic biomass: a review, in Enzymatic Conversion of Biomass for Fuels Production, Himmel, M. E., Baker, J. O., and Overend, R. P., eds., ACS Symposium Series 566, American Chemical Society, Washington, D.C., chapter 15; Gong, C. S., Cao, N. J., Du, J., and Tsao, G. T., 1999, Ethanol production from renewable resources, in Advances in Biochemical Engineering/Biotechnology, Scheper, T., ed., Springer-Verlag Berlin Heidelberg, Germany, 65: 207-241; Olsson and Hahn-Hagerdal, 1996, Fermentation of lignocellulosic hydrolysates for ethanol production, Enz. Microb. Tech. 18: 312-331; and Vallander and Eriksson, 1990, Production of ethanol from lignocellulosic materials: State of the art, Adv. Biochem. Eng./Biotechnol. 42: 63-95).

Saccharification.

In the hydrolysis step, also known as saccharification, the cellulosic material, e.g., pretreated, is hydrolyzed to break down cellulose and alternatively also hemicellulose to fermentable sugars, such as glucose, cellobiose, xylose, xylulose, arabinose, mannose, galactose, and/or soluble oligosaccharides. The hydrolysis is performed enzymatically by an enzyme composition in the presence of a polypeptide having cellulolytic enhancing activity and an organic compound. The enzyme and protein components of the compositions can be added sequentially.

Enzymatic hydrolysis is preferably carried out in a suitable aqueous environment under conditions that can be readily determined by one skilled in the art. In a preferred aspect, hydrolysis is performed under conditions suitable for the activity of the enzyme(s), i.e., optimal for the enzyme(s). The hydrolysis can be carried out as a fed batch or continuous process where the pretreated cellulosic material (substrate) is fed gradually to, for example, an enzyme containing hydrolysis solution.

The saccharification is generally performed in stirred-tank reactors or fermentors under controlled pH, temperature, and mixing conditions. Suitable process time, temperature and pH conditions can readily be determined by one skilled in the art. For example, the saccharification can last up to 200 hours, but is typically performed for preferably about 12 to about 96 hours, more preferably about 16 to about 72 hours, and most preferably about 24 to about 48 hours. The temperature is in the range of preferably about 25° C. to about 70° C., more preferably about 30° C. to about 65° C., and more preferably about 40° C. to 60° C., in particular about 50° C. The pH is in the range of preferably about 3 to about 8, more preferably about 3.5 to about 7, and most preferably about 4 to about 6, in particular about pH 5. The dry solids content is in the range of preferably about 5 to about 50 wt %, more preferably about 10 to about 40 wt %, and most preferably about 20 to about 30 wt %.

The optimum amounts of the enzymes and polypeptides having cellulolytic enhancing activity depend on several factors including, but not limited to, the mixture of component cellulolytic enzymes, the cellulosic substrate, the concentration of cellulosic substrate, the pretreatment(s) of the cellulosic substrate, temperature, time, pH, and inclusion of fermenting organism (e.g., yeast for Simultaneous Saccharification and Fermentation).

In one aspect, an effective amount of cellulolytic or hemicellulolytic enzyme protein to cellulosic material is about 0.5 to about 50 mg, preferably at about 0.5 to about 40 mg, more preferably at about 0.5 to about 25 mg, more preferably at about 0.75 to about 20 mg, more preferably at about 0.75 to about 15 mg, even more preferably at about 0.5 to about 10 mg, and most preferably at about 2.5 to about 10 mg per g of cellulosic material.

In another aspect, an effective amount of a polypeptide having cellulolytic enhancing activity to cellulosic material is about 0.01 to about 50.0 mg, preferably about 0.01 to about 40 mg, more preferably about 0.01 to about 30 mg, more preferably about 0.01 to about 20 mg, more preferably about 0.01 to about 10 mg, more preferably about 0.01 to about 5 mg, more preferably at about 0.025 to about 1.5 mg, more preferably at about 0.05 to about 1.25 mg, more preferably at about 0.075 to about 1.25 mg, more preferably at about 0.1 to about 1.25 mg, even more preferably at about 0.15 to about 1.25 mg, and most preferably at about 0.25 to about 1.0 mg per g of cellulosic material.

In another aspect, an effective amount of a polypeptide having cellulolytic enhancing activity to cellulolytic enzyme protein is about 0.005 to about 1.0 g, preferably at about 0.01 to about 1.0 g, more preferably at about 0.15 to about 0.75 g, more preferably at about 0.15 to about 0.5 g, more preferably at about 0.1 to about 0.5 g, even more preferably at about 0.1 to about 0.5 g, and most preferably at about 0.05 to about 0.2 g per g of cellulolytic enzyme protein.

Fermentation.

The fermentable sugars obtained from the hydrolyzed cellulosic material can be fermented by one or more (e.g., several) fermenting microorganisms capable of fermenting the sugars directly or indirectly into a desired fermentation product. “Fermentation” or “fermentation process” refers to any fermentation process or any process comprising a fermentation step. Fermentation processes also include fermentation processes used in the consumable alcohol industry (e.g., beer and wine), dairy industry (e.g., fermented dairy products), leather industry, and tobacco industry. The fermentation conditions depend on the desired fermentation product and fermenting organism and can easily be determined by one skilled in the art.

In the fermentation step, sugars, released from cellulosic material as a result of the pretreatment and enzymatic hydrolysis steps, are fermented to a product, e.g., ethanol, by a fermenting organism, such as yeast. Hydrolysis (saccharification) and fermentation can be separate or simultaneous, as described herein.

Any suitable hydrolyzed cellulosic material can be used in the fermentation step in practicing the present invention. The material is generally selected based on the desired fermentation product, i.e., the substance to be obtained from the fermentation, and the process employed, as is well known in the art.

The term “fermentation medium” is understood herein to refer to a medium before the fermenting microorganism(s) is(are) added, such as, a medium resulting from a saccharification process, as well as a medium used in a simultaneous saccharification and fermentation process (SSF).

“Fermenting microorganism” refers to any microorganism, including bacterial and fungal organisms, suitable for use in a desired fermentation process to produce a fermentation product. The fermenting organism can be C₆ and/or C₅ fermenting organisms, or a combination thereof. Both C₆ and C₅ fermenting organisms are well known in the art. Suitable fermenting microorganisms are able to ferment, i.e., convert, sugars, such as glucose, xylose, xylulose, arabinose, maltose, mannose, galactose, or oligosaccharides, directly or indirectly into the desired fermentation product.

Examples of bacterial and fungal fermenting organisms producing ethanol are described by Lin et al., 2006, Appl. Microbiol. Biotechnol. 69: 627-642.

Examples of fermenting microorganisms that can ferment C₆ sugars include bacterial and fungal organisms, such as yeast. Preferred yeast includes strains of the Saccharomyces spp., preferably Saccharomyces cerevisiae.

Examples of fermenting organisms that can ferment C₅ sugars include bacterial and fungal organisms, such as some yeast. Preferred C₅ fermenting yeast include strains of Pichia, preferably Pichia stipitis, such as Pichia stipitis CBS 5773; strains of Candida, preferably Candida boidinii, Candida brassicae, Candida sheatae, Candida diddensii, Candida pseudotropicalis, or Candida utilis.

Other fermenting organisms include strains of Zymomonas, such as Zymomonas mobilis; Hansenula, such as Hansenula anomala; Kluyveromyces, such as K. fragilis; Schizosaccharomyces, such as S. pombe; E. coli, especially E. coli strains that have been genetically modified to improve the yield of ethanol; Clostridium, such as Clostridium acetobutylicum, Chlostridium thermocellum, and Chlostridium phytofermentans; Geobacillus sp.; Thermoanaerobacter, such as Thermoanaerobacter saccharolyticum; and Bacillus, such as Bacillus coagulans.

In a preferred aspect, the yeast is a Saccharomyces spp. In a more preferred aspect, the yeast is Saccharomyces cerevisiae. In another more preferred aspect, the yeast is Saccharomyces distaticus. In another more preferred aspect, the yeast is Saccharomyces uvarum. In another preferred aspect, the yeast is a Kluyveromyces. In another more preferred aspect, the yeast is Kluyveromyces marxianus. In another more preferred aspect, the yeast is Kluyveromyces fragilis. In another preferred aspect, the yeast is a Candida. In another more preferred aspect, the yeast is Candida boidinii. In another more preferred aspect, the yeast is Candida brassicae. In another more preferred aspect, the yeast is Candida diddensii. In another more preferred aspect, the yeast is Candida pseudotropicalis. In another more preferred aspect, the yeast is Candida utilis. In another preferred aspect, the yeast is a Clavispora. In another more preferred aspect, the yeast is Clavispora lusitaniae. In another more preferred aspect, the yeast is Clavispora opuntiae. In another preferred aspect, the yeast is a Pachysolen. In another more preferred aspect, the yeast is Pachysolen tannophilus. In another preferred aspect, the yeast is a Pichia. In another more preferred aspect, the yeast is a Pichia stipitis. In another preferred aspect, the yeast is a Bretannomyces. In another more preferred aspect, the yeast is Bretannomyces clausenii (Philippidis, G. P., 1996, Cellulose bioconversion technology, in Handbook on Bioethanol: Production and Utilization, Wyman, C. E., ed., Taylor & Francis, Washington, D.C., 179-212).

Bacteria that can efficiently ferment hexose and pentose to ethanol include, for example, Zymomonas mobilis, Clostridium acetobutylicum, Clostridium thermocellum, Chlostridium phytofermentans, Geobacillus sp., Thermoanaerobacter saccharolyticum, and Bacillus coagulans (Philippidis, 1996, supra).

In a preferred aspect, the bacterium is a Zymomonas. In a more preferred aspect, the bacterium is Zymomonas mobilis. In another preferred aspect, the bacterium is a Clostridium. In another more preferred aspect, the bacterium is Clostridium thermocellum.

Commercially available yeast suitable for ethanol production includes, e.g., ETHANOL RED™ yeast (available from Fermentis/Lesaffre, USA), FALI™ (available from Fleischmann's Yeast, USA), SUPERSTART™ and THERMOSACC™ fresh yeast (available from Ethanol Technology, WI, USA), BIOFERM™ AFT and XR (available from NABC—North American Bioproducts Corporation, GA, USA), GERT STRAND™ (available from Gert Strand AB, Sweden), and FERMIOL™ (available from DSM Specialties).

In a preferred aspect, the fermenting microorganism has been genetically modified to provide the ability to ferment pentose sugars, such as xylose utilizing, arabinose utilizing, and xylose and arabinose co-utilizing microorganisms.

The cloning of heterologous genes into various fermenting microorganisms has led to the construction of organisms capable of converting hexoses and pentoses to ethanol (cofermentation) (Chen and Ho, 1993, Cloning and improving the expression of Pichia stipitis xylose reductase gene in Saccharomyces cerevisiae, Appl. Biochem. Biotechnol. 39-40: 135-147; Ho et al., 1998, Genetically engineered Saccharomyces yeast capable of effectively cofermenting glucose and xylose, Appl. Environ. Microbiol. 64: 1852-1859; Kotter and Ciriacy, 1993, Xylose fermentation by Saccharomyces cerevisiae, Appl. Microbiol. Biotechnol. 38: 776-783; Walfridsson et al., 1995, Xylose-metabolizing Saccharomyces cerevisiae strains overexpressing the TKL1 and TAL1 genes encoding the pentose phosphate pathway enzymes transketolase and transaldolase, Appl. Environ. Microbiol. 61: 4184-4190; Kuyper et al., 2004, Minimal metabolic engineering of Saccharomyces cerevisiae for efficient anaerobic xylose fermentation: a proof of principle, FEMS Yeast Research 4: 655-664; Beall et al., 1991, Parametric studies of ethanol production from xylose and other sugars by recombinant Escherichia coli, Biotech. Bioeng. 38: 296-303; Ingram et al., 1998, Metabolic engineering of bacteria for ethanol production, Biotechnol. Bioeng. 58: 204-214; Zhang et al., 1995, Metabolic engineering of a pentose metabolism pathway in ethanologenic Zymomonas mobilis, Science 267: 240-243; Deanda et al., 1996, Development of an arabinose-fermenting Zymomonas mobilis strain by metabolic pathway engineering, Appl. Environ. Microbiol. 62: 4465-4470; WO 2003/062430, xylose isomerase).

In a preferred aspect, the genetically modified fermenting microorganism is Saccharomyces cerevisiae. In another preferred aspect, the genetically modified fermenting microorganism is Zymomonas mobilis. In another preferred aspect, the genetically modified fermenting microorganism is Escherichia coli. In another preferred aspect, the genetically modified fermenting microorganism is Klebsiella oxytoca. In another preferred aspect, the genetically modified fermenting microorganism is Kluyveromyces sp.

It is well known in the art that the organisms described above can also be used to produce other substances, as described herein.

The fermenting microorganism is typically added to the degraded lignocellulose or hydrolysate and the fermentation is performed for about 8 to about 96 hours, such as about 24 to about 60 hours. The temperature is typically between about 26° C. to about 60° C., in particular about 32° C. or 50° C., and at about pH 3 to about pH 8, such as around pH 4-5, 6, or 7.

In a preferred aspect, the yeast and/or another microorganism is applied to the degraded cellulosic material and the fermentation is performed for about 12 to about 96 hours, such as typically 24-60 hours. In a preferred aspect, the temperature is preferably between about 20° C. to about 60° C., more preferably about 25° C. to about 50° C., and most preferably about 32° C. to about 50° C., in particular about 32° C. or 50° C., and the pH is generally from about pH 3 to about pH 7, preferably around pH 4-7. However, some fermenting organisms, e.g., bacteria, have higher fermentation temperature optima. Yeast or another microorganism is preferably applied in amounts of approximately 105 to 10¹², preferably from approximately 10⁷ to 10¹⁰, especially approximately 2×10⁸ viable cell count per ml of fermentation broth. Further guidance in respect of using yeast for fermentation can be found in, e.g., “The Alcohol Textbook” (Editors K. Jacques, T. P. Lyons and D. R. Kelsall, Nottingham University Press, United Kingdom 1999), which is hereby incorporated by reference.

For ethanol production, following the fermentation the fermented slurry is distilled to extract the ethanol. The ethanol obtained according to the methods of the invention can be used as, e.g., fuel ethanol, drinking ethanol, i.e., potable neutral spirits, or industrial ethanol.

A fermentation stimulator can be used in combination with any of the processes described herein to further improve the fermentation process, and in particular, the performance of the fermenting microorganism, such as, rate enhancement and ethanol yield. A “fermentation stimulator” refers to stimulators for growth of the fermenting microorganisms, in particular, yeast. Preferred fermentation stimulators for growth include vitamins and minerals. Examples of vitamins include multivitamins, biotin, pantothenate, nicotinic acid, meso-inositol, thiamine, pyridoxine, para-aminobenzoic acid, folic acid, riboflavin, and Vitamins A, B, C, D, and E. See, for example, Alfenore et al., Improving ethanol production and viability of Saccharomyces cerevisiae by a vitamin feeding strategy during fed-batch process, Springer-Verlag (2002), which is hereby incorporated by reference. Examples of minerals include minerals and mineral salts that can supply nutrients comprising P, K, Mg, S, Ca, Fe, Zn, Mn, and Cu.

Fermentation Products:

A fermentation product can be any substance derived from the fermentation. The fermentation product can be, without limitation, an alcohol (e.g., arabinitol, n-butanol, isobutanol, ethanol, glycerol, methanol, ethylene glycol, 1,3-propanediol [propylene glycol], butanediol, glycerin, sorbitol, and xylitol); an alkane (e.g., pentane, hexane, heptane, octane, nonane, decane, undecane, and dodecane), a cycloalkane (e.g., cyclopentane, cyclohexane, cycloheptane, and cyclooctane), an alkene (e.g. pentene, hexene, heptene, and octene); an amino acid (e.g., aspartic acid, glutamic acid, glycine, lysine, serine, and threonine); a gas (e.g., methane, hydrogen (H₂), carbon dioxide (CO₂), and carbon monoxide (CO)); isoprene; a ketone (e.g., acetone); an organic acid (e.g., acetic acid, acetonic acid, adipic acid, ascorbic acid, citric acid, 2,5-diketo-D-gluconic acid, formic acid, fumaric acid, glucaric acid, gluconic acid, glucuronic acid, glutaric acid, 3-hydroxypropionic acid, itaconic acid, lactic acid, malic acid, malonic acid, oxalic acid, oxaloacetic acid, propionic acid, succinic acid, and xylonic acid); and polyketide. The fermentation product can also be protein as a high value product.

In a preferred aspect, the fermentation product is an alcohol. It will be understood that the term “alcohol” encompasses a substance that contains one or more hydroxyl moieties. In a more preferred aspect, the alcohol is n-butanol. In another more preferred aspect, the alcohol is isobutanol. In another more preferred aspect, the alcohol is ethanol. In another more preferred aspect, the alcohol is methanol. In another more preferred aspect, the alcohol is arabinitol. In another more preferred aspect, the alcohol is butanediol. In another more preferred aspect, the alcohol is ethylene glycol. In another more preferred aspect, the alcohol is glycerin. In another more preferred aspect, the alcohol is glycerol. In another more preferred aspect, the alcohol is 1,3-propanediol. In another more preferred aspect, the alcohol is sorbitol. In another more preferred aspect, the alcohol is xylitol. See, for example, Gong, C. S., Cao, N. J., Du, J., and Tsao, G. T., 1999, Ethanol production from renewable resources, in Advances in Biochemical Engineering/Biotechnology, Scheper, T., ed., Springer-Verlag Berlin Heidelberg, Germany, 65: 207-241; Silveira, M. M., and Jonas, R., 2002, The biotechnological production of sorbitol, Appl. Microbiol. Biotechnol. 59: 400-408; Nigam, P., and Singh, D., 1995, Processes for fermentative production of xylitol—a sugar substitute, Process Biochemistry 30 (2): 117-124; Ezeji, T. C., Qureshi, N. and Blaschek, H. P., 2003, Production of acetone, butanol and ethanol by Clostridium beijerinckii BA101 and in situ recovery by gas stripping, World Journal of Microbiology and Biotechnology 19 (6): 595-603.

In another preferred aspect, the fermentation product is an alkane. The alkane can be an unbranched or a branched alkane. In another more preferred aspect, the alkane is pentane. In another more preferred aspect, the alkane is hexane. In another more preferred aspect, the alkane is heptane. In another more preferred aspect, the alkane is octane. In another more preferred aspect, the alkane is nonane. In another more preferred aspect, the alkane is decane. In another more preferred aspect, the alkane is undecane. In another more preferred aspect, the alkane is dodecane.

In another preferred aspect, the fermentation product is a cycloalkane. In another more preferred aspect, the cycloalkane is cyclopentane. In another more preferred aspect, the cycloalkane is cyclohexane. In another more preferred aspect, the cycloalkane is cycloheptane. In another more preferred aspect, the cycloalkane is cyclooctane.

In another preferred aspect, the fermentation product is an alkene. The alkene can be an unbranched or a branched alkene. In another more preferred aspect, the alkene is pentene. In another more preferred aspect, the alkene is hexene. In another more preferred aspect, the alkene is heptene. In another more preferred aspect, the alkene is octene.

In another preferred aspect, the fermentation product is an amino acid. In another more preferred aspect, the organic acid is aspartic acid. In another more preferred aspect, the amino acid is glutamic acid. In another more preferred aspect, the amino acid is glycine. In another more preferred aspect, the amino acid is lysine. In another more preferred aspect, the amino acid is serine. In another more preferred aspect, the amino acid is threonine. See, for example, Richard, A., and Margaritis, A., 2004, Empirical modeling of batch fermentation kinetics for poly(glutamic acid) production and other microbial biopolymers, Biotechnology and Bioengineering 87 (4): 501-515.

In another preferred aspect, the fermentation product is a gas. In another more preferred aspect, the gas is methane. In another more preferred aspect, the gas is H₂. In another more preferred aspect, the gas is CO₂. In another more preferred aspect, the gas is CO. See, for example, Kataoka, N., A. Miya, and K. Kiriyama, 1997, Studies on hydrogen production by continuous culture system of hydrogen-producing anaerobic bacteria, Water Science and Technology 36 (6-7): 41-47; and Gunaseelan V. N. in Biomass and Bioenergy, Vol. 13 (1-2), pp. 83-114, 1997, Anaerobic digestion of biomass for methane production: A review.

In another preferred aspect, the fermentation product is isoprene.

In another preferred aspect, the fermentation product is a ketone. It will be understood that the term “ketone” encompasses a substance that contains one or more ketone moieties. In another more preferred aspect, the ketone is acetone. See, for example, Qureshi and Blaschek, 2003, supra.

In another preferred aspect, the fermentation product is an organic acid. In another more preferred aspect, the organic acid is acetic acid. In another more preferred aspect, the organic acid is acetonic acid. In another more preferred aspect, the organic acid is adipic acid. In another more preferred aspect, the organic acid is ascorbic acid. In another more preferred aspect, the organic acid is citric acid. In another more preferred aspect, the organic acid is 2,5-diketo-D-gluconic acid. In another more preferred aspect, the organic acid is formic acid. In another more preferred aspect, the organic acid is fumaric acid. In another more preferred aspect, the organic acid is glucaric acid. In another more preferred aspect, the organic acid is gluconic acid. In another more preferred aspect, the organic acid is glucuronic acid. In another more preferred aspect, the organic acid is glutaric acid. In another preferred aspect, the organic acid is 3-hydroxypropionic acid. In another more preferred aspect, the organic acid is itaconic acid. In another more preferred aspect, the organic acid is lactic acid. In another more preferred aspect, the organic acid is malic acid. In another more preferred aspect, the organic acid is malonic acid. In another more preferred aspect, the organic acid is oxalic acid. In another more preferred aspect, the organic acid is propionic acid. In another more preferred aspect, the organic acid is succinic acid. In another more preferred aspect, the organic acid is xylonic acid. See, for example, Chen, R., and Lee, Y. Y., 1997, Membrane-mediated extractive fermentation for lactic acid production from cellulosic biomass, Appl. Biochem. Biotechnol. 63-65: 435-448.

In another preferred aspect, the fermentation product is polyketide.

Recovery.

The fermentation product(s) can be optionally recovered from the fermentation medium using any method known in the art including, but not limited to, chromatography, electrophoretic procedures, differential solubility, distillation, or extraction. For example, alcohol is separated from the fermented cellulosic material and purified by conventional methods of distillation. Ethanol with a purity of up to about 96 vol. % can be obtained, which can be used as, for example, fuel ethanol, drinking ethanol, i.e., potable neutral spirits, or industrial ethanol.

The present invention is further described by the following examples that should not be construed as limiting the scope of the invention.

### EXAMPLES

### Example 1: Methods of Evaluating the Effect of Organic Compounds on GH61 Polypeptides Having Cellulolytic Enhancing Activity

The effect of various carboxylic acids, dicarboxylic acids, hydroxy-acids, keto-acids, and hydroxyl-containing compounds with pKa values below or sufficiently near the assay pH of 5.0 with some fraction deprotonated (i.e., an organic compound or fully or partially deprotonated carboxylic acid groups or hydroxyl groups, designated herein as organic compounds) on the cellulolytic enhancing activity of GH61 polypeptides was evaluated according to the procedures described below.

Microcrystalline cellulose was used as the source of cellulosic material. Microcrystalline cellulose (AVICEL® PH101) was obtained from Sigma-Aldrich (St. Louis, Mo., USA).

A Trichoderma reesei cellulase preparation (CELLUCLAST® supplemented with Aspergillus oryzae beta-glucosidase, available from Novozymes A/S, Bagsvaerd, Denmark) was used as the cellulase preparation. The cellulase preparation is designated herein in the Examples as “Trichoderma reesei cellulase composition”.

The hydrolysis of AVICEL® was conducted using 2.0 ml deep-well plates (Axygen Scientific, Union City, Calif., USA) in a total reaction volume of 1.0 ml. Each hydrolysis was performed with 29.5 mg of AVICEL® per ml of 50 mM sodium acetate pH 5.0 buffer containing 1 mM manganese sulfate and the T. reesei cellulase composition at 4 mg protein per gram of cellulose with and without an organic compound at the specified concentration, usually 1 mM, and with and without GH61 polypeptide having cellulolytic enhancing activity at 2 mg per g cellulose (unless otherwise specified). The plate was then sealed using an ALPS-300™ or ALPS-3000™ plate heat sealer (Abgene, Epsom, United Kingdom), mixed thoroughly, and incubated at 50° C. for 7 days in an Isotemp Plus incubator (Thermo Fisher Scientific Inc., Waltham, Mass., USA). All experiments were performed at least in duplicate.

At the indicated times following initiation of hydrolysis, typically 3 and 7 days, aliquots were removed and filtered using a 0.45 μm MULTISCREEN® 96-well filter plate (Millipore, Bedford, Mass., USA), and the filtrates were analyzed for sugar content as described below. When not used immediately, filtered aliquots were frozen at −20° C. The sugar concentrations of samples, diluted to appropriate concentrations in 0.005 M H₂SO₄, were measured using a 4.6×250 mm AMINEX® HPX-87H column (Bio-Rad Laboratories, Inc., Hercules, Calif., USA) by elution with 0.05% (w/w) benzoic acid in 0.005 M H₂SO₄ at 65° C. at a flow rate of 0.6 ml per minute, and quantitated by integration of the glucose and cellobiose signals from refractive index detection (CHEMSTATION®, AGILENT® 1100 HPLC, Agilent Technologies, Santa Clara, Calif., USA) calibrated by pure sugar samples. The resultant glucose and cellobiose equivalents were used to calculate the extent of cellulose conversion for each reaction. Measured sugar concentrations were adjusted for the appropriate dilution factor. Data were processed using MICROSOFT EXCEL™ software (Microsoft, Richland, Wash., USA) or Kaleidagraph™ (Synergy software, Reading, Pa., USA).

The extent of cellulose conversion was calculated based on the mass ratio of solubilized glucosyl units to the initial mass of insoluble cellulose. The fraction of total cellulose conversion was calculated using the following equation:

$$fractional hydrolysis = \frac{\begin{matrix}( ( 1.053 \times [ cellobiose ] ( mg \text{/} ml ) ) + \\ [ glucose ] ( mg / m ) ) ) \text{/} 1.111 )\end{matrix}}{[ cellulose ] ( mg \text{/} ml )} \qquad ( Equation 1 )$$

The 1.111 and 1.053 factors for glucose and cellobiose, respectively, take into account the increase in mass when the glucosyl units in cellulose (average molecular mass of 162 daltons) are converted to glucose (molecular mass of 180 daltons) or cellobiose glucosyl units (average molecular mass of 171 daltons).

The organic compounds evaluated include methyl-4-hydroxybenzoate, 3,5-dihydroxybenzoic acid, 4-hydroxybenzoic acid, glutamate, tartrate, 4-hydroxy-3-methoxycinnamic acid, methyl-3,4,5-trihydroxybenzoate, 3,4-dihydroxybenzoic acid, ascorbate, 4-methyl hexanoic acid, 2-methyl hexanoic acid, pyrocatechol, pyrogallol, and dehydroascorbate. The compounds were obtained from Sigma-Aldrich Co. (St. Louis, Mo., USA).

### Example 2: Preparation of Thermoascus aurantiacus GH61A Polypeptide Having Cellulolytic Enhancing Activity

Thermoascus aurantiacus GH61A polypeptide having cellulolytic enhancing activity (SEQ ID NO: 13 (DNA sequence] or SEQ ID NO: 14 [deduced amino acid sequence]) was recombinantly prepared according to WO 2005/074656 using Aspergillus oryzae JaL250 as a host.

The recombinantly produced T. aurantiacus GH61A polypeptide was first concentrated from 60 ml to 7 ml, by ultrafiltration using a 10 kDa membrane (VIVASPIN®, GE Healthcare, Piscataway, N.J., USA), buffer exchanged into 20 mM Tris-HCl plus 150 mM NaCl pH 8.0, and then purified using a 320 ml SUPERDEX® 75 column (GE Healthcare, Piscataway, N.J., USA) equilibrated with 20 mM Tris-HCl plus 150 mM NaCl pH 8.0 at a flow rate of 1 ml per minute. Fractions of 5 ml were collected and pooled based on SDS-PAGE.

Protein concentration was determined using a Microplate BCA™ Protein Assay Kit (Thermo Fisher Scientific Inc., Rockford, Ill., USA) in which bovine serum albumin was used as a protein standard.

### Example 3: Effect of Thermoascus aurantiacus GH61 Polypeptide Having Cellulolytic Enhancing Activity on Hydrolysis of Microcrystalline Cellulose by the Trichoderma reesei Cellulase Composition

The effect of the T. aurantiacus GH61A polypeptide on the hydrolysis of AVICEL® by the T. reesei cellulase composition was determined using the same experimental conditions and procedures described in Example 1 in the absence of a heterocyclic compound.

The presence of the T. aurantiacus GH61A polypeptide did not enhance hydrolysis of AVICEL® by the T. reesei cellulase composition. Percent conversion of AVICEL® was 16±1%, 31±4%, and 45±3% at 1, 3, and 7 days, respectively, in the absence of the T. aurantiacus GH61A polypeptide compared to 16±1%, 30±4%, and 45±4% at 1, 3, and 7 days, respectively, in the presence of the T. aurantiacus GH61A polypeptide.

### Example 4: Effect of Organic Compounds on Thermoascus aurantiacus GH61A Polypeptide During Hydrolysis of Microcrystalline Cellulose by the Trichoderma reesei Cellulase Composition

The effect of methyl-4-hydroxybenzoate, 3,5-dihydroxybenzoic acid, 4-hydroxybenzoic acid, glutamate, tartrate, 4-hydroxy-3-methoxycinnamic acid, methyl-3,4,5-trihydroxybenzoate, 3,4-dihydroxybenzoic acid, ascorbate, 4-methyl hexanoic acid, 2-methyl hexanoic acid, pyrocatechol, pyrogallol, and dehydroascorbate on the cellulolytic enhancing activity of the T. aurantiacus GH61A polypeptide during hydrolysis of AVICEL® by the T. reesei cellulase composition was determined using the experimental conditions and procedures described in Example 1. The concentration of each organic compound was 1 mM.

The effect of an organic compound on the hydrolysis of a cellulosic material by the T. reesei cellulase composition in the absence of a GH61 polypeptide was quantified by determining the ratio of percent conversion of the cellulosic material in the presence of the heterocyclic compound to the percent conversion of the cellulosic material in the absence of the heterocyclic compound:

$$Organic compound {effect}_{( no GH 6 \; 1 )} = \frac{\% {conversion}_{( no GH 61 + organic compound )}}{\% {conversion}_{( no GH 61 no organic compound )}} \qquad ( Equation 2 )$$

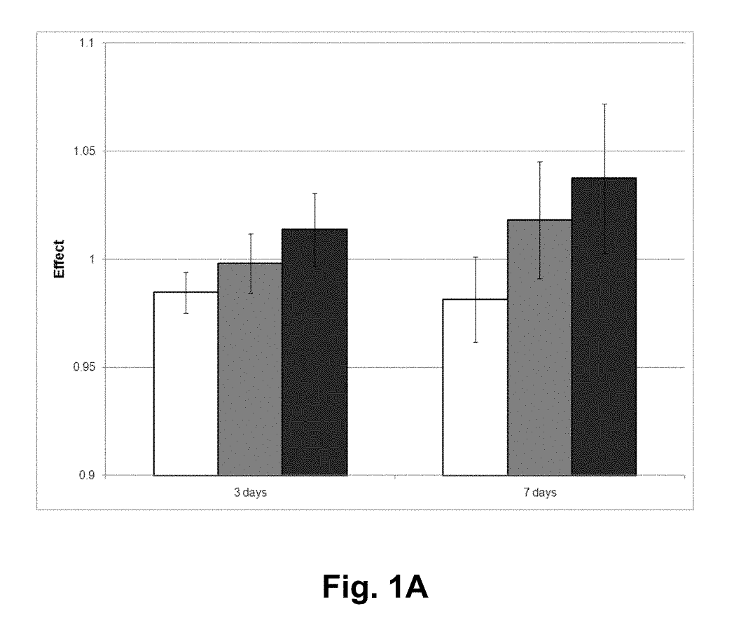

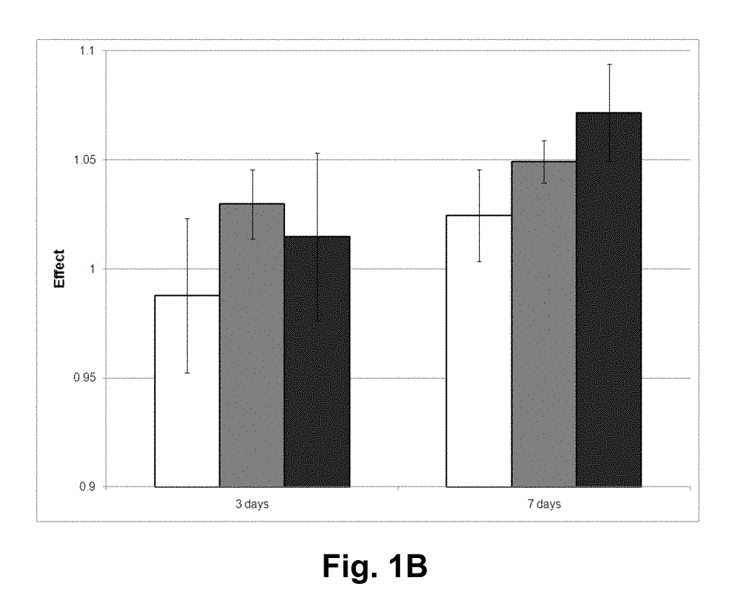

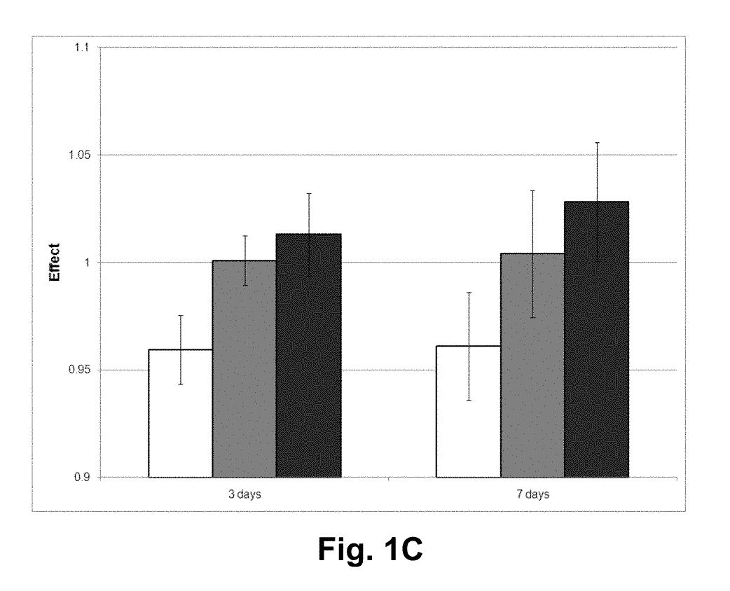

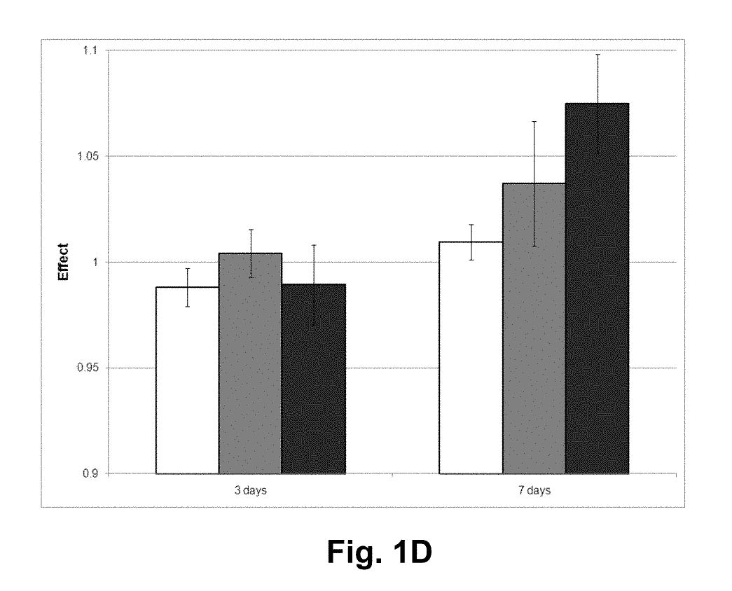

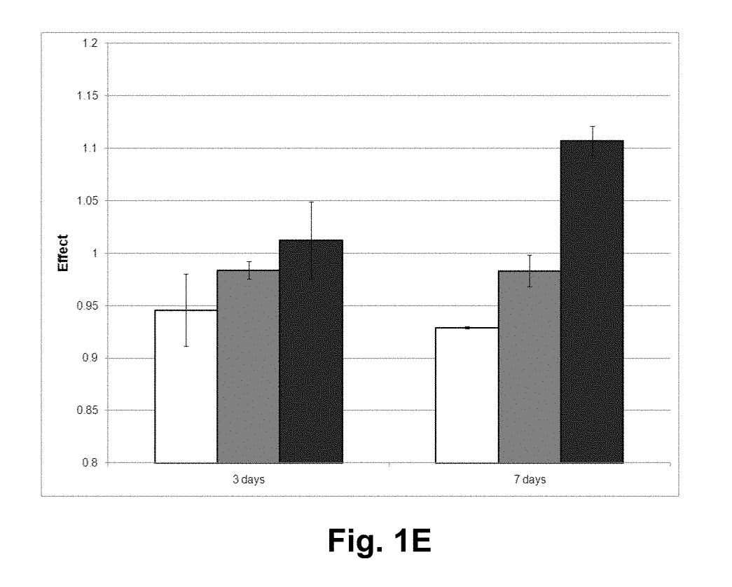

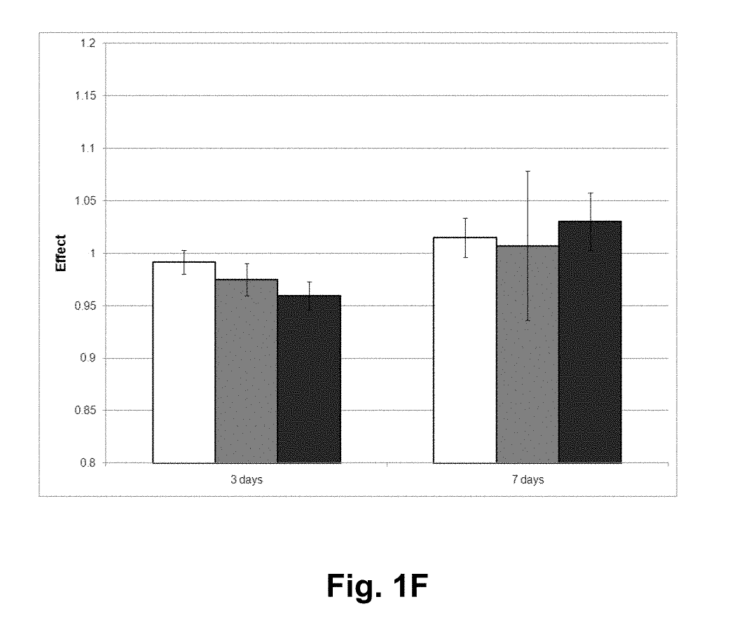

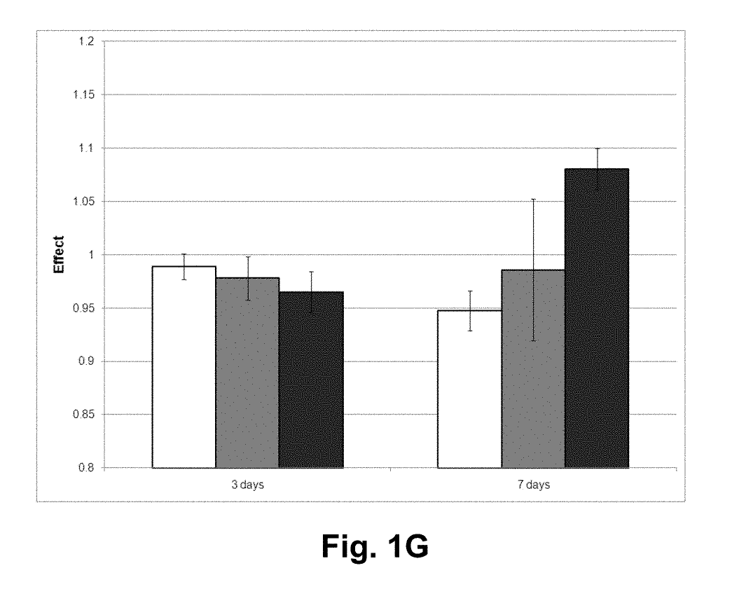

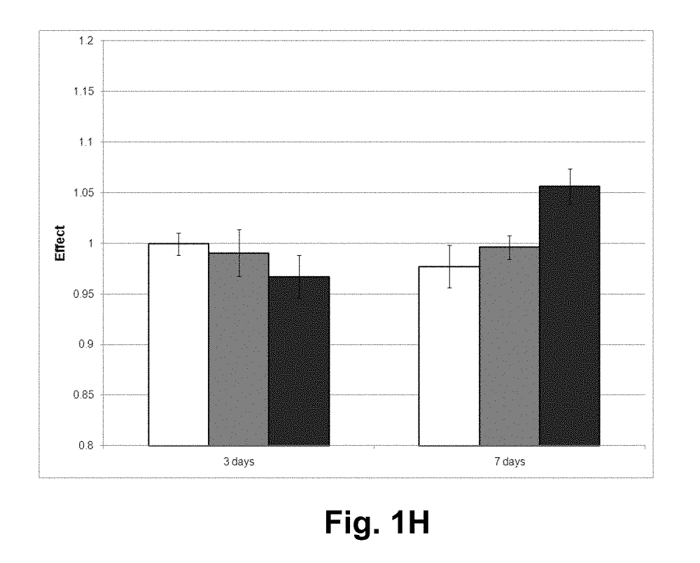

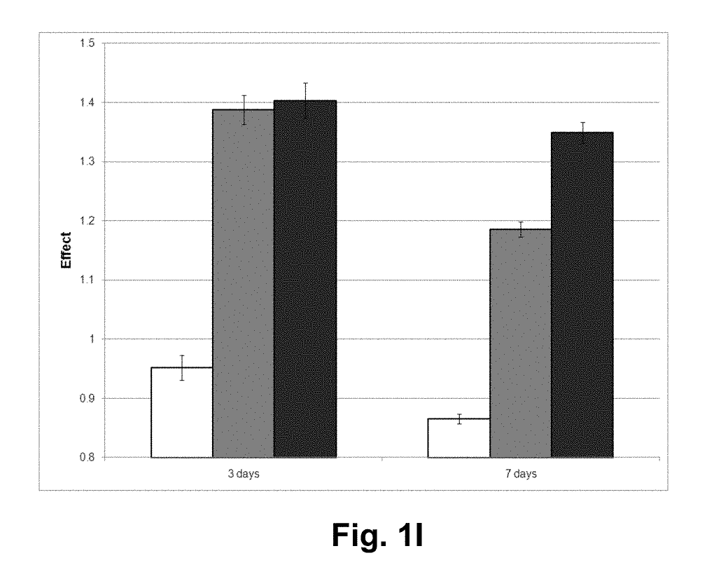

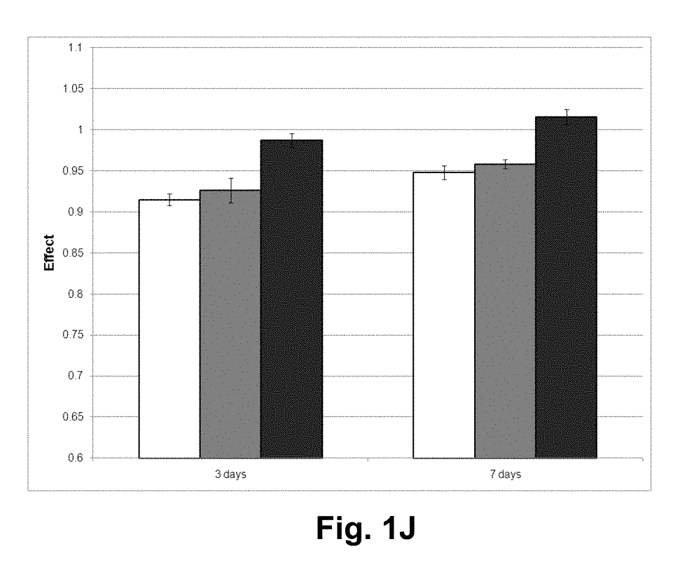

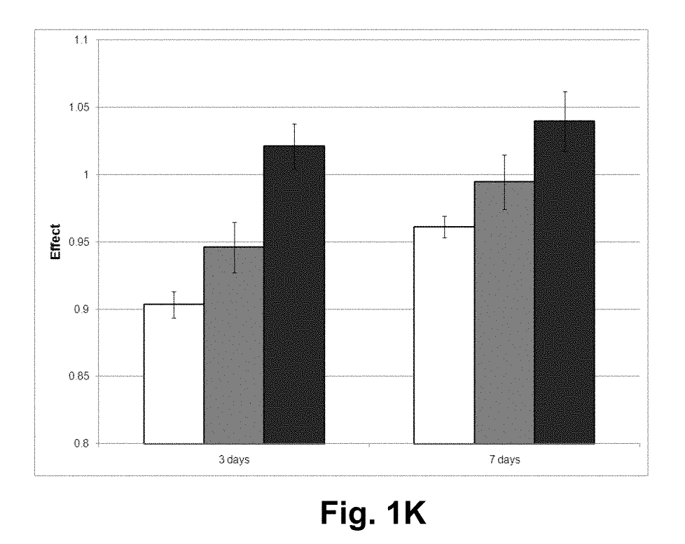

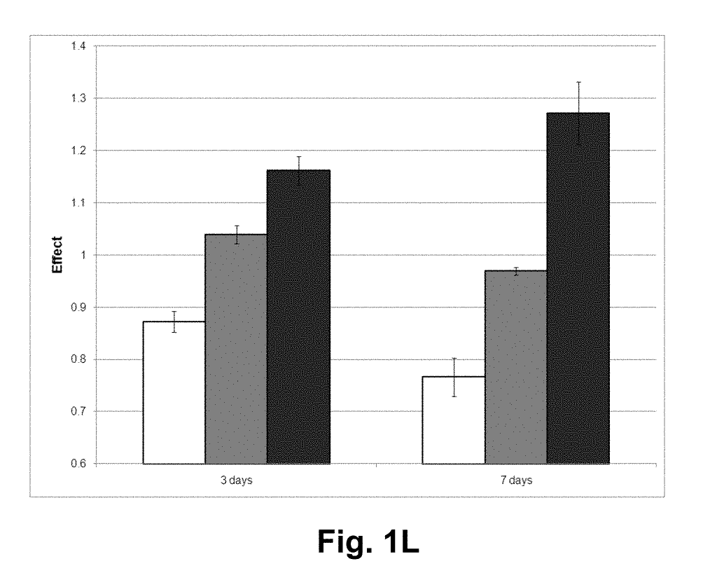

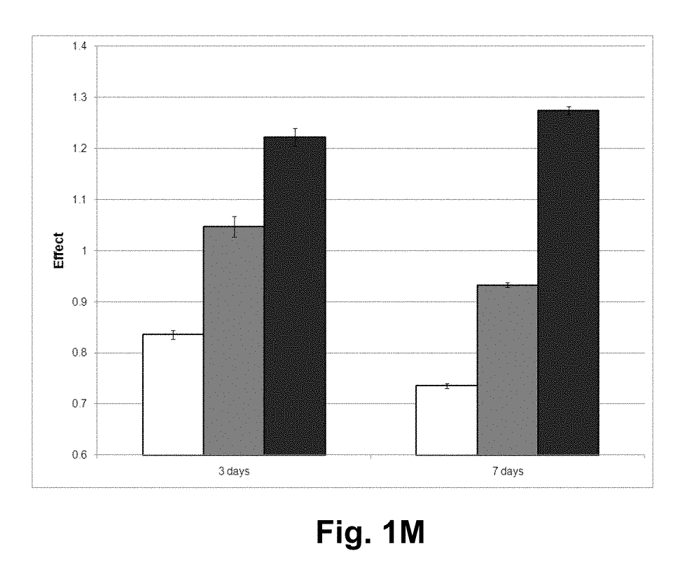

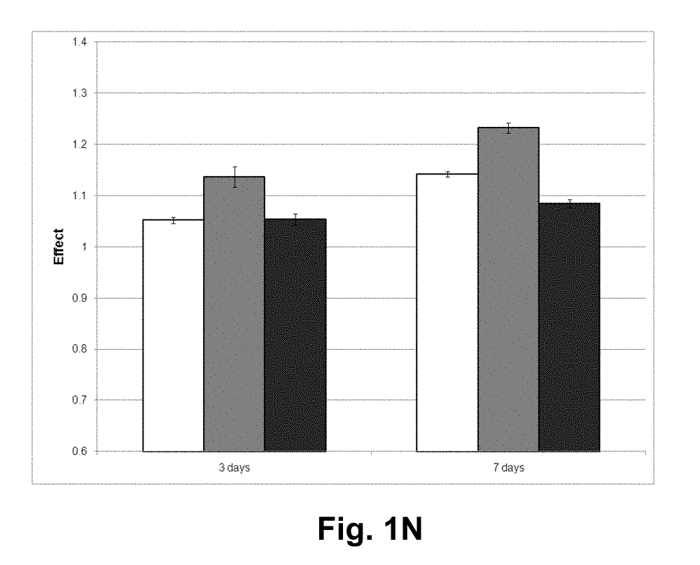

Stimulation of hydrolysis by the organic compound yields a ratio >1; inhibition of hydrolysis yields a ratio <1, and no effect on hydrolysis yields a ratio=1 (FIG. 1, white bars).

The effect of an organic compound on hydrolysis of a cellulosic material by the T. reesei cellulase composition in the presence of a GH61 polypeptide was quantified by determining the ratio of percent conversion of the cellulosic material in the presence of the heterocyclic compound to the percent conversion of the cellulosic material in the absence of the heterocyclic compound:

$$Organic compound {effect}_{( + GH 6 \; 1 )} = \frac{\% {conversion}_{( + GH 61 + organic compound )}}{\% {conversion}_{( + GH 61 no organic compound )}} \qquad ( Equation 3 )$$

Stimulation of hydrolysis by the organic compound in the presence of the GH61 polypeptide yields a ratio >1; inhibition of hydrolysis yields a ratio <1, and no effect on hydrolysis yields a ratio=1 (FIG. 1, grey bars).

The effect of a GH61 polypeptide on hydrolysis of a cellulosic material by the T. reesei cellulase composition in the presence of an organic compound was quantified by determining the ratio of percent conversion of the cellulosic material in the presence of the GH61 polypeptide to the percent conversion of the cellulosic material in the absence of the GH61 polypeptide:

$$GH 61 effect = \frac{\% {conversion}_{( + GH 61 + organic compound )}}{\% {conversion}_{( no GH 61 + organic compound )}} \qquad ( Equation 4 )$$

Enhancement of hydrolysis by the GH61 polypeptide yields a ratio >1; inhibition of hydrolysis yields a ratio <1, and no effect on hydrolysis yields a ratio=1 (FIG. 1, black bars).

FIGS. 1A (methyl-4-hydroxybenzoate), 1B (3,5-dihydroxybenzoic acid), 1C (4-hydroxybenzoic acid), 1D (glutamate), 1E (tartrate), 1F (4-hydroxy-3-methoxycinnamic acid), and 1G (methyl-3,4,5-trihydroxybenzoate), 1H (3,4-dihydroxybenzoic acid), 1I (ascorbate), 1J (4-methyl hexanoic acid), 1K (2-methyl hexanoic acid), 1L (pyrocatechol), 1M (pyrogallol), and 1N (dehydroascorbate) show (1) the effect of an organic compound on hydrolysis of AVICEL® by the Trichoderma reesei cellulase composition in the absence of a GH61 polypeptide (organic compound effect_{(no GH61)}, white bars), (2) the effect of an organic compound on hydrolysis of AVICEL® by the T. reesei cellulase composition in the presence of a GH61 polypeptide (organic compound effect_{(+GH61)}, grey bars), and (3) the effect of a GH61 polypeptide on hydrolysis of AVICEL® by the T. reesei cellulase composition in the presence of an organic compound (GH61 effect, black bars) for 3 and 7 days.

Hydrolysis of AVICEL® by the T. reesei cellulase composition was slightly inhibited by the presence of methyl-4-hydroxybenzoate in the absence of the T. aurantiacus GH61A polypeptide as quantified by the organic compound effect_{(no GH61)} (FIG. 1A, white bars), as defined by Equation 2. In the presence of the T. aurantiacus GH61A polypeptide, hydrolysis was enhanced by addition of methyl-4-hydroxybenzoate, as indicated by an organic compound effect_{(+GH61)} greater than 1 (FIG. 1A, grey bars), as defined by Equation 3. Furthermore, the effect of the T. aurantiacus GH61A polypeptide was greater than 1 (GH61 effect, Equation 4), indicating that the T. aurantiacus GH61A polypeptide enhanced hydrolysis when methyl-4-hydroxybenzoate was present (FIG. 1A, black bars), whereas the T. aurantiacus GH61A polypeptide did not enhance hydrolysis of microcrystalline cellulose in the absence of methyl-4-hydroxybenzoate (Example 3).

Hydrolysis of AVICEL® by the T. reesei cellulase composition was modestly increased by the presence of 3,5-dihydroxybenzoic acid and by the combination of 3,5-dihydroxybenzoic acid and the T. aurantiacus GH61A polypeptide as indicated by an organic compound effect_{(+GH61)} and an organic compound effect_{(no GH61)} of greater than 1 at 7 days of hydrolysis (FIG. 1B, grey bars and white bars). Furthermore, the effect of the T. aurantiacus GH61A polypeptide was greater than 1 (GH61 effect, Equation 3), indicating that the T. aurantiacus GH61A polypeptide enhanced hydrolysis when 3,5-dihydroxybenzoic acid was present (FIG. 1B, black bars), whereas the T. aurantiacus GH61A polypeptide did not enhance hydrolysis of microcrystalline cellulose in the absence of 3,5-dihydroxybenzoic acid (Example 3).

Hydrolysis of AVICEL® by the T. reesei cellulase composition was reduced in the presence of 4-hydroxybenzoic acid without the T. aurantiacus GH61A polypeptide with an organic compound effect_{(no GH61)} of 0.959±0.0162 and 0.961±0.0250 at 3 and 7 days of hydrolysis, respectively (Equation 2). Hydrolysis of AVICEL® in the presence of 4-hydroxybenzoic acid and the T. aurantiacus GH61A polypeptide was enhanced compared to hydrolysis in the presence of 4-hydroxybenzoic acid alone, as quantified by an organic compound effect_{(+GH61)} of approximately 1 (grey and white bars in FIG. 1C) as defined by Equation 3. The effect of the T. aurantiacus GH61A polypeptide (GH61 effect) was slightly greater than 1 (Equation 3), indicating that the T. aurantiacus GH61A polypeptide enhanced hydrolysis of microcrystalline cellulose when 4-hydroxybenzoic acid was present (FIG. 1C, black bars), whereas the T. aurantiacus GH61A polypeptide did not enhance hydrolysis of microcrystalline cellulose in the absence of 4-hydroxybenzoic acid (Example 3).

Hydrolysis of AVICEL® by the T. reesei cellulase composition was unchanged by the presence of glutamate without the T. aurantiacus GH61A polypeptide, as indicated by an organic compound effect_{(no GH61)} slightly below 1 at 3 days and slightly above 1 at 7 days of saccharification (FIG. 1D, white bars). In the presence of the T. aurantiacus GH61A polypeptide, glutamate modestly enhanced hydrolysis at 7 days of hydrolysis, producing an organic compound effect_{(+GH61)} of 1.04±0.0226. The effect of the T. aurantiacus GH61A polypeptide (GH61 effect) was greater than 1 at 7 days of hydrolysis, 1.07±0.0223 (Equation 4), indicating that the T. aurantiacus GH61A polypeptide enhanced hydrolysis of microcrystalline cellulose when glutamate was present (FIG. 1D, black bars), whereas the T. aurantiacus GH61A polypeptide did not enhance hydrolysis of microcrystalline cellulose in the absence of glutamate (Example 3).

Hydrolysis of AVICEL® by the T. reesei cellulase composition was slightly reduced by the presence of tartrate with and without the T. aurantiacus GH61A polypeptide, as indicated by an organic compound effect_{(no GH61)} and organic compound effect_{(+GH61)} of slightly below 1 at 3 and 7 days of saccharification (FIG. 1E, white bars and gray bars). Hydrolysis of AVICEL® by the T. reesei cellulase composition in the presence of both the T. aurantiacus GH61A polypeptide polypeptide and tartrate was higher than hydrolysis in the presence of tartrate alone The GH61 effect was greater than 1, 1.11±0.0139 (Equation 4) at 7 days of hydrolysis (FIG. 1E, black bars). This result indicated that the T. aurantiacus GH61A polypeptide enhanced hydrolysis of microcrystalline cellulose when tartrate was present, whereas the T. aurantiacus GH61A polypeptide did not enhance hydrolysis of microcrystalline cellulose in the absence of tartrate (Example 3).

Hydrolysis of AVICEL® by the T. reesei cellulase composition was slightly reduced by the presence of 4-hydroxy-3-methoxycinnamic acid with and without the T. aurantiacus GH61A polypeptide, as indicated by an organic compound effect_{(no GH61)} and organic compound effect_{(+GH61)} of slightly below 1 at 3 days of saccharification (FIG. 1F, white bars and gray bars), but by 7 days of hydrolysis both these values were approximately 1. Hydrolysis of AVICEL® by the T. reesei cellulase composition in the presence of both the T. aurantiacus GH61A polypeptide polypeptide and 4-hydroxy-3-methoxycinnamic acid was higher than hydrolysis in the presence of 4-hydroxy-3-methoxycinnamic acid alone, thus the GH61 effect was greater than 1, (Equation 4) at 7 days of hydrolysis (FIG. 1F, black bars). This result indicated that the T. aurantiacus GH61A polypeptide enhanced hydrolysis of microcrystalline cellulose when 4-hydroxy-3-methoxycinnamic acid was present, whereas the T. aurantiacus GH61A polypeptide did not enhance hydrolysis of microcrystalline cellulose in the absence of 4-hydroxy-3-methoxycinnamic acid (Example 3).

Hydrolysis of AVICEL® by the T. reesei cellulase composition was slightly decreased by the presence of methyl-3,4,5-trihydroxybenzoate without T. aurantiacus GH61A polypeptide, as indicated by an organic compound effect_{(no GH61)} of less than 1 (FIG. 1G, white bars), 0.989±0.0121 and 0.947±0.00189 at 3 and 7 days of hydrolysis, respectively. In the presence of the T. aurantiacus GH61A polypeptide, methyl-3,4,5-trihydroxybenzoate was significantly less inhibitory to hydrolysis, producing an organic compound effect_{(+GH61)} of 0.988±0.0157 at 7 days of hydrolysis (FIG. 1G, gray bars). The effect of the T. aurantiacus GH61A polypeptide (GH61 effect) was therefore much greater than 1 (Equation 4), 1.08±0.0193 at 7 days of hydrolysis, indicating that the T. aurantiacus GH61A polypeptide enhanced hydrolysis of microcrystalline cellulose when methyl-3,4,5-trihydroxybenzoate was present (FIG. 1G, black bars), whereas the T. aurantiacus GH61A polypeptide did not enhance hydrolysis of microcrystalline cellulose in the absence of methyl-3,4,5-trihydroxybenzoate (Example 3).

Hydrolysis of AVICEL® by the T. reesei cellulase composition was unchanged by the presence of 3,4-dihydroxybenzoic acid and the T. aurantiacus GH61A polypeptide, as indicated by an organic compound effect_{(+GH61)} of approximately 1 (FIG. 1H, gray bars), while hydrolysis was slightly reduced in the absence of the GH61 polypeptide (FIG. 1H, white bars). In the absence of the T. aurantiacus GH61A polypeptide, the organic compound effect_{(no GH61)} was 0.977±0.0208 at 7 days of hydrolysis. The effect of the T. aurantiacus GH61A polypeptide (GH61 effect) was therefore greater than 1 (Equation 4), 1.06±0.0171 at 7 days of hydrolysis, indicating that the T. aurantiacus GH61A polypeptide enhanced hydrolysis of microcrystalline cellulose when 3,4-dihydroxybenzoic acid was present (FIG. 1H, black bars), whereas the T. aurantiacus GH61A polypeptide did not enhance hydrolysis of microcrystalline cellulose in the absence of 3,4-dihydroxybenzoic acid (Example 3).

Hydrolysis of AVICEL® by the T. reesei cellulase composition was substantially decreased by the presence of ascorbate without the T. aurantiacus GH61A polypeptide, as indicated by an organic compound effect_{(no GH61)} less than 1 (FIG. 1I, white bars), 0.952±0.0211 and 0.865±0.00792 at 3 and 7 days of hydrolysis, respectively. In the presence of the T. aurantiacus GH61A polypeptide, however, ascorbate enhanced hydrolysis, producing an organic compound effect_{(+GH61)} of 1.388±0.0241 and 1.19±0.129 at 3 and 7 days of hydrolysis, respectively (FIG. 1I, gray bars). The effect of the T. aurantiacus GH61A polypeptide (GH61 effect) was therefore much greater than 1 (Equation 4), 1.40±0.0294 at 3 days and 1.35±0.0175 at 7 days of hydrolysis indicating that the T. aurantiacus GH61A polypeptide enhanced hydrolysis of microcrystalline cellulose when ascorbate was present (FIG. 1I, black bars), whereas the T. aurantiacus GH61A polypeptide did not enhance hydrolysis of microcrystalline cellulose in the absence of ascorbate (Example 3).

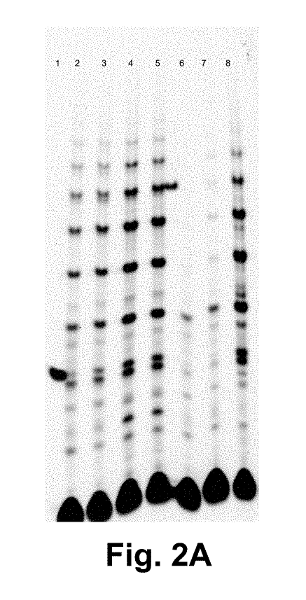

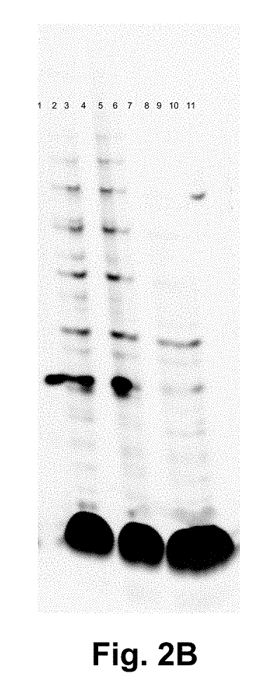

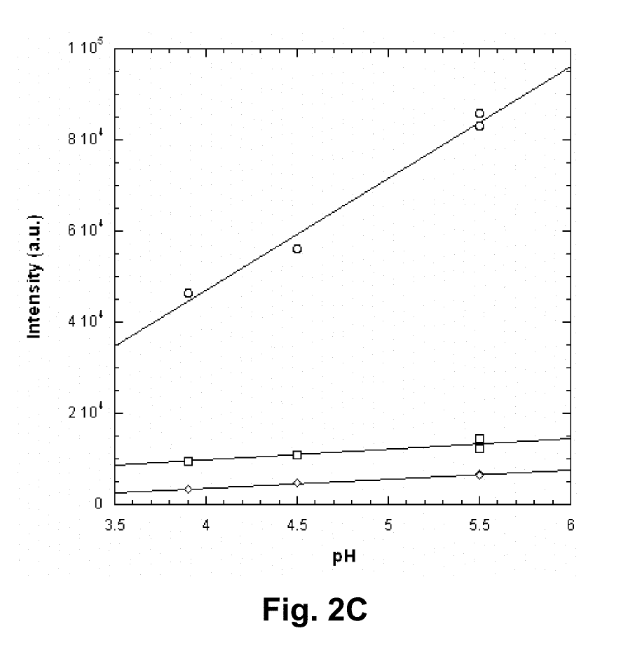

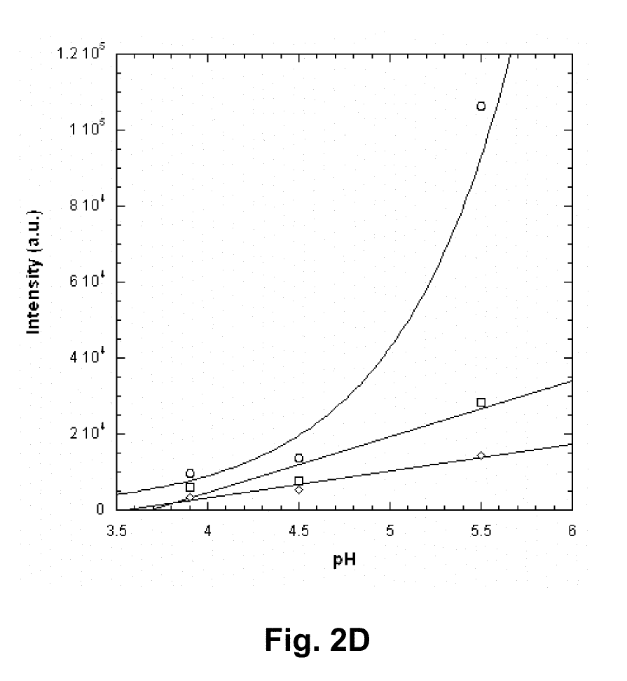

Hydrolysis of AVICEL® by the T. reesei cellulase composition was reduced by the presence of 4-methyl hexanoic acid (FIG. 1J) or 2-methyl hexanoic acid (FIG. 1K) in the absence of the T. aurantiacus GH61A polypeptide, as indicated by an organic compound effect_{(no GH61)} of less than 1 (FIGS. 1J and 1K, white bars). In the presence of T. aurantiacus GH61A polypeptide, hydrolysis was reduced less than hydrolysis in the absence of the GH61 polypeptide (FIGS. 1J and 1K, comparing gray bars to white bars). The effect of the T. aurantiacus GH61A polypeptide (GH61 effect) was greater than 1 (Equation 4), 1.02±0.00904 and 1.04±0.0223 at 7 days of hydrolysis for 4-methyl hexanoic acid and 2-methyl hexanoic acids, respectively (FIGS. 1J and 1K, black bars). These results indicated that the T. aurantiacus GH61A polypeptide enhanced hydrolysis of microcrystalline cellulose when either 4-methyl hexanoic acid or 2-methyl hexanoic acids was present, whereas the T. aurantiacus GH61A polypeptide did not enhance hydrolysis of microcrystalline cellulose in the absence of either of these organic compounds (Example 3).

Hydrolysis of AVICEL® by the T. reesei cellulase composition was substantially decreased by the presence of either pyrocatechol (FIG. 1L) or pyrogallol (FIG. 1M) without the T. aurantiacus GH61A polypeptide, as indicated by an organic compound effect_{(no GH61)} of less than 1 (FIGS. 1L and 1M, white bars), 0.766±0.0361 and 0.735±0.00518 at 7 days of hydrolysis, respectively. In the presence of the T. aurantiacus GH61A polypeptide, however, both pyrocatechol and pyrogallol enhanced hydrolysis early in saccharification, producing an organic compound effect_{(+GH61)} of 1.04±0.0177 and 1.05±0.0198 at 3 days of hydrolysis, respectively, and were only slightly inhibitory later in saccharification with an organic compound effect_{(+GH61)} of 0.969±0.00741 and 0.933±0.00452, respectively (FIGS. 1L and 1M, gray bars). The effect of the T. aurantiacus GH61A polypeptide (GH61 effect) was therefore much greater than 1 (Equation 4), 1.27±0.0603 and 1.27±0.00827 at 7 days of hydrolysis for the 2 compounds. These results indicated that the T. aurantiacus GH61A polypeptide enhanced hydrolysis of microcrystalline cellulose when either pyrocatechol or pyrogallol were present (FIGS. 1L and 1M, black bars), whereas the T. aurantiacus GH61A polypeptide did not enhance hydrolysis of microcrystalline cellulose in the absence of ascorbate (Example 3).

Hydrolysis of AVICEL® by the T. reesei cellulase composition was enhanced by the presence of dehydroascorbate without the T. aurantiacus GH61A polypeptide, as indicated by an organic compound effect_{(no GH61)} greater than 1 (FIG. 1N, white bars), 1.05±0.00580 and 1.14±0.00522 at 3 and 7 days of hydrolysis, respectively. In the presence of the T. aurantiacus GH61A polypeptide and dehydroascorbate an even greater enhancement of hydrolysis was observed, producing an organic compound effect_{(+GH61)} of 1.14±0.0203 and 1.23±0.0101 at 3 and 7 days of hydrolysis, respectively (FIG. 1N, gray bars). The effect of the T. aurantiacus GH61A polypeptide (GH61 effect) was therefore much greater than 1 (Equation 4), 1.05±0.0104 at 3 days and 1.08±0.00817 at 7 days of hydrolysis indicating that the T. aurantiacus GH61A polypeptide enhanced hydrolysis of microcrystalline cellulose when dehydroascorbate was present (FIG. 1N, black bars), whereas the T. aurantiacus GH61A polypeptide did not enhance hydrolysis of microcrystalline cellulose in the absence of ascorbate (Example 3).

The overall results demonstrated that cellulolytic enhancing activity of the GH61 polypeptide was apparent in the presence of an organic compound during hydrolysis of AVICEL® by the T. reesei cellulase composition. However, the T. aurantiacus GH61A polypeptide had no detectable effect on hydrolysis of AVICEL® by the T. reesei cellulase composition in the absence of an organic compound.

### Example 4: Preparation of Phosphoric Acid Swollen Cellulose (PASC)

Phosphoric acid swollen cellulose (PASC) slurry was prepared from AVICEL® PH101 using the prortocol described by Zhang et al., 2006, Biomacromolecules 7: 644-648.

### Example 5: Effect of pH on Apparent Cleavage of Phosphoric Acid Swollen Cellulose (PASC) by Ascorbate or Cysteine and the Thermoascus aurantiacus GH61A Polypeptide

Polysaccharide-analysis-using-carbohydrate-gel-electrophoresis (PACE) was applied to analyze the products from incubation of cellulose in the presence of Ca(II) and ascorbate, cysteine or gallic acid and the T. aurantiacus GH61A polypeptide.

To obtain reaction products, 5 g of phosphoric acid swollen cellulose (PASC) per liter, 10 mM ascorbate or 10 mM cysteine, 2 mM CaCl₂, and 50 mM of the indicated buffers, with or without 0.2 g of the T. aurantiacus 61A polypeptide per liter were incubated at 50° C. for 3 days. The mixture was filtered, and the filtrate (90 μl) was subjected to PACE analysis as described below. Buffers were varied to obtain the following pH values: formic acid at pH 4, acetic acid at pH 5, triethylammonium acetate at pH 6-7, and ammonium acetate at pH 8-9. The pH was measured before and after the PASC incubations, and the acidity was found to vary by less than 0.5 during the incubations. Alternatively, 5 mM ascorbate or 6 mM gallic acid was incubated with 0.2 g of the T. aurantiacus GH6A polypeptide per liter, 5 g of PASC per liter in 2 mM CaCl₂ in 25 mM triethylammonium acetate at pH values between 4 and 6 overnight at 50° C. with shaking at 1400 rpm in a Thermomixer Comfort (Eppendorf, Hamburg, Germany), followed by quenching of the reaction at 99° C. for 10 minutes.

Carbohydrate products from the reaction were derivatized with 8-aminonaphthalene-1,3,6-trisulfonic acid (ANTS) fluorophore, applied to a polyacrylamide gel, and subjected to PACE, according to protocols adapted from (Goubet et al, 2002, Analytical Biochemistry 300: 53-68). Briefly, the derivatization was conducted by first drying 45 μl of the reaction products in a Jouan RC1010 vacuum concentrator system (Jouan Inc., Vallensbaekvej, Denmark) at 40° C. for about 3 hours, then mixing the dried products with 5 μl of 0.2 M ANTS in 15% acetic acid and 5 μl of 1 M NaBH₃CN in DMSO, incubation at 37° C. in a thermomixer overnight followed by exposure to air for about 30 minutes, and then drying in a vacuum concentrator at 40° C. for about 4 hours. The ANTS-labeled samples were mixed with 20 μl of 6 M urea prior to PACE analysis.

Gels were cast according to Goubet et al., 2002, supra. The electrophoresis was performed with an SE 660 vertical electrophoresis system (GE Healthcare, Park Alle, Denmark) in a Tris/Borate buffer system at 200 V for 30 minutes and then at 1000 V for almost 2 hours at +5° C., followed by visualization using a UVP Biolmaging System (UVP, LLC, Upland, Calif., USA) with UV transilluminator (Wavelength 365 nm UV-A) and EpiChem Darkroom combined with a camera system using a SYBR Gold™ emission filter (Syngene, Cambridge, United Kingdom).

Densitometry was performed by visual definition of a minimal space containing the band of interest, and integration of total pixel intensity within the space using a series of MatLab® scripts (The Mathworks, Natick, Mass., USA).

FIGS. 2A and 2B show PACE gels of the products of the T. aurantiacus GH61A polypeptide incubation with PASC and ascorbate or gallic acid (FIG. 2A) or cysteine (FIG. 2B) at various pH values. On PACE, products similar to cellodextrins having 1-7 glucosyl units were detected when either cysteine or ascorbate was included in the reaction, and the incubation was performed at or above the pKa value of the ascorbate or cysteine, respectively. As the pH was increased above the pKa value of the organic compound, the amount of product increased. These apparent cellodextrin products indicated fragmentation of PASC by the combination of the organic compound, the GH61 polypeptide, and the buffer components. In the absence of the T. aurantiacus GH61A polypeptide, or the presence of ascorbate at a pH value below the pKa, no product was detected.

FIG. 2C (ascorbatic acid) and 2D (gallic acid) show the results of densitometric analysis of the PACE gel shown in FIG. 2A. The band intensity is plotted as a function of pH for bands corresponding to cellodextrins of apparent degree of polymerization of 4 (circles), 8 (squares), and 9 (diamonds). It is clear for both gallic acid and ascorbic acid that products for all degrees of polymerization examined increased in intensity with increasing pH. The increase in band intensity with pH for gallic acid incubations was greater than that for ascorbate in the range of pH values examined. The band intensities for gallic acid incubations at the lowest pH, 3.9, were near zero and a sharp increase in product intensity was noted between pH values of 4.5 and 5.5, particularly for the DP 4 product band (circles). The pKa for gallic acid is 4.5, so this transition is consistent with deprotonation of gallic acid to the conjugate base, gallate. Conversely, product bands were apparent for ascorbic acid incubations at all pH values examined, and the band intensities of each appeared to increase linearly with pH. The pKa of ascorbic acid is 4.1, thus even at the lowest pH value examined, 3.9, a substantial fraction of the ascorbic acid was expected to be deprotonated to the conjugate base, ascorbate.

### Example 6: Purification of Acid-Pretreated Xylan Stover Liquor Components that, in Combination with GH61 Polypeptide, could be Shown to Result in Enhanced Cellulolysis of Microcrystalline Cellulose by a T. reesei Cellulase Composition

Beechwood xylan was acid-pretreated and then fractionated by HPLC chromatography as follows. Acid pretreated xylan was generated by incubating 45 g of beechwood xylan with 400 g of 1.1% H₂SO₄ at 190° C. for 2 minutes in a 1 gallon, high-pressure horizontal stirred reactor, (Parr Instrument Company, Moline, Ill., USA). The acid-pretreated xylan was filtered with a 3 kDa MWCO VIVASPIN 20® centrifuge filter (GE Healthcare, Piscataway, N.J., USA), applied to a 8.0 mm×300 mm Shodex Sugar SP0810 chromatography column (Showa Denka America, Inc., NY, USA) using an AGILENT® 1100 HPLC and CHEMSTATION® software (Agilent Technologies, Santa Clara, Calif., USA), and separated by isocratic elution with water at a flow rate of 0.5 ml per minute at 80° C. for 50 minutes, collecting 250 μl fractions in 2.0 ml, 96-deep well plates (Axygen, Union City, Calif., USA). Repeated 100 μl injections of the liquor followed by repeated fraction collection yielded approximately 5 ml of each fraction, which were pooled and lyophilized to dryness. Later separations included an overnight incubation of the acid-pretreated xylan with hemicellulase (SHEARZYME™, Novozymes A/S, Bagsvaerd, Denmark) in 50 mM sodium acetate pH 5.0 at 50° C. to hydrolyze xylo-oligomers, followed by removal of the enzyme with a 3 kDa MWCO VIVASPIN 20® centrifuge filter prior to chromatography. Thermoascus aurantiacus GH61A polypeptide titrations to AVICEL® hydrolysis reactions containing either the hemicellulase-treated or the untreated xylan liquor were performed according to Example 1 and confirmed that hemicellulase treatment had not altered the effects of acid-pretreated xylan on GH61 polypeptide enhancement of cellulolysis in these samples. Absorbance of the eluted fractions was not determined. Fractions were assayed by dissolution of the lyophilized fractions in 200 μl of water, and addition of 100 μl to saccharification reactions according to Example 1, containing 4 mg of the T. reesei cellulase composition and 1 mg of the T. aurantiacus GH61A polypeptide, or no GH61 polypeptide.

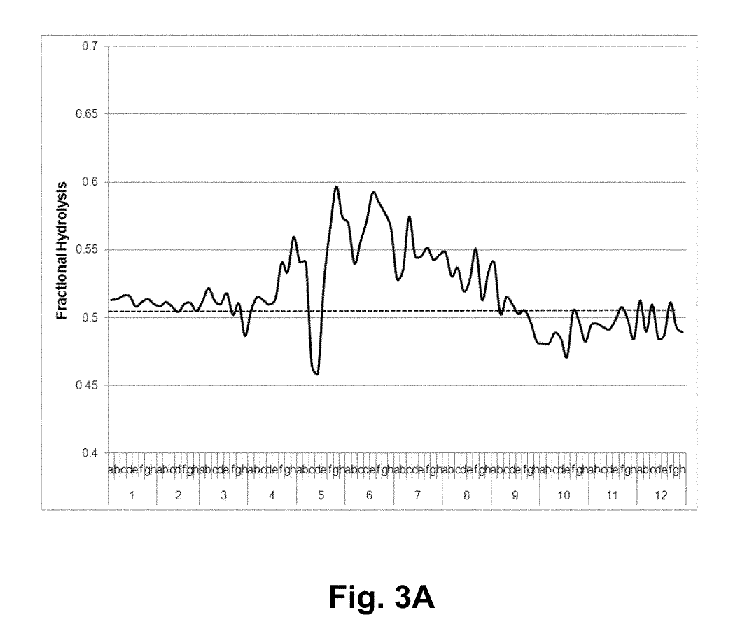

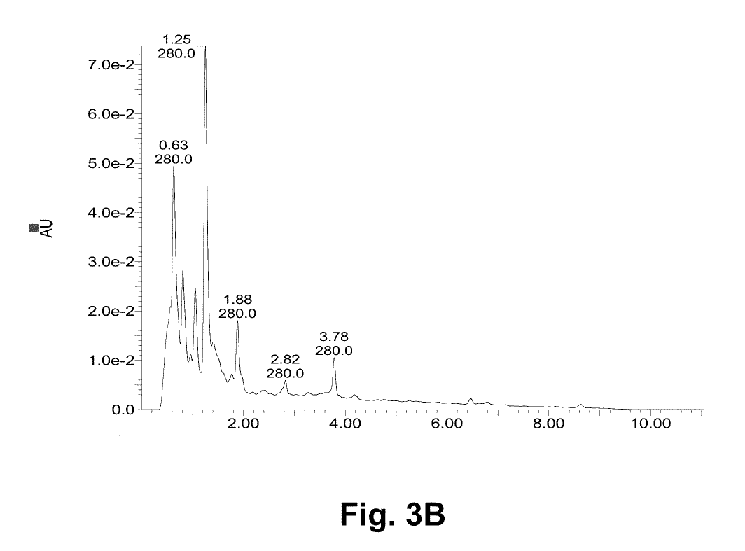

FIG. 3A shows the hydrolysis of AVICEL® by the T. reesei cellulase composition with supplemented T. aurantiacus GH61A polypeptide and the HPLC fractions as indicated. The elution profile demonstrated a broad elution peak of fractions that in the presence of the GH61 polypeptide resulted in higher hydrolysis by the T. reesei cellulase composition. Thus, these fractions contained compounds that in the presence of GH61 polypeptide promoted GH61 polypeptide-dependent cellulolytic enhancement. Several inhibitory fractions were also observed, as is evident in FIG. 3, fraction 5D.

The fractions that had higher fractional hydrolysis in the presence of the T. aurantiacus GH61A polypeptide were analyzed by LC-MS in the following manner. Samples were diluted in 0.1% formic acid to 20 μg per ml approximate concentrations. Samples were then either filtered using Ultrafree-MC centrifugal filter devices (Millipore Corporation, Billerica, Mass., USA) or were centrifuged for 10 minutes at 21,000×g and then stored at 4° C. if analysis was not performed immediately. UPLC® tandem mass spectrometry, was performed using a Q-Tof Micro™ hybrid orthogonal quadrupole time-of-flight mass spectrometer (Waters Micromass® MS Technologies, Milford, Mass., USA) using MASSLYNX™ software version 4.1 (Waters Micromass MS Technologies, Milford, Mass., USA). The Q-TOF MICRO™ was fitted with an ACQUITY UPLC® using a 1.0×50 mm, C18, 1.7 m, BEH Acquity column (Waters Corp, Milford, Mass., USA) to permit chromatographic separation of analytes. The following elution gradient was applied over a 37 minute interval at a flow rate of 100 μl per minute: 0-15 minutes from 1-15% acetonitrile with 0.1% formic acid, 15-20 minutes from 15-40% acetonitrile with 0.1% formic acid, 20-25 minutes from 40-80% acetonitrile with 0.1% formic acid, 25-30 minutes with 80% acetonitrile with 0.1% formic acid, 30-31 minutes from 80-1% acetonitrile with 0.1% formic acid, and 31-37 minutes with 1% acetonitrile with 0.1% formic acid. Elution was monitored at 280 nm through a diode array detector and eluents from the column were introduced directly into the Q-TOF MICRO™ via electrospray ion source. A cone voltage of 20 volts was typically used and the collision energy was varied in the range of 5-15 volts. Data were acquired in survey scan mode from a mass range of m/z 50 to 1000 with switching criteria for MS to MS/MS that included an ion intensity of greater than 50.0 counts per second. The acquired spectra were combined, smoothed, and centered in an automated fashion. Analytes were identified by comparison to spectra of standard compounds when possible. Standard compounds were analyzed before and after the samples to confirm the mass accuracy and retention time stability of the sample analyses.

FIG. 3B shows a representative LC-MS chromatogram of a T. aurantiacus GH61A polypeptide cellulolytic enhancing acid-pretreated xylan HPLC fraction. It is clear from the number of peaks that significant heterogeneity was observed. The bulk of the components were eluted from the LC in the first 6-minutes, suggesting a charged composition.

The present invention is further described by the following numbered paragraphs:

[1] An aqueous composition comprising: (a) a polypeptide having cellulolytic enhancing activity and (b) an organic compound, wherein the combination of the polypeptide having cellulolytic enhancing activity and the organic compound enhances hydrolysis of a cellulosic material by a cellulolytic enzyme.

[2] The composition of paragraphs 1, wherein the organic compound comprises a pKa that is less than or about equal to the pH of the composition.

[3] The composition of paragraphs 1, wherein the organic compound comprises a pKa that is less than the pH of the composition.

[4] The composition of any one of paragraphs 1-3, wherein the pH of the composition is greater than or equal to about 3.0, 3.5, 4.0, 4.5, 5.0, 5.5, 6.0, 6.5, 7.0, 7.5, or 8.0.

[5] The composition of any one of paragraphs 1-3, wherein the pH of the composition is greater than or about equal to about 5.0.

[6] The composition of any one of paragraphs 1-5, wherein the organic compound comprises a pKa less than or equal to about 8.0, 7.5, 7.0, 6.5, 6.0, 5.5, 5.0, 4.5, 3.5, 3.0, or 2.5.

[7] The composition of any one of paragraphs 1-5, wherein the organic compound comprises a pKa less than or about equal to about 5.0.

[8] The composition of any one of paragraphs 1-7, wherein greater than or about 95%, 90%, 85%, 80%, 75%, 70%, 65%, 60%, 55%, 50%, 45%, 40%, 35%, 30%, 25%, 20%, 15%, 10%, 5%, 2.5%, or 1% of the organic compound is in proton dissociated form.

[9] The composition of paragraphs 8, wherein the proton dissociated form of the organic compound is neutral with respect to the proton dissociated moiety.

[10] The composition of paragraphs 8, wherein the proton dissociated form of the organic compound is anionic with respect to the proton dissociated moiety.

[11] The composition of any one of paragraphs 1-10, wherein less than or about 1%, 2.5%, 5%, 10%, 15%, 20%, 25%, 30%, 35%, 40%, 45%, 50%, 55%, 60%, 65%, 70%, 75%, 80%, 85%, 90%, or 95% of the organic compound is in proton associated form.

[12] The composition of paragraphs 11, wherein the proton associated form of the organic compound is neutral.

[13] The composition of paragraphs 11, wherein the proton associated form of the organic compound is cationic.

[14] The composition of any one of paragraphs 1-13, wherein the organic compound comprises a carboxylic acid moiety.

[15] The composition of any one of paragraphs 1-14, wherein the organic compound comprises a lactone moiety.

[16] The composition of any one of paragraphs 1-15, wherein the organic compound comprises a phenolic moiety.

[17] The composition of any one of paragraphs 1-16, wherein the organic compound comprises a flavonoid moiety.

[18] The composition of paragraphs 17, wherein the organic compound comprises an isoflavonoid moiety.

[19] The composition of paragraphs 17, wherein the organic compound comprises a flavylium ion moiety.

[20] The composition of paragraphs 17, wherein the organic compound comprises an anthocyanidin or anthocyanin moiety.

[21] The composition of any one of the preceding paragraphs, wherein the organic compound is selected from the group consisting of: methyl-4-hydroxybenzoate; 3,5-dihydroxybenzoic acid; 4-hydroxybenzoic acid; glutamic acid; tartaric acid; 4-hydroxy-3-methoxycinnamic acid; methyl-3,4,5-trihydroxy-benzoate; 3,4-dihydroxybenzoic acid; ascorbic acid; 4-methyl hexanoic acid; 2-methyl hexanoic acid; pyrocatechol; pyrogallol; dehydroascorbate; cysteine; gallic acid; a compound of Table 1; and a salt or solvate thereof.

[22] The composition of any one of paragraphs 1-21 which further comprises (c) one or more enzymes selected from the group consisting of a cellulase, a hemicellulase, an esterase, an expansin, a laccase, a ligninolytic enzyme, a pectinase, a peroxidase, a protease, and a swollenin.

[23] The composition of paragraphs 22, wherein the cellulase is one or more enzymes selected from the group consisting of an endoglucanase, a cellobiohydrolase, and a beta-glucosidase.

[24] The composition of paragraphs 22, wherein the hemicellulase is one or more enzymes selected from the group consisting of a xylanase, an acetyxylan esterase, a feruloyl esterase, an arabinofuranosidase, a xylosidase, and a glucuronidase.

[25] A method for degrading or converting a cellulosic material, comprising: treating the cellulosic material with an enzyme composition in the presence of a polypeptide having cellulolytic enhancing activity and an organic compound, wherein the combination of the polypeptide having cellulolytic enhancing activity and the organic compound enhances hydrolysis of the cellulosic material by the enzyme composition.

[26] The method of paragraphs 25, wherein the organic compound comprises a pKa that is less than or about equal to the pH of the composition.

[27] The method of paragraphs 25, wherein the organic compound comprises a pKa that is less than the pH of the composition.

[28] The method of any one of paragraphs 25-27, wherein the pH of the composition is greater than or equal to about 3.0, 3.5, 4.0, 4.5, 5.0, 5.5, 6.0, 6.5, 7.0, 7.5, or 8.0.

[29] The method of any one of paragraphs 25-27, wherein the pH of the composition is greater than or about equal to about 5.0.

[30] The method of any one of paragraphs 25-29, wherein the organic compound comprises a pKa less than or equal to about 8.0, 7.5, 7.0, 6.5, 6.0, 5.5, 5.0, 4.5, 3.5, 3.0, or 2.5.

[31] The method of any one of paragraphs 25-29, wherein the organic compound comprises a pKa less than or about equal to about 5.0.

[32] The method of any one of paragraphs 25-31, wherein greater than or about 95%, 90%, 85%, 80%, 75%, 70%, 65%, 60%, 55%, 50%, 45%, 40%, 35%, 30%, 25%, 20%, 15%, 10%, 5%, 2.5%, or 1% of the organic compound is in proton dissociated form.

[33] The method of paragraphs 32, wherein the proton dissociated form of the organic compound is neutral with respect to the proton dissociated moiety.

[34] The method of paragraphs 32, wherein the proton dissociated form of the organic compound is anionic with respect to the proton dissociated moiety.

[35] The method of any one of paragraphs 25-34, wherein less than or about 1%, 2.5%, 5%, 10%, 15%, 20%, 25%, 30%, 35%, 40%, 45%, 50%, 55%, 60%, 65%, 70%, 75%, 80%, 85%, 90%, or 95% of the organic compound is in proton associated form.

[36] The method of paragraphs 35, wherein the proton associated form of the organic compound is neutral.

[37] The method of paragraphs 35, wherein the proton associated form of the organic compound is cationic.

[38] The method of any one of paragraphs 25-37, wherein the organic compound comprises a carboxylic acid moiety.

[39] The method of any one of paragraphs 25-38, wherein the organic compound comprises a lactone moiety.

[40] The method of any one of paragraphs 25-39, wherein the organic compound comprises a phenolic moiety.

[41] The method of any one of paragraphs 25-40, wherein the organic compound comprises a flavonoid moiety.

[42] The method of paragraphs 41, wherein the organic compound comprises an isoflavonoid moiety.

[43] The method of paragraphs 41, wherein the organic compound comprises a flavylium ion moiety.

[44] The method of paragraphs 41, wherein the organic compound comprises an anthocyanidin or anthocyanin moiety.

[45] The method of any one of paragraphs 25-44, wherein the organic compound is selected from the group consisting of: methyl-4-hydroxybenzoate; 3,5-dihydroxybenzoic acid; 4-hydroxybenzoic acid; glutamic acid; tartaric acid; 4-hydroxy-3-methoxycinnamic acid; methyl-3,4,5-trihydroxy-benzoate; 3,4-dihydroxybenzoic acid; ascorbic acid; 4-methyl hexanoic acid; 2-methyl hexanoic acid; pyrocatechol; pyrogallol; dehydroascorbate; cysteine; gallic acid; a compound of Table 1; and a salt or solvate thereof.

[46] The method of any one of paragraphs 25-45, wherein the cellulosic material is pretreated.

[47] The method of any one of paragraphs 25-46, further comprising recovering the degraded cellulosic material.

[48] The method of any of one of paragraphs 25-47, wherein the enzyme composition comprises one or more enzymes selected from the group consisting of a cellulase, a hemicellulase, an esterase, an expansin, a laccase, a ligninolytic enzyme, a pectinase, a peroxidase, a protease, and a swollenin.

[49] The method of paragraphs 48, wherein the cellulase one or more enzymes selected from the group consisting of an endoglucanase, a cellobiohydrolase, and a beta-glucosidase.

[50] The method of paragraphs 48, wherein the hemicellulase is one or more enzymes selected from the group consisting of a xylanase, an acetyxylan esterase, a feruloyl esterase, an arabinofuranosidase, a xylosidase, and a glucuronidase.

[51] The method of any one of paragraphs 25-49, wherein the degraded cellulosic material is a sugar.

[52] The method of paragraphs 51, wherein the sugar is selected from the group consisting of glucose, xylose, mannose, galactose, and arabinose.

[53] A method for producing a fermentation product, comprising:

(a) saccharifying a cellulosic material with an enzyme composition in the presence of a polypeptide having cellulolytic enhancing activity and an organic compound, wherein the combination of the polypeptide having cellulolytic enhancing activity and the organic compound enhances hydrolysis of the cellulosic material by the enzyme composition;

(b) fermenting the saccharified cellulosic material with one or more fermenting microorganisms to produce the fermentation product; and

(c) recovering the fermentation product from the fermentation.

[54] The method of paragraphs 53, wherein the organic compound comprises a pKa that is less than or about equal to the pH of the composition.

[55] The method of paragraphs 53, wherein the organic compound comprises a pKa that is less than the pH of the composition.

[56] The method of any one of paragraphs 53-55, wherein the pH of the composition is greater than or equal to about 3.0, 3.5, 4.0, 4.5, 5.0, 5.5, 6.0, 6.5, 7.0, 7.5, or 8.0.

[57] The method of any one of paragraphs 53-55, wherein the pH of the composition is greater than or about equal to about 5.0.

[58] The method of any one of paragraphs 53-57, wherein the organic compound comprises a pKa less than or equal to about 8.0, 7.5, 7.0, 6.5, 6.0, 5.5, 5.0, 4.5, 3.5, 3.0, or 2.5.

[59] The method of any one of paragraphs 53-57, wherein the organic compound comprises a pKa less than or about equal to about 5.0.

[60] The method of any one of paragraphs 53-59, wherein greater than or about 95%, 90%, 85%, 80%, 75%, 70%, 65%, 60%, 55%, 50%, 45%, 40%, 35%, 30%, 25%, 20%, 15%, 10%, 5%, 2.5%, or 1% of the organic compound is in proton dissociated form.

[61] The method of paragraphs 60, wherein the proton dissociated form of the organic compound is neutral with respect to the proton dissociated moiety.

[62] The method of paragraphs 60, wherein the proton dissociated form of the organic compound is anionic with respect to the proton dissociated moiety.

[63] The method of any one of paragraphs 53-62, wherein less than or about 1%, 2.5%, 5%, 10%, 15%, 20%, 25%, 30%, 35%, 40%, 45%, 50%, 55%, 60%, 65%, 70%, 75%, 80%, 85%, 90%, or 95% of the organic compound is in proton associated form.

[64] The method of paragraphs 63, wherein the proton associated form of the organic compound is neutral.

[65] The method of paragraphs 63, wherein the proton associated form of the organic compound is cationic.

[66] The method of any one of paragraphs 53-65, wherein the organic compound comprises a carboxylic acid moiety.

[67] The method of any one of paragraphs 53-66, wherein the organic compound comprises a lactone moiety.

[68] The method of any one of paragraphs 53-67, wherein the organic compound comprises a phenolic moiety.

[69] The method of any one of paragraphs 53-68, wherein the organic compound comprises a flavonoid moiety.

[70] The method of paragraphs 69, wherein the organic compound comprises an isoflavonoid moiety.

[71] The method of paragraphs 69, wherein the organic compound comprises a flavylium ion moiety.

[72] The method of paragraphs 69, wherein the organic compound comprises an anthocyanidin or anthocyanin moiety.

[73] The method of any one of paragraphs 53-72, wherein the organic compound is selected from the group consisting of: methyl-4-hydroxybenzoate; 3,5-dihydroxybenzoic acid; 4-hydroxybenzoic acid; glutamic acid; tartaric acid; 4-hydroxy-3-methoxycinnamic acid; Methyl-3,4,5-trihydroxy-benzoate; 3,4-dihydroxybenzoic acid; ascorbic acid; 4-methyl hexanoic acid; 2-methyl hexanoic acid; pyrocatechol; pyrogallol; dehydroascorbate; cysteine; gallic acid; a compound of Table 1; or a salt or solvate thereof.

[74] The method of any one of paragraphs 53-73, wherein the cellulosic material is pretreated.

[75] The method of any one of paragraphs 53-74, wherein the enzyme composition comprises one or more enzymes selected from the group consisting of a cellulase, a hemicellulase, an esterase, an expansin, a laccase, a ligninolytic enzyme, a pectinase, a peroxidase, a protease, and a swollenin.

[76] The method of paragraphs 75, wherein the cellulase is one or more enzymes selected from the group consisting of an endoglucanase, a cellobiohydrolase, and a beta-glucosidase.

[77] The method of paragraphs 75, wherein the hemicellulase is one or more enzymes selected from the group consisting of a xylanase, an acetyxylan esterase, a feruloyl esterase, an arabinofuranosidase, a xylosidase, and a glucuronidase.

[78] The method of any one of paragraphs 53-77, wherein steps (a) and (b) are performed simultaneously in a simultaneous saccharification and fermentation.

[79] The method of any one of paragraphs 53-78, wherein the fermentation product is an alcohol, an alkane, a cycloalkane, an alkene, an amino acid, a gas, isoprene, a ketone, an organic acid, or polyketide.

[80] A method of fermenting a cellulosic material, comprising: fermenting the cellulosic material with one or more fermenting microorganisms, wherein the cellulosic material is saccharified with an enzyme composition in the presence of a polypeptide having cellulolytic enhancing activity and an organic compound, wherein the combination of the polypeptide having cellulolytic enhancing activity and the organic compound enhances hydrolysis of the cellulosic material by the enzyme composition.

[81] The method of paragraphs 80, wherein the organic compound comprises a pKa that is less than or about equal to the pH of the composition.

[82] The method of paragraphs 80, wherein the organic compound comprises a pKa that is less than the pH of the composition.

[83] The method of any one of paragraphs 80-82, wherein the pH of the composition is greater than or equal to about 3.0, 3.5, 4.0, 4.5, 5.0, 5.5, 6.0, 6.5, 7.0, 7.5, or 8.0.

[84] The method of any one of paragraphs 80-82, wherein the pH of the composition is greater than or about equal to about 5.0.

[85] The method of any one of paragraphs 80-84, wherein the organic compound comprises a pKa less than or equal to about 8.0, 7.5, 7.0, 6.5, 6.0, 5.5, 5.0, 4.5, 3.5, 3.0, or 2.5.

[86] The method of any one of paragraphs 80-84, wherein the organic compound comprises a pKa less than or about equal to about 5.0.

[87] The method of any one of paragraphs 80-86, wherein greater than or about 95%, 90%, 85%, 80%, 75%, 70%, 65%, 60%, 55%, 50%, 45%, 40%, 35%, 30%, 25%, 20%, 15%, 10%, 5%, 2.5%, or 1% of the organic compound is in proton dissociated form.

[88] The method of paragraphs 87, wherein the proton dissociated form of the organic compound is neutral with respect to the proton dissociated moiety.

[89] The method of paragraphs 87, wherein the proton dissociated form of the organic compound is anionic with respect to the proton dissociated moiety.

[90] The method of any one of paragraphs 80-89, wherein less than or about 1%, 2.5%, 5%, 10%, 15%, 20%, 25%, 30%, 35%, 40%, 45%, 50%, 55%, 60%, 65%, 70%, 75%, 80%, 85%, 90%, or 95% of the organic compound is in proton associated form.

[91] The method of paragraphs 90, wherein the proton associated form of the organic compound is neutral.

[92] The method of paragraphs 90, wherein the proton associated form of the organic compound is cationic.

[93] The method of any one of paragraphs 80-92, wherein the organic compound comprises a carboxylic acid moiety.

[94] The method of any one of paragraphs 80-93, wherein the organic compound comprises a lactone moiety.

[95] The method of any one of paragraphs 80-94, wherein the organic compound comprises a phenolic moiety.

[96] The method of any one of paragraphs 80-95, wherein the organic compound comprises a flavonoid moiety.

[97] The method of paragraphs 96, wherein the organic compound comprises an isoflavonoid moiety.

[98] The method of paragraphs 96, wherein the organic compound comprises a flavylium ion moiety.

[99] The method of paragraphs 96, wherein the organic compound comprises an anthocyanidin or anthocyanin moiety.

[100] The method of any one of paragraphs 80-99, wherein the organic compound is selected from the group consisting of: methyl-4-hydroxybenzoate; 3,5-dihydroxybenzoic acid; 4-hydroxybenzoic acid; glutamic acid; tartaric acid; 4-hydroxy-3-methoxycinnamic acid; methyl-3,4,5-trihydroxy-benzoate; 3,4-dihydroxybenzoic acid; ascorbic acid; 4-methyl hexanoic acid; 2-methyl hexanoic acid; pyrocatechol; pyrogallol; dehydroascorbate; cysteine; gallic acid; a compound of Table 1; and a salt or solvate thereof.

[101] The method of any one of paragraphs 80-100, wherein the cellulosic material is pretreated before saccharification.

[102] The method of any one of paragraphs 80-101, wherein the enzyme composition comprises one or more enzymes selected from the group consisting of a cellulase, a hemicellulase, an esterase, an expansin, a laccase, a ligninolytic enzyme, a pectinase, a peroxidase, a protease, and a swollenin.

[103] The method of paragraphs 102, wherein the cellulase is one or more enzymes selected from the group consisting of an endoglucanase, a cellobiohydrolase, and a beta-glucosidase.

[104] The method of paragraphs 102, wherein the hemicellulase is one or more enzymes selected from the group consisting of a xylanase, an acetyxylan esterase, a feruloyl esterase, an arabinofuranosidase, a xylosidase, and a glucuronidase.

[105] The method of any one of paragraphs 80-104, wherein the fermenting of the cellulosic material produces a fermentation product.

[106] The method of paragraphs 105, further comprising recovering the fermentation product from the fermentation.

[107] The method of paragraphs 105 or 106, wherein the fermentation product is an alcohol, an alkane, a cycloalkane, an alkene, an amino acid, a gas, isoprene, a ketone, an organic acid, or polyketide.

[108] The method of any one of paragraphs 25-107, wherein an effective amount of the organic compound to cellulosic material as a molar ratio to glucosyl units of cellulose is about 10⁻⁶ to about 10, e.g., about 10⁻⁶ to about 7.5, about 10⁻⁶ to about 5, about 10⁻⁶ to about 2.5, about 10⁻⁶ to about 1, about 10⁻⁶ to about 1, about 10⁻⁵ to about 10⁻¹, about 10⁻⁴ to about 10⁻¹, about 10⁻³ to about 10⁻¹, or about 10⁻³ to about 10⁻².

[109] The method of any one of paragraphs 25-108, wherein an effective amount of the organic compound to cellulose is about 10⁻⁶ to about 10 per g of cellulose, e.g., about 10⁻⁶ to about 7.5, about 10⁻⁶ to about 5, about 10⁻⁶ to about 2.5, about 10⁻⁶ to about 1, about 10⁻⁵ to about 1, about 10⁻⁵ to about 10⁻¹, about 10⁻⁴ to about 10⁻¹, about 10⁻³ to about 10⁻¹, or about 10⁻³ to about 10⁻² per g of cellulose.

[110] The method of any one of paragraphs 25-109, wherein an effective amount of the organic compound is about 0.1 μM to about 1 M, e.g., about 0.5 μM to about 0.75 M, about 0.75 μM to about 0.5 M, about 1 μM to about 0.25 M, about 1 μM to about 0.1 M, about 5 μM to about 50 mM, about 10 μM to about 25 mM, about 50 μM to about 25 mM, about 10 μM to about 10 mM, about 5 μM to about 5 mM, or about 0.1 mM to about 1 mM.

The invention described and claimed herein is not to be limited in scope by the specific aspects herein disclosed, since these aspects are intended as illustrations of several aspects of the invention. Any equivalent aspects are intended to be within the scope of this invention. Indeed, various modifications of the invention in addition to those shown and described herein will become apparent to those skilled in the art from the foregoing description. Such modifications are also intended to fall within the scope of the appended claims. In the case of conflict, the present disclosure including definitions will control.

1-24. (canceled)

25. A method for producing a fermentation product, comprising:
(a) saccharifying a cellulosic material with a cellulolytic enzyme composition in the presence of (i) a GH61 polypeptide having cellulolytic enhancing activity and (ii) an organic compound comprising a carboxylic acid moiety, a lactone moiety, a phenolic moiety, a flavonoid moiety, or a combination thereof, wherein the combination of the GH61 polypeptide having cellulolytic enhancing activity and the organic compound enhances hydrolysis of the cellulosic material by the cellulolytic enzyme compared to the GH61 polypeptide alone or the organic compound alone, and wherein the proton dissociated form of the organic compound is neutral or anionic with respect to the proton dissociated moiety;
(b) fermenting the saccharified cellulosic material with one or more fermenting microorganisms to produce the fermentation product; and
(c) recovering the fermentation product from the fermentation.

26. The method of claim 25, wherein steps (a) and (b) are performed simultaneously in a simultaneous saccharification and fermentation.

27. The method of claim 25, wherein the organic compound comprises a pKa that is less than or about equal to the pH of the composition.

28. The method of claim 25, wherein the pH of the composition is greater than or equal to about 3.0.

29. The method of claim 25, wherein the pH of the composition is greater than or equal to about 4.0.

30. The method of claim 25, wherein the pH of the composition is greater than or equal to about 5.0.

31. The method of claim 25, wherein the pH of the composition is greater than or equal to about 6.0.

32. The method of claim 25, wherein the pH of the composition is greater than or equal to about 7.0.

33. The method of claim 25, wherein the pH of the composition is greater than or equal to about 8.0.

34. The method of claim 25, wherein the organic compound comprises a pKa less than or equal to about 8.0.

35. The method of claim 25, wherein the organic compound comprises a pKa less than or about equal to about 7.0.

36. The method of claim 25, wherein the organic compound comprises a pKa less than or equal to about 6.0.

37. The method of claim 25, wherein the organic compound comprises a pKa less than or equal to about 5.0.

38. The method of claim 25, wherein the organic compound comprises a pKa less than or equal to about 3.0.

39. The method of claim 25, wherein greater than or about 95% of the organic compound is in proton dissociated form.

40. The method of claim 25, wherein greater than or about 75% of the organic compound is in proton dissociated form.

41. The method of claim 25, wherein greater than or about 50% of the organic compound is in proton dissociated form.

42. The method of claim 25, wherein greater than or about 25% of the organic compound is in proton dissociated form.

43. The method of claim 25, wherein greater than or about 5% of the organic compound is in proton dissociated form.

44. The method of claim 25, wherein the organic compound is selected from the group consisting of: methyl-4-hydroxybenzoate; 3,5-dihydroxybenzoic acid; 4-hydroxybenzoic acid; glutamic acid; tartaric acid; 4-hydroxy-3-methoxycinnamic acid; methyl-3,4,5-trihydroxybenzoate; 3,4-dihydroxybenzoic acid; ascorbic acid; 4-methyl hexanoic acid; 2-methyl hexanoic acid; pyrocatechol; pyrogallol; dehydroascorbate; cysteine; gallic acid; fumaric acid; trans-aconitic acid; methylmalonic acid; oxalic acid; sorbic acid; maleic acid; caproic acid; 4-hydroxy-3-methoxybenzoic acid; 2,6-dimethoxyphenol; abietic acid; 3,5-di-tert-butyl-4-hydroxybenzoic acid; 2-aminophenol; butyric acid; epicatechin; morin; naringenin; quercetin; and a salt or solvate thereof.

45. The method of claim 25, wherein the combination of the GH61 polypeptide having cellulolytic enhancing activity and the organic compound enhances hydrolysis of a cellulosic material by a cellulolytic enzyme according to the following formula with a ratio greater than 1:
$$GH 61 effect = \frac{\% {conversion}_{( + GH 61 + organic compound )}}{\% {conversion}_{( no GH 61 + organic compound )}} .$$

46. The method of claim 25, wherein the effective amount of the organic compound to the cellulosic material as a molar ratio to glucosyl units of cellulose is about 10⁻⁶ to about 10.

47. The method of claim 25, wherein an effective amount of the organic compound to the cellulosic material as a molar ratio to glucosyl units of cellulose is about 10⁻⁵ to about 1.

48. The method of claim 25, wherein an effective amount of the organic compound to the cellulosic material as a molar ratio to glucosyl units of cellulose is about 10⁻⁴ to about 10⁻¹.

49. The method of claim 25, wherein an effective amount of the organic compound to the cellulosic material as a molar ratio to glucosyl units of cellulose is about 10⁻³ to about 10⁻².

50. The method of claim 25, wherein the organic compound comprises a carboxylic acid moiety.

51. The method of claim 25, wherein the organic compound comprises a lactone moiety.

52. The method of claim 25, wherein the organic compound comprises a phenolic moiety.

53. The method of claim 25, wherein the organic compound comprises a flavonoid moiety.

54. The method of claim 25, wherein the enzyme composition comprises one or more enzymes selected from the group consisting of a cellulase, a hemicellulase, an esterase, an expansin, a laccase, a ligninolytic enzyme, a pectinase, a peroxidase, a protease, and a swollenin.

55. The method of claim 54, wherein the cellulase is one or more enzymes selected from the group consisting of an endoglucanase, a cellobiohydrolase, and a beta-glucosidase.

56. The method of claim 54, wherein the hemicellulase is one or more enzymes selected from the group consisting of an acetylmannan esterase, an acetylxylan esterase, an arabinanase, an arabinofuranosidase, a coumaric acid esterase, a feruloyl esterase, a galactosidase, a glucuronidase, a glucuronoyl esterase, a mannanase, a mannosidase, a xylanase, and a xylosidase.

57. The method of claim 25, wherein the cellulosic material is pretreated.
